SEQUENCE LISTING (179 sequences)

SEQ ID NO: 1 - DNA, 1746 bp, synthetic construct
  source 1..1746: mol_type = other DNA
atgacaaaccccgccagcaggcctaaggcctactcctacatcagaatgtcctccgccatccagatcaagggcgactccttccggcggcaggccgaggcttccgccaagtacgctgccgagcacgacctggatctgatcgacgattacaaactggccgatctgggggtgtccgccttcaagtccgacaacctgaccaccggcgctctggggcggttcgtggccgagtgcgaggcgggagaaatcgaggctggatcctttctgctgatcgaatccctggacaggctgtcgagagacaagatcctggacgccttcagcctgtttgccagaattctgaaaaccggtgttaagatcgtcaccctgtctgacggccaagtgtacgacggctccagcgaccaggtgggctctatctactacgctatcagcgtgatgatccggagcaacgacgagtctaaaatcaagtccaccagaggactggccaactggtcccagaagagaaagctggctgcagaacacggcgtgaagatgtcctcccagtgtcccgcctggctgaagctgtctgtggatagaaagtcctacctgatcgacaaggaaagggctaagatcgtgcagagaatcttcgaggcctctgcctctggcaaaggcgccaatctgatcaccaaggaactgaaccgggacaaggtgcctaccttcggcagaggcgccctgtgggccgaagcctttgtgtccaagaccctgcggaaccgggccgtgttaggagagttccagcctggccagtacgtgtctggtaagagacagcccgctggcgacccaatccctggctacttccctcctgtgatcgaagaggagctgttcgatatcgtgcaagcctccctgagaggccgcctcctcgctggcggcagaagaggcgagggccagtccaacatcttcacccatgtagccttctgcggctactgcggctccaagatgagacacagaagcaagggcagcagagtgaagggcaacccccctcacagatacctgacctgtttcaacagattcaacggcccaggctgcgactgcaagcccctgccttacgccgctttcgagcgctctttcctgactttcgtgcgggatgtggacctgagaggcctgctggaaggcgccaagagaaagtccgaggccaagaccatcgctgacagaatcaccgtgaacgaggaaaaagtcagaaaagctgatgagagaatccgcgactacctgatcaagatcgaaggagctcctgacctggccgagatcttcatggaacggatcagagagctgaaggctgagaaggacgacctggtcagatctatcgaagagtccaacgacgctctgtccaagatcaaatctgacaacgtgacagacgaggagctggctagcttgatctctacctttcagaacccttgcggagagaatcggatcagactggccgaccggataaagtccatcatcgagagaatcgacgtgtatcccaacggcgaaatccggaaggacgaccctgccatcgatctggtccgggcttctggcgatcctgacgctgagaagatcatcgccgccatgaacgccggctctagactgaaggacgacccttacttcatcgtgaccttccggaatggcgctgtgcagaccgtggtgcctaacccttccaaccctgatgatattcgggtttctgtgtacgcaggcgaaaagacccgacgggtggaaggctctgcctatgagtacgagtccgat

SEQ ID NO: 2 - DNA, 1527 bp, synthetic construct
  source 1..1527: mol_type = other DNA
atgcctatcgcccctgagttcctgtctctggcctaccccggacaagagttccctgcctacctgtacggcagagcctctagagatcctaagcggaagggcagatctgtgcagagccagctggacgaaggcagagccacatgcctggatgccggctggcctattgccggcgaatttaaggacgtggatcggtccgcttctgcttacgccagacggacacgggacgaattcgaggagatgatcgctggcatccaggccggagagtgcaggattctggtcgccttcgaggcaagcagatactaccgggacctggaggcttatgttcggctgcggagagtgtgcagagaggccggcgtcctcctgtgctacaacggccaggtgtacgacctgtccaagtccgccgacagaaaggccaccgctcaggacgctgtgaacgccgagggagaagctgacgacatcagagaacggaacctgagaaccaccagactgaatgctaagagaggcggcgcccacggccctgtgcctgatggctacaagagaagatacgaccccgactctggcgacctggtggaccagatccctcatcctgatagagcgggcctgatcaccgagatcttccggcgcgctgccgctgctgagcccctggctgctatctgtcgggatctgaacgagagaggcgagacaacccacaggggaaaagcttggcagagacaccacctgcacgccatcctgagaaatcccgcctacatcggccaccggaggcatctgggcgtggacaccggcaaaggtatgtgggctcctatctgcgacgacgaggacttcgccgaaaccttccaggccgtgcaggagatcttatctttgccaggcagacagctgtctcctggcccagaagctcagcacctgcagaccggaatcgccctgtgtggcgagcaccctgacgagcctcctctgagatccgtgaccgtgcgcggccggaccaactacaactgctccaccagatatgatgtggccatgagagaagatcggatggacgccttcgtggaagagtccgtgatcacctggctggcctccgacgaagccgtggctgcctttgaggacaacaccgacgatgagcggacacggaaggcccggatccggctgaaggtgctggaggaacagctggaagccgcccagaagcaggctagaaccctgcggcctgacggcatgggcatgctgctgtccatcgactccctggctggcctggaagccgagctgacccctcaaatcgacaaggccagacaagaatcccggagcctgcacgtgcccgctctgctgagagatctgctgggcaagcctagagccgacgtcgaccgggcctggaacgaggctctaaccctgccccagcggcggatgatcctaagaatggtggtgaccatcagactgttcaaagctggctctagaggcgtgcgggccatcgagcctggccggatcaccctgtcctacgtgggcgagccaggcttcaagcccgtgggcggcaaccgggccaagcag

SEQ ID NO: 3 - DNA, 1413 bp, synthetic construct
  source 1..1413: mol_type = other DNA
atgagaaaggtggccatctacagccgggtgtccaccatcaaccaggccgaagagggctattctatccagggccaaatcgaggccctgaccaagtactgcgaggctatggaatggaagatctacaaaaactactccgacgccggcttctccggaggcaagctcgaaagacccgctataaccgagctgattgaggacggcaagaacaacaagtttgacaccatcctggtgtacaagctggatcggctgtcccggaacgtgaaggacacactctacctggttaaagatgtgttcaccgctaacaacatccacttcgtgtctcttaaggagaacatcgatacttcctctgccatgggaaacctgttcctgaccctgctgtctgctatcgccgagttcgagagagaacagatcaaggagcggatgcagttcggtgtgatgaaccgggctaagtccggcaaaacaacagcttggaaaacccctccttacggctacagatacaacaaggacgaaaagaccctgtctgtcaacgagctggaagccgccaacgtcagacagatgttcgacatgatcatctccggctgtagcatcatgtccatcaccaactacgcccgggacaactttgtgggcaacacctggacccacgtgaaggtgaagcggatcctggaaaacgaaacctacaagggcctggtcaagtacagagagcagacattttctggcgaccaccaggcaatcatcgatgagaaaacctacaataaggcccagatcgctctggctcatagaaccgacaccaagacaaacaccagaccattccagggcaagtacatgctgtctcatatcgccaagtgcggctactgtggcgctcctctgaaagtgtgcaccggcagagccaagaacgatggcaccagacggcaaacctacgtgtgcgtgaacaagaccgagtccctggccagaaggagcgtgaataattataacaaccagaagatctgcaacaccggccgctacgagaagaagcacatcgagaagtatgtgatcgacgtgctgtacaagctgcagcacgacaaagagtacctgaaaaagatcaaaaaggacgataatatcatcgacatcacccctctgaagaaagaaatcgagatcatcgacaagaagatcaacagactgaacgacctgtacatcaacgatctgatcgatctgcccaagctgaaaaaggatatcgaggaactgaaccacctgaaggacgactacaacaaggccatcaagctgaactacctggacaagaagaatgaggattctctgggcatgctgatggacaacctggacatccggaaaagctcctacgacgtgcagtccagaatcgtgaagcagctgatcgacagagtggaagtgaccatggacaatatcgacattatcttcaagttc

SEQ ID NO: 4 - DNA, 1431 bp, synthetic construct
  source 1..1431: mol_type = other DNA
atgatcacaaccagaaaggttgccatctatgtgagagtgtccaccaccaaccaggctgaagaaggctactccatccagggccagatcgactccctgattaagtactgcgaggctatgggctggatcatctacgaggagtacaccgacgctggcttctccggcggaaaaatcgatcggcctgccatgagtaagctgatcaccgatgccaagcacaagagattcgatacaatcctggtgtacaagctggacagactgagcagatccgtgcgggacacactgtacctggtcaaggatgtgttcaaccagaacaacatccacttcgtgtccctgcaggagaatatcgacacctccagcgccatgggaaacctgttcctgaccctgctctctgctatcgccgagttcgagagagagcagatcaccgagcggatgaccatgggcaagatcggcagagccaagtctggcaagaccatggcctggacctacaccccttttggctacgactataacaaagagaagggcgagctgatcctggatcctgctaaggcccccatcgtgaagatgatctacaccgactacctgaagggtatgagcatccaaaagatcgtggacaaactaaacaagatggactacaacggcaaggactgcacctggttcccacacggcgtgaaacatctgctggacaatcctgtgtactacggcatgactagatataacaacaagctgtttcctggcaaccaccagccaatcatcaccaaggaactgtttgacaagacccagcgcgagagacagagaagaaggctgggcatcgaagagaatcactacaccatacctttccaggccaaatacatgctgtctaagttcctgagatgcagacagtgcggctctagaatgggcctggagctgggcagacctcggaagaaagagggaaagcggtccaagaagtactactgtctgaactccaggcccaagagaaccgcctcctgcgacacccctctgtacgatgctgaaaccctggaagattacgtgctgcacgagatcgccaaaatccagaaggacccttctatcgcttctcggcaaaaacacatcgaagatcatgaattgaaatacaagcgggaacggatcgaggccaacatcaacaagaccgtgaaccagctgtccaagctgaacaacctgtacctgaatgacctgatcaccctcgaggacctgaaaacccagaccaacaccctgattgctaagaagcgactgctggaaaacgagctggacaagacctgtgacaacgacgacgagctcgacagacaagagacaatcgccgacttcctggctctgcctgacgtgtggacaatggattacgagggccagaagtacgccgtggaactgctggtgcagagagtgaaggtggaccgggacaacatcgacatccactggaccttc

SEQ ID NO: 5 - DNA, 1656 bp, synthetic construct
  source 1..1656: mol_type = other DNA
atgcctggcatgaccaccgaaaccggccccgatcctgccggcctgatcgacctgttctgcagaaaaagcaaagctgtcaagtccagagccaatggcgctggacagcggagaaagcaagaaatctccatcgccgcccaggaaaccctgggccgaaaggtggctgccctgctcggcatgcaggtgcggcatgtgtggaaggaagtgggatctgcttctcggtttagaaagggcaaggctcgggacgaccagtccaaggccctgaaggccctggaatctggcgaggtgggcgctctgtggtgctaccggctggatagatgggacagaggcggcgctggagccatcctgaagatcatcgagcctgaggacggcatgccccggcggctgctgtttggctgggatgaggacaccggcagacctgtcctggactccaccaacaagcgggatcggggcgagctgattagacgggccgaggaggccagagaagaagccgaaaagctgtccgagagagtcagagatacaaaagcccaccagagagagaacggcgagtgggtgaacgccagagccccttacggcctgagagtggtgctggtgaccgtgtccgacgaggaaggcgacgagtacgacgagcggaagctggctgccgacgatgaggacgctggcggccctgacggtctgaccaaggctgaagccgctagactggtgttcaccctgcctgtgaccgacagactctcttacgccggcaccgctcacgccatgaacaccagagagatcccatctcccaccggcggaccctggatcgccgttaccgtgcgggacatgatccagaaccccgcctacgctggctggcagaccacaggcagacaggacggcaagcagcggagactgaccttctataacggcgaaggcaaacgcgtgtccgtgatgcacggccctcctctggtcacagacgaggagcaggaagccgccaaggcagccgtgaagggagaggatggcgtgggcgtgccactggacggctctgaccacgacacccggcggaagcacctgctgtctggccggatgcggtgtcctggctgtggcggcagctgctcctactccggcaacggctacagatgctggcggtcctccgtgaagggcggctgccctgctccaacctacgtggctcgcaagtctgtggaagagtatgtggccttccggtgggctgccaagctggccgcctccgagcctgacgatcctttcgtgatcgccgtggccgatcggtgggccgctctgacccaccctcaggcttccgaagatgagaagtacgccaaggccgcagtgagggaggccgagaagaacctgggcagactgctaagagacagacagaatggcgtgtacgatggacctgccgaacagttcttcgcccctgcttaccaggaggctctgtctacactgcaggccgctaaggacgccgtgtctgagtcctccgcctctgccgctgtggacgtgagctggatcgtggacagcagcgactacgaggaactgtggctgagagctacccctaccatgagaaacgctatcatcgacacatgcatcgacgagatctgggtcgcgaaaggccagagaggcagacctttcgacggggacgagagagtgaagatcaagtgggccgctaggact

SEQ ID NO: 6 - DNA, 1557 bp, synthetic construct
  source 1..1557: mol_type = other DNA
atgcagctggacgccaccctgacactgcgggacgagggcctgagcgctttccaccagagacacatcaagcagggtgctctgggagtgttcctgagagctatcgaggacggccggatccagcctggctccgtgctgatcgtggaaggcctggacagactctctagagccgagcccatccaagctcaggcccagctggcccagatcatcaacgccggcatcaccgtggtgaccgcctctgatggccgagagtacaaccgggaaagactgaaagcccaacctatggaccttgtgtactccctgctggtgatgatcagagctcacgaggaatccgacaccaagtccaagcgggtgaaggccgccatcaggcggcagtgcgagggctgggtcgctggcacatggcggggcatcatccggaacggcaaggaccctcactgggtcagactgggcgagcacggcaagttcgagcatgtgcctgagcgggtgctggctgtgcggacaatgatcgacctgttcctggaaggccacggcgccatcgagatcaccaggcggctgaccgagcagaacctgtacgtgtccaacgccggcaactactctgtgcacatgtacagaatcgtgagaaaccaggctctgatcggcgagaagagaatctccgtggatggagaagagttccggctggacggctactaccctccaatcctgaccagagaagaatttgccgaactgcagcagaccatgtccgagagaggcagacggaagggcaaaggcgagatccctaacatcatcacaggactgtccatcacagtgtgcggctattgtggcagagccatgaccacccagaactctaaggctcgcgcccctaagggaaaaagcgtggtcagacggctgtcctgccccatgaattccttcaacgagggatgtcctatcggcggctcttgcgagtctgagatcgtcgagagagccctcatgagatactgctccgaccagttcaatctgtctcggttgctggagggcgacgacggcaccgcccggcggaccgctcaactggctgtggctagacaaagagcatctgacatcgaagcccagatccagcgcgtgaccgacgccctcctgagcgacgacggcaaggctcctgccgcctttacccgcagagctcgcgagctggaaacccagctggaggaacagagaagagagatcgaggctctggaacaccagatcgccgctagctctgctcatggcatccccgccgccgctgaggcctgggctcagctggttgacggcgtgctggccctggactacgatgctcggatgaaggccagacagctggtggccgataccttcagaaagatcgtggtgtaccagaggggcttcgccccaatcgacgatgctgctgccgacagatggaagagatccggcaccatcggcctgatgctggtcaccaagagaggaggcatgcggctgctgaacgtggaccggagaaccggctgctggcaggccgaggatgacctggatccttctctgattccttccgatggcctgcccatgctgcctctggatgcc

SEQ ID NO: 7 - DNA, 1386 bp, synthetic construct
  source 1..1386: mol_type = other DNA
atgaaagtggccatctacgtgcgggtttccaccgacgagcaggccaaagaaggtttcagcatccctgctcaaagagagcggctgagagccttctgcgcctctcaaggctgggagatcgtgcaggagtacatcgaggagggctggtccgctaaggatctggacagacctcagatgcagcggctgctgaaggacatcaagaagggcaatatcgatatcgtgctggtgtacagactggataggctgaccagatctgtgctggatctgtacctgctgctccagaccttcgagaagtacaacgtggcctttcggtctgccaccgaggtgtacgatacaagcaccgccatgggcagactgtttatcactctggtcgctgctctggctcagtgggaaagagagaacctggccgagagagtgaagttcggcatcgaacagatgatcgacgagggcaagaagccaggcggccattctccttacggctacaagtttgacaaggatttcaactgtaccatcatcgaggaagaagctgatgtggtgcggatgatttacagaatgtactgcgacggctatggctatagatccatcgccgacagactgaacgagctgatggttaagcctagaatcgccaaggagtggaaccacaactccgtcagagatattctgaccaacgacatctacatcggcacctacagatggggcgacaaggtggtgcctaacaaccacccccccatcatctccgagacactgtttaagaaggcccagaaagagaaggagaagcggggagtggaccggaagagagtgggcaagttcctgttcaccggcctgctgcagtgtggcaactgcggcggacacaagatgcagggccacttcgacaagcgcgagcagaaaacctactaccggtgcaccaagtgccaccggatcaccaacgagaagaacatcttggaacctctgctggatgagatccagctgctgatcacctctaaggagtacttcatgtccaagttcagcgacagatacgaccagcaagaagtggtcgacgtgtccgctctcacaaaagagctcgagaagatcaagcggcagaaggaaaagtggtacgacctgtacatggacgaccggaatcctatccccaaagaggagctgttcgccaagatcaacgagctgaacaagaaagaagaggaaatctactccaagctgtctgaagtggaagaggacaaagagcctgtggaagaaaagtacaacagactgtccaagatgatcgacttcaagcagcagttcgagcaggctaatgacttcaccaaaaaggaactgctgttctctatcttcgagaagatcgtgatctatcgggagaagggaaagctgaaaaagattacactggactacaccctgaag

SEQ ID NO: 8 - DNA, 1383 bp, synthetic construct
  source 1..1383: mol_type = other DNA
atgaaagtggccgtgtactgcagagtgtccaccctcgagcagaaggagcacggccattctattgaggaacaagagcggaagctgaagtccttctgcgacatcaacgactggacagtgtacgacacctacatcgacgctggatactctggcgccaagcgggacagacctgagctgcagcggctgatgaacgatatcaacaagttcgacctggtgctggtctacaagctggaccggctgaccagaaacgtgcgggatctgctggacctgctggaaatcttcgagaagaacgacgtcagcttcagatccgccaccgaggtgtacgacaccaccaccgctatgggccggctgttcgtgaccctggtgggcgctatggccgagtgggagagagagacaatcagagaacggacccagatgggcaagctggccgctctgagaaagggcatcatgctgaccacaccacctttttactacgacagagtggacaacaagttcgtgcctaacaagtacaaggacgtgatcctgtgggcctacgacgaggccatgaagggccagtccgctaaggccatcgccaggaagctgaacaactccgacatccctccccctaacaatacccagtggcagggcagaaccattacccacgccctgcgcaaccctttcaccagaggccacttcgattggggcggcgtgcacatcgaaaataaccatgagcctatcatcaccgatgagatgtacgagaaagtcaaggatagactgaatgagagagtgaacaccaagaaggtccgacacacctccatcttcagaggaaagctcgtgtgtcctgtgtgcaacgccagactgacactgaattctcacaagaagaagtccaactccggctacatctttgtgaagcagtactactgtaacaactgcaaggtgacccctaacctgaaacctgtgtacatcaaagagaaagaagtgatcaaagtgttctacaactacctgaaaagattcgacctggaaaagtacgaagtgacacagaaacagaacgaacctgagatcaccatcgatatcaataaggtgatggaacagcggaagagataccacaagctgtacgcctctggactgatgcaagaagatgaactgtttgatctgatcaaggaaaccgaccagaccatcgctgagtacgagaagcagaacgagaaccgggaggtgaaacagtatgacatcgaagatatcaagcagtataaggacctgctgctggaaatgtgggacatctcctctgacgaggacaaggaggacttcatcaagatggctatcaagaacatctacttcgagtatatcatcggcaccggcaacacctctcggaagcggaacagcctaaagatcactagcatcgagttctac

SEQ ID NO: 9 - DNA, 1383 bp, synthetic construct
  source 1..1383: mol_type = other DNA
atgaaagtggctatctacaccagagtgtccacactggaacagaaagagaagggccactccatcgaggagcaggaaagaaagctgagagcctactccgacatcaacgactggaagatccacaaggtgtacacagatgctggctactctggcgctaagaaagatagacctgccctgcaagagatgctgaacgagatcgacaacttcgacctggtgctggtttataagctggaccggctgacaagatccgtgaaagatctgctggaaatcctggaactgttcgagaacaagaacgtgttgttcagatccgccaccgaggtgtacgacaccaccagcgctatgggcagactgtttgtgaccctggtcggcgccatggctgagtgggaacggaccaccatccaggagagaaccgccatgggcagacgggcctctgctagaaaaggcctggccaagaccgtgcctccattctactacgaccgggtgaacgataagttcgtgcccaacgagtacaagaaggtgctgcggttcgccgtggaagaggccaagaagggcacctctctgagagagatcaccatcaaacttaacaactctaagtacaaggcccctctgggtaagaactggcaccggtctgtgatcggcaacgctctgacctcccctgtggccaggggccatctggtgttcggcgacatcttcgtggaaaacacccacgaggctatcatctctgaggaagaatatgaagagatcaaactgcgcatctccgaaaagaccaacagcaccatcgtgaagcacaacgccatcttccggtccaagctcctgtgccccaattgtaaccagaagctcacactgaacaccgtgaagcacacccctaaaaacaaggaagtgtggtacagcaagctgtacttttgctccaactgcaagaataccaagaacaagaatgcctgcaatatcgatgagggcgaggtcctgaaacagttctacaactacctgaagcagtttgatctgacctcctacaagatcgagaaccagcctaaggagatcgaggacgtgggaatcgacattgaaaagctgcggaaagagcgggccagatgtcagactctgttcatcgaaggaatgatggacaaggacgaggccttccctatcatcagccggatcgacaaggaaatccatgagtacgagaagcggaaggataatgacaagggaaagacattcaactacgagaagatcaagaacttcaaatactctctgctgaacggctgggagctgatggaggacgagctgaaaaccgaatttatcaagatggccatcaagaacatccacttcgagtacgtcaagggcatcaagggcaagagacagaactccctgaagatcaccggcatcgagttctat

SEQ ID NO: 10 - DNA, 1443 bp, synthetic construct
  source 1..1443: mol_type = other DNA
atgatcacaaccaacaaggtggctatctacgtcagagtgtccaccacaaatcaagtggaagaaggctactccatcgacgagcagaaggacaagctctcctcctactgtgacatcaaggattggaacgtgtacaaggtgtacaccgacggcggcttttccggaagcaacaccgatagacctgccctggaatctctgatcaaggatgcaaagaagcggaagttcgacaccgtgctggtgtacaagctggacagactgtccagatcccagaaggacaccctgcacctgatcgaggacgtgttcatcaagaacggcatcgagtttctgtccctgcaagagaacttcgatacatctaccccattcggcaaggccatgatcggtctgctgtctgtgttcgcccagctggagagagaacagatcaaagagcggatgcagctcggcaagctgggcagagctaagtctggaaagtccatgatgtgggccaaaaccagctacggctacgactaccacaaggaaaccggcaccgtgacgatcaaccccgctcaggctctgacaatcaagtttatcttcgagtcttacctgagaggcagatccatcaccaagctgagagatgacctgaacgagaagtaccctaagcacgtgccttggtcctacagagccgtgagaaccatcctggacaatcctgtgtactgtggcttcaaccagtacaagggcgagatctaccccggcaaccacgagcctatcatctccaaagaggagtacgacaagacccagtccgagctgaagatccggcagcggaccgctgctgagaacgtgaaccctcgccccttccaggccaagtacatcctgtctggcattgcccagtgcggatattgcggcgctcctctgaaaatcatgctgggcgtcaagagaaaggacggatctcggctgaagaaatacgagtgccaccagagacatcctagaaccctgagaggcgtgaccacctacaacgacaataagaagtgcgactcgggcttctactacaaggacaagctcgaggcctatgtgctgaaggaaatctctaagctgcaggacgacgccgattacctggataagatcttcagcggcgacaacgccgagacaatcgaccgcgagagctataagaagcagatcgaagaactgtccaaaaaactgagcagactgaacgacctgtacatcgacgaccggatcaccctggaggaactgcagtctaagtctgccgaattcatctccatgcggggcaccctggaaaccgagttggaaaacgatcctgctctgcggaagaacaagcggaaagccgacatgagaaagctgctgaacgctgaaaaggtgttctctatggactacgagtcccagaaagttctggtgcggagactgatcaacaaagtgaaggtcaccgccgaggatatcgtgatcaactggaagatc

SEQ ID NO: 11 - DNA, 1341 bp, synthetic construct
  source 1..1341: mol_type = other DNA
atgctgagatgcgccatctacatcagagtgtccaccgaggagcaggccatgcacggcctgtccatggacgctcagaaagccgatctgaccgactacgctaagaagcacaactacgagatcatcgactactacgtggactccggcaagaccgccagaaagagactgtccaagcgcaaggacctgcagcggatgatcgaggacgtcaagctgaacaagatcgacatcatcatctttaccaagctggacaggtggttccggaacgtgcgggactactacaagatccaagaggtgctggaggaccacaacgtcgactggaaaaccatcttcgagaattacgatacctctaccgctaacggcagactgcacatcaacatcatgctgtccgtggctcaggacgaggccgacagaacctccgaaagaatcaaacgggtgttcgagaacaagctgaagaacaacgagcctacatctggctctctgcctatcggctacaagatcaaagagaagtccatcattatcgatgaggaaaaggcccctatcgccaaggatgtgttcgatttctactactaccaccagtcccagaccaaggtgttcaaagaaatcctcaacaaatacaacctgtctctgtgcgaaaagaccatccggagaatgctggagaataagctgtacatcggcatctacagagagcacgagaacttctgtcctcctctgatcgacaagaacaagttcgacgaagtgcagctgattctgaagaggcggaacatcaagtatatccctactaagcggatctttctgttcaccagcctgctgatctgcaaggagtgtagacataagatgatcggcaacgcccagatcagaaacacaaaggctggaaagatcgagtacatcttgtaccggtgcaaccaatcttacgctcggcacacctgcaaccacagaaaggtgatctatgaaaacaagatcgaaacctatctgctgaacaacatcgagtccgagctgaaaaagtttatctacgactacgagctggaagatatccccaaggtgaagaacaaagtgaacaaaacaaatatcaagcggaagctggaaaagctgaaagaactgtacatcaacgacctcatcgacatcgacatgtacaaagaggattacaagaagtacaccgagatcctgaataccaaagaagaaaagatcgaacagagaaacctgcagcctctgaaggacttcctgaactccgacttcaagtctctgtactcctccatctctagagaagagaagcggctgctgtggagaggcataatcagcgagatccagatcgactgcaataacgatatcaccatcatcccccatcca

SEQ ID NO: 12 - DNA, 1380 bp, synthetic construct
  source 1..1380: mol_type = other DNA
atgaaggtggccatctacactagagtgtcctcggctgagcaggccaacgagggatactccatccacgagcaaaagaagaagctcatctcctactgcgaaatccacgactggaacgagtacaaagtgttcaccgacgccggcatctctggcggctctatgaagcggcctgctctgcagaaactgatgaaacatctgtctagcttcgacctggtgctggtgtacaagctggacagactgaccagaaacgtgcgcgacctgctggatatgctcgaagaattcgagcagtacaacgtatctttcaagtccgccaccgaagtgttcgacaccacctctgctatcggcaagctgttcatcaccatggtgggcgctatggccgagtgggaaagagaaaccatcagagagcggagcctgtttggatctcgggccgctgtgcgggaaggcaactacatcagagaggctcctttctgctacgacaacatcgagggcaagctgcatccaaacgaatacgccaaggtgatcgatctgatcgtgtccatgttcaagaagggcatctccgccaatgagatcgccagacggctgaactcctccaaggtgcacgtgcctaacaaaaagtcctggaaccggaacagcctgatccggctcatgagatctcccgttctgcggggccacaccaagtacggcgacatgctgatcgagaacacccatgagcctgtgctgtccgaacacgactacaatgctatcaataatgccatctccagcaagacccacaagtccaaggtcaagcaccacgccatcttcagaggagccctggtgtgtcctcagtgcaacagaaggctgcacctgtacgctggcacagtgaaggaccggaagggctacaagtacgatgtcagaagatacaagtgcgagacatgttctaagaacaaggacgtgaagaacgtgtccttcaacgagtctgaggtggaaaacaagttcgtgaacctgctgaagtcttacgagctgaacaagttccacatccggaaagtggaacccgtgaaaaagatcgagtatgatatcgacaagatcaacaagcagaagatcaactacaccagatcttggtccctgggctatatcgaggacgacgagtacttcgagctgatggaggagatcaacgccacaaagaagatgatcgaggaacagacaaccgagaacaagcagtctgtcagcaaagagcagatccagtccatcaacaactttatcctgaaaggctgggaggaactgaccatcaaggataaagaggagctgatcctgtccaccgtggacaagatagagttcaatttcattcctaaggataagaagcacaaaaccaacaccctggacatcaacaacatccactttaagttt

SEQ ID NO: 13 - DNA, 1422 bp, synthetic construct
  source 1..1422: mol_type = other DNA
atggccgtgggcatctacatcagagtgtccacccaggagcaggcctctgaaggccattccatcgagtcccagaaaaagaaactggcttcctactgcgagatccagggctgggacgactaccggttctacatcgaggaaggcatctccggcaagaacacaaatcggcctaagctgaagctgctgatggaacacatcgagaagggaaagatcaacatcctgctggtgtacagactggatagactgacaagatctgtgatcgacctgcacaagctgctgaacttcctgcaagagcacggctgtgccttcaagtccgccaccgagacatacgacaccaccactgccaacggcagaatgtccatgggcatcgtgtccctgctggctcagtgggaaaccgagaacatgtccgagcggatcaagttgaatctggaacataaggtgctggtcgagggcgaaagagtgggagccatcccttacggcttcgacctgtctgatgatgaaaagctggtgaagaacgagaagtctgctatcctgctggacatggtcgaacgggtggaaaatggatggtccgtgaacagaatcgtgaactatctgaacctgaccaacaacgaccgcaactggagccctaacggcgtgctgaggctgctgcggaatcctgctctgtacggcgctaccagatggaacgataagatcgccgagaacacccacgagggaatcatcagcaaagagagattcaaccggctgcagcagatcctcgccgacagatccatccaccaccggcgggacgtgaagggcacctatatcttccaaggcgtgctgagatgtcctgtgtgcgaccagaccctgtccgtgaaccggtttattaagaagagaaaggacggcaccgagtattgtggtgtgctgtaccggtgccagccttgcatcaagcagaacaagtacaacctggccatcggcgaggccagatttctgaaggccctgaacgagtacatgtctaccgtggaattccagacggttgaagatgaggtgatacccaagaagtctgagagagagatgctggagtctcagctgcagcagatcgctcggaagcgggaaaagtaccagaaggcttgggccagtgatctgatgagcgatgacgagttcgagaagctgatggtggaaaccagagaaacctacgacgagtgcaagcagaagctcgagtcctgcgaggacccaatcaaaatcgacgaaacctacctgaaagaaatcgtgtacatgttccaccagacattcaacgacctggaatccgagaagcagaaagagttcatcagcaagttcatcagaaccatcagatacaccgtgaaggagcagcagcccatcagacctgacaagtctaagaccggcaagggcaaacaaaaagtgatcatcaccgaagtggaattttaccag

SEQ ID NO: 14 - DNA, 1413 bp, synthetic construct
  source 1..1413: mol_type = other DNA
atgacagtgggcatctatatcagagtgtccaccgaggaacaggtcaaggagggcttctccattagcgctcagaaagaaaagctgaaggcctactgcaccgctcaaggctgggaggacttcaagttctacgtggacgaaggcaagtctgccaaggacatgcaccggcccctgctccaagagatgatctctcatatcaagaagggactgatcgataccgtgctggtgtacaagctggacagactgacaagatccgtggtggatctgcacaacctgctgtccatcttcgacgaattcaactgcgccttcaagtccgccacagaagtgtacgacacctccagcgccatgggcagattcttcatcacaatcatctcctccgtggcccagttcgagcgcgaaaacacctccgaaagagtgagctttggcatggccgagaaggtcagacagggcgagtacatccctctggctcctttcggctataccaagggcaccgacggaaagctgatcgtcaacaagatcgagaaagaaatcttcctgcaggtggttgagatggtgtctaccggctactctctgcggcagacctgcgagtacctgaccaacatcggcctgaaaacccggagatctaatgatgtgtggaaggtgagcaccctgatctggatgctgaagaaccccgccgtgtacggcgccatcaagtggaataacgagatctacgagaacacccacgagcctctgatcgacaaggctaccttcaacaaagtggctaagatcctgtctatcagatccaagtccaccacctctagaagaggccacgtgcaccatatctttaagaaccggcttatctgcccagcatgtggaaagcggctgtctggcctgcggaccaagtacatcaacaagaataaggaaactttctacaacaacaactacagatgtgctacctgcaaggagcacagacggcctgctgtgcagatctccgagcagaagatcgagaaggcctttatcgactacatctccaactacaccctgaacaaggccaacatcagctctaagaagctggacaacaacttaaggaagcaggaaatgatccagaaagagatcatcagcctgcagcggaagagagagaagttccagaaagcctgggccgccgacctgatgaacgacgatgagttctccaaactgatgatcgatacaaagatggaaatcgacgctgctgaggaccggaagaaagaatacgacgtgtccctcttcgtgtctcctgaagatatcgccaagcggaacaacatcctgcgggagctgaagatcaactggacctctctgtcccctaccgagaaaaccgattttatttccatgttcatcgaaggcatcgagtacgtgaaggacgacgagaataaggctgtgatcaccaagatctctttcctg

SEQ ID NO: 15 - DNA, 1356 bp, synthetic construct
  source 1..1356: mol_type = other DNA
atgaaggccgccatctatatcagagtgtccacccaggaacagatcgagaattacagtatccaggctcagaccgagaaactgaccgctctgtgcagatccaaggactgggacgtgtacgatatcttcatcgacggaggctactctggctccaacatgaacagacccgccctgaatgagatgctgtctaagctgcacgaaatcgacgccgtggtggtgtacaggctggacagactgtccagatcccagagagataccatcacactgatcgaagagtacttcctgaagaacaacgtggaattcgtgtccctcagcgaaaccctggacactagctctccatttggcagagccatgatcggcatcctgtctgtgttcgcccagctggaaagagagacaatccgggacagaatggtcatgggcaagatcaagcggatcgaggctggcctgcctctgacaaccgccaagggcagaacattcggctatgatgtgatcgacaccaagctgtacatcaacgaggaagaagctaagcagctgcagatgatctacgacattttcgaggaagagaagtccatcaccaccctgcagaagagactcaaaaaactgggcttcaaggtgaagtcctactcctcctacaacaactggctgaccaacgacctgtactgcggctacgtgtcctacgccgacaaagtccataccaagggcgtgcacgagcctatcatctctgaagaacagttctacagagtgcaggagatcttcagccggatgggcaaaaatcctaacatgaaccgggattctgctagcctgctcaacaatctggtcgtttgtggcaagtgtggactgggatttgtgcacagaagaaaggacaccatctccagaggtaagaagtaccactaccggtactacagctgcaagacctacaagcacacccatgagctggagaagtgcggcaacaagatctggcgggctgacaagctggaagaattgatcatcgatcgcgtgaacaactattccttcgcttctcggaacgtggacaaagaggacgagctggacaacctgaacgagaagctgaaaaccgagcacaagaaaaagaagcggctgttcgacctgtacatctccggctcttacgaggtgtctgagctggatgctatgatggccgacatcgatgcccaaatcaactactacgaggcccagatcgaagccaacgaggaactgaagaagaacaagaaaattcaagagaatctggctgatctggccaccgtggactttgactccctagagttccgggaaaagcagctgtacctgaagtctctgatcaacaagatctacatcgacggcgagcaggtgaccatcgagtggctg

SEQ ID NO: 16 - DNA, 1410 bp, synthetic construct
  source 1..1410: mol_type = other DNA
atgaagggcgagtctgagctggacaagaaggccgccatctacatcagagtttctacacaagagcaggctacagagggctattcgatccaggcacaaaccgacagactgatcaagtacgtggaagccaaggactttatcctgtataagaagtatatcgacgccggctacagcgcttctaagctcgaaagacccgctatgcaggatctcatccaggacgtccaaagcaagaaagtggacgtggtcatcgtgtacaagctggatagactgtctagatctcagaaggataccatgtacctgatcgaggacatcttccggcctaacgacgtggaactgatctctatgcaggaaagctttgacacctccaccgccttcggctctgccaccgtgggcatgctgtccgtgttcgcccaactggagaggaagtccatctccgaaagaatgatcacaggcagagtggagcgggctaagaaaggcttctaccacaccggcggccaggacagacctccagctggctaccagttcaactccgacaaccagctgatcatcaacgagtacgaggccgctgctatcaaggacctgtttcggctgtacaacgacggcctgggaaagtctagcatctccgagtacctgaagaagaactaccccggaaaaaacaagtggctgccttcttctatcgatcggatgctgaagaactccctgtacatcggcaaggtgaagttctccggcgccgagtacgacggcatccatgagcctatcatagacgaagtgaccttctacaagacccagaaggagatcgccagacggaagcagaccaacaccaagagatacaactacgtggccctgctgggcggcctgtgcgagtgcggcatctgtggcgctaagatggccaacagacgggccgtgggacgcaagggtaaggtgtaccggtactacagatgctactccaagaaaggatctcctaagcacatgatgaaaaccgatggctgctcctccaaggcccagcagcagttcatcatcgacgaggctgtgattaacaacctgaagaacatcgacgtcgaagccgaactgaaacgcagatctgctcctcagaccaatacctctctgatctccagccagatcgagagcatcgataagcagattaacaagctgatcgacctgttccaggtggactccatgcctctggatgtgatcagcgagaagatcgataagctgaacaaagagaagcagtccatggaaaaactgctggaacggaagaataagctggacaaaaccgagctgcagcacagattcgatgtgctgaagtccttcgactgggacaattccagtatcgagtccaagcgggtggtgatcgagatgctggtgcagaaagtgatcattcacgacaactccatcgaaatcatcctggtggaa

SEQ ID NO: 17 - DNA, 1536 bp, synthetic construct
  source 1..1536: mol_type = other DNA
atgcggaccaacgagcacaacttccacaacatcgaggaggagattaagcacgtggccgtgtacctgagactgtcccggggtgaggatgagagcgagctggataaccacaagactcggctgctgaacagatgtgaactcaacaactggtcctacgagctgtataaggaaatcggatctggctctaccatcgatgatagacctgtgatgcagaaactgctgaccgatgtggaaaagaacctgtacgacgccgtgctggtggtggacctggataggctgtcgagaggcaacggcaccgacaacgacagaatcctgtattccatgaaagtgtccgaaaccctgatcgtggtggaatccccctaccaggtgctggacgctaacaacgagtccgacgaagagatcatcctgtttaagggcttcttcgcccggttcgagttcaagcagatcaataagcggatgagagagggcaagaagctggctcagagcagaggccagtgggtcaactccgtgacaccctacggctacatcgttaacaagaccaccaagaaactgaccccttctgaagaggaagccaaagtggtgatcatgatcaaggacttcttctttgaaggcaagagcacctccgacatcgcttgggagctgaacaagagaaagatcaagcctagacgggctacagaatggcggtcctcctctatcgccaatatcctgcagaatgaagtgtacgtgggcaacatcgtgtacaacaagtctgtcggaaacaagaagccctctaagtccaagaccagagtgaccaccccatacagacggctgcctgaggaggagtggcggcgcgtgtacaacgcccaccagcctctgtactctaaggaagagttcgaccggatcaagcagtacttcgagtgcaacgtcaagagccataagggatccgaggtgcgcacctacgccctgaccggcctgtgcaagacccctgacggcaagaccatgagagtgacccagggcaagaagggcaccgacgacgacctgtatctgttccctaagaagaacaagcacggcgacagcagtatctacaagggcatttcctacaacgtcgtgtacgagacactcaaagaggtgatcttgcaagtgaaagactacctggactctgtgctggaccagaacgaaaataaggacctggtggaagaactgaaagaggaactgatgaagaaggaggatgaactggaaacaatccagaaggccaagaatcggatcgtgcaaggctttctgatcggcctgtacgacgagcaggactccatcgagttgaaggtggagaaggagaaagagatcgacgaaaaggaaaaggagatcgaggctatcaagatgaagatcgacaatgcaaaaaccgtgaacaactccatcaaaaaaaccaagatcgagagactgctgtctgacgtgcagtctgccgagtctgagaaagaaatcaaccggttctacaagaccctgatcaaggagatcatcgtggatagaaccgatgaaaacgaggctaagatcaaggtcaacttcctg

SEQ ID NO: 18 - DNA, 1425 bp, synthetic construct
  source 1..1425: mol_type = other DNA
atgatcacaacaaacaaggtggccatctacgtgcgggtgtctaccaccaaccaagtggaggaaggctactccatcgacgagcagaaggacaagctggaggcttactgcaagatcaaagactggaagatctacgatgtgtacgtggatggcggcttcagcggcgccaacacccagcggcctgagctggaacggctgatctccgacgtgaagcggaagaaggtggacatcgtgctggtgtataagctggacagactgtctagatcccagaaggacacactgtttctgatcgaggatgtgttcgccaagaacgacgtggctttcatcagcctgcaggagaacttcgacacctccacccctttcggaaaggcctctataggcatgctgtctgtgtttgctcagctggagcgggagcagatcaaggaaagaatgatgctgggcaaagaaggcagagccaagaatggcaagtccatgtcttggaccaccatcgccttcggctacgactactctaaggaaaccggcgtgctgtccgtgaaccctacccaggctctgatcgtcaaccggatcttcaccgagtacctgaacggcaagcctgtggtgaaaatcatccgggacctgaacgccgagggccatgtgggcagaaagcggccttggggcgagacaatcaccaagtacctgctgaagaacgagacatacctgggcaaggttaagtataaagacaaggtgtacgagggccagcacgagcccatcatcacccaagagctgttcgatctggtgcagctggaagtggagcggagacagatctccgcctacgaaaagtacaacaaccccagaccattcagagctaagtacatgctgagcggcctgatgaagtgcggatactgtggcgcttctctgggcctgagatacaccagaaaggacaagaacggcatctctcaccacaagtaccagtgccggaatcggcactccaaggacctggaaaaaagatgcgagtctggctggtactccaaagaggaactcgagcgcggagtgatcaaggaactggaacgtatcaagttcgatcctaagtataagaatgaaaccctggccaagaaagaggaaaccatcaaagtggaagagatcaagaagcagctggagcggatcaacaaccaggtgtccaaactgaccgagctgtacctcgatgagatcatcaccaggaaggagcttgatgaaaagaacgacaagatcaagaccgaaagacaattcctggaggagcagctggagaaccagaagtccaacgtgctctccatcagaaagcggaaactgaccagactgctgaaggattttgacgtcgagaagctgtcctacgaggacgcctctaagattgtcaagaacatcatcaaagaaatcatcgtgactaaggacggcatgtccatcaccctggacttc

SEQ ID NO: 19 - DNA, 1386 bp, synthetic construct
  source 1..1386: mol_type = other DNA
atgggcaagtctatcaccgtgatcccagctaaaaaagtgcagacctctgtgctgcatcaagaccggaagaagatcaaggtggccgcctactgtcgggtgtccaccgaccaggaggagcagctgtcctcctatgaaaaccaggtgaactactacagagagttcatctccaagcacgaggactacgagctggtggacatctacgccgacgagggcatctccgcaaccaacaccaagaagcgggacgccttcaaccggctgatccaagactgtagggccggaaaggtcgacagaatactggtgaagtccatctcgagattcgccagaaacacactggattgcatcaagtacgtgcgggagctgaaggaactgggcgtgggcgtgaccttcgagaaagagaacatcgacagcctggatagtaagggcgaggttctgctgaccattctgagctctctggctcaggacgagtctcgatctatctctgagaacgccacctggggcatcagaaagaagttcgagagaggcgaagtgcgcgtcaatacaacaaagttcatgggctacgacaaggacgagaacggcagactgatcatcaaccctcaacaggctgaaaccgtcaagtttatctacgagaaatttctggagggctactcccccgagtccatcgccaagtacctgaacgacaatgagatccctggctggaccggcaaggccaactggtacccttctgccatccagaagatgctgcagaacgagaagtacaagggcgacgctctgctgcagaaaacctttaccgtggacttcctgaccaagaagagagtgcagaacgatggacaggtgaaccagtactacgtggaaaattctcacgaggccatcatcgacgaagagacatgggaaacagtgcagctcgagatggccagaagaaagacctacagagatgagcaccagctgaaatcctacatcatgcagtccgaggataaccccttcaccaccaaggtgttctgcggcgcttgtggctccgctttcggccggaagaactgggctacctccagaggaaagcggaaagtgtggcagtgcaacaacagataccggatcaagggagtcgaaggctgctacagctcccacctggacgaggctaccctcgaacagatcttcctgaaagccctggaactgctgtccgaaaacatcgacctgctggatggcaagtgggagaagatcctggccgagaacagactgcttgataagcactatagcatggctttatctgatctgctgcggcaggaacagatcgacttcaatccttccgacatgtgcagagtgctggaccacatccggatcggcctggatggcgaaatcaccgtgtgcctgctggaaggtaccgaggtggacctg

SEQ ID NO: 20 - DNA, 1233 bp, synthetic construct
  source 1..1233: mol_type = other DNA
atgagaacagtcagacgcatccagcctatcaagtctccttgcaagcctagattcaaagtggccgcctatgctagagtgtccgactcacgcctgcaccactctctgtccacccagatctcctactacaacagactgatccaggcccatcctgattgggagttggtcggaatctactacgacgagggaatttccggcaaagagcagtccaacagacagggcttcctgaatctgatcaaggactgcgaggacggcaagatcgatagaatcatcaccaagtccatcgccagatttggacggaacaccgtggaactgctgaccaccgtgcggcagctgagactgaagaacatcggcgtgaccttcgagaaggaaaacatcgacagcctgtcctctgaaggcgagctgatgctgacactgctggcttctgtggcccaggaagagtcccagaacctgtctgagaatatcagatggcggatccagaagaagttcgaaaagggaatccctcacacccctcaggacatgtacggctatcggtgggatggcgaacagtaccagatcgaacccaacgaggccaaggtgatccggaaggtgttcaagtggtacctggacggcgactccgtgcagcagatcgtggacaagctgaaccaggagcaggtgctgacccggctcggcaaccccttcaccgtggctagcatcagagagttcttcaagcaggaagcttactttggtagactcgtgctgcagaaaacctacagagaagccttctccagaaatccaaagaggaacaaaggccagagaaacaagtacatcatcgagaacgctcacgagcccatcgttacaaaggaatacttcgacctggtgctgcatgagaaagagcgaagaaaccaactgatgcaccaagagtctcacctgaacaagggcatcttccgggataagatctcttgctccgagtgcggctgtctgatgatcgtgaaagtcgattccaagcaagtgaacaagaccgtgcggtactactgcagaaccagaaaccggttcggcgcttcttcctgcagctgtcggaccctgggcgagaagcggctgctggccagctttaaatccaagctgggcatcgtgcctgacaaggagtgggtggaaaacaacatcaagcacatcgagtacgacttcggctaccggatcctgcgggtgacacctgtgaagggcagaaagtacctgatcgagatcagagagggcagatac

SEQ ID NO: 21 - DNA, 1419 bp, synthetic construct
  source 1..1419: mol_type = other DNA
atgcggaacaaggttgccatctacgtccgggtgtccacagctagccaggccgacgagggctactccatcgacgaacagaaaagcaagctggaggcctactgcgagatcaaggactggaagatctacgacacctacatcgatggcggcttctccggggccaacacccagaggcccgaactggaacggctgatttctgatgccaagcggaagaagattgatatcgtgctggtgtacaagctggacagactgtccagatctcaaaaggacacactgttcctgatcgaggatgtgttcgctaagaacgacgtggctttcatcagcctgcaggagaacttcgacacctctacccctttcggcaaggcctccatcggcatgctgtccgtgttcgcccagctggagcgcgaacagatcaaagagcggatgatgctgggcaaagagggcagagccaagaatggcaagtccatgtcttggaccaccatcccttttggctacgactactccaaagagacaggcatcctgagcgtgaaccccacccaagctctgatcgtgaagagaatcttcaccgagtacctgaacggcaaatctgtggtgaagatcatccgggacctgaatgccgagggccatgtgggccggaagcggccttggggcgaaaccatcaccaagtatctgctgaaaaacgaaacctacctcggaaagtctaagtataagggcaaggtattcgaaggccagcacgacgccatcatctctcaggaactgtttgatctggtgcagctggaagtggagaagagacagatctccgccttcgagaagtacaacaaccctagacctttccgggctaagtacatgctgtctggcctaatgaagtgcggctactgcggcgcttctctgggactctacgtggcccctaagaacaagaacggcgtgagcaagtacaagtaccagtgtagacaccggtaccacaaggacaaagccatcagatgcaactccggatggtactccaaggacgagctggagaaaagagtgatcaaagagctcgagcggctgaagttcgatcctaagtacaagaaagaaaccctggccaagaaagatgagacaattaaggtggaggacatcaagaagcagctggaaagaatcaataagcaggtgtccaagctgaccgagctgtacctggacgaggtgatcaccagaaaggacctggacgaaaagaacgccaagatcaagaccgaaagacagtacctggaggagcagctggagaaccagaagtccaacgtgatgtccatccgaaagcggaagctgtctagactgctgaaggacttcgacatcgagaagctgtcctacgaggaagcttctaagatcgtgaagtccgtcatcaaggaaatcgtcgtgaccaaggacgacatgaccatcactctggatttt

SEQ ID NO: 22 - DNA, 1389 bp, synthetic construct
  source 1..1389: mol_type = other DNA
atgaaggtggccacttacgtgcgcgtgtccaccgacgagcaggctaaggagggcttctccatccccgcccaaagagagcggctgagagccttctgcgagtctcagggatgggaaatcgtggaagagtacatcgaagagggctggtccgccaaagacctggacagacctcagatgcagcggctgctcaaggatatcaagaagggcaatatcgacatcgtgctggtgtacaggctggatagactgacccggtctgtgctggatctgtacctgctgctgcagacctttgagaagtacaacgtggctttcagatccgctaccgaggtgtacgacacctctaccgccatgggcagactgttcattacccttgtggccgccctggctcagtgggagcgggagaacctggccgagagagtgaagttcggcatcgagcagatgatcgacgagggaaagaagcctggcggccactctccatacggatacaagtttgacaaggacttcaactgcaccatcatcgaggatgaggccaacaccgtgcggatgatttacagaatgtactgcgacggctacggctaccactccatcgctaagcgcctgaatgagctgggcatcaagcctagaatcgccaaagagtggaaccacaacagcgtccgggacatcctgaccaacgacatctacatcggcacctatagatggggcaacaaggttgtgctgaacaaccatcctcctatcatctccgagacactgttcagaaaggtgcagaaagaaaaagaaaagcggcgggtggaccggaccagagtgggcaagtttctgctgacaggcctgctgtactgtggcaattgcaacggccacaagatgcagggcacctttgacaaaagagaacagaaaacctactaccggtgtctgaagtgcaaccggatcaccaacgagaagaacatcctggaacctctgctggatgagatccagctgctgatcacatctaaagagtacttcatgtccaagttctccgaccagtacgatcaaaaggaggaagtggacgtgtctgctctgaagaaggagctcgaaaagatcaagagacagaaggaaaagtggtacgacctgtacatggacgacagaaaccccatccctaaggaagatctgttcgccaagatcaacgagctgaacaagaaggaagaagagatctataacaagctgaacgaggtcgaacccgaggacaaggagcctgtcgaagaaaagtacaacagactgagcaagatgatcgacttcaagcagcagttcgagcaagctaatgatttcaccaagaaagaactgctgttcagcatcttcgaaaaaatcgtgatctatcgggagaagggcaagctgaaaaagatcaccctggactacaccctgaag

SEQ ID NO: 23 - DNA, 1356 bp, synthetic construct
  source 1..1356: mol_type = other DNA
atgctgcgcgtggctctgtatatcagagtgtctaccgaggagcaggccctgaacggcgacagcatccggacccagatcgaggccctggaacagtactccaaggagaacgacttcaacatcgtgggcaagtacatcgacgagggctgttctgccaccaacctgaagcggcctaatctgcaaagactgctgcgggacgtggaaaaagacaaagtggacctggtgctgatgactaagatcgatcggctgtctagaggagtcaagaactactacaagatcatggaaacactggagaagcacaagtgcgactggaaaaccatcctggaaaactacgactcctccaccgccgctggcagactccacatcaacatcatgctgtccgtggccgagaacgaggctgctcagacctccgagagaatcaagttcgtgttccaggacaagttgagaagaaaggaagtgatctctggtacaatccccatcggctacaaaatcgagaataagcatctggtgatcgataaagagaagaagtacattgtgaaggccatcttcgacgagtacgagaagtctggctccgttaggaccctgatcgaaaccatcaacaacctgcacggcgaactgtactcctataacaagatcaagaacatcctgagaaacgagctgtacatcggcatttacaataagagaggcttctacgtggaggactactgcgagcctatcatcagcaagaagcagttcaagcagatccagcggatcctggaaaagaataagaaaaccacaccaaacaagaacatccactaccacatcttcagcggcctgctcaagtgcaaggagtgtggctacaccctgaagggcaactcctccaacgtgggagagaagctgtacctgtcttacagatgctccaccttttacctgaacaagaactgcgtgcacaacgtgacccacaacgagaagcatattgagaactatctgctgaccaacctgaagcctcagctgcacaagcacatggtgaagctggaagcccagaacgaaaagatcagacggaacaaaaagtccaacaagaaggatgagaaaaagaaaatcatgaagaaactggataagatcaaggacctgtacctggaggacctgatcgataaagaaacctaccggaaggactacgagaagctgcagtcccagctggacaacatcaccgaggaacaagagtctcagatcatcgacacctctcacatcaagaagtttctggacatcgacatcaatgagatgtactctgatctgagcagagtcgagcggcggagattctggctgtccatcatagactacatcgagatcgataacaacaaaaacatcaccatcaacttcatc

SEQ ID NO: 24 - DNA, 1776 bp, synthetic construct
  source 1..1776: mol_type = other DNA
atgaagatcaccctgctgtactacatcaagaagttcaacatctactgcaacagatacctgagccagcagatcaacatctccgtggacatcatcggcttctaccagttcaagaacgtcaccaactctgtgaccgacgtgctgaagagaggtgataatctggacagaatctgtatctacctgcggaagtccagagccgatgaagaactggaaaagaccatcggagtgggcgaaaccctgagcaagcacagaaaggctctgctgaagttcgccaaggaaaagaagctgaatatcatggaaatcaaagaggaaatagtgtccgctgactccatcttcttcagacctaagatgatcgaactgctgaaggaggtggagaacaaccagtacaccggcgtgctggttatggacatccagagactgggcagaggcgacaccgaggaccagggcatcattgctagaatcttcaaggagtctcacaccaagatcatcacccctatgaaaacctacgacctggacgacgatttggacgaggactactttgagttcgagagtttcatgggccgcaaagagtacaagatgatcaagaagcggatgcagggcggcagagtgcggtccgtggaagatggcaactacatcgccaccaatcctccatttggctacgacatccactggatcaacaagtccaggacactgaagttcaactccaaggaatctgagatcgtgaaactgatctttaaactgtataccgagggaaatggcgctggcaccatctccaactacctgaactccctgggctataagaccaagttcggcaacaacttcagcaactcttctatcatcttcatcctgaagaaccctgtgtacatcggaaagatcacctggaagaagaaggacatcagaaagtccaaggatcctcacaaggtcaaagatacccggaccagagacaagtccgagtggatcatcgccgacggcaagcacgagcctatcatcgacgaaaagatctggaacaaggctcaagagatcctgaacaacaagtaccacatcccttacaagatcgccaacggccccgctaaccctctggccggagtggtgatctgctccaagtgcaactccaaaatggtgatgcggaagtacggcaagaagctgcctcatctgatctgcaataacaaggagtgtaacaataagtccgccagattcgactacatcgagaaggccgtgctggaaggcctggacgagtatctgaagaactacaaagtgaacgtgaaggccaacaacaaaaccagcgatatcgagccctacgagcagcagtctaacgccctgaacaaagagctgatcctcctgaacgagcagaaactgaagctattcgactttttggaaagagagatctacacagaagagatctttcttgagagatctaagaacctggatgagcggatcaacaccaccacactggctataaacaagatcaagaaaattctggacaacgagaaaaagaagaacaacaagaacgacatcgtcaagttcgagaaaatcctggaaggctacaagaaaaccaacgatatccagaagaaaaatgaactgatgaaatctctggtgttcaagatcgagtataagaaagaacagcaccagcggaacgacggcctgctgtacatctacttcctgagcttctgcgtgcggtgcatctcctacctgacacaattcatttccttcttcgtgtacccctaccggatcctggagatctacctgaccttctcttttttcatcatctcttacgagcat

SEQ ID NO: 25 - DNA, 1512 bp, synthetic construct
  source 1..1512: mol_type = other DNA
atgcggatctgcatgtacctgcggaagtccagagctgatgaggaactggaaaagaccctgggcgaaggcgagactctgagcaagcacagaaaggctctgctgaagttcgccaaggagaaaaatctgaatatcgtggagatcaaagaggaaatcgtgtctggtgagtccctgttcttcagacctaagatgctggaactgctgaaagaaatcgagaacaaacagtactccggcgtgctcgtgatggacatgcagagactgggaaggggaaacatgcaggaccagggcatcatcctcgagacatttaagaaatctaacaccaagatcatcacccctatgaaaacctacgacctgtctaacgacttcgacgaagagtactctgagttcgaggccttcatgtcccggaaggaacttaagatgatcaatcggcggatgcaaggcggcagagtgcggagcgtcgaggacggcaactacatcgctaccaacgccccctacggctacgacatccactggatcaacaaggccagaaccctgaagcccaaccagaaggaatctgaaatcgtcaagctgatcttcaagctctacatcgagggcaacggcgctggcaccatcgctaagcatctgaacagcctgggctataagaccaagttcggcaactccttcaacaactcctccatcatcttcattctgaaaaaccctgtgtatatcggcaagatcacctggaagaaaaaggacattcggaagtccaaggatcctaacaaagtgaaggacacccggaccagagacaagtctgagtggatcatcgtggacggcaagcacgaccctatcatcgaccagatcacctggaagcaggctcaagagatcctgaataaccggtaccacgtgccttacaagctggtcaacggccctgccaaccccctggccggcctgatcatctgtaccacctgcaagtccaagatggtgatgagaaagctgagaggcaccgacagaatcctgtgcaagaacaacaagtgcaacaacatctccaacagattcgatgccgtggaaaagtccgtggtggaatctctggaaaactacctgaaggcctacaaggtgaacctgcctgagctgaacaagacctccaacctgaaactgtacgagcagcagatcagcacactgaagaaagaactgaaaattttgaacgaacagaaactgaagctgttcgattttctggagcgcggaatctacgacgaggataccttcctgaagagatctaagaacctggacgagagaatcgagatcaccaacgagtctctgtctaatctgaatcagatcatcgccaaggagaacaaggccatcaagaaagaagatatcatcaagtttgagaaggtgctggatagctacaagtccaccgctgacatccggctgaaaaacgagctgatgaaaaccttaatcttcaagatcgagtacaccaagaacaagaagggcaatgacttcaagatcaaggtgttccctaagctgaagccactgaacatc

SEQ ID NO: 26 - DNA, 1635 bp, synthetic construct
  source 1..1635: mol_type = other DNA
atgatcgccgctatctactctagaaagtctaaattcaccggcaagggcgagtccgtggaaaaccagatcgaaatgtgcaaggaatacctgaagagaaacttcaataacatcgatgacatcgaaatctacgaggacgagggcttctctggcaaggacaccaaccggcccaagtttaagaagatgatcaaggccgctaaaaacaagaagttcaacatcctcatctgctaccggctggacagaatctctcgcaacgtggctgatttcagcaataccatcgaggagctgcagaaatacaacatcgactttatatccatcaaggagcagttcgataccagcaccccaatgggcagagccatgatgaacatcgctgctgtgttcgcccagctggagcgggaaaccatcgccgagcggatcaaggacaacatggtggaactggccaagaccggacggtggctgggcggcacctctcctctgggctacaagtccgaacccatcgagtactccaatgaggacggcaagtccaagaagatgtacaagctgaccgaggttgagaacgagatgaacatcgtgaagctgatctacaagctgtacctggagaagagaggctttagctctgtcgccacctacctgtgcaagaacaagtacaaaggcaagaacggcggcgagttctccagagagacagctaggcaaatcgtgatcaatcctgtgtactgtatctccgacaagacaatcttcaagtggttcaaatccaagggcgctaccacctacggcacacctgacggaattcacggcctgatggtgtacaacaagcgggaaggcggaaagaaggacaagcctatcaacgagtggatcatcgccgtgggcaagcatagaggagtcatctcctctgatatctggctgaagtgccaaaatctgatccagcagaacaacgctaagtcctcccctagatccggtactggagagaagtttctgctgtccggcatggtggtgtgtaaggagtgcggctccggcatgagctcctggagccacttcaacaaaaaaaccaacttcatggaaagatactacagatgcaacctgcggaatagagcctccaaccggtgttccaccaagatgctgaatgcctacaaggccgaggaatacgtggccaactacctcaaggaactagatatcaacgccattaaaaagatgtaccactctaacaagaagaacatcatcgactatgacgccaagtatgaggtgaacaagctgaacaagagcatcgaggagaacaagaagatcatccagggcatcatcaagaagatcgctctgttcgacgacctggatatcctgggcatgctgaagaacgaactggagagactgaaaaaagaaaacgacgagatgaagatcaaactgaaagaactgaagtccatcctggaattggaggatgaagaggagatcttcctgtctaccatggaggagaacatctctaacttcaaaaagttctacgacttcgtgaacatcacccagaagcggattctgatcaagggcctggtggaaagtatcgtgtgggacacaggcggtgaggaaaagatcctggagatcaacctgatcggctctaacaccaagctgccttccggcaaggtgaagcgaagagag

SEQ ID NO: 27 - DNA, 1455 bp, synthetic construct
  source 1..1455: mol_type = other DNA
atgtccaaaaaggtggccatctatacaagagtgtccaccaccaaccaggccgaggaaggctactccatcgacgagcagatcgacaagctgaaaatgtactgcgaggccatggactggaaggtgtctgagatctacaccgacgccggcttcactggctccaagctgaccagacctgccatggaaaagatgatcaccgacatcggcctgaagaagttcgataccgtgatcgtgtacaagctggacagactgtccaggtccgtgcgggataccctgtacctggtcaaggatgtgttcaccaagaatgagatcgactttatcagcctgtctgagtctattgacacctcctccgctatgggttctctgttcctgacaatcctgagcgctatcaacgagttcgagagggagaacataaaagaacggatgaccatgggcaagatcggcagagccaagtctggaaagtccatgatgtgggctaagaccgccttcggctactctcacaaccaagagacaggcatcctggaaatcaaccctctggaagcttccatcgtggaacagatcttcaacgagtacctgaagggcacctctatcacaaagctgcgggacaagctgaacgaggatggccacatcgccaaggagctgccttggtcctacagaaccatcagacagaccctggacaaccccgtgtactgtggatacatcaagtacaaaaacaacacctttgagggcctgcacaagcccatcatctcccacgaaacctacctcagcgtgcagaaagaactggaagccagacaacagcagacctatgagaagaacaataatcctagaccatttcaagccaagtatctgctgtctggcatcgctagatgcggatactgtggcgctcctctccggatcgtgctgggccatcgccggaaggacggcagtagaaccatgaagtaccagtgcgtgaacagattccctcgcaaaaccaagggcgtgaccacatacaacgataacaagaagtgcgactccggcgcttacgacatgcagtggatcgaggacatcgtgctgaaaaccctgaacggcttccagaagtccgacaaaaagctgcggaagatcctgaatatcaaggaagagtccaaggtggacaccagcggatttcagaagcagctgaagtccatcaacaataagatccagaagaactccgatctgtacctcaacgacttcatcaccatggacgacctgaaaaagcggaccgagatgctgcagggcgagaagaaactgatccaggccagaatcaacgaagtggataagccttccacatctgagatcttcgacctggtcaagtctgagctgggcgaaaccaccatctctaagatctcctacgaagataagaagaagatcgtcaacaacctgatctctaaagttgacgtgaccgccgacaacatcgatatcatcttcaagttccagctggct

SEQ ID NO: 28 - DNA, 1374 bp, synthetic construct
  source 1..1374: mol_type = other DNA
atgaacgagcaaaaggacaagctgaagaaatactgcgagattaaggactggaccatcgtcaaagagtacgtcgatcctggccggagcggctccaacatcaacagaccatccatgcagcagctcattaaggacgccgataccggcctgtacgacgctgtgctggtgtacaagctggaccggctgtctagatctcagaaggacaccctatatctgatcgaggacgtgttccagaagaacaacatccacttcatctctctgtccgagaacttcgacacctccaccgcctttggaaaggccatgatcggcatcctctccgtgttcgcccagctggaaagagagcagatcaaagagcggatgtctatgggcagagtgggcagagccaaatccggcaaaatcatggaattcaacaaccccgcctttggttacgaggtggatggcgacaactacaaagtggacccactgcgggccgagatcgtgaagagaatctacaagatgtacctgagcggcacctctatcaacaagatcaaggaaaccctgaacctggaaggccacatcggcaacaagaagaactggtccgacaccagaatcagatatatcctgtccaatcccacctacctgggaaagatccggtacgacggcaaaacctacgacggcaagttctcccctatcatcgacgaggaaaccttcaacaagacccagaacgaactgaaagagagacagaccgctacatacaagagattcaacatgaagctacgcccctttcagtctaagtacatgctgtctggcctgctgaggtgcggctactgcggcgctaccctgttcgtgaactcctatgtgtacaacggcaagcggaagctgcgatacaactgtccttctacctacaagtccaagcaaaaaacacggacatacaagatcatggaccccaactgccctttcaagctggtgtacgccaaggatctggaacctgctgtgatcaacgagatcaagaatctggctctgaaccctcagtccatccagaagcctgtgaagaagaaacctgatatcgatgtggaagccatccagaaagagctggccaaggtgcggaagcagcagcagagactgatcgatctgtacgtgatcagcgacgacgtgaatatcgacaatatcagcaagaagtctgccgacctgaagctgcaagaggagacactgaagaagcagctggctcctctggaggagcctaacgacgacgataagatcgtggccttcaatgagattctggctcagatcaaggatatcgactccctggactacgataagcagaagttcatcgtcaagaagctgatcaagaaaatcgacgtgtggaacgacaacaagatcaagatccattggaacatc

SEQ ID NO: 29 - DNA, 1545 bp, synthetic construct
  source 1..1545: mol_type = other DNA
atgaaaaccgccatctacctgagaaagtctagagccgatctggaggccgaggctagaggcgagggcgagacactggccaagcaccggtcgacactcctgaagatcgccaaggagatgaacctgaacgtgctgtctgtgagagaagaaatcgtgtccggcgagtctctggtcaagcggcccgagatgctggctctgctggaagagatcgaggacaacaagtacgacgccgtgctgtgcatggatatggacagactgggaaggggcggcatgaaggaacagggaatcatcctggaaaccttcaagcggtccaacaccaagatcatgacccctagaaagacctacgacctgaacgacgagtgggacgaggagtactctgagtttgaggccttcatggccagaaaagaacttaagatcatcaccagaagaatgcagagaggccggatcgccagcgtggaagctggcaactatctcggcacccacgctccattcggctatgatatccaccggctgaacaaaagagagagaaccctgacaatcaactccgaggaggcctccgtggtgcggatgatcttcgactggtacgccaacgaggacatgggcgccagtgctatccggaacaagctgaacgacttgggctacaagtccaagctgggcaatgactggaacccctactccatcctggatatcctgaagaacaacatctacatcggcaaagtcacctggcagaaacgtaaggaagtgaagcggcctgatgctgtcaagagatcctgtgccagacaggacaagtccgattggatcatcgctgacggcaagcacgagcctatcatccctgagtccctgttcgagcaggcccaagagaagctgaattctcggtaccacgtgccatacaataccaacggcattaagaaccctctggctggaatcattaagtgtagcaagtgcggctactccatggtgcagagataccctaagaatcggaaggaaaccatggactgcaagcatagaggctgcgagaacaagtctagctacaccgagctgatcgagaagcgcctcctggaagctctgaaggaatggtacatcaactacaaggctgactttgaagctcacaagcagggcgacaagctgaaggagacacaagtgatccagatgaacgaggctgccctgcggaagctggaaaaagaactggtggacgtgcagaagcagaagaacaacctgcacgacctgctggagcggggcgtgtacaccgtggacatgttcctggaaagatctcaggtgatctccgaccggatcaacgagatcacctctaccatggaaaacctgaaaaaggagatcaagaccgaaatcaagaaggagaaagtgaagaaggacaccatcccccaggtggagcatgtgctggacctgtacttcaagactgacgatcctaagaaaaagaattctctgctgaagtccgtgctggaaaaggccgtgtacaagaaagaaaaatggcagagactggacgacttcgagctggttctgtaccctaagctgcctcaggatggagacatc

SEQ ID NO: 30 - DNA, 1548 bp, synthetic construct
  source 1..1548: mol_type = other DNA
atgtaccggccagagagcctggacgtgtgcatctatctgcgcaagtctcggaaagatgtggaagaagaacggcgggctattgaagagggctcctcctacaacgccctggaaagacacagaaagagactgttcgccatcgctaaggccgagaaccacaacatcatcgacatcttcgaggaagtggcctctggggagtctatccaagagcggcctcagatgcagcagctgctgcggaagttggaaggcaacgagattgacggagtgctggtcatcgatctggataggctgggcagaggcgatatgctggacgctggcatgattgacagagccttcagatactcctctaccaagatcatcacccctaccgacgtgtacgaccccgacgacgagtcctgggagctcgtgttcggcatcaagagcctgatctccagacaagaactgaagtccatcaccaagaggctgcagaacggccggatcgattccgtgaaagaaggcaagcacatcggtaagaaaccaccttacggctacctgaaggatgagaacctgagactgtaccctgatcctgagaaagcttggatcgtgaagaagatcttcgagctgatgtgcgacggcaaaggcagacagatgatcgccgctgagctggacagactgggcatcgaccctcctgtgaccaagcggggcgcctgggactcttctacaattacctctatcatcaagaacgaggtgtacaccggcgtgatcgtgtggggaaagttcaagcacaagaagcggaacggcaagtacaccagacataagaatcctcaagagaagtggatcatgtacgagaacgctcacgagcctatcatctctaaggaactgttcgacgccgccaacgaggcccattcttcgagacacaagcccgccgtgatcacttccaagaaactgaccaaccccctggccggcatcctgaagtgcaagctgtgtggctacaccatgctgatccagacccggaaggaccggcctcacaactacctgagatgcaacaaccccgcctgtaaaggcaagcagaagcagtctgtgttcaacctggttgaggaaaagctcctgtatagcctgcagcagatcgtggacgagtaccaggctcagaaggtggaagaagtggagatcgacgattccaagctgatctccttcaaggagaaggctatcatctccaaggagaaggaactcaaagaactgcaggcccagaagggcaacctgcacgacctgctggaacagggcatctacacagtcgagatctttctggaaagacagaagaatctggtcgaaagaatcacctccatcgagaacgacatcgaggtgctgcagaaggagatcgagacagagcagatcaaggagcacaacaagaccgagtttatccctgctctgaaaacagtgatcgagagctaccataagaccaccaatatcgagctgaagaatcagctgctgaaaaccatcctgtccaccgtgacctactacagacaccctgactggaaaaccaacgagttcgaaatccaggtgtactttaaaatc

SEQ ID NO: 31 - DNA, 1539 bp, synthetic construct
  source 1..1539: mol_type = other DNA
atgaagtacctggctctgcatgagaactcccggatcgccgtgtacagccggaagtccagagaggacagagactccgaggataccctggccaagcaccggaacgagctggaatacctgatcaagagagaaaacttcaaaaacgtgcagtggttcgagaaggtggtgtccggcgaaaccatcgacgagcggcctatgttctccctgctgctgcctagaattgaaaacggcgagttcgacgctgtgtgtgccgtggccatggaccggctgtctagaggctcccagatcgattctggaagaatcctggaggcctttaagcagtccggcaccctgttcatcacccctaagaaaacctacgacctgtccatcgagggcgacgagatgctgtccgagttcgaatccatcatcgccagatctgagtacagagctatcaagcggagaaccatcaacggcaagaagaatgccacccgcgaaggccggctgcacagcggatccgtgccttatggttacaagtgggacaagaacctgaaagctgctgtcgtggtggaagagaagaagaagatctatcggatgatgattaagtggtttctggaagaagagtactcctgcaccgtgatcgctgagatgctgaatgaactcaaggtgccctccccctcaggcagatctatctggtacggcgaggtggtgtctgagatcctgtccaacgacttccacagaggatacgtgtggttcggcaagtataagaagtccaagagcaacaacagcatcgtgcagaacaagaacctggatgaggttctcatcgccaagggccaccatgaaaccatgaaaaccgatgaggagcacgccctgatcctgaaccggatcgagaagctgcggacctacaaggtggctggcagacggctgaacatgaacacccatagactgtccggcatcgtgcggtgtccttactgccacaaggctcaggccatcgagcaaccaaagggcagacggaagcacgtgagaaagtgcctgagaaagtccgctgagaggaccaaagagtgcgaggaaacaaaaggcatccacgaggaagtgctgtttcagtctatcatgaaagagatcaagaaatacaatgagtctctgttctctcctaccgagcaggacgtgaacgacgactcctacactgcccagctgatcggcctgagggagaaggccgtgaagaaggctaagggccgcatcgagcggatcaaagagatgtacctggacggagacatctccaaaaccgagtacaaggaaaagctgaagatcagccaagagacactgcagaaggctgagaacgaacttgccgaactgatagcctctacagagttccagaacgccctgtctgccgagacaaagaaagagaagtggtcccaccacaaggtgcaggaaatgatcgagagcaccgacggcatgtccaactctgaaatcaacttgatcctgaagatgctgatctctcacgtgacctacaccgtcgaagatctgggcgatggcaccaagaatctgaacatcaaggtgtactacaac

SEQ ID NO: 32 - DNA, 1740 bp, synthetic construct
  source 1..1740: mol_type = other DNA
atggaccctcagcacaagcctacccgggctctgatcgtgatccggctgtcccggctgacagacgaaaccacctctcctgagcggcagctggaggcctgcgagagattctgcgccgcaagaggctgggaggtcgtgggcgtggctgaagatctggacgtgtctgctggaaccaccagccccttcgagcggccttctctgagccagtggatcggcgatggtaaggacaacccaggaagaatcggcgagttcgacaccgtggttttctacagagtggatcggctcgtgcggagagtgcggcacctgcacgacgtgatcgcctggagcgagcgcttcgatgtgaacatggtgtccgccaccgagtctcacttcgacctgtccacaaccattggcgctctgatcgctcagctggtggcctccttcgccgagatggaactggagggcatctctcagagagctacctctgctcacagacacaacgtgcagctgggcaagttcgtgggcggctcccctcctttcggctacatgcctgaagaaacccctgatggctggcggctggtgcacgatcccgacgtcgtgcccatcatcctcgaggtggtggacagagtcctggaaggcgaacccctgagaagaatcaccgacgatctgaacgcccggggcgctacaaccgcccgggacctggtgaagcagagaaagggcaaagaaaccgagggccacaagtggcactccaacgtgctgaagcggcggctgatgtcccctgccatgctgggctacgccctgagaagagaacctctgaccgactccaagggcaagcccaaactgtctgccaagggcgccaagctgtacggccctgaggaaatcgtgagaggacctgacggcctgcctgtgcaacgcgctgagcctatcctgcctaagcctctgttcgaccgggtggtggctgagctagaagctagagagctacagaaagagcctaccaagcggatcaactccatgctgttgagagtgctgtactgcggcgtgtgtggccagcctgtctaccgggcaaaaggacagggcggtagatccgacagataccggtgcagatccatccaggatggcgccaactgtggcaacccctccgtgctgacctatgagctggacgacctggtggaagagtctatcctggtgctgatgggcgactctgagagactggcccatgtgtggaaccctggcgaggacaatgctagcgagctggctgaagtggaagcccggctggccgacagaaccggcctgatcggagtgggagcctacaaggctggcaccccccagagagccaccctggataccctgatcgaggctgatgccaagctgtacgagaggctgaaggccgccacccctagacctgctggctggacctgggaaccaacaggcgaaaccttcgccgagtggtgggctgctctggacaccggcgccagaaatgtgtacctgcggaacatgggggtcagagtcacctacgacaagcggcctgtgccagagcaggtgtccgccggcgagaagcctagagtgcatctggaactgggcgaagtgcggaagatggccgaacaagtggctgtgaccggcaccatcggaacactgaccagaaactacacaagactgggagagatcggcatcacccacgtggacatcgacgccggatctggcaaggccgtgtttgtgacaaagtccggcgagcggttcgagctccctctgaacatccctgaggaa

SEQ ID NO: 33 - DNA, 1620 bp, synthetic construct
  source 1..1620: mol_type = other DNA
atgaaggctatcgccatctacgccagaaagtctctgttcaccggcaagggcgactccattggcgcccaggtggacacctgcaagcggttcatcgactacaagttcgccaatgaggactatgagatccggacatttaaggacgaaggctggtccggcaagaccactgacagaccagattttaccaacatggtgaacctgatcaagtctaagaagatcgactatgtcatcacctacaagctggaccggatcggccggacagctcgggacctgcacaacttcctgtacgagctggataatctgggaatcgtgtacctgagcgccaccgagccttacgacacaaccacatctgccggaagattcatgatcagcattctggctgctatggctcagatggaacgcgaaagactggccgagagagtgaagtccggcatgatccagatcgccaagaagggaagatggctgggcggccagtgtcctctgggcttcgactctaagagagagatctacatcgatgacatggggaaagagcggcagatgatgcggctgacccctaacaaggaggaaatcaagatcgtgaagctgatctacgacaagtacctggagatgggatccatgtcccaagtgcggaagtactgcctggaaaactccatcagaggcaagaacggcggcgacttctccacaaacaccctgaagcagctgctgacctctcctatctacgtcaagtcctccgacaacatcttcaagtacctggagtctcagaatatcaatgtgttcggcacccccaacggcaacggcatgctgaccttcaacaagaccaaagagatcaggatcgagcgggacaagtccgagtggattgctgctgtgggcaagcacaagggcatcatcgacgataacaagtggctgcagatccagcagcagctgcagcagcagtctgaaaagcagatcaagagctctggcagacagggcacgacctccaccggcctgctgtccggcatcatcaagtgctccaagtgcggcaacaacctgctgatcaagaccggacacaagtccaagaaaaaccctggcaccacctactcctactacgtgtgtggcaagaaggataactcttacggccataagtgcgacaacaaaaacgtgagaaccgacgaggccgactccgccgtgatcacccagctgaaactgtacaacaaagaactgctcatcaaaaatctcaaggaagccctgatccaaaacgaaaagaccgataccgacaacatcgagatcctggagtccaaattaaaagaaaaagagaaggccgtgtccaacctggtgaaaaagctgtctctgatcgacgacgagtccatcagcaatatcatcctgaacgaggttaccaatatcaacaaggaaatcaacgacatcaagctgcaattgtctaacgagacactgaagatcaacgaagtgaccaaggccacactggataccgagatctacatcaagatcctggagaactttaacaagaagatcgacgatatcaccgaccccatcgaaaagatgaacttgctgaagtctgctctggaatccgtggaatggaacggcgattctggcgagttcaagatcaacctgatcggcagcaaaaagaaa

SEQ ID NO: 34 - DNA, 1380 bp, synthetic construct
  source 1..1380: mol_type = other DNA
atgaaggttgctatctacaccagagtgtccaccctggagcagcgggaaaagggacactctatcgacgagcaagagcggaaactgagatctttctgcgacattaacgactggaccgtgaaagatgtgtacgtggatgctggcttctccggagccaagcgggacagacctgagctgaccagactcctggacgacatctccgagttcgacctggtgctggtctacaagctggaccggctgacaagaagcgtcagagatctgctggacctgctggaagtgttcgagaacaataacgtggccttcagatctgctaccgaggtgtacgacaccaccaccgccatcggcagactgttcgtgacactcgtgggcgccatggccgagtgggagagagagacaatccgggaaagaagcctgatgggcaagagagccgctattaagaagggcatgatcctgaccgctccacccttctactacgacagggtgaacaacacctacatccctaaccagtataaggatgtggtcctcgatgtgtacaacaaggtcaagaaaggctactccatcgctcatatcgccagactgtacaacaactccgacgtgaagcctcctaacggcaacgaggaatggaccacccggatgctgatgcacgccctgagaaaccctgtgacccggggccactaccagtggggcgagatctacatcgaggactctcatgagcctatcatcacagacgagatgtacaatacaatcatcgaccgcctggacaagcacaccaacaccaaggtggtggcccacacctccgtgtttcggggcaagctgatctgccccaactgtggctacgctctgaccctgaacagccagaagagaaagcggaagaacgacaccatcgtgtacaagacctattactgcaataactgcaagatcaccaagggcatgaagcctcaccacatcaccgagacagaaaccctgcgggtgttcaaggaccacctgtccaagatcgacctgaaacagtacgaaacccaagagaaagagaagcagtctcacgtgaccatcgatctgtctaaagtgatggaacagagaaagagataccacaagctgtacgcctctggcatgatgcaggaaaacgagctgtttgaactgatcaaggaaaccgacgaaatgatcgaagagtacgagaagcagcgcaagcaggtggacgtgaaagagttcgacatctgtaagatcaaagaaatcaaggatgtgctgctgaagtcctgggacatcttcaccctggaagataaggccgacttcatccagatgtccatcaaggctatcaacatcgagtataccaagctgaagcggggaaagagctctaattccatgaagatcaaggatatcgagttttac

SEQ ID NO: 35 - DNA, 1029 bp, synthetic construct
  source 1..1029: mol_type = other DNA
atgtccaatctgctgaccgtgcaccagaacctgcctgctctgcccgtggacgccaccagcgacgaggtgcgcaagaacctgatggacatgttccgcgaccgccaggccttcagcgagcacacctggaagatgctgctgagcgtgtgccgcagctgggccgcctggtgcaagctgaacaaccgcaagtggttccccgccgagcccgaggacgtgcgcgactacctgctgtacctgcaggcccgcggcctggccgtgaaaaccatccagcagcacctgggccagctgaacatgctgcaccgccgcagcggcctgcctaggccatctgactctaatgccgtgtctctggtcatgcggcggatccggaaagaaaacgtggacgccggcgagagagctaagcaggctctggctttcgagagaaccgacttcgaccaagtgcggtccctgatggaaaactccgaccggtgccaggatatccggaacctggcttttctgggaatcgcctacaacaccctgctgcggatcgctgagatcgcccggatcagagtgaaggacatctctagaaccgacggcggcagaatgctgatccacatcggcagaacaaagaccctggtgtccacagctggcgtggaaaaggctctgtctctgggcgtgaccaagctggtggaacggtggatttctgtgtccggcgtggccgacgatcccaacaactacctgttctgcagagtccggaagaacggcgtggcagccccttctgctacatcccagctgtctacaagagccctggaaggcatcttcgaggctacccacagactgatctacggcgccaaggacgatagcggccagagatatttggcttggagcggccactccgctagagtgggagctgctagagatatggctagagccggcgtgtccattcctgagatcatgcaagctggcggctggaccaacgtgaacatcgtgatgaactacatccgcaacctggactccgagacaggcgctatggttcgactgctggaagatggcgac

SEQ ID NO: 36 - DNA, 1296 bp, synthetic construct
  source 1..1296: mol_type = other DNA
atggccccaaagaagaagcggaaagtgatgtctcagttcgacatcctgtgcaagacccctcctaaggttctggtcagacagttcgtggaacggttcgagcggccttctggcgaaaagatcgcttcctgtgccgctgagctgacctacctgtgctggatgatcacccacaacggcaccgccatcaaacgggccaccttcatgtcctacaacaccatcatctccaactccctgtccttcgacatcgtgaacaagtctctgcagttcaagtacaagacacagaaggctaccatcctggaagcttccctcaagaagctgatccctgcttgggagttcacaatcatcccctataatggccaaaagcaccagtctgatattacagatatcgtgtccagcctgcagctgcagtttgagtcctccgaggaggccgacaagggcaatagtcactccaagaagatgctgaaagccctgctgtctgaaggcgagtccatctgggaaatcaccgagaagatcctgaactccttcgagtacaccagcagattcaccaaaaccaagaccctgtaccagttcctcttcctggctaccttcatcaactgcggcagattcagcgatatcaagaatgtggaccccaagagcttcaagctggtgcagaacaagtacctgggcgtgatcatccagtgcctggtgaccgaaaccaagacctctgtgagcaggcacatctacttcttttctgctagaggcagaatcgacccactggtgtacctggacgagttcctgagaaactccgagcctgtgctgaagagagtgaacagaaccggcaactcttcttccaacaagcaggagtatcaactgctgaaagacaacctggtgcggtcctacaacaaggctctgaagaaaaatgctccttaccctatcttcgccatcaagaacggacctaagagccatatcggcagacacctgatgacctcctttctgtccatgaaaggcctgacagaactgaccaacgtggtgggcaactggtccgacaagagagcctccgccgtggcccggaccacctacactcatcagatcacagccatccctgatcactactttgccctggtgtccagatactacgcctacgaccctatctccaaagagatgatcgccttgaaggacgaaaccaaccccatcgaggaatggcagcacatcgagcagctgaagggatctgcggagggctccatccggtaccccgcttggaacggcatcatcagccaagaggtgctggactacctgtcctcttacatcaaccgccggatc

SEQ ID NO: 37 - DNA, 1527 bp, synthetic construct
  source 1..1527: mol_type = other DNA
atgagagccctggtggtcatccggctgtctagagtgaccgacgctaccacctctcctgagcggcagctggaatcttgccagcagctgtgtgctcagcgcggatgggatgttgtgggagtcgctgaggacctggatgtgtctggtgccgtggatcctttcgaccggaagcggaggcctaacctggctagatggctggcctttgaggaacagcccttcgacgtgatcgtggcctacagagtggaccggctgacccggtctatcagacatctgcagcagctggtccactgggccgaagatcacaagaaactggtggtgtccgccaccgaggctcacttcgataccaccacaccttttgccgccgtcgtgatcgctctgatgggaaccgttgctcagatggaactggaagccatcaaagagcggaacagatccgccgctcacttcaacatcagagccggcaagtaccggggctctttgcctccttggggctacctgccaacaagagtggatggcgaatggcggctggtgcctgatcctgtgcagcgggaaagaatcctggaagtgtaccacagagtggtggacaaccacgagcctctgcacctggtggcccacgacttgaatagaagaggcgtgctgtcccctaaggactacttcgcccagctgcagggcagagagcctcagggaagagagtggagcgctaccgctctgaagcggtccatgatctctgaggccatgctgggctacgctaccctgaatggaaagaccgtgcgggacgatgatggcgcccctcttgttagagccgagcctatcctgaccagagagcagctcgaagccctgagagctgagctggtcaagacctccagagccaagcctgctgtgtctacccctagcctgctgctgagagtgctgttctgtgctgtgtgtggcgagcccgcctacaagtttgctggcggcggaagaaagcaccccagataccggtgtcggtccatgggcttccctaagcactgtggcaatggcaccgtggccatggctgagtgggatgccttctgcgaagaacaggtgctggatctgctgggcgacgccgagagactggaaaaagtgtgggtggccggctccgactctgctgtggaactggctgaagtgaacgccgagctggtggacctgacctctctgatcggctctcccgcttatagagctggctcccctcagagagaagccctggacgctagaatcgctgccctggctgctagacaagaggaactcgaaggcctggaagctcggccttcaggatgggagtggcgagagacaggccagagatttggcgactggtggcgcgagcaagataccgccgctaagaacacctggctgcggtctatgaatgtgcggctgaccttcgatgtgcgcggaggactgaccagaaccatcgacttcggcgacctgcaagagtacgagcagcatctgagactgggctccgtggtggaaagactgcacaccggcatgtccggttcaccaaagaaaaagcggaaagtg

SEQ ID NO: 38 - DNA, 1500 bp, synthetic construct
  source 1..1500: mol_type = other DNA
atgagagcactggtggtcatccgactgagtagggtcacagacgcaacaacaagccccgagaggcagctggaatcatgtcagcagctgtgcgcacagcgaggatgggacgtggtcggagtggcagaggatctggacgtgagcggcgctgtcgatccattcgacagaaagcggaggcccaacctggcaaggtggctggctttcgaggaacagccctttgatgtgatcgtcgcctacagagtggacaggctgacacgctctattcgacatctgcagcagctggtgcattgggccgaggaccacaagaaactggtggtcagtgcaactgaagcccacttcgataccacaactccttttgccgctgtggtcatcgcactgatgggcaccgtggcccagatggagctggaagctatcaaggagcgaaaccggagtgcagcccatttcaatattcgggccgggaaatacagaggatcactgcccccttggggctatctgcctacccgggtggatggggagtggagactggtgccagaccccgtccagagagagaggattctggaagtgtaccacagggtggtcgataaccacgaaccactgcatctggtcgcccacgacctgaataggcgcggcgtgctgagcccaaaagattattttgctcagctgcagggaagggagccacagggacgagaatggtccgctaccgccctgaagcggagcatgatcagtgaggctatgctgggctacgcaactctgaatgggaaaaccgtccgggacgatgacggagcaccactggtgagggctgagcctattctgacacgcgagcagctggaagctctgcgggcagaactggtgaaaacctccagagccaaacctgccgtgagcaccccaagcctgctgctgagggtgctgttctgcgccgtctgtggggagccagcatacaagtttgccggcgggggaagaaaacatccccgctatcgatgccggtctatgggattccctaagcactgtggaaacggcactgtggctatggccgagtgggacgccttttgtgaggaacaggtgctggatctgctgggagacgccgagaggctggaaaaagtgtgggtcgctggcagcgactccgctgtggagctggcagaagtcaatgccgagctggtggatctgacctccctgatcggatctcctgcatatagggcaggctcaccacagcgagaagctctggacgcacgaattgctgcactggcagctcgacaggaggaactggaggggctggaagcacgacctagcggatgggagtggcgagaaacaggccagcggtttggggattggtggagagagcaggacacagcagccaagaacacttggctgagaagtatgaatgtcaggctgactttcgatgtgcgcggcgggctgacccgaacaatcgattttggcgacctgcaggagtatgaacagcacctgagactggggagcgtggtcgaaagactgcacactgggatgtca

SEQ ID NO: 39 - protein, 582 aa, synthetic construct
MTNPASRPKAYSYIRMSSAIQIKGDSFRRQAEASAKYAAEHDLDLIDDYKLADLGVSAFKSDNLTTGALGRFVAECEAGEIEAGSFLLIESLDRLSRDKILDAFSLFARILKTGVKIVTLSDGQVYDGSSDQVGSIYYAISVMIRSNDESKIKSTRGLANWSQKRKLAAEHGVKMSSQCPAWLKLSVDRKSYLIDKERAKIVQRIFEASASGKGANLITKELNRDKVPTFGRGALWAEAFVSKTLRNRAVLGEFQPGQYVSGKRQPAGDPIPGYFPPVIEEELFDIVQASLRGRLLAGGRRGEGQSNIFTHVAFCGYCGSKMRHRSKGSRVKGNPPHRYLTCFNRFNGPGCDCKPLPYAAFERSFLTFVRDVDLRGLLEGAKRKSEAKTIADRITVNEEKVRKADERIRDYLIKIEGAPDLAEIFMERIRELKAEKDDLVRSIEESNDALSKIKSDNVTDEELASLISTFQNPCGENRIRLADRIKSIIERIDVYPNGEIRKDDPAIDLVRASGDPDAEKIIAAMNAGSRLKDDPYFIVTFRNGAVQTVVPNPSNPDDIRVSVYAGEKTRRVEGSAYEYESD

SEQ ID NO: 40 - protein, 509 aa, synthetic construct
MPIAPEFLSLAYPGQEFPAYLYGRASRDPKRKGRSVQSQLDEGRATCLDAGWPIAGEFKDVDRSASAYARRTRDEFEEMIAGIQAGECRILVAFEASRYYRDLEAYVRLRRVCREAGVLLCYNGQVYDLSKSADRKATAQDAVNAEGEADDIRERNLRTTRLNAKRGGAHGPVPDGYKRRYDPDSGDLVDQIPHPDRAGLITEIFRRAAAAEPLAAICRDLNERGETTHRGKAWQRHHLHAILRNPAYIGHRRHLGVDTGKGMWAPICDDEDFAETFQAVQEILSLPGRQLSPGPEAQHLQTGIALCGEHPDEPPLRSVTVRGRTNYNCSTRYDVAMREDRMDAFVEESVITWLASDEAVAAFEDNTDDERTRKARIRLKVLEEQLEAAQKQARTLRPDGMGMLLSIDSLAGLEAELTPQIDKARQESRSLHVPALLRDLLGKPRADVDRAWNEALTLPQRRMILRMVVTIRLFKAGSRGVRAIEPGRITLSYVGEPGFKPVGGNRAKQ

SEQ ID NO: 41 - protein, 471 aa, synthetic construct
MRKVAIYSRVSTINQAEEGYSIQGQIEALTKYCEAMEWKIYKNYSDAGFSGGKLERPAITELIEDGKNNKFDTILVYKLDRLSRNVKDTLYLVKDVFTANNIHFVSLKENIDTSSAMGNLFLTLLSAIAEFEREQIKERMQFGVMNRAKSGKTTAWKTPPYGYRYNKDEKTLSVNELEAANVRQMFDMIISGCSIMSITNYARDNFVGNTWTHVKVKRILENETYKGLVKYREQTFSGDHQAIIDEKTYNKAQIALAHRTDTKTNTRPFQGKYMLSHIAKCGYCGAPLKVCTGRAKNDGTRRQTYVCVNKTESLARRSVNNYNNQKICNTGRYEKKHIEKYVIDVLYKLQHDKEYLKKIKKDDNIIDITPLKKEIEIIDKKINRLNDLYINDLIDLPKLKKDIEELNHLKDDYNKAIKLNYLDKKNEDSLGMLMDNLDIRKSSYDVQSRIVKQLIDRVEVTMDNIDIIFKF

SEQ ID NO: 42 - protein, 477 aa, synthetic construct
MITTRKVAIYVRVSTTNQAEEGYSIQGQIDSLIKYCEAMGWIIYEEYTDAGFSGGKIDRPAMSKLITDAKHKRFDTILVYKLDRLSRSVRDTLYLVKDVFNQNNIHFVSLQENIDTSSAMGNLFLTLLSAIAEFEREQITERMTMGKIGRAKSGKTMAWTYTPFGYDYNKEKGELILDPAKAPIVKMIYTDYLKGMSIQKIVDKLNKMDYNGKDCTWFPHGVKHLLDNPVYYGMTRYNNKLFPGNHQPIITKELFDKTQRERQRRRLGIEENHYTIPFQAKYMLSKFLRCRQCGSRMGLELGRPRKKEGKRSKKYYCLNSRPKRTASCDTPLYDAETLEDYVLHEIAKIQKDPSIASRQKHIEDHELKYKRERIEANINKTVNQLSKLNNLYLNDLITLEDLKTQTNTLIAKKRLLENELDKTCDNDDELDRQETIADFLALPDVWTMDYEGQKYAVELLVQRVKVDRDNIDIHWTF

SEQ ID NO: 43 - protein, 552 aa, synthetic construct
MPGMTTETGPDPAGLIDLFCRKSKAVKSRANGAGQRRKQEISIAAQETLGRKVAALLGMQVRHVWKEVGSASRFRKGKARDDQSKALKALESGEVGALWCYRLDRWDRGGAGAILKIIEPEDGMPRRLLFGWDEDTGRPVLDSTNKRDRGELIRRAEEAREEAEKLSERVRDTKAHQRENGEWVNARAPYGLRVVLVTVSDEEGDEYDERKLAADDEDAGGPDGLTKAEAARLVFTLPVTDRLSYAGTAHAMNTREIPSPTGGPWIAVTVRDMIQNPAYAGWQTTGRQDGKQRRLTFYNGEGKRVSVMHGPPLVTDEEQEAAKAAVKGEDGVGVPLDGSDHDTRRKHLLSGRMRCPGCGGSCSYSGNGYRCWRSSVKGGCPAPTYVARKSVEEYVAFRWAAKLAASEPDDPFVIAVADRWAALTHPQASEDEKYAKAAVREAEKNLGRLLRDRQNGVYDGPAEQFFAPAYQEALSTLQAAKDAVSESSASAAVDVSWIVDSSDYEELWLRATPTMRNAIIDTCIDEIWVAKGQRGRPFDGDERVKIKWAART

SEQ ID NO: 44 - protein, 519 aa, synthetic construct
MQLDATLTLRDEGLSAFHQRHIKQGALGVFLRAIEDGRIQPGSVLIVEGLDRLSRAEPIQAQAQLAQIINAGITVVTASDGREYNRERLKAQPMDLVYSLLVMIRAHEESDTKSKRVKAAIRRQCEGWVAGTWRGIIRNGKDPHWVRLGEHGKFEHVPERVLAVRTMIDLFLEGHGAIEITRRLTEQNLYVSNAGNYSVHMYRIVRNQALIGEKRISVDGEEFRLDGYYPPILTREEFAELQQTMSERGRRKGKGEIPNIITGLSITVCGYCGRAMTTQNSKARAPKGKSVVRRLSCPMNSFNEGCPIGGSCESEIVERALMRYCSDQFNLSRLLEGDDGTARRTAQLAVARQRASDIEAQIQRVTDALLSDDGKAPAAFTRRARELETQLEEQRREIEALEHQIAASSAHGIPAAAEAWAQLVDGVLALDYDARMKARQLVADTFRKIVVYQRGFAPIDDAAADRWKRSGTIGLMLVTKRGGMRLLNVDRRTGCWQAEDDLDPSLIPSDGLPMLPLDA

SEQ ID NO: 45 - protein, 462 aa, synthetic construct
MKVAIYVRVSTDEQAKEGFSIPAQRERLRAFCASQGWEIVQEYIEEGWSAKDLDRPQMQRLLKDIKKGNIDIVLVYRLDRLTRSVLDLYLLLQTFEKYNVAFRSATEVYDTSTAMGRLFITLVAALAQWERENLAERVKFGIEQMIDEGKKPGGHSPYGYKFDKDFNCTIIEEEADVVRMIYRMYCDGYGYRSIADRLNELMVKPRIAKEWNHNSVRDILTNDIYIGTYRWGDKVVPNNHPPIISETLFKKAQKEKEKRGVDRKRVGKFLFTGLLQCGNCGGHKMQGHFDKREQKTYYRCTKCHRITNEKNILEPLLDEIQLLITSKEYFMSKFSDRYDQQEVVDVSALTKELEKIKRQKEKWYDLYMDDRNPIPKEELFAKINELNKKEEEIYSKLSEVEEDKEPVEEKYNRLSKMIDFKQQFEQANDFTKKELLFSIFEKIVIYREKGKLKKITLDYTLK

SEQ ID NO: 46 - protein, 461 aa, synthetic construct
MKVAVYCRVSTLEQKEHGHSIEEQERKLKSFCDINDWTVYDTYIDAGYSGAKRDRPELQRLMNDINKFDLVLVYKLDRLTRNVRDLLDLLEIFEKNDVSFRSATEVYDTTTAMGRLFVTLVGAMAEWERETIRERTQMGKLAALRKGIMLTTPPFYYDRVDNKFVPNKYKDVILWAYDEAMKGQSAKAIARKLNNSDIPPPNNTQWQGRTITHALRNPFTRGHFDWGGVHIENNHEPIITDEMYEKVKDRLNERVNTKKVRHTSIFRGKLVCPVCNARLTLNSHKKKSNSGYIFVKQYYCNNCKVTPNLKPVYIKEKEVIKVFYNYLKRFDLEKYEVTQKQNEPEITIDINKVMEQRKRYHKLYASGLMQEDELFDLIKETDQTIAEYEKQNENREVKQYDIEDIKQYKDLLLEMWDISSDEDKEDFIKMAIKNIYFEYIIGTGNTSRKRNSLKITSIEFY

SEQ ID NO: 47 - protein, 461 aa, synthetic construct
MKVAIYTRVSTLEQKEKGHSIEEQERKLRAYSDINDWKIHKVYTDAGYSGAKKDRPALQEMLNEIDNFDLVLVYKLDRLTRSVKDLLEILELFENKNVLFRSATEVYDTTSAMGRLFVTLVGAMAEWERTTIQERTAMGRRASARKGLAKTVPPFYYDRVNDKFVPNEYKKVLRFAVEEAKKGTSLREITIKLNNSKYKAPLGKNWHRSVIGNALTSPVARGHLVFGDIFVENTHEAIISEEEYEEIKLRISEKTNSTIVKHNAIFRSKLLCPNCNQKLTLNTVKHTPKNKEVWYSKLYFCSNCKNTKNKNACNIDEGEVLKQFYNYLKQFDLTSYKIENQPKEIEDVGIDIEKLRKERARCQTLFIEGMMDKDEAFPIISRIDKEIHEYEKRKDNDKGKTFNYEKIKNFKYSLLNGWELMEDELKTEFIKMAIKNIHFEYVKGIKGKRQNSLKITGIEFY

SEQ ID NO: 48 - protein, 481 aa, synthetic construct
MITTNKVAIYVRVSTTNQVEEGYSIDEQKDKLSSYCDIKDWNVYKVYTDGGFSGSNTDRPALESLIKDAKKRKFDTVLVYKLDRLSRSQKDTLHLIEDVFIKNGIEFLSLQENFDTSTPFGKAMIGLLSVFAQLEREQIKERMQLGKLGRAKSGKSMMWAKTSYGYDYHKETGTVTINPAQALTIKFIFESYLRGRSITKLRDDLNEKYPKHVPWSYRAVRTILDNPVYCGFNQYKGEIYPGNHEPIISKEEYDKTQSELKIRQRTAAENVNPRPFQAKYILSGIAQCGYCGAPLKIMLGVKRKDGSRLKKYECHQRHPRTLRGVTTYNDNKKCDSGFYYKDKLEAYVLKEISKLQDDADYLDKIFSGDNAETIDRESYKKQIEELSKKLSRLNDLYIDDRITLEELQSKSAEFISMRGTLETELENDPALRKNKRKADMRKLLNAEKVFSMDYESQKVLVRRLINKVKVTAEDIVINWKI

SEQ ID NO: 49 - protein, 447 aa, synthetic construct
MLRCAIYIRVSTEEQAMHGLSMDAQKADLTDYAKKHNYEIIDYYVDSGKTARKRLSKRKDLQRMIEDVKLNKIDIIIFTKLDRWFRNVRDYYKIQEVLEDHNVDWKTIFENYDTSTANGRLHINIMLSVAQDEADRTSERIKRVFENKLKNNEPTSGSLPIGYKIKEKSIIIDEEKAPIAKDVFDFYYYHQSQTKVFKEILNKYNLSLCEKTIRRMLENKLYIGIYREHENFCPPLIDKNKFDEVQLILKRRNIKYIPTKRIFLFTSLLICKECRHKMIGNAQIRNTKAGKIEYILYRCNQSYARHTCNHRKVIYENKIETYLLNNIESELKKFIYDYELEDIPKVKNKVNKTNIKRKLEKLKELYINDLIDIDMYKEDYKKYTEILNTKEEKIEQRNLQPLKDFLNSDFKSLYSSISREEKRLLWRGIISEIQIDCNNDITIIPHP

SEQ ID NO: 50 - protein, 460 aa, synthetic construct
MKVAIYTRVSSAEQANEGYSIHEQKKKLISYCEIHDWNEYKVFTDAGISGGSMKRPALQKLMKHLSSFDLVLVYKLDRLTRNVRDLLDMLEEFEQYNVSFKSATEVFDTTSAIGKLFITMVGAMAEWERETIRERSLFGSRAAVREGNYIREAPFCYDNIEGKLHPNEYAKVIDLIVSMFKKGISANEIARRLNSSKVHVPNKKSWNRNSLIRLMRSPVLRGHTKYGDMLIENTHEPVLSEHDYNAINNAISSKTHKSKVKHHAIFRGALVCPQCNRRLHLYAGTVKDRKGYKYDVRRYKCETCSKNKDVKNVSFNESEVENKFVNLLKSYELNKFHIRKVEPVKKIEYDIDKINKQKINYTRSWSLGYIEDDEYFELMEEINATKKMIEEQTTENKQSVSKEQIQSINNFILKGWEELTIKDKEELILSTVDKIEFNFIPKDKKHKTNTLDINNIHFKF

SEQ ID NO: 51 - protein, 474 aa, synthetic construct
MAVGIYIRVSTQEQASEGHSIESQKKKLASYCEIQGWDDYRFYIEEGISGKNTNRPKLKLLMEHIEKGKINILLVYRLDRLTRSVIDLHKLLNFLQEHGCAFKSATETYDTTTANGRMSMGIVSLLAQWETENMSERIKLNLEHKVLVEGERVGAIPYGFDLSDDEKLVKNEKSAILLDMVERVENGWSVNRIVNYLNLTNNDRNWSPNGVLRLLRNPALYGATRWNDKIAENTHEGIISKERFNRLQQILADRSIHHRRDVKGTYIFQGVLRCPVCDQTLSVNRFIKKRKDGTEYCGVLYRCQPCIKQNKYNLAIGEARFLKALNEYMSTVEFQTVEDEVIPKKSEREMLESQLQQIARKREKYQKAWASDLMSDDEFEKLMVETRETYDECKQKLESCEDPIKIDETYLKEIVYMFHQTFNDLESEKQKEFISKFIRTIRYTVKEQQPIRPDKSKTGKGKQKVIITEVEFYQ

SEQ ID NO: 52 - protein, 471 aa, synthetic construct
MTVGIYIRVSTEEQVKEGFSISAQKEKLKAYCTAQGWEDFKFYVDEGKSAKDMHRPLLQEMISHIKKGLIDTVLVYKLDRLTRSVVDLHNLLSIFDEFNCAFKSATEVYDTSSAMGRFFITIISSVAQFERENTSERVSFGMAEKVRQGEYIPLAPFGYTKGTDGKLIVNKIEKEIFLQVVEMVSTGYSLRQTCEYLTNIGLKTRRSNDVWKVSTLIWMLKNPAVYGAIKWNNEIYENTHEPLIDKATFNKVAKILSIRSKSTTSRRGHVHHIFKNRLICPACGKRLSGLRTKYINKNKETFYNNNYRCATCKEHRRPAVQISEQKIEKAFIDYISNYTLNKANISSKKLDNNLRKQEMIQKEIISLQRKREKFQKAWAADLMNDDEFSKLMIDTKMEIDAAEDRKKEYDVSLFVSPEDIAKRNNILRELKINWTSLSPTEKTDFISMFIEGIEYVKDDENKAVITKISFL

SEQ ID NO: 53 - protein, 452 aa, synthetic construct
MKAAIYIRVSTQEQIENYSIQAQTEKLTALCRSKDWDVYDIFIDGGYSGSNMNRPALNEMLSKLHEIDAVVVYRLDRLSRSQRDTITLIEEYFLKNNVEFVSLSETLDTSSPFGRAMIGILSVFAQLERETIRDRMVMGKIKRIEAGLPLTTAKGRTFGYDVIDTKLYINEEEAKQLQMIYDIFEEEKSITTLQKRLKKLGFKVKSYSSYNNWLTNDLYCGYVSYADKVHTKGVHEPIISEEQFYRVQEIFSRMGKNPNMNRDSASLLNNLVVCGKCGLGFVHRRKDTISRGKKYHYRYYSCKTYKHTHELEKCGNKIWRADKLEELIIDRVNNYSFASRNVDKEDELDNLNEKLKTEHKKKKRLFDLYISGSYEVSELDAMMADIDAQINYYEAQIEANEELKKNKKIQENLADLATVDFDSLEFREKQLYLKSLINKIYIDGEQVTIEWL

SEQ ID NO: 54 - protein, 470 aa, synthetic construct
MKGESELDKKAAIYIRVSTQEQATEGYSIQAQTDRLIKYVEAKDFILYKKYIDAGYSASKLERPAMQDLIQDVQSKKVDVVIVYKLDRLSRSQKDTMYLIEDIFRPNDVELISMQESFDTSTAFGSATVGMLSVFAQLERKSISERMITGRVERAKKGFYHTGGQDRPPAGYQFNSDNQLIINEYEAAAIKDLFRLYNDGLGKSSISEYLKKNYPGKNKWLPSSIDRMLKNSLYIGKVKFSGAEYDGIHEPIIDEVTFYKTQKEIARRKQTNTKRYNYVALLGGLCECGICGAKMANRRAVGRKGKVYRYYRCYSKKGSPKHMMKTDGCSSKAQQQFIIDEAVINNLKNIDVEAELKRRSAPQTNTSLISSQIESIDKQINKLIDLFQVDSMPLDVISEKIDKLNKEKQSMEKLLERKNKLDKTELQHRFDVLKSFDWDNSSIESKRVVIEMLVQKVIIHDNSIEIILVE

SEQ ID NO: 55 - protein, 512 aa, synthetic construct
MRTNEHNFHNIEEEIKHVAVYLRLSRGEDESELDNHKTRLLNRCELNNWSYELYKEIGSGSTIDDRPVMQKLLTDVEKNLYDAVLVVDLDRLSRGNGTDNDRILYSMKVSETLIVVESPYQVLDANNESDEEIILFKGFFARFEFKQINKRMREGKKLAQSRGQWVNSVTPYGYIVNKTTKKLTPSEEEAKVVIMIKDFFFEGKSTSDIAWELNKRKIKPRRATEWRSSSIANILQNEVYVGNIVYNKSVGNKKPSKSKTRVTTPYRRLPEEEWRRVYNAHQPLYSKEEFDRIKQYFECNVKSHKGSEVRTYALTGLCKTPDGKTMRVTQGKKGTDDDLYLFPKKNKHGDSSIYKGISYNVVYETLKEVILQVKDYLDSVLDQNENKDLVEELKEELMKKEDELETIQKAKNRIVQGFLIGLYDEQDSIELKVEKEKEIDEKEKEIEAIKMKIDNAKTVNNSIKKTKIERLLSDVQSAESEKEINRFYKTLIKEIIVDRTDENEAKIKVNFL

SEQ ID NO: 56 - protein, 475 aa, synthetic construct
MITTNKVAIYVRVSTTNQVEEGYSIDEQKDKLEAYCKIKDWKIYDVYVDGGFSGANTQRPELERLISDVKRKKVDIVLVYKLDRLSRSQKDTLFLIEDVFAKNDVAFISLQENFDTSTPFGKASIGMLSVFAQLEREQIKERMMLGKEGRAKNGKSMSWTTIAFGYDYSKETGVLSVNPTQALIVNRIFTEYLNGKPVVKIIRDLNAEGHVGRKRPWGETITKYLLKNETYLGKVKYKDKVYEGQHEPIITQELFDLVQLEVERRQISAYEKYNNPRPFRAKYMLSGLMKCGYCGASLGLRYTRKDKNGISHHKYQCRNRHSKDLEKRCESGWYSKEELERGVIKELERIKFDPKYKNETLAKKEETIKVEEIKKQLERINNQVSKLTELYLDEIITRKELDEKNDKIKTERQFLEEQLENQKSNVLSIRKRKLTRLLKDFDVEKLSYEDASKIVKNIIKEIIVTKDGMSITLDF

SEQ ID NO: 57 - protein, 462 aa, synthetic construct
MGKSITVIPAKKVQTSVLHQDRKKIKVAAYCRVSTDQEEQLSSYENQVNYYREFISKHEDYELVDIYADEGISATNTKKRDAFNRLIQDCRAGKVDRILVKSISRFARNTLDCIKYVRELKELGVGVTFEKENIDSLDSKGEVLLTILSSLAQDESRSISENATWGIRKKFERGEVRVNTTKFMGYDKDENGRLIINPQQAETVKFIYEKFLEGYSPESIAKYLNDNEIPGWTGKANWYPSAIQKMLQNEKYKGDALLQKTFTVDFLTKKRVQNDGQVNQYYVENSHEAIIDEETWETVQLEMARRKTYRDEHQLKSYIMQSEDNPFTTKVFCGACGSAFGRKNWATSRGKRKVWQCNNRYRIKGVEGCYSSHLDEATLEQIFLKALELLSENIDLLDGKWEKILAENRLLDKHYSMALSDLLRQEQIDFNPSDMCRVLDHIRIGLDGEITVCLLEGTEVDL

SEQ ID NO: 58 - protein, 411 aa, synthetic construct
MRTVRRIQPIKSPCKPRFKVAAYARVSDSRLHHSLSTQISYYNRLIQAHPDWELVGIYYDEGISGKEQSNRQGFLNLIKDCEDGKIDRIITKSIARFGRNTVELLTTVRQLRLKNIGVTFEKENIDSLSSEGELMLTLLASVAQEESQNLSENIRWRIQKKFEKGIPHTPQDMYGYRWDGEQYQIEPNEAKVIRKVFKWYLDGDSVQQIVDKLNQEQVLTRLGNPFTVASIREFFKQEAYFGRLVLQKTYREAFSRNPKRNKGQRNKYIIENAHEPIVTKEYFDLVLHEKERRNQLMHQESHLNKGIFRDKISCSECGCLMIVKVDSKQVNKTVRYYCRTRNRFGASSCSCRTLGEKRLLASFKSKLGIVPDKEWVENNIKHIEYDFGYRILRVTPVKGRKYLIEIREGRY

SEQ ID NO: 59 - protein, 473 aa, synthetic construct
MRNKVAIYVRVSTASQADEGYSIDEQKSKLEAYCEIKDWKIYDTYIDGGFSGANTQRPELERLISDAKRKKIDIVLVYKLDRLSRSQKDTLFLIEDVFAKNDVAFISLQENFDTSTPFGKASIGMLSVFAQLEREQIKERMMLGKEGRAKNGKSMSWTTIPFGYDYSKETGILSVNPTQALIVKRIFTEYLNGKSVVKIIRDLNAEGHVGRKRPWGETITKYLLKNETYLGKSKYKGKVFEGQHDAIISQELFDLVQLEVEKRQISAFEKYNNPRPFRAKYMLSGLMKCGYCGASLGLYVAPKNKNGVSKYKYQCRHRYHKDKAIRCNSGWYSKDELEKRVIKELERLKFDPKYKKETLAKKDETIKVEDIKKQLERINKQVSKLTELYLDEVITRKDLDEKNAKIKTERQYLEEQLENQKSNVMSIRKRKLSRLLKDFDIEKLSYEEASKIVKSVIKEIVVTKDDMTITLDF

SEQ ID NO: 60 - protein, 463 aa, synthetic construct
MKVATYVRVSTDEQAKEGFSIPAQRERLRAFCESQGWEIVEEYIEEGWSAKDLDRPQMQRLLKDIKKGNIDIVLVYRLDRLTRSVLDLYLLLQTFEKYNVAFRSATEVYDTSTAMGRLFITLVAALAQWERENLAERVKFGIEQMIDEGKKPGGHSPYGYKFDKDFNCTIIEDEANTVRMIYRMYCDGYGYHSIAKRLNELGIKPRIAKEWNHNSVRDILTNDIYIGTYRWGNKVVLNNHPPIISETLFRKVQKEKEKRRVDRTRVGKFLLTGLLYCGNCNGHKMQGTFDKREQKTYYRCLKCNRITNEKNILEPLLDEIQLLITSKEYFMSKFSDQYDQKEEVDVSALKKELEKIKRQKEKWYDLYMDDRNPIPKEDLFAKINELNKKEEEIYNKLNEVEPEDKEPVEEKYNRLSKMIDFKQQFEQANDFTKKELLFSIFEKIVIYREKGKLKKITLDYTLK

SEQ ID NO: 61 - protein, 452 aa, synthetic construct
MLRVALYIRVSTEEQALNGDSIRTQIEALEQYSKENDFNIVGKYIDEGCSATNLKRPNLQRLLRDVEKDKVDLVLMTKIDRLSRGVKNYYKIMETLEKHKCDWKTILENYDSSTAAGRLHINIMLSVAENEAAQTSERIKFVFQDKLRRKEVISGTIPIGYKIENKHLVIDKEKKYIVKAIFDEYEKSGSVRTLIETINNLHGELYSYNKIKNILRNELYIGIYNKRGFYVEDYCEPIISKKQFKQIQRILEKNKKTTPNKNIHYHIFSGLLKCKECGYTLKGNSSNVGEKLYLSYRCSTFYLNKNCVHNVTHNEKHIENYLLTNLKPQLHKHMVKLEAQNEKIRRNKKSNKKDEKKKIMKKLDKIKDLYLEDLIDKETYRKDYEKLQSQLDNITEEQESQIIDTSHIKKFLDIDINEMYSDLSRVERRRFWLSIIDYIEIDNNKNITINFI

SEQ ID NO: 62 - protein, 592 aa, synthetic construct
MKITLLYYIKKFNIYCNRYLSQQINISVDIIGFYQFKNVTNSVTDVLKRGDNLDRICIYLRKSRADEELEKTIGVGETLSKHRKALLKFAKEKKLNIMEIKEEIVSADSIFFRPKMIELLKEVENNQYTGVLVMDIQRLGRGDTEDQGIIARIFKESHTKIITPMKTYDLDDDLDEDYFEFESFMGRKEYKMIKKRMQGGRVRSVEDGNYIATNPPFGYDIHWINKSRTLKFNSKESEIVKLIFKLYTEGNGAGTISNYLNSLGYKTKFGNNFSNSSIIFILKNPVYIGKITWKKKDIRKSKDPHKVKDTRTRDKSEWIIADGKHEPIIDEKIWNKAQEILNNKYHIPYKIANGPANPLAGVVICSKCNSKMVMRKYGKKLPHLICNNKECNNKSARFDYIEKAVLEGLDEYLKNYKVNVKANNKTSDIEPYEQQSNALNKELILLNEQKLKLFDFLEREIYTEEIFLERSKNLDERINTTTLAINKIKKILDNEKKKNNKNDIVKFEKILEGYKKTNDIQKKNELMKSLVFKIEYKKEQHQRNDGLLYIYFLSFCVRCISYLTQFISFFVYPYRILEIYLTFSFFIISYEH

SEQ ID NO: 63 - protein, 504 aa, synthetic construct
MRICMYLRKSRADEELEKTLGEGETLSKHRKALLKFAKEKNLNIVEIKEEIVSGESLFFRPKMLELLKEIENKQYSGVLVMDMQRLGRGNMQDQGIILETFKKSNTKIITPMKTYDLSNDFDEEYSEFEAFMSRKELKMINRRMQGGRVRSVEDGNYIATNAPYGYDIHWINKARTLKPNQKESEIVKLIFKLYIEGNGAGTIAKHLNSLGYKTKFGNSFNNSSIIFILKNPVYIGKITWKKKDIRKSKDPNKVKDTRTRDKSEWIIVDGKHDPIIDQITWKQAQEILNNRYHVPYKLVNGPANPLAGLIICTTCKSKMVMRKLRGTDRILCKNNKCNNISNRFDAVEKSVVESLENYLKAYKVNLPELNKTSNLKLYEQQISTLKKELKILNEQKLKLFDFLERGIYDEDTFLKRSKNLDERIEITNESLSNLNQIIAKENKAIKKEDIIKFEKVLDSYKSTADIRLKNELMKTLIFKIEYTKNKKGNDFKIKVFPKLKPLNI

SEQ ID NO: 64 - protein, 545 aa, synthetic construct
MIAAIYSRKSKFTGKGESVENQIEMCKEYLKRNFNNIDDIEIYEDEGFSGKDTNRPKFKKMIKAAKNKKFNILICYRLDRISRNVADFSNTIEELQKYNIDFISIKEQFDTSTPMGRAMMNIAAVFAQLERETIAERIKDNMVELAKTGRWLGGTSPLGYKSEPIEYSNEDGKSKKMYKLTEVENEMNIVKLIYKLYLEKRGFSSVATYLCKNKYKGKNGGEFSRETARQIVINPVYCISDKTIFKWFKSKGATTYGTPDGIHGLMVYNKREGGKKDKPINEWIIAVGKHRGVISSDIWLKCQNLIQQNNAKSSPRSGTGEKFLLSGMVVCKECGSGMSSWSHFNKKTNFMERYYRCNLRNRASNRCSTKMLNAYKAEEYVANYLKELDINAIKKMYHSNKKNIIDYDAKYEVNKLNKSIEENKKIIQGIIKKIALFDDLDILGMLKNELERLKKENDEMKIKLKELKSILELEDEEEIFLSTMEENISNFKKFYDFVNITQKRILIKGLVESIVWDTGGEEKILEINLIGSNTKLPSGKVKRRE

SEQ ID NO: 65 - protein, 485 aa, synthetic construct
MSKKVAIYTRVSTTNQAEEGYSIDEQIDKLKMYCEAMDWKVSEIYTDAGFTGSKLTRPAMEKMITDIGLKKFDTVIVYKLDRLSRSVRDTLYLVKDVFTKNEIDFISLSESIDTSSAMGSLFLTILSAINEFERENIKERMTMGKIGRAKSGKSMMWAKTAFGYSHNQETGILEINPLEASIVEQIFNEYLKGTSITKLRDKLNEDGHIAKELPWSYRTIRQTLDNPVYCGYIKYKNNTFEGLHKPIISHETYLSVQKELEARQQQTYEKNNNPRPFQAKYLLSGIARCGYCGAPLRIVLGHRRKDGSRTMKYQCVNRFPRKTKGVTTYNDNKKCDSGAYDMQWIEDIVLKTLNGFQKSDKKLRKILNIKEESKVDTSGFQKQLKSINNKIQKNSDLYLNDFITMDDLKKRTEMLQGEKKLIQARINEVDKPSTSEIFDLVKSELGETTISKISYEDKKKIVNNLISKVDVTADNIDIIFKFQLA

SEQ ID NO: 66 - protein, 458 aa, synthetic construct
MNEQKDKLKKYCEIKDWTIVKEYVDPGRSGSNINRPSMQQLIKDADTGLYDAVLVYKLDRLSRSQKDTLYLIEDVFQKNNIHFISLSENFDTSTAFGKAMIGILSVFAQLEREQIKERMSMGRVGRAKSGKIMEFNNPAFGYEVDGDNYKVDPLRAEIVKRIYKMYLSGTSINKIKETLNLEGHIGNKKNWSDTRIRYILSNPTYLGKIRYDGKTYDGKFSPIIDEETFNKTQNELKERQTATYKRFNMKLRPFQSKYMLSGLLRCGYCGATLFVNSYVYNGKRKLRYNCPSTYKSKQKTRTYKIMDPNCPFKLVYAKDLEPAVINEIKNLALNPQSIQKPVKKKPDIDVEAIQKELAKVRKQQQRLIDLYVISDDVNIDNISKKSADLKLQEETLKKQLAPLEEPNDDDKIVAFNEILAQIKDIDSLDYDKQKFIVKKLIKKIDVWNDNKIKIHWNI

SEQ ID NO: 67 - protein, 515 aa, synthetic construct
MKTAIYLRKSRADLEAEARGEGETLAKHRSTLLKIAKEMNLNVLSVREEIVSGESLVKRPEMLALLEEIEDNKYDAVLCMDMDRLGRGGMKEQGIILETFKRSNTKIMTPRKTYDLNDEWDEEYSEFEAFMARKELKIITRRMQRGRIASVEAGNYLGTHAPFGYDIHRLNKRERTLTINSEEASVVRMIFDWYANEDMGASAIRNKLNDLGYKSKLGNDWNPYSILDILKNNIYIGKVTWQKRKEVKRPDAVKRSCARQDKSDWIIADGKHEPIIPESLFEQAQEKLNSRYHVPYNTNGIKNPLAGIIKCSKCGYSMVQRYPKNRKETMDCKHRGCENKSSYTELIEKRLLEALKEWYINYKADFEAHKQGDKLKETQVIQMNEAALRKLEKELVDVQKQKNNLHDLLERGVYTVDMFLERSQVISDRINEITSTMENLKKEIKTEIKKEKVKKDTIPQVEHVLDLYFKTDDPKKKNSLLKSVLEKAVYKKEKWQRLDDFELVLYPKLPQDGDI

SEQ ID NO: 68 - protein, 516 aa, synthetic construct
MYRPESLDVCIYLRKSRKDVEEERRAIEEGSSYNALERHRKRLFAIAKAENHNIIDIFEEVASGESIQERPQMQQLLRKLEGNEIDGVLVIDLDRLGRGDMLDAGMIDRAFRYSSTKIITPTDVYDPDDESWELVFGIKSLISRQELKSITKRLQNGRIDSVKEGKHIGKKPPYGYLKDENLRLYPDPEKAWIVKKIFELMCDGKGRQMIAAELDRLGIDPPVTKRGAWDSSTITSIIKNEVYTGVIVWGKFKHKKRNGKYTRHKNPQEKWIMYENAHEPIISKELFDAANEAHSSRHKPAVITSKKLTNPLAGILKCKLCGYTMLIQTRKDRPHNYLRCNNPACKGKQKQSVFNLVEEKLLYSLQQIVDEYQAQKVEEVEIDDSKLISFKEKAIISKEKELKELQAQKGNLHDLLEQGIYTVEIFLERQKNLVERITSIENDIEVLQKEIETEQIKEHNKTEFIPALKTVIESYHKTTNIELKNQLLKTILSTVTYYRHPDWKTNEFEIQVYFKI

SEQ ID NO: 69 - protein, 513 aa, synthetic construct
MKYLALHENSRIAVYSRKSREDRDSEDTLAKHRNELEYLIKRENFKNVQWFEKVVSGETIDERPMFSLLLPRIENGEFDAVCAVAMDRLSRGSQIDSGRILEAFKQSGTLFITPKKTYDLSIEGDEMLSEFESIIARSEYRAIKRRTINGKKNATREGRLHSGSVPYGYKWDKNLKAAVVVEEKKKIYRMMIKWFLEEEYSCTVIAEMLNELKVPSPSGRSIWYGEVVSEILSNDFHRGYVWFGKYKKSKSNNSIVQNKNLDEVLIAKGHHETMKTDEEHALILNRIEKLRTYKVAGRRLNMNTHRLSGIVRCPYCHKAQAIEQPKGRRKHVRKCLRKSAERTKECEETKGIHEEVLFQSIMKEIKKYNESLFSPTEQDVNDDSYTAQLIGLREKAVKKAKGRIERIKEMYLDGDISKTEYKEKLKISQETLQKAENELAELIASTEFQNALSAETKKEKWSHHKVQEMIESTDGMSNSEINLILKMLISHVTYTVEDLGDGTKNLNIKVYYN

SEQ ID NO: 70 - protein, 580 aa, synthetic construct
MDPQHKPTRALIVIRLSRLTDETTSPERQLEACERFCAARGWEVVGVAEDLDVSAGTTSPFERPSLSQWIGDGKDNPGRIGEFDTVVFYRVDRLVRRVRHLHDVIAWSERFDVNMVSATESHFDLSTTIGALIAQLVASFAEMELEGISQRATSAHRHNVQLGKFVGGSPPFGYMPEETPDGWRLVHDPDVVPIILEVVDRVLEGEPLRRITDDLNARGATTARDLVKQRKGKETEGHKWHSNVLKRRLMSPAMLGYALRREPLTDSKGKPKLSAKGAKLYGPEEIVRGPDGLPVQRAEPILPKPLFDRVVAELEARELQKEPTKRINSMLLRVLYCGVCGQPVYRAKGQGGRSDRYRCRSIQDGANCGNPSVLTYELDDLVEESILVLMGDSERLAHVWNPGEDNASELAEVEARLADRTGLIGVGAYKAGTPQRATLDTLIEADAKLYERLKAATPRPAGWTWEPTGETFAEWWAALDTGARNVYLRNMGVRVTYDKRPVPEQVSAGEKPRVHLELGEVRKMAEQVAVTGTIGTLTRNYTRLGEIGITHVDIDAGSGKAVFVTKSGERFELPLNIPEE

SEQ ID NO: 71 - protein, 540 aa, synthetic construct
MKAIAIYARKSLFTGKGDSIGAQVDTCKRFIDYKFANEDYEIRTFKDEGWSGKTTDRPDFTNMVNLIKSKKIDYVITYKLDRIGRTARDLHNFLYELDNLGIVYLSATEPYDTTTSAGRFMISILAAMAQMERERLAERVKSGMIQIAKKGRWLGGQCPLGFDSKREIYIDDMGKERQMMRLTPNKEEIKIVKLIYDKYLEMGSMSQVRKYCLENSIRGKNGGDFSTNTLKQLLTSPIYVKSSDNIFKYLESQNINVFGTPNGNGMLTFNKTKEIRIERDKSEWIAAVGKHKGIIDDNKWLQIQQQLQQQSEKQIKSSGRQGTTSTGLLSGIIKCSKCGNNLLIKTGHKSKKNPGTTYSYYVCGKKDNSYGHKCDNKNVRTDEADSAVITQLKLYNKELLIKNLKEALIQNEKTDTDNIEILESKLKEKEKAVSNLVKKLSLIDDESISNIILNEVTNINKEINDIKLQLSNETLKINEVTKATLDTEIYIKILENFNKKIDDITDPIEKMNLLKSALESVEWNGDSGEFKINLIGSKKK

SEQ ID NO: 72 - protein, 460 aa, synthetic construct
MKVAIYTRVSTLEQREKGHSIDEQERKLRSFCDINDWTVKDVYVDAGFSGAKRDRPELTRLLDDISEFDLVLVYKLDRLTRSVRDLLDLLEVFENNNVAFRSATEVYDTTTAIGRLFVTLVGAMAEWERETIRERSLMGKRAAIKKGMILTAPPFYYDRVNNTYIPNQYKDVVLDVYNKVKKGYSIAHIARLYNNSDVKPPNGNEEWTTRMLMHALRNPVTRGHYQWGEIYIEDSHEPIITDEMYNTIIDRLDKHTNTKVVAHTSVFRGKLICPNCGYALTLNSQKRKRKNDTIVYKTYYCNNCKITKGMKPHHITETETLRVFKDHLSKIDLKQYETQEKEKQSHVTIDLSKVMEQRKRYHKLYASGMMQENELFELIKETDEMIEEYEKQRKQVDVKEFDICKIKEIKDVLLKSWDIFTLEDKADFIQMSIKAINIEYTKLKRGKSSNSMKIKDIEFY

SEQ ID NO: 73 - protein, 343 aa, synthetic construct
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 74 - protein, 432 aa, synthetic construct
MAPKKKRKVMSQFDILCKTPPKVLVRQFVERFERPSGEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 75 - protein, 509 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMSGSPKKKRKV

SEQ ID NO: 76 - protein, 500 aa, synthetic construct [sequence omitted]

SEQ ID NO: 77 - protein, 21 aa, synthetic construct
CCAAAGAAAAAGCGGAAAGTG

SEQ ID NO: 78 - protein, 7 aa, synthetic construct
PKKKRKV

SEQ ID NO: 79 - DNA, 62 bp, unidentified
  source 1..62: mol_type = genomic DNA; note = Rhodobacter Sphaeroides 2.4.1
ggaactccgccgggcccatctggtcgaagaagatgaaggggcccaccatctgcctccgggcc

SEQ ID NO: 80 - DNA, 62 bp, unidentified
  source 1..62: mol_type = genomic DNA; note = Rhodobacter Sphaeroides 2.4.1
atggggtcacaataccaatcatgttcaagaatgtgaagggtattttacccttgtcgtttcag

SEQ ID NO: 81 - DNA, 59 bp, unidentified
  source 1..59: mol_type = genomic DNA; note = Streptomyces scabiei 87.22
ggacggcgcagaaggggagtagctcttcgccggaccgtcgacatactgctcagctcgtc

SEQ ID NO: 82 - DNA, 63 bp, unidentified
  source 1..63: mol_type = genomic DNA; note = Streptomyces scabiei 87.22
gctcatgtatgtgtctacgcgagattctcgcccgagaacttctgcaaggcactgctcttggct

SEQ ID NO: 83 - DNA, 62 bp, unidentified
  source 1..62: mol_type = genomic DNA; note = Phi370.1
gtttgtaaaggagactgataatggcatgtacaactatactcgtcggtaaaaaggcatcttat

SEQ ID NO: 84 - DNA, 65 bp, unidentified
  source 1..65: mol_type = genomic DNA; note = Phi370.1
atggataaaaaaatacagcgtttttcatgtacaactatactagttgtagtgcctaaataatgctt

SEQ ID NO: 85 - DNA, 66 bp, unidentified
  source 1..66: mol_type = genomic DNA; note = Streptococcus equi subsp. equi 4047
ttccaaagagcgcccaacgcgacctgaaatttgaataagactgctgcttgtgtaaaggcgatgatt

SEQ ID NO: 86 - DNA, 63 bp, unidentified
  source 1..63: mol_type = genomic DNA; note = Streptococcus equi subsp. equi 4047
caaaaattacaaagttttcaacccttgatttgaattagcggtcaaataatttgtaattcgttt

SEQ ID NO: 87 - DNA, 61 bp, unidentified
  source 1..61: mol_type = genomic DNA; note = PhiK38
gagcgccggatcagggagtggacggcctgggagcgctacacgctgtggctgcggtcggtgc

SEQ ID NO: 88 - DNA, 60 bp, unidentified
  source 1..60: mol_type = genomic DNA; note = PhiK38
ccctaatacgcaagtcgataactctcctgggagcgttgacaacttgcgcaccctgatctg

SEQ ID NO: 89 - DNA, 62 bp, unidentified
  source 1..62: mol_type = genomic DNA; note = Burkholderia multivorans ATCC 17616
gatacggatgttcgtcgccggcacgctggtcacgctcggcaatcccaagatcatgctgttct

SEQ ID NO: 90 - DNA, 62 bp, unidentified
  source 1..62: mol_type = genomic DNA; note = Burkholderia multivorans ATCC 17616
agttgtctgataatatattttcggacacgctcggcaacccgaacgagagtcaaaatacattt

SEQ ID NO: 91 - DNA, 62 bp, unidentified
  source 1..62: mol_type = genomic DNA; note = Geobacillus sp. Y412MC61
agacgagaaacgttccgtccgtctgggtcagttgggcaaagttgatgaccgggtcgtccgtt

SEQ ID NO: 92 - DNA, 66 bp, unidentified
  source 1..66: mol_type = genomic DNA; note = Geobacillus sp. Y412MC61
gtgttataaacctgtgtgagagttaagtttacatgcctaaccttaacttttacgcaggttcagctt

SEQ ID NO: 93 - DNA, 65 bp, unidentified
  source 1..65: mol_type = genomic DNA; note = Staphylococcus haemolyticus JCSC1435
caatcatcagataactatggcggcacgtgcattaaccacggttgtatcccgtctaaagtactcgt

SEQ ID NO: 94 - DNA, 64 bp, unidentified
  source 1..64: mol_type = genomic DNA; note = Staphylococcus haemolyticus JCSC1435
ttaataaactatggaagtatgtacagtcttgcaatgttgagtgaacaaacttccataataaaat

SEQ ID NO: 95 - DNA, 65 bp, unidentified
  source 1..65: mol_type = genomic DNA; note = Staphylococcus aureus str. Newman
tttatattgcgaaaaataattggcgaacgaggtaactggatacctcatccgccaattaaaatttg

SEQ ID NO: 96 - DNA, 62 bp, unidentified
  source 1..62: mol_type = genomic DNA; note = Staphylococcus aureus str. Newman
gtggttgtttttgttggaagtgtgtatcaggtatctgcatagttattccgaacttccaatta

SEQ ID NO: 97 - DNA, 62 bp, unidentified
  source 1..62: mol_type = genomic DNA; note = Streptococcus pneumoniae 670-6B
agcacgctgataatcagcaagaccaccaacatttccaccaatgtaaaagctttaaccttagc

SEQ ID NO: 98 - DNA, 62 bp, unidentified
  source 1..62: mol_type = genomic DNA; note = Streptococcus pneumoniae 670-6B
ggaaaatataaataattttagtaacctacatctcaatcaaggatagtaaaactctcactctt

SEQ ID NO: 99 - DNA, 65 bp, unidentified
  source 1..65: mol_type = genomic DNA; note = Clostridium botulinum B str. Eklund 17B
atggattttgcagattcccagatgcccctacagaaagaggtacaaaacatttattggaattaatt

SEQ ID NO: 100 - DNA, 65 bp, unidentified
  source 1..65: mol_type = genomic DNA; note = Clostridium botulinum B str. Eklund 17B
gtttatatgtttactaataagacgctctcaacccataaagtcttattagtaaacatatttcaact

SEQ ID NO: 101 - DNA, 66 bp, unidentified
  source 1..66: mol_type = genomic DNA; note = Staphylococcus lugdunensis N920143
gttcgtggtaactatgggtggtacaggtgccacattagttgtaccatttatgtttatgtggttaac

SEQ ID NO: 102 - DNA, 65 bp, unidentified
  source 1..65: mol_type = genomic DNA; note = Staphylococcus lugdunensis N920143
tttttgtatgttagttgtgtcactgggtagacctaaatagtgacacaactgctattaaaatttaa

SEQ ID NO: 103 - DNA, 65 bp, unidentified
  source 1..65: mol_type = genomic DNA; note = Bacillus cytotoxicus NVH 391-98
cgcatacattgttgttgtttttccagatccagttggtcctgtaaatataagcaatccatgtgagt

SEQ ID NO: 104 - DNA, 64 bp, unidentified
  source 1..64: mol_type = genomic DNA; note = Bacillus cytotoxicus NVH 391-98
caataacggttgtatttgtagaacttgaccagttgttttagtaacataaatacaactccgaata

SEQ ID NO: 105 - DNA, 62 bp, unidentified
  source 1..62: mol_type = genomic DNA; note = Listeria innocua Clip11262
ttattgcaagaaaaatgggttataagtacacatcaggttatagtaatatcgaaaaaggaagc

SEQ ID NO: 106 - DNA, 62 bp, unidentified
  source 1..62: mol_type = genomic DNA; note = Listeria innocua Clip11262
ttatataaaatagtgtttttgtaaagtacacatcaccatatttgacaaaaaacctataaata

SEQ ID NO: 107 - DNA, 62 bp, unidentified
  source 1..62: mol_type = genomic DNA; note = A118
ctgtaactttttcggatcaagctatgagggacgcaaagagggaactaaacacttaattggtg

SEQ ID NO: 108 - DNA, 62 bp, unidentified
  source 1..62: mol_type = genomic DNA; note = A118
ttgtttagtccctcgttttctctcgttggacggagacgaatcgagaaactaaaattataaat

SEQ ID NO: 109 - DNA, 62 bp, unidentified
  source 1..62: mol_type = genomic DNA; note = Enterococcus faecalis D32
ttctggaccatgatgcgccacttccgaaatttcaaaaagatcagtggtcaaacggctcatta

SEQ ID NO: 110 - DNA, 62 bp, unidentified
  source 1..62: mol_type = genomic DNA; note = Enterococcus faecalis D32
gtatcttgatgtacaacattactctttattttcaaatacagaataatgttgcatataatatt

SEQ ID NO: 111 - DNA, 61 bp, unidentified
  source 1..61: mol_type = genomic DNA; note = Staphylococcus epidermidis RP62A
acttccaattaacccttcaccagccctataccaagttcctgtcgcgcatcctccagctaat

SEQ ID NO: 112 - DNA, 61 bp, unidentified
  source 1..61: mol_type = genomic DNA; note = Staphylococcus epidermidis RP62A
ttatatttcgacttaattaagtacagttccacctagagatagactaaataaagtattatta

SEQ ID NO: 113 - DNA, 62 bp, unidentified
  source 1..62: mol_type = genomic DNA; note = Streptococcus pneumoniae JJA
tctggtgtagacgttaaacgtccaatcaagataactttattatacatattttcttcctccta

SEQ ID NO: 114 - DNA, 62 bp, unidentified
  source 1..62: mol_type = genomic DNA; note = Streptococcus pneumoniae JJA
tatttctgtattttagtcaaagtaattaagataagttagagttagtaacagtattttaactt

SEQ ID NO: 115 - DNA, 62 bp, unidentified
  source 1..62: mol_type = genomic DNA; note = PhiM46.1
gtagatttgtttccccagacgcacacgtggagtgtgtaagtttacttgagaaacggagttaa

SEQ ID NO: 116 - DNA, 62 bp, unidentified
  source 1..62: mol_type = genomic DNA; note = PhiM46.1
tattagtatagaagaaagctctcagcacacgtggagtgtgttgctctctgctcgtaaagcct

SEQ ID NO: 117 - DNA, 62 bp, unidentified
  source 1..62: mol_type = genomic DNA; note = Streptococcus suis D12
cttccagcacatcacccacatggtctgtgtcggtgtgcgtcagcactagactatcaatccta

SEQ ID NO: 118 - DNA, 62 bp, unidentified
  source 1..62: mol_type = genomic DNA; note = Streptococcus suis D12
ggtattgtatcaatttcagaactcacacttcggtatgcgtactcaatttgatacaattacaa

SEQ ID NO: 119 - DNA, 63 bp, unidentified
  source 1..63: mol_type = genomic DNA; note = Streptococcus mitis B6
taggaggaaaaaatatgtataataaagttatcatgattgggcgtttgacgtctacaccagaat

SEQ ID NO: 120 - DNA, 61 bp, unidentified
  source 1..61: mol_type = genomic DNA; note = Streptococcus mitis B6
gttaataatatgtatttaagtctaacttatcatgacaaatttgactaaaatacaaaaaggc

SEQ ID NO: 121 - DNA, 61 bp, unidentified
  source 1..61: mol_type = genomic DNA; note = Geobacillus thermoglucosidasius C56-YS93
caagaaacgttccgtctgtttgtgtcagctgcgcgaaattaatgaccggatcgtttgttcc

SEQ ID NO: 122 - DNA, 62 bp, unidentified
  source 1..62: mol_type = genomic DNA; note = Geobacillus thermoglucosidasius C56-YS93
ttataaacctgttttaaagttaactttacatgcctaacattaactcttatacaggttaaggt

SEQ ID NO: 123 - DNA, 61 bp, unidentified
  source 1..61: mol_type = genomic DNA; note = Clostridium difficile 630
tattctaagtaatgtagttttaccacatccactaggtccgagtaaacatagaaattcccct

SEQ ID NO: 124 - DNA, 64 bp, unidentified
  source 1..64: mol_type = genomic DNA; note = Clostridium difficile 630
tatataattatttggactaacatatagtatccacttggctattattagttagtccaaataaata

SEQ ID NO: 125 - DNA, 62 bp, unidentified
  source 1..62: mol_type = genomic DNA; note = Clostridium botulinum H04402 065
actacttaatatatccataagagaaatttcatttccttctttgtctacccctataggatctt

SEQ ID NO: 126 - DNA, 62 bp, unidentified
  source 1..62: mol_type = genomic DNA; note = Clostridium botulinum H04402 065
gttaggtgtatatcatacctaacgcaattcattacatcacatatgttatacacctactttaa

SEQ ID NO: 127 - DNA, 61 bp, unidentified
  source 1..61: mol_type = genomic DNA; note = Clostridium botulinum A str. ATCC 19397
tattcaattatgtgtcgtaatttttatctattgcgacgaaaaaacaccataaaattctaac

SEQ ID NO: 128 - DNA, 62 bp, unidentified
  source 1..62: mol_type = genomic DNA; note = Clostridium botulinum A str. ATCC 19397
tatatacttatagatactaaatatttttgtattgcgtaacttcttctacacctgtaatatct

SEQ ID NO: 129 - DNA, 61 bp, unidentified
  source 1..61: mol_type = genomic DNA; note = Clostridium botulinum F str. Langeland
agaaatagacctttcaactggacaaggtgctgataaaactatgcagcaagtcttaagtaaa

SEQ ID NO: 130 - DNA, 62 bp, unidentified
  source 1..62: mol_type = genomic DNA; note = Clostridium botulinum F str. Langeland
aaatataacctgtgtattgaaacaaggtgctgataaaaccctttcataaacacaagtaaata

SEQ ID NO: 131 - DNA, 62 bp, unidentified
  source 1..62: mol_type = genomic DNA; note = High similarity to TP901-1
aatactaataatagctagtacaattaacatctctatcaaagtaaaagcttttagctctttct

SEQ ID NO: 132 - DNA, 60 bp, unidentified
  source 1..60: mol_type = genomic DNA; note = High similarity to TP901-1
cgcttaattgcgagtttttatttcgcttatctcaattaaggtaactaaaaaactcctttt

SEQ ID NO: 133 - DNA, 62 bp, unidentified
  source 1..62: mol_type = genomic DNA; note = Lactobacillus reuteri DSM 20016
aagtgtccaagctggcccccgatcccagtttcaatagtttggggaatctttgtaagtggtaa

SEQ ID NO: 134 - DNA, 62 bp, unidentified
  source 1..62: mol_type = genomic DNA; note = Lactobacillus reuteri DSM 20016
tataatttcgtatattagatataaccggtttcaattggaaatacctaatatacgaaaaaagg

SEQ ID NO: 135 - DNA, 62 bp, unidentified
  source 1..62: mol_type = genomic DNA; note = Bacillus weihenstephanensis KBAB4
tttgtagccattaggcgcattaggttgacgccattaagccctaaagcatcattcgtcgaaac

SEQ ID NO: 136 - DNA, 62 bp, unidentified
  source 1..62: mol_type = genomic DNA; note = Bacillus weihenstephanensis KBAB4
cgtcaccttgttggcgtaattagatttactccaacagggtgatgacaaagctaatgaatttt

SEQ ID NO: 137 - DNA, 62 bp, unidentified
  source 1..62: mol_type = genomic DNA; note = Bacillus cereus AH187
gtaatatgtttggatatggggaagtgaatcagtacaaccgccacagtaccctcatgtcagcc

SEQ ID NO: 138 - DNA, 62 bp, unidentified
  source 1..62: mol_type = genomic DNA; note = Bacillus cereus AH187
ataatagtgtatatggtagagaattaaaccagtttaatactccaccatgtacacgcagtgag

SEQ ID NO: 139 - DNA, 61 bp, unidentified
  source 1..61: mol_type = genomic DNA; note = Bacillus amyloliquefaciens XH7
ttttttccgcctgtcgtaaccggatctgttgtaacgattatcggaatgaccttgatgccgg

SEQ ID NO: 140 - DNA, 62 bp, unidentified
  source 1..62: mol_type = genomic DNA; note = Bacillus amyloliquefaciens XH7
cttttttgttgtacttaaacaataatgcttgtaagaattattgattgagtacgacataaacc

SEQ ID NO: 141 - DNA, 62 bp, unidentified
  source 1..62: mol_type = genomic DNA; note = Rhodococcus jostii RHA1
atcgcgcagaacggtgcggtgatcagtgagtacgcaccgggcacgacaccggcgaagcatcg

SEQ ID NO: 142 - DNA, 59 bp, unidentified
  source 1..59: mol_type = genomic DNA; note = Rhodococcus jostii RHA1
ctatgtggtggtaatagcgagtaggggactactcgctccaggtacattaacaccatgga

SEQ ID NO: 143 - DNA, 62 bp, unidentified
  source 1..62: mol_type = genomic DNA; note = Clostridium botulinum A2 str. Kyoto
acgaaataaaagattgtatagatgctggtaggaaacatgcccttgtcatttagctgaaacag

SEQ ID NO: 144 - DNA, 62 bp, unidentified
  source 1..62: mol_type = genomic DNA; note = Clostridium botulinum A2 str. Kyoto
aaaagaatccaaattatcgtactttaacatagtgaatactgtccatcatgtataaaagtacg

SEQ ID NO: 145 - DNA, 64 bp, unidentified
  source 1..64: mol_type = genomic DNA; note = Staphylococcus lugdunensis HKU09-01
aatctgcaaacatgtatggcggtacatgtatcaacattggttgtattcctacaaagacactcat

SEQ ID NO: 146 - DNA, 61 bp, unidentified
  source 1..61: mol_type = genomic DNA; note = Staphylococcus lugdunensis HKU09-01
atttttgtacggaagtagatactatctttcaatatccatgttacttagtgccatacaaaaa

SEQ ID NO: 147 - DNA, 50 bp, unidentified
  source 1..50: mol_type = genomic DNA; note = Bxb1
tcggccggcttgtcgacgacggcggtctccgtcgtcaggatcatccgggc

SEQ ID NO: 148 - DNA, 58 bp, unidentified
  source 1..58: mol_type = genomic DNA; note = Bxb1
gtcgtggtttgtctggtcaaccaccgcggtctcagtggtgtacggtacaaaccccgac

SEQ ID NO: 149 - DNA, 48 bp, Saccharomyces cerevisiae
  source 1..48: mol_type = genomic DNA
gaagttcctattccgaagttcctattctctagaaagtataggaacttc

SEQ ID NO: 150 - DNA, 48 bp, synthetic construct
  source 1..48: mol_type = other DNA
gaagttcctattccgaagttcctattcttcaaatagtataggaacttc

SEQ ID NO: 151 - DNA, 48 bp, synthetic construct
  source 1..48: mol_type = other DNA
gaagttcctattccgaagttcctattcttcaaaaggtataggaacttc

SEQ ID NO: 152 - DNA, 48 bp, synthetic construct
  source 1..48: mol_type = other DNA
gaagttcctattccgaagttcctattctatcagaagtataggaacttc

SEQ ID NO: 153 - DNA, 48 bp, synthetic construct
  source 1..48: mol_type = other DNA
gaagttcctattccgaagttcctattcttataggagtataggaacttc

SEQ ID NO: 154 - DNA, 34 bp, unidentified
  source 1..34: mol_type = genomic DNA; note = Bacteriophage P1
ataacttcgtataatgtatgctatacgaagttat

SEQ ID NO: 155 - DNA, 34 bp, synthetic construct
  source 1..34: mol_type = other DNA
ataacttcgtatagtataccttatacgaagttat

SEQ ID NO: 156 - DNA, 34 bp, synthetic construct
  source 1..34: mol_type = other DNA
ataacttcgtataaagtatcctatacgaagttat

SEQ ID NO: 157 - DNA, 34 bp, synthetic construct
  source 1..34: mol_type = other DNA
taccgttcgtataatgtatgctatacgaagttat

SEQ ID NO: 158 - DNA, 34 bp, synthetic construct
  source 1..34: mol_type = other DNA
ataacttcgtataatgtatgctatacgaacggta

SEQ ID NO: 159 - DNA, 34 bp, synthetic construct
  source 1..34: mol_type = other DNA
ataacttcgtataatgtatgctataccttgttat

SEQ ID NO: 160 - DNA, 578 bp, synthetic construct
  source 1..578: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatg

SEQ ID NO: 161 - DNA, 571 bp, synthetic construct
  source 1..571: mol_type = other DNA
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatagttatc

SEQ ID NO: 162 - protein, 19 aa, synthetic construct
ATNFSLLKQAGDVEENPGP

SEQ ID NO: 163 - protein, 18 aa, synthetic construct
EGRGSLLTCGDVEENPGP

SEQ ID NO: 164 - DNA, 1131 bp, synthetic construct
  source 1..1131: mol_type = other DNA
atggcctcttatcctggacaccagcacgccagcgcctttgatcaggctgccagatctagaggccacagcaacagaagaacagccctgcggcctcggagacagcaagaggctacagaagttcggcccgagcagaagatgcccacactgctgagagtgtacatcgacggccctcacggcatgggcaagaccacaacaacacagctgctggtggccctgggcagcagagatgatatcgtgtacgtgcccgagcctatgacctattggagagtgctgggcgccagcgagacaatcgccaacatctacaccacacagcaccggctggatcagggcgaaatttctgctggcgacgccgccgtggttatgacatctgcccagatcaccatgggcatgccttacgccgtgacagatgctgtgctggcccctcacattggcggagaagccggatcttctcatgcccctccaccagctctgaccctgatcttcgacagacaccctatcgctcatctgctgtgctaccctgccgccagatacctgatgggcagcatgacacctcaggccgtgctggctttcgtggccctgattcctcctacactgcccggcaccaatatcgtgctgggagccctgcctgaggaccggcacattgatagactggccaagagacagcggcctggcgagagactggatctggctatgctggccgccatcagaagagtgtacggcctgctggccaacaccgtgcggtatcttcaatgtggcggctcttggagagaggactggggacagctttctggcacagcagttcctccacaaggcgccgagcctcagtctaatgctggacccagacctcacatcggcgacaccctgtttaccctgttcagagcccctgagctgctggctcctaacggcgacctgtacaacgtgttcgcctgggctcttgacgtgctggcaaagcggctgagatccatgcacgtgttcatcctggactacgatcagtcccctgccggctgtagagatgctctgctgcagctgacaagcggcatggtgcagacccacgttacaacccctggcagcatccccaccatctgtgacctggccagaaccttcgccagagagatgggcgaagccaactga

SEQ ID NO: 165 - protein, 376 aa, synthetic construct
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLRSMHVFILDYDQSPAGCRDALLQLTSGMVQTHVTTPGSIPTICDLARTFAREMGEAN

SEQ ID NO: 166 - DNA, 50 bp, unidentified
  source 1..50: mol_type = genomic DNA; note = Bxb1
tcggccggcttgtcgacgacggcggactccgtcgtcaggatcatccgggc

SEQ ID NO: 167 - DNA, 58 bp, unidentified
  source 1..58: mol_type = genomic DNA; note = Bxb1
gtcgtggtttgtctggtcaaccaccgcggactcagtggtgtacggtacaaaccccgac

SEQ ID NO: 168 - protein, 7 aa, synthetic construct
PKKKRKV

SEQ ID NO: 169 - protein, 10 aa, synthetic construct
PYLNKRKGKP

SEQ ID NO: 170 - protein, 9 aa, synthetic construct
PAAKRVKLD

SEQ ID NO: 171 - protein, 16 aa, synthetic construct
KRPAATKKAGQAKKKK

SEQ ID NO: 172 - protein, 9 aa, synthetic construct
PAAKKKKLD

SEQ ID NO: 173 - protein, 20 aa, synthetic construct
RKPVTAQERQREREEKRRRR

SEQ ID NO: 174 - protein, 7 aa, synthetic construct
KSRKRKL

SEQ ID NO: 175 - protein, 7 aa, synthetic construct
GKKRSKV

SEQ ID NO: 176 - protein, 4 aa, synthetic construct
GGGS

SEQ ID NO: 177 - protein, 5 aa, synthetic construct
GGGGS

SEQ ID NO: 178 - protein, 6 aa, synthetic construct
SGGGGS

SEQ ID NO: 179 - protein, 8 aa, synthetic construct
GGSGGGGS

(19) United States
(12) Patent Application Publication
(10) Pub. No.: US 2024/0409906 A1
(43) Pub. Date: Dec. 12, 2024

(54) INTEGRASES, LANDING PAD ARCHITECTURES, AND ENGINEERED CELLS COMPRISING THE SAME

(71) Applicant: Asimov Inc., Boston, MA (US)

(72) Inventors: Michael T. Leonard, Boston, MA (US); Jeremy J. Gam, Somerville, MA (US); Christopher S. Stach, Boston, MA (US); Alec A.K. Nielsen, Brookline, MA (US)

(73) Assignee: Asimov Inc., Boston, MA (US)

(21) Appl. No.: 18/700,583

(22) PCT Filed: Oct. 13, 2022
(86) PCT No.: PCT/US2022/078064

§ 371 Date: Apr. 11, 2024

Related U.S. Application Data
(60) Provisional application No. 63/255,661, filed on Oct. 14, 2021.

(51) Int. Cl. C12N 9/12; C12N 5/071; C12N 15/90

(52) U.S. Cl. CPC C12N 9/1241; C12N 5/0682; C12N 15/90; C07K 2319/09; C12N 2830/48; C12Y 207/07

(57) ABSTRACT

Described herein are modified bacteriophage serine integrases that function in mammalian cells. Also described herein are landing pad architectures. Engineered cells comprising these integrases and landing pads are also described, which facilitate site-specific genomic integration of pay load molecules.

### FIELD

Described herein are modified bacteriophage serine integrases that function in mammalian cells. Also described herein are landing pad architectures. Engineered mammalian cells comprising these integrases and landing pads are also described, which facilitate site-specific genomic integration of payload molecules.

### RELATED APPLICATIONS

This application claims the benefit under 35 U.S.C. § 119 of U.S. provisional application Ser. No. 63/255,661, filed Oct. 14, 2021, the entire contents of which are incorporated by reference herein.

### REFERENCE TO AN ELECTRONIC SEQUENCE LISTING

The contents of the electronic sequence listing (A121070005WO00-SEQ-ARM.xml; Size: 250,175 bytes; and Date of Creation: Oct. 13, 2022) is herein incorporated by reference in its entirety.

### BACKGROUND

Integrases, which are also referred to in the art as DNA recombinases, mediate genetic recombination at specific sequence motifs known as recombination sites. Integrases can perform crossover events between linear chromosomes, integration events between a circular DNA sequence and a linear sequence, excision events between consecutive recombination sites in the same orientation, or inversion events between consecutive recombination sites in opposing orientations. Recombinase complexes typically bind to two pairs of inverted, short recognition site repeats that are separated by a spacer sequence. While the exact mechanisms may differ, the spacer sequence is ultimately cleaved at both strands, and those DNA strands are exchanged.

### SUMMARY

In some aspects, the disclosure relates to a polynucleic acid encoding an polypeptide having integrase activity, wherein the polynucleic acid comprises an expression cassette comprising, from 5′ to 3′: (i) a nucleic acid sequence of any one of SEQ ID NOs: 2-5, 7-16, 18, 21-23, 26, 27, 29, 30, 32, and 34 or a nucleic acid sequence having at least 95% identity with any one of SEQ ID NOs: 2-5, 7-16, 18, 21-23, 26, 27, 29, 30, 32, and 34; (ii) a nucleic acid sequence encoding a GS linker; and (iii) a nucleic acid sequence encoding a nuclear localization signal (NLS).

In some aspects, the disclosure relates a polynucleic acid encoding an polypeptide having integrase activity, wherein the polynucleic acid comprises an expression cassette comprising, from 5′ to 3′: (i) a nucleic acid sequence encoding a nuclear localization signal (NLS) (ii) a nucleic acid sequence encoding a GS linker; and (iii) a nucleic acid sequence of any one of SEQ ID NOs: 2-5, 7-16, 18, 21-23, 26, 27, 29, 30, 32, and 34 or a nucleic acid sequence having at least 95% identity with any one of SEQ ID NOs: 2-5, 7-16, 18, 21-23, 26, 27, 29, 30, 32, and 34.

In some embodiments, the nucleic acid sequence encoding the GS linker comprises or consists essentially of the nucleic acid sequence GGTTCA. In some embodiments, the nucleic acid sequence encoding the NLS comprises or consists essentially of the nucleic acid sequence of any one of SEQ ID NOs: 77-78 and 168-174.

In some aspects, the present disclosure relates to a polypeptide having integrase activity and comprising, from N- to C-terminus: (i) an amino acid sequence of any one of SEQ ID NOs: 40-43, 45-54, 56, 59-61, 64, 65, 67, 68, 70, and 72 or an amino acid sequence having at least 95% identity with the amino acid sequence of any one of SEQ ID NOs: 40-43, 45-54, 56, 59-61, 64, 65, 67, 68, 70, and 72; (ii) an amino acid sequence of a GS linker; and (iii) an amino acid sequence of a nuclear localization signal (NLS).

In some aspects, the present disclosure relates to a polypeptide having integrase activity and comprising, from N- to C-terminus: (i) an amino acid sequence of a nuclear localization signal (NLS) (ii) an amino acid sequence of a GS linker; and (iii) an amino acid sequence of any one of SEQ ID NOs: 40-43, 45-54, 56, 59-61, 64, 65, 67, 68, 70, and 72 or an amino acid sequence having at least 95% identity with the amino acid sequence of any one of SEQ ID NOs: 40-43, 45-54, 56, 59-61, 64, 65, 67, 68, 70, and 72. In some embodiments, the GS linker is gly ser. In some embodiments, the amino acid sequence of the NLS comprises or consists essentially of the amino acid sequence of any one of SEQ ID NOs: 77-78 and 168-174.

In some aspects, the present disclosure relates a polynucleic acid encoding the polypeptide of any of the aspects and embodiments disclosed above. In some aspects, the present disclosure relates to an engineered cell comprising a chromosomal integration of a landing pad, wherein the landing pad comprises an expression cassette comprising, from 5′ to 3′: (i) a nucleic acid sequence of a promoter; (ii) a nucleic acid sequence of a first recombination site; and (iii) a nucleic acid sequence encoding for a landing pad marker, which is operably linked to the promoter of (i). In some embodiments, the landing pad further comprises (iv) a nucleic acid sequence of a second recombination site, wherein the nucleic acid sequence of the second recombination site is positioned 3′ to the nucleic acid sequence encoding for the landing pad marker. In some embodiments, the landing pad marker comprises an antibiotic resistance protein. In some embodiments, the landing pad marker comprises a fluorescent protein. In some embodiments, the landing pad further comprises (v) a nucleic acid sequence encoding for a Woodchuck Hepatitis Virus Post-transcriptional Regulatory Element (WPRE) or a nucleic acid sequence encoding a polyA, which is operably linked to the nucleic acid sequence encoding for the landing pad marker. In some embodiments, the landing pad comprises a nucleic acid sequence of a second recombination site, wherein the nucleic acid sequence of the second recombination site is positioned 5′ to the nucleic acid sequence encoding for the WPRE.

In some embodiments, the expression cassette comprises, from 5′ to 3′: (i) the nucleic acid of the promoter; (ii) the nucleic acid sequence of the first recombination site; (iii) the nucleic acid sequence encoding for the landing pad marker; (iv) a nucleic acid sequence of a second recombination site; and (v) the nucleic acid sequence encoding for the WPRE. In some embodiments, the engineered cell is derived from a HEK293 cell. In some embodiments, the landing pad is integrated at a safe harbor locus selected from the group consisting of AAVS1, ROSA26, CCR5, and LiPS-A3S. In some embodiments, the engineered cell is derived from a CHO cell. In some embodiments, the landing pad is integrated at a safe harbor locus selected from the group consisting of ROSA26, COSMIC, and H11.

In some embodiments, the engineered cell further comprises an integrase molecule comprising a nucleic acid sequence of a promoter operably linked to a nucleic acid sequence encoding for an integrase that binds to a recombination site of the landing pad. In some embodiments, the promoter of the integrase molecule is a constitutive promoter. In some embodiments, the integrase is a serine integrase. In some embodiments, the integrase is a tyrosine integrase. In some embodiments, the integrase comprises an amino acid sequence of any one of SEQ ID NOs: 40-43, 45-54, 56, 59-61, 64, 65, 67, 68, 70, and 72 or an amino acid sequence having at least 95% identity with the amino acid sequence of any one of SEQ ID NOs: 40-43, 45-54, 56, 59-61, 64, 65, 67, 68, 70, and 72.

In some embodiments, the integrase further comprises the amino acid sequence of a nuclear localization signal (NLS). In some embodiments, the NLS comprises or consists essentially of the amino acid sequence of any one of SEQ ID NOs: 77-78 and 168-174. In some embodiments, the integrase further comprises a GS linker.

In some aspects, the present disclosure relates to a kit comprising: (a) an engineered cell of as described above; and (b) a donor molecule comprising from 5′ to 3′: (i) a nucleic acid sequence of a first recombination site, which corresponds to the first recombination site of the landing pad of the engineered cell; and (ii) a multiple cloning site. In some aspects, the present disclosure relates to a kit comprising: (a) an engineered cell of as described above; (b) a donor molecule comprising from 5′ to 3′: (i) a nucleic acid sequence of a first recombination site, which corresponds to the first recombination site of the landing pad of the engineered cell; and (ii) a multiple cloning site; and (c) an integrase molecule comprising: (i) a nucleic acid sequence encoding for an integrase that binds to the first recombination sites of the landing pad and the donor molecule; or (ii) an amino acid sequence of an integrase that binds to the first recombination sites of the landing pad and the donor molecule; optionally wherein a single polynucleic acid comprises the donor molecule and the integrase molecule. In some embodiments, the integrase molecule comprises a nucleic acid sequence of a promoter operably linked to a nucleic acid sequence encoding for an integrase, and wherein the promoter of the integrase molecule is a constitutive promoter.

In some embodiments, the integrase is a serine integrase. In some embodiments, the integrase is a tyrosine integrase. In some embodiments, the integrase comprises an amino acid sequence of any one of SEQ ID NOs: 40-43, 45-54, 56, 59-61, 64, 65, 67, 68, 70, and 72 or an amino acid sequence having at least 95% identity with the amino acid sequence of any one of SEQ ID NOs: 40-43, 45-54, 56, 59-61, 64, 65, 67, 68, 70, and 72. In some embodiments, the integrase further comprises the amino acid sequence of a nuclear localization signal (NLS). In some embodiments, the NLS comprises or consists essentially of the amino acid sequence of any one of SEQ ID NOs: 77-78 and 168-174. In some embodiments, the integrase further comprises a GS linker.

In some embodiments, the landing pad of the engineered cell comprises a nucleic acid sequence of a second recombination site, wherein the nucleic acid sequence of the second recombination site is positioned 3′ to the nucleic acid sequence encoding for the landing pad marker; and the donor molecule further comprises a nucleic acid sequence of a second recombination site, which corresponds to the second recombination site of the landing pad of the engineered cell. In some embodiments, the integrase binds to the first and second recombination sites of the landing pad and the donor molecule.

In some embodiments, the kit comprises: a first integrase molecule comprising: (i) a nucleic acid sequence encoding for a first integrase that binds to the first recombination sites of the landing pad and the donor molecule; (ii) or an amino acid sequence of a first integrase that binds to the first recombination sites of the landing pad and the donor molecule; and a second integrase molecule comprising: (i) a nucleic acid sequence encoding for a second integrase that binds to the second recombination sites of the landing pad and the donor molecule; (ii) or an amino acid sequence of a second integrase that binds to the second recombination sites of the landing pad and the donor molecule. In some embodiments, a single polynucleic acid comprises the first integrase molecule and the second integrase molecule.

In some aspects, the present disclosure relates to a method of integrating a nucleic acid sequence of interest into a cell genome, the method comprising: (a) introducing a donor molecule into the engineered cell of any one of claims C12-C19, wherein the donor molecule comprises from 5′ to 3′: (i) a nucleic acid sequence of a first recombination site, which corresponds to the first recombination site of the landing pad of the engineered cell; and (ii) a nucleic acid sequence of interest; (b) expressing the integrase of the integrase molecule, thereby inducing integration of the nucleic acid sequence of interest of the donor molecule into the landing pad of the engineered cell; wherein (a) occurs prior to, concurrently with, or after (b); wherein, after integration, the nucleic acid sequence of interest is operably linked to the promoter of the landing pad of the engineered cell; optionally, wherein, prior to integration, the nucleic acid sequence of interest is not operably linked to a promoter.

In some aspects, the present disclosure relates to a method of integrating a nucleic acid sequence of interest into the genome of a cell comprising: (a) introducing a donor molecule into the engineered cell of any one of claims C1-C11, wherein the donor molecule comprises from 5′ to 3′: (i) a nucleic acid sequence of a first recombination site, which corresponds to the first recombination site of the landing pad of the engineered cell; and (ii) a nucleic acid sequence of interest; (b) introducing an integrase molecule into the engineered cell, wherein the integrase molecule comprises: (i) a nucleic acid sequence encoding for an integrase that binds to the first recombination sites of the landing pad and the donor molecule; or (ii) an amino acid sequence of an integrase that binds to the first recombination sites of the landing pad and the donor molecule; thereby inducing integration of the nucleic acid sequence of interest of the donor molecule into the landing pad of the engineered cell; wherein, after integration, the nucleic acid sequence of interest is operably linked to the promoter of the landing pad of the engineered cell. In some embodiments, prior to integration, the nucleic acid sequence of interest is not operably linked to a promoter; and wherein (a) occurs prior to, concurrently with, or after (b).

In some embodiments, the integrase molecule comprises a nucleic acid sequence of a promoter operably linked to a nucleic acid sequence encoding for an integrase, and wherein the promoter of the integrase molecule is a constitutive promoter. In some embodiments, the integrase is a serine integrase. In some embodiments, the integrase is a tyrosine integrase. In some embodiments, the integrase comprises an amino acid sequence of any one of SEQ ID NOs: 40-43, 45-54, 56, 59-61, 64, 65, 67, 68, 70, and 72 or an amino acid sequence having at least 95% identity with the amino acid sequence of any one of SEQ ID NOs: 40-43, 45-54, 56, 59-61, 64, 65, 67, 68, 70, and 72.

In some embodiments, the integrase further comprises the amino acid sequence of a nuclear localization signal (NLS). In some embodiments, the NLS comprises or consists essentially of the amino acid sequence of any one of SEQ ID NOs: 77-78 and 168-174.

In some embodiments, the integrase further comprises a GS linker.

In some embodiments, the landing pad of the engineered cell comprises a nucleic acid sequence of a second recombination site, wherein the nucleic acid sequence of the second recombination site is positioned 3′ to the nucleic acid sequence encoding for the landing pad marker; and the donor molecule further comprises a nucleic acid sequence of a second recombination site, which corresponds to the second recombination site of the landing pad of the engineered cell. In some embodiments, the integrase binds to the first and second recombination sites of the landing pad and the donor molecule.

In some embodiments, the present disclosure related to a kit for performing the method of claim E10, wherein the kit comprises: a first integrase molecule comprising: (i) a nucleic acid sequence encoding for a first integrase that binds to the first recombination sites of the landing pad and the donor molecule; (ii) or an amino acid sequence of a first integrase that binds to the first recombination sites of the landing pad and the donor molecule; and a second integrase molecule comprising: (i) a nucleic acid sequence encoding for a second integrase that binds to the second recombination sites of the landing pad and the donor molecule; (ii) or an amino acid sequence of a second integrase that binds to the second recombination sites of the landing pad and the donor molecule. In some embodiments, a single polynucleic acid comprises the first integrase molecule and the second integrase molecule. In some embodiments, the landing pad comprises from 5′ to 3′: (i) a nucleic acid sequence of a first recombination site; (ii) a landing pad marker comprising the nucleic acid sequence of a counter-selection marker; and (iii) a nucleic acid sequence of a second recombination site; wherein the landing pad further comprises (iv) a nucleic acid sequence of a promoter positioned 5′ or 3′ to the first recombination site and which is operably linked to the nucleic acid sequence of the counter-selection marker.

In some embodiments, the nucleic acid sequence of the promoter is positioned 5′ to the nucleic acid sequence of the first recombination site. In some embodiments, the promoter is a constitutive promoter. In some embodiments, the landing pad marker further comprises a nucleic acid sequence encoding for an antibiotic resistance protein, a fluorescent protein, or both. In some embodiments, the landing pad marker further comprises a nucleic acid sequence encoding for a viral 2A peptide. In some embodiments, the landing pad marker is encoding on a polycistronic mRNA comprising, from 5′ to 3′: (i) a nucleic acid sequence encoding for a fluorescent protein; (ii) a nucleic acid sequence encoding for an antibiotic resistance protein; (iii) a nucleic acid sequence encoding for a viral 2A peptide; and (iv) a nucleic acid sequence encoding for the counter-selection marker. In some embodiments, the counter-selection marker comprises HSV-TK.

In some embodiments, the engineered cell is derived from a HEK293 cell, HeLa S3 cell, T-cell, induced pluripotent stem cell (iPSC), natural killer (NK) cell or human embryonic stem cell. In some embodiments, the landing pad is integrated at a safe harbor locus selected from the group consisting of AAVS1, ROSA26, CCR5, and LiPS-A3S. In some embodiments, the engineered cell is derived from a CHO cell. In some embodiments, the landing pad is integrated at a safe harbor locus selected from the group consisting of ROSA26, COSMIC, and H11. In some embodiments, the engineered cell further comprises a first integrase molecule comprising a nucleic acid sequence of a promoter operably linked to a nucleic acid sequence encoding for a first integrase that binds to a recombination site of the landing pad. In some embodiments, the promoter of the first integrase molecule is a constitutive promoter. In some embodiments, the first integrase is a serine integrase. In some embodiments, the first integrase is a tyrosine integrase. In some embodiments, the first integrase comprises an amino acid sequence of any one of SEQ ID NOs: 40-43, 45-54, 56, 59-61, 64, 65, 67, 68, 70, and 72 or an amino acid sequence having at least 95% identity with the amino acid sequence of any one of SEQ ID NOs: 40-43, 45-54, 56, 59-61, 64, 65, 67, 68, 70, and 72.

In some embodiments, the first integrase further comprises the amino acid sequence of a nuclear localization signal (NLS). In some embodiments, the NLS comprises or consists essentially of the amino acid sequence of any one of SEQ ID NOs: 77-78 and 168-174.

In some embodiments, the first integrase further comprises a GS linker.

In some embodiments, the engineered cell further comprises a second integrase molecule, wherein the second integrase molecule comprises a nucleic acid sequence of a promoter operably linked to a nucleic acid sequence encoding for a second integrase that binds to a recombination site of the landing pad. In some embodiments, the first integrase and the second integrase bind to orthogonal recombination sites.

In some aspects, the present disclosure relates a kit comprising: (a) an engineered cell of any one of claims F12-F21; and (b) a donor molecule comprising from 5′ to 3′: (i) a nucleic acid sequence of a first recombination site, which corresponds to the first recombination site of the landing pad of the engineered cell; (ii) a multiple cloning site; and (iii) a nucleic acid sequence of a second recombination site, which corresponds to the second recombination site of the landing pad of the engineered cell.

In some embodiments, a kit comprises: (a) an engineered cell of any one of claims F1 -F11; and (b) a donor molecule comprising from 5′ to 3′: (i) a nucleic acid sequence of a first recombination site, which corresponds to the first recombination site of the landing pad of the engineered cell; (ii) a multiple cloning site; and (iii) a nucleic acid sequence of a second recombination site, which corresponds to the second recombination site of the landing pad of the engineered cell; and (c) an integrase molecule comprising: (i) a nucleic acid sequence encoding for an integrase that binds to recombination sites of the landing pad and the donor molecule; or (ii) an amino acid sequence of an integrase that binds to the first recombination sites of the landing pad and the donor molecule. In some embodiments, a single polynucleic acid comprises the donor molecule and the integrase molecule.

In some embodiments, the donor molecule further comprises an expression cassette comprising a nucleic acid sequence of a promoter operably linked to a nucleic acid sequence of a counter-selection marker. In some embodiments, the counter-selection marker is HSV-TK, and wherein the kit further comprises ganciclovir. In some embodiments, the promoter of the integrase molecule is a constitutive promoter. In some embodiments, the integrase is a serine integrase. In some embodiments, the integrase is a tyrosine integrase. In some embodiments, the integrase comprises an amino acid sequence of any one of SEQ ID NOs: 40-43, 45-54, 56, 59-61, 64, 65, 67, 68, 70, and 72 or an amino acid sequence having at least 95% identity with the amino acid sequence of any one of SEQ ID NOs: 40-43, 45-54, 56, 59-61, 64, 65, 67, 68, 70, and 72.

In some embodiments, the integrase further comprises the amino acid sequence of a nuclear localization signal (NLS).

In some embodiments, the NLS comprises or consists essentially of the amino acid sequence of any one of SEQ ID NOs: 77-78 and 168-174. In some embodiments, the integrase further comprises a GS linker.

In some aspects, the present disclosure relates to a method of integrating a nucleic acid sequence of interest into a cell genome, the method comprising: (a) introducing a donor molecule into the engineered cell of any one of claims F12-F19, wherein the donor molecule comprises from 5′ to 3′: (i) a nucleic acid sequence of a first recombination site, which corresponds to the first recombination site of the landing pad of the engineered cell; (ii) a nucleic acid sequence of interest; and (iii) a nucleic acid sequence of a second recombination site, which corresponds to the second recombination site of the landing pad of the engineered cell; and (b) expressing the integrase of the integrase molecule, thereby inducing integration of the nucleic acid sequence of interest of the donor molecule into the landing pad of the engineered cell; wherein (b) occurs prior to, concurrently with, or after (a).

In some embodiments, a method of integrating a nucleic acid sequence of interest into a cell genome comprises: (a) introducing a donor molecule into the engineered cell of any one of claims F1-F11, wherein the donor molecule comprises from 5′ to 3′: (i) a nucleic acid sequence of a first recombination site, which corresponds to the first recombination site of the landing pad of the engineered cell; (ii) a nucleic acid sequence of interest; and (iii) a nucleic acid sequence of a second recombination site, which corresponds to the second recombination site of the landing pad of the engineered cell; (b) introducing an integrase molecule into the engineered cell, wherein the integrase molecule comprises: (i) a nucleic acid sequence encoding for an integrase that binds to recombination sites of the landing pad and the donor molecule; or (ii) an amino acid sequence of an integrase that binds to the first recombination sites of the landing pad and the donor molecule; thereby inducing integration of the nucleic acid sequence of interest of the donor molecule into the landing pad of the engineered cell; wherein (a) occurs prior to, concurrently with, or after (b).

In some embodiments, the integrase molecule comprises a nucleic acid sequence of a promoter operably linked to a nucleic acid sequence encoding for an integrase, and wherein promoter of the integrase molecule is a constitutive promoter. In some embodiments, the integrase is a serine integrase. In some embodiments, the integrase is a tyrosine integrase. In some embodiments, the integrase comprises an amino acid sequence of any one of SEQ ID NOs: 40-43, 45-54, 56, 59-61, 64, 65, 67, 68, 70, and 72 or an amino acid sequence having at least 95% identity with the amino acid sequence of any one of SEQ ID NOs: 40-43, 45-54, 56, 59-61, 64, 65, 67, 68, 70, and 72. In some embodiments, the integrase further comprises the amino acid sequence of a nuclear localization signal (NLS). In some embodiments, the NLS comprises or consists essentially of the amino acid sequence of any one of SEQ ID NOs: 77-78 and 168-174. In some embodiments, the integrase further comprises a GS linker.

In some embodiments, the donor molecule further comprises an expression cassette comprising a nucleic acid sequence of a promoter operably linked to a nucleic acid sequence of a counter-selection marker. In some embodiments: (i) the counter-selection marker of the landing pad of the engineered cell is HSV-TK; (ii) the counter-selection marker of the donor molecule is HSV-TK; or (iii) a combination of (i) and (ii).

In some embodiments, the method further comprises contacting the engineered cell with ganciclovir. In some aspects the present disclosure relates to an engineered cell comprising a chromosomal integration of a landing pad, wherein the landing pad comprises, from 5′ to 3′: (i) a nucleic acid sequence of a first recombination site; (ii) a nucleic sequence encoding for an integrase; and (iii) a nucleic acid sequence of a second recombination site; wherein the landing pad further comprises (iv) a nucleic acid sequence of a first promoter positioned 5′ or 3′ to the nucleic acid sequence of the first recombination site and which is operably linked to the nucleic acid sequence encoding for the integrase.

In some embodiments, the landing pad comprises, from 5′ to 3′: (i) a nucleic acid sequence of a first recombination site; (ii) a nucleic sequence encoding for a polycistronic mRNA comprising the nucleic acid sequence of the integrase and a nucleic acid sequence encoding for a landing pad marker; and (iii) a nucleic acid sequence of a second recombination site; wherein the landing pad further comprises (iv) a nucleic acid sequence of a first promoter positioned 5′ or 3′ to the nucleic acid sequence of the first recombination site and which is operably linked to the nucleic acid sequence encoding for the polycistronic mRNA. In some embodiments, the nucleic acid sequence of a first promoter is positioned 5′ to the nucleic acid sequence of the first recombination site. In some embodiments, the landing pad marker comprises: an antibiotic resistance protein; a fluorescent protein; a counter-selection marker; or a combination thereof. In some embodiments, the landing pad marker comprises: a viral 2A peptide; an IRES; or a combination thereof. In some embodiments, the polycistronic mRNA further comprises: a nucleic acid sequence encoding for a viral 2A peptide; a nucleic acid sequence encoding for an IRES; or a combination thereof.

In some embodiments, the polycistronic mRNA comprises, from 5′ to 3′: (i) a nucleic acid sequence encoding for the landing pad marker; (ii) a nucleic acid sequence encoding for an IRES; and (iii) the nucleic acid sequence encoding for the integrase.

In some embodiments, the landing pad comprises: (a) a first expression cassette comprising the nucleic acid sequence of the first promoter and the nucleic acid sequence encoding for the integrases; and (b) a second expression cassette comprising a nucleic acid sequence of a second promoter operably linked to a nucleic acid sequence encoding for a landing pad marker. In some embodiments, the landing pad marker comprises: an antibiotic resistance protein; a fluorescent protein; a counter-selection marker; or a combination thereof. In some embodiments, the landing pad marker further comprises: a viral 2A peptide; an IRES; or a combination thereof. In some embodiments, the first expression cassette is 5′ to the second expression cassette. In some embodiments, the first expression cassette is 3′ to the second expression cassette. In some embodiments, the first expression cassette and the second expression cassette are encoded in the same orientation. In some embodiments, the first expression cassette and the second expression cassette are encoded in opposite orientations.

In some embodiments, the landing pad comprises: (a) a first expression cassette comprising the nucleic acid sequence of the first promoter and the nucleic acid sequence encoding for the integrases; (b) a second expression cassette comprising a nucleic acid sequence of a second promoter operably linked to a nucleic acid sequence encoding for a landing pad marker; and (c) a third expression cassette comprising a nucleic acid sequence of a third promoter operably linked to a nucleic acid sequence encoding for an auxiliary gene. In some embodiments, the landing pad marker comprises: an antibiotic resistance protein; a fluorescent protein; a counter-selection marker; or a combination thereof. In some embodiments, the landing pad marker further comprises: a viral 2A peptide; an IRES; or a combination thereof. In some embodiments, the auxiliary gene comprises a counter-selection marker.

In some embodiments, the first expression cassette is 5′ to one or both of the second expression cassette and the third expression cassette. In some embodiments, the second expression cassette is 5′ to one or both of the first expression cassette and the third expression cassette. In some embodiments, the third expression cassette is 5′ to one or both of the first expression cassette and the second expression cassette. In some embodiments, the first expression cassette, the second expression cassette, and the third expression cassette are encoded in the same orientation. In some embodiments, the first expression cassette, the second expression cassette, and the third expression cassette are not all encoded in the same orientation. In some embodiments, the first expression cassette, the second expression cassette, and the third expression cassette are encoded in alternating orientations.

In some embodiments, the first promoter is a chemically inducible promoter.

In some embodiments, the landing pad further comprises a nucleic acid sequence encoding for a transcriptional activator that binds to the chemically inducible promoter when expressed in the presence of a small molecule inducer.

In some aspects, the present disclosure related to an engineered cell comprising a chromosomal integration of a landing pad, wherein the landing pad comprises, from 5′ to 3′: (a) a first expression cassette comprising a nucleic acid sequence of a first promoter operably linked to a nucleic acid sequence encoding for a polycistronic mRNA, wherein the polycistronic mRNA comprises: (i) a nucleic acid sequence encoding for a landing pad marker; and (ii) a nucleic acid sequence encoding for a transcriptional activator; (b) a second expression cassette comprising a nucleic acid sequence of a second promoter operably linked to a nucleic acid sequence encoding for an integrase, wherein the second promoter is a chemically inducible promoter that is bound by the transcriptional activator of (a), when the transcriptional activator is expressed in the presence of a small molecule inducer; wherein the landing pad further comprises: (c) a first recombination site positioned 5′ to the nucleic acid sequence encoding for the polycistronic mRNA of (a); and (d) a second recombination site positioned 3′ to the second expression cassette of (b). In some embodiments, the second recombination site is positioned 3′ to the first promoter. In some embodiments, the landing pad marker comprises: an antibiotic resistance protein; a fluorescent protein; a counter-selection marker; or a combination thereof.

In some embodiments, the landing pad marker further comprises: a viral 2A peptide; an IRES; or a combination thereof. In some embodiments, the nucleic acid sequence encoding for the landing pad marker and the nucleic acid sequence encoding for the transcriptional activator are separated by a nucleic acid sequence encoding for a viral 2A peptide or an IRES.

In some embodiments, the first expression cassette and the second expression cassette are in the same orientation. In some embodiments, the first expression cassette and the second expression cassette are in opposite orientations.

In some aspects, the present disclosure relates to an engineered cell comprising a chromosomal integration of a landing pad, wherein the landing pad comprises: (a) a first expression cassette comprising a nucleic acid sequence of a first promoter operably linked to a nucleic acid sequence encoding for a landing pad marker; (b) a second expression cassette comprising a nucleic acid sequence of a second promoter operably linked to a nucleic acid sequence encoding for a transcriptional activator; (c) a third expression cassette comprising a nucleic acid sequence of a third promoter operably linked to a nucleic acid sequence of an integrase, wherein the third promoter is a chemically inducible promoter that is bound by the transcriptional activator of (b), when the transcriptional activator is expressed in the presence of a small molecule inducer; wherein the third expression cassette is 3′ to the first expression set, the second expression cassette, or both; and wherein the landing pad further comprises: (d) a first recombination; and (e) a second recombination site; wherein cassette exchange at the first and second recombination sites results in excision of: the nucleic acid sequence encoding for a landing pad marker; the nucleic acid sequence encoding for a transcriptional activator; and the third expression cassette.

In some embodiments, cassette exchange at the first and second recombination sites also results in excision of the first promoter, optionally wherein cassette exchange also results in excision of the second promoter. In some embodiments, cassette exchange at the first and second recombination sites also results in excision of the second promoter, optionally wherein cassette exchange also results in excision of the first promoter. In some embodiments, the first expression cassette and the second expression cassette are 5′ to the expression cassette. In some embodiments, the third expression cassette is 5′ to the second expression cassette. In some embodiments, the third expression cassette is 5′ to the first expression cassette. In some embodiments, the landing pad marker comprises: an antibiotic resistance protein; a fluorescent protein; a counter-selection marker or a combination thereof.

In some embodiments, the landing pad marker further comprises: a viral 2A peptide; an IRES; or a combination thereof. In some embodiments, the second expression cassette comprises a nucleic acid sequence encoding for a polycistronic mRNA comprising the nucleic acid sequence of the transcriptional activator and a nucleic acid sequence of a counter-selection marker. In some embodiments, the polycistronic mRNA further comprises a nucleic acid sequence encoding for a viral 2A peptide, a nucleic acid sequence encoding for an IRES, or a combination thereof.

In some embodiments, the first expression cassette, the second expression cassette, and the third expression cassette are in the same orientation. In some embodiments, the first expression cassette, the second expression cassette, and the third expression cassette are not in the same orientation. In some embodiments, the first expression cassette, the second expression cassette, and the third expression cassette are in alternating orientations.

In some embodiments, the integrase is a serine integrase. In some embodiments, the integrase is a tyrosine integrase.

In some embodiments, the landing pad marker is encoding on a polycistronic mRNA comprising, from 5′ to 3′: (i) a nucleic acid sequence encoding for a fluorescent protein; (ii) a nucleic acid sequence encoding for an antibiotic resistance protein; (iii) a nucleic acid sequence encoding for a viral 2A peptide; and (iv) a nucleic acid sequence encoding for the counter-selection marker.

In some embodiments, the engineered cell is derived from a HEK293 cell, HeLa S3 cell, T-cell, induced pluripotent stem cell (iPSC), natural killer (NK) cell or human embryonic stem cell. In some embodiments, the landing pad is integrated at a safe harbor locus selected from the group consisting of AAVS1, ROSA26, CCR5, and LiPS-A3S. In some embodiments, the engineered cell is derived from a CHO cell. In some embodiments, the landing pad is integrated at a safe harbor locus selected from the group consisting of ROSA26, COSMIC, and H11.

In some aspects, the present disclosure relates to a kit comprising: (a) an engineered cell of any one of claims I1-I51; and (b) a donor molecule comprising from 5′ to 3′: (i) a nucleic acid sequence of a first recombination site, which corresponds to the first recombination site of the landing pad of the engineered cell; (ii) a multiple cloning site; and (iii) a nucleic acid sequence of a second recombination site, which corresponds to the second recombination site of the landing pad of the engineered cell. In some embodiments, the integrase is a serine integrase. In some embodiments, the serine integrase comprises any one of SEQ ID NOs: 40-43, 45-54, 56, 59-61, 64, 65, 67, 68, 70, 72, 75 and 76. In some embodiments, the integrase is a tyrosine integrase.

In some embodiments, the landing pad marker is encoding on a polycistronic mRNA comprising, from 5′ to 3′: (i) a nucleic acid sequence encoding for a fluorescent protein; (ii) a nucleic acid sequence encoding for an antibiotic resistance protein; (iii) a nucleic acid sequence encoding for a viral 2A peptide; and (iv) a nucleic acid sequence encoding for the counter-selection marker.

In some aspects, the present disclosure relates to a method of integrating a nucleic acid sequence of interest into a cell genome, the method comprising: (a) introducing a donor molecule into the engineered cell of any one of claims I1-I51; wherein the donor molecule comprises from 5′ to 3′: (i) a nucleic acid sequence of a first recombination site, which corresponds to the first recombination site of the landing pad of the engineered cell; (ii) a nucleic acid sequence of interest; and (iii) a nucleic acid sequence of a second recombination site, which corresponds to the second recombination site of the landing pad of the engineered cell; and (b) expressing the integrase, thereby inducing integration of the nucleic acid sequence of interest of the donor molecule into the landing pad of the engineered cell; wherein (b) occurs prior to, concurrently with, or after (a). In some embodiments, the integrase is a serine integrase. In some embodiments, the serine integrase comprises any one of SEQ ID NOs: 40-43, 45-54, 56, 59-61, 64, 65, 67, 68, 70, 72, 75 and 76. In some embodiments, the integrase is a tyrosine integrase.

In some embodiments, the landing pad marker is encoding on a polycistronic mRNA comprising, from 5′ to 3′: (i) a nucleic acid sequence encoding for a fluorescent protein; (ii) a nucleic acid sequence encoding for an antibiotic resistance protein; (iii) a nucleic acid sequence encoding for a viral 2A peptide; and (iv) a nucleic acid sequence encoding for the counter-selection marker.

In some embodiments, the present disclosure relates to an engineered cell comprising a chromosomal integration of a first landing pad, wherein the first landing pad comprises a nucleic acid sequence of a first recombination site having the nucleic acid sequence having at least 80%, at least 85%, at least 90%, at least 95%, at least 96%, at least 97%, at least 98%, or at least 99% identity with of any one of SEQ ID NOs: 79-148; and (ii) a nucleic acid sequence of a second recombination site, wherein the second recombination site is orthogonal to the first recombination site.

In some embodiments, the second recombination site comprises a nucleic acid having at least 80%, at least 85%, at least 90%, at least 95%, at least 96%, at least 97%, at least 98%, or at least 99% identity with of any one of SEQ ID NOs: 79-159, 166, and 167. In some embodiments, the first nucleic acid sequence and the second nucleic acid sequence share at least 80%, at least 85%, at least 90%, at least 95%, at least 96%, at least 97%, at least 98%, or at least 99% identity.

In some embodiments, the nucleic acid sequence of the first recombination site and the nucleic acid sequence of the second recombination site differ. In some embodiments, the first recombination site and the second recombination site are recognized by the same integrase. In some embodiments, the first recombination site and the second recombination site are recognized by different integrases.

In some embodiments, The engineered comprises a chromosomal integration of a second landing pad, wherein the second landing pad comprises: (i) a nucleic acid sequence of a third recombination site; and (ii) a nucleic acid sequence of a fourth recombination site. In some embodiments, the first recombination site, the second recombination site, the third recombination site, and the fourth recombination site are all orthogonal with respect to each other. In some embodiments, the third recombination site comprises a nucleic acid of any one of SEQ ID NOs: 79-159, 166, and 167. In some embodiments, the fourth recombination site comprises a nucleic acid of any one of SEQ ID NOs: 79-159, 166, and 167. In some embodiments, the first landing pad comprises a first expression cassette, the second landing pad comprises a second expression cassette, or a combination thereof.

In some embodiments, the engineered cell is derived from a HEK293 cell. In some embodiments, the engineered cell comprises a first landing pad and a second landing pad, and wherein the first landing pad and/or second landing pad is integrated at a safe harbor locus selected from the group consisting of AAVS1, ROSA26, CCR5, and LiPS-A3S, wherein the first landing pad and second landing are not integrated at the same locus. In some embodiments, the engineered cell is derived from a CHO cell. In some embodiments, engineered cell comprises a first landing pad and a second landing pad, and wherein the first landing pad and/or second landing pad is integrated at a safe harbor locus selected from the group consisting of ROSA26, COSMIC, and H11, wherein the first landing pad and second landing are not integrated at the same locus.

In some embodiments, the engineered cell comprises a polynucleotide comprising a nucleic acid sequence of a promoter operably linked to a nucleic acid sequence encoding for a first integrase that binds to the first recombination site of the first landing pad, the second recombination site of the first landing pad, or a combination thereof.

In some embodiments, the first integrase binds to the first recombination site and the second recombination site of the first landing pad. In some embodiments, the first integrase comprises an amino acid sequence of any one of SEQ ID NOs: 39-72 or an amino acid sequence having at least 95% identity with the amino acid sequence of any one of SEQ ID NOs: 39-72.

In some embodiments, the first integrase comprises an amino acid sequence of any one of SEQ ID NOs: 39-43, 45-54, 56, 59-61, 64, 65, 67, 68, 70, and 72 or an amino acid sequence having at least 95% identity with the amino acid sequence of any one of SEQ ID NOs: 40-43, 45-54, 56, 59-61, 64, 65, 67, 68, 70, and 72. In some embodiments, the first integrase comprises the amino acid sequence of a nuclear localization signal (NLS). In some embodiments, the NLS comprises or consists essentially of the amino acid sequence of any one of SEQ ID NOs: 77-78 and 168-174.

In some embodiments, the first integrase further comprises a GS linker.

In some embodiments, the engineered cell further comprises: a polynucleotide comprising a nucleic acid sequence of a promoter operably linked to a nucleic acid sequence encoding for a first integrase that binds to the first recombination site of the first landing pad; and a polynucleotide comprising a nucleic acid sequence of a promoter operably linked to a nucleic acid sequence encoding for a second integrase that binds to the second recombination site of the first landing pad.

In some aspects, the present disclosure relates to a kit comprising: (a) an engineered cell of any one of claims L1-L23; and (b) a donor molecule comprising from 5′ to 3′: (i) a nucleic acid sequence of a first recombination site, which corresponds to the first recombination site of the first landing pad of the engineered cell; (ii) a multiple cloning site; and (iii) a nucleic acid sequence of a second recombination site, which corresponds to the second recombination site of the first landing pad of the engineered cell.

In some aspects, the present disclosure relates to a method of integrating a nucleic acid sequence of interest into a cell genome, the method comprising: (a) introducing a donor molecule into the engineered cell of any one of claims L16-L22; wherein the donor molecule comprises from 5′ to 3′: (i) a nucleic acid sequence of a first recombination site, which corresponds to the first recombination site of a first landing pad of the engineered cell; (ii) the first nucleic acid sequence of interest; and (ii) a nucleic acid sequence of a second recombination site, which corresponds to the second recombination site of the first landing pad of the engineered cell; (b) expressing the first integrase, thereby inducing integration of the first nucleic acid sequence of interest of the first donor molecule into the first landing pad of the engineered cell; wherein (b) occurs prior to, concurrently with, or after (a).

In some aspects, the present disclosure relates to a method of integrating a nucleic acid sequence of interest into a cell genome, the method comprising: (a) introducing a donor molecule into the engineered cell of claim L23; wherein the donor molecule comprises from 5′ to 3′: (i) a nucleic acid sequence of a first recombination site, which corresponds to the first recombination site of a first landing pad of the engineered cell; (ii) the first nucleic acid sequence of interest; and (ii) a nucleic acid sequence of a second recombination site, which corresponds to the second recombination site of the first landing pad of the engineered cell; (b) expressing the first integrase and the second integrase, thereby inducing integration of the first nucleic acid sequence of interest of the first donor molecule into the first landing pad of the engineered cell; wherein (b) occurs prior to, concurrently with, or after (a).

In some aspects, the present disclosure relates to a method of integrating a nucleic acid sequence of interest into a cell genome, the method comprising: (a) introducing a donor molecule into the engineered cell of any one of claims L1-L15, wherein the donor molecule comprises from 5′ to 3′: (i) a nucleic acid sequence of a first recombination site, which corresponds to the first recombination site of the first landing pad of the engineered cell; (ii) a nucleic acid sequence of interest; and (iii) a nucleic acid sequence of a second recombination site, which corresponds to the second recombination site of the first landing pad of the engineered cell; (b) introducing an integrase molecule into the engineered cell, wherein the integrase molecule comprises: (i) a nucleic acid sequence encoding for an integrase that binds to the first recombination site and the second recombination site of the first landing pad and the first recombination site and the second recombination site of the donor molecule; or (ii) an amino acid sequence of an integrase that binds to the first recombination site and the second recombination site of the first landing pad and the first recombination site and the second recombination site of the donor molecule; thereby inducing integration of the nucleic acid sequence of interest of the donor molecule into the landing pad of the engineered cell; wherein (a) occurs prior to, concurrently with, or after (b).

In some aspects, the present disclosure relates to a method of integrating a nucleic acid sequence of interest into a cell genome, the method comprising: (a) introducing a donor molecule into the engineered cell of any one of claims L1-L15, wherein the donor molecule comprises from 5′ to 3′: (i) a nucleic acid sequence of a first recombination site, which corresponds to the first recombination site of the first landing pad of the engineered cell; (ii) a nucleic acid sequence of interest; and (iii) a nucleic acid sequence of a second recombination site, which corresponds to the second recombination site of the first landing pad of the engineered cell; (b) introducing one or more polynucleotides into the engineered cell, collectively comprising: (i) a nucleic acid sequence encoding for a first integrase that binds to the first recombination site of the first landing pad and the first recombination site of the donor molecule; and (ii) a nucleic acid sequence encoding for a second integrase that binds to the second recombination site of the first landing pad and the second recombination site of the donor molecule; thereby inducing integration of the nucleic acid sequence of interest of the donor molecule into the landing pad of the engineered cell; wherein (a) occurs prior to, concurrently with, or after (b).

In some aspects, the present disclosure relates to a method of integrating a nucleic acid sequence of interest into a cell genome, the method comprising: (a) introducing a donor molecule into the engineered cell of any one of claims L1-L15, wherein the donor molecule comprises from 5′ to 3′: (i) a nucleic acid sequence of a first recombination site, which corresponds to the first recombination site of the first landing pad of the engineered cell; (ii) a nucleic acid sequence of interest; and (iii) a nucleic acid sequence of a second recombination site, which corresponds to the second recombination site of the first landing pad of the engineered cell; (b) introducing: (i) a polypeptide comprising an amino acid sequence of a first integrase that binds to the first recombination site of the first landing pad and the first recombination site of the donor molecule; or (ii) a polypeptide comprising an amino acid sequence of a second integrase that binds to the second recombination site of the first landing pad and the second recombination site of the donor molecule; thereby inducing integration of the nucleic acid sequence of interest of the donor molecule into the landing pad of the engineered cell; wherein (a) occurs prior to, concurrently with, or after (b).

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 shows plasmid schematics of transient vectors to test mammalian integrases. The hEF1a promoter and SV40 polyA terminator sequence flank each integrase (upper track) or reporter cassette (middle track). A Kozak sequence (GCCACC) is located upstream of all coding sequences for mammalian expression. The reporter fluorescence protein EGFP is flanked by attB and attP sites in opposite orientations. Upon recombination (lower track), the recombinase ‘flips’ EGFP into the correct orientation in frame with the hEF1a promoter, resulting in EGFP expression and the attL and attR recombined sites.

FIG. 2 shows reporter expression levels in mammalian recombination analyses. 31 of the 34 novel integrases were tested for their ability to recombine a reporter plasmid to express EGFP. Of the tested set, 24 were able to drive EGFP expression in a range of 68% to nearly 100% of all transfected cells, determined by a TagBFP transfection marker. The integrases Int17, Int19, Int20, Int25, Int28, Int31, and Int33 were determined to not be functional in mammalian cells by this assay. Integrase Int24 was not tested in this experiment.

FIG. 3 shows plasmid schematics of stable vectors to test mammalian integrases for genomic integration. The same transient plasmids can be used to express the integrases in a stable cell line, consisting of a hEF1a promoter and SV40 polyA terminator sequence flanking each integrase (upper track). A landing pad consisting of an attP integration site cassette can be stably integrated by low MOI lentiviral transduction (second track). The landing pad expresses EYFP and puromycin as selectable markers. A payload can be co-transfected with each integrase, consisting of an attB integration site cassette followed by hygromycin and TagBFP (third track with expanded cassette). Integrases proven to not be functional were removed from the cassette (Int1, Int6, Int17, Int19, Int20, Int25, Int28, Int31, and Int33). Upon recombination, the recombinase inserts the payload marker (and the entire bacterial backbone of the payload) between the hEF1a promoter and landing pad marker, greatly diminishing the expression of the landing pad marker (lower track) and initiating expression of the payload marker.

FIG. 4 shows plasmid schematics of initial landing pads for lentiviral genomic integration. A transient plasmid expresses the integrase from a strong constitutive promoter hEF1a at the time of payload recombination (first track). The full landing pad sequence is flanked by lentiviral long terminal repeats (LTRs) and virus titer is improved by the Woodchuck Hepatitis Virus Post-transcriptional Regulatory Element (WPRE). The landing pad itself consists of the hEF1a promoter followed by an integrase recombination site, an expression cassette, and optionally a second recombination site for recombinase-mediated cassette exchange (RMCE, second track). The landing pad expression cassette produces the fluorescent protein EYFP and a puromycin antibiotic resistance gene as selectable markers, linked by a P2A cleavage site. A payload will be co-transfected with each integrase, consisting of a recombination site followed by a promoter-less expression cassette, and optionally a second recombination site for RMCE (third track). The payload itself does not contain a promoter, but once integrated, the landing pad promoter drives expression of the fluorescent protein TagBFP and a hygromycin antibiotic resistance gene as selectable markers. The recombinase either mediates insertion of the full payload plasmid (fourth track), or RMCE of the payload marker cassette (fifth track), when designed with only a single recombination site or dual recombination sites, respectively. Both avenues of integration result in stable expression of the payload marker and either greatly diminished or no expression of the landing pad marker.

FIGS. 5A-5B show stable insertion (“single lox landing pad”) or cassette exchange (“double lox landing pad”) of a TagBFP expressing payload marker mediated by Cre recombinase. Negative controls replaced the Cre recombinase with an inert plasmid co-transfected with the same single-lox (“single lox-no integrase” in FIG. 5A) or double-lox (“double lox-no integrase” in FIG. 5A) payloads. The TagBFP payload could be seen to replace the landing pad marker EYFP after 4 days post-transfection, indicated by a rise in the percentage of cells that expressed the TagBFP payload marker and lost expression of the EYFP landing pad marker. This population was stable after 8 days post-transfection in both percentage of the total population (FIG. 5A) and brightness of the TagBFP payload marker (FIG. 5B).

FIG. 6 shows viability for cells under hygromycin selection for Cre mediated stable insertion (“single lox landing pad”) or cassette exchange (“double lox landing pad”) of a hygromycin resistance cassette 2A linked to a TagBFP expressing payload marker. Negative controls replaced the Cre recombinase with an inert plasmid co-transfected with the same single-lox (“single lox-no integrase”) or double-lox (“double lox-no integrase”) payloads. Recombinase mediated integration samples reached lowest viability after 13 days and recovered after 19 days. Negative control samples reached lowest viability after 19 days, and recovered after 26 days, presumably due to randomly integrated payload.

FIG. 7 shows schematics of the Bxb1 integrase expressing plasmid, landing pad plasmid, payload plasmid, and final RMCE product. The Bxb1 integrase is mammalian codon optimized and expressed using the hEF1a promoter. The landing pad is flanked by two different attP sites and contains a fusion protein of EGFP-Puromycin selectable marker translationally linked using a 2A sequence to the Herpes Simplex Virus-1 Thymidine Kinase (HSV-TK) counter selectable marker all driven by the hEF1a promoter and terminated by a strong polyadenylation signal. The payload plasmid contains iRFP translationally linked using a 2A sequence to a glutamine synthetase gene for selection. The payload is flanked by two attB sites which target the attP sites within the landing pad for integration. The payload plasmid lacks a promoter to drive expression of the fluorescent and selection markers and also includes, outside of the payload sequence, an HSV-TK counter selectable marker so that selection and counterselection can be used to isolate clones that have undergone successful RMCE. The final product will contain attL and attR sequences flanking the integrated sequence and expression of the payload sequence will be driven by the landing pad hEF1a promoter.

FIGS. 8A-8B. FIG. 8A shows a generalized workflow for the testing of the Bxb1 double att-site constructs. FIG. 8B shows a PCR screen of the sixty-six surviving clones indicating the presence of a 490 bp band in all clones which indicates successful RMCE. PCR bands absent from parental cell line and landing pad only cell pool demonstrating specificity to PCR screen to successful RMCE target.

FIG. 9 shows plasmid schematics of landing pads for site-specific genomic integration. Each landing pad design can be compared to a version similar to previous designs that express the integrase by co-transfection at the time of payload recombination (first track). The full landing pad sequence is flanked by left or right homology arms (LHA, RHA) and a CTCF insulator. The landing pad itself consists of the hEF1a promoter followed by an integrase recombination site, an expression cassette, and a second recombination site for RMCE. The landing pad expression cassette produces a hygromycin resistance gene fused to the fluorescent protein TagBFP as selectable markers, linked by a 2A cleavage site to the HSV-TK counter-selectable marker. Additionally, a constitutive or inducible integrase is expressed in the landing pad. The constitutive design expresses the integrase on the same transcript as the selectable and counter-selectable marker by an IRES linker (second track). An inducible design implements the same IRES linker arrangement to express the TetOn reverse tetracycline-controlled transactivator (rtTA) for a tetracycline response element (TRE) inducible promoter. Differences in various inducible designs are highlighted in red. The integrase is inducibly expressed by a TRE promoter in a second transcription unit downstream of the expression cassette, either in forward orientation (third track) or reverse orientation (fourth track). Transcription readthrough from the landing pad expression cassette or any downstream transcription units may raise the basal expression of the inducible integrase, and lead to leaky expression prior to induction, and possibly genomic instability if the integrase is thought to be toxic. A final design re-introduces the 2A linker between the hygromycin resistance gene and the fluorescent marker TagBFP, since this configuration was confirmed to express as expected in prior payload designs (lower track). This final design splits the expression cassette and counter-selection cassettes into two transcription units flanking the inducible integrase, with the TetOn rtTA linked to HSV-TK by a 2A linker.

FIG. 10 shows an exemplary payload for the landing pad design of FIG. 9. The payload contains a recombination site followed by a promoter-less expression cassette, and a second recombination site for RMCE (upper track). The payload also contains a second transcription unit for counter-selection. The payload itself does not contain a promoter, but once integrated, the landing pad promoter drives expression of the fluorescent protein EYFP and a puromycin antibiotic resistance gene as selectable markers. The recombinase mediates exchange of the payload marker cassette into the landing pad between the two recombined sites (lower track), resulting in stable expression of the payload marker and no expression of the landing pad marker after counter-selection.

### DETAILED DESCRIPTION

Serine and tyrosine recombinases have been shown to be functional in mammalian systems. One such use of these recombinases is the creation of a “landing pad” sequence that harbors a “payload” sequence to a specific locus (or multiple loci) in a mammalian genome. A fixed integration site is desirable to reduce the variability between experiments that may be caused by positional epigenetic effects or proximal regulatory elements. The ability to control payload copy number is also desirable to modulate expression levels of the payload without changing any genetic components.

In addition to genomic integration, the inversion and excision activity of recombinases can also be used to mediate synthetic logic functions such as switches, logic gates, memory, and combinations thereof to achieve programmable genetic circuits within the host cell.

Described herein are integrases and polynucleic acids encoding the same. Also described herein are landing pad architectures. Engineered mammalian cells comprising these integrases and landing pads are also described, which facilitate site-specific genomic integration of payload molecules.

### I. Integrases and Polynucleic Acids Encoding the Same

In some aspects, the disclosure relates to integrases and polynucleic acids encoding the same. As used herein, the term “integrase” refers to an enzyme that catalyzes the integration of a first polynucleic acid (e.g., a donor polynucleic acid) into a second polynucleic acid (e.g., a chromosome of a host cell). Integration occurs at a “recombination site” or a pair of recombination sites. Recombination sites may mediate inversion, integration/excision, or cassette exchange. Recombined sites are present after recombination occurs. Integrases can be categorized within the family of serine recombinases or tyrosine recombinases. Stark, W. Marshall. “Making serine integrases work for us.” Current opinion in microbiology 38 (2017): 130-136.

Tyrosine recombinases mediate recombination between two identical recombination sites, which results in the same recombination motif after recombination occurs. Since the motifs do not change, the strand exchange may be reversed to the original orientation by a subsequent recombination event. The reversible nature of tyrosine recombinases can be thought to result in lower efficiency for inversion and crossover events, because the outcome of an even number of recombination at a site is the same as if no recombination occurred at all. However, excision events are reversed less frequently because the recombinase machinery is required to be in close proximity to both sites. The reversibility of tyrosine recombinases can be mitigated by introducing asymmetrical mutations to one or both recognition sites that are tolerated prior to recombination, but that cannot be recognized by the recombinase after recombination occurs.

Serine recombinases inherently mediate DNA strand exchange between asymmetric recognition sites, which are named after the bacterial recombination site (attB) and phage recombination site (attP). After recombination occurs, the sites are recombined to no longer be recognized by the recombinase without additional host factors. The unrecognizable sites are named after being on the left (attL) and right (attR) of the integrated phage genome. The natural directionality and high efficiency of serine recombinases make them especially useful as tools for synthetic biology.

Various integrases have been identified previously and include, but are not limited to, Bxb1 integrase, lambda-integrase, Cre recombinase, Flp recombinase, gamma-delta resolvase, Tn3 resolvase, φC31 integrase, or R4 integrase. See e.g., Xu et al., BMC Biotechnol. 2013 Oct. 20; 13: 87; Innis et al., Biotechnol. Bioeng. 2017 August; 114(8): 1837-46; Yang et al., Nat. Methods. 2014 December; 11(12): 1261-66; U.S. Pat. No. 6,746,870 B1; U.S. Pat. No. 6,632,672 B2; U.S. Pat. No. 10,081,817 B2; U.S. Pat. No. 7,282,326 B2; Pub. No.: US 2017/211061 A1; Pub. No.: US 2011/0136237 A1; Pub. No.: US 2015/275232 A1—the entireties of which are incorporated herein by reference. In some of the embodiments described herein, an integrase is selected from the group consisting of Bxb1 integrase, lambda-integrase, Cre recombinase, Flp recombinase, gamma-delta resolvase, Tn3 resolvase, φC31 integrase, and R4 integrase.

### A. Polypeptides Having Integrase Activity

In some aspects, the disclosure relates to polypeptides having integrase activity. In some embodiments, a polypeptide having integrase activity comprises an amino acid sequence of any one of SEQ ID NOs: 39-76 or an amino acid sequence having at least 80% identity with any one of SEQ ID NOs: 39-76. In some embodiments, a polypeptide having integrase activity comprises an amino acid sequence having at least 85%, at least 90%, at least 95%, at least 96%, at least 97%, at least 98%, or at least 99% identity with any one of SEQ ID NOs: 39-76. Methods of determining the extent of identity between two sequences (e.g., two amino acid sequences or two polynucleic acids) are known to those having ordinary skill in the art. One exemplary method is the use of Basic Local Alignment Search Tool (BLAST®) software with default parameters (blast.ncbi.nlm.nih.gov/Blast.cgi).

In some embodiments, a polypeptide has integrase activity in a mammalian cell. For example, in some embodiments, a polypeptide having integrase activity comprises an amino acid sequence of any one of SEQ ID NOs: 40-43, 45-54, 56, 59-61, 64, 65, 67, 68, 70, and 72-76 or an amino acid sequence having at least 80% identity with any one of SEQ ID NOs: 40-43, 45-54, 56, 59-61, 64, 65, 67, 68, 70, and 72-76. In some embodiments, the polypeptide having integrase activity has at least 85%, at least 90%, at least 95%, at least 96%, at least 97%, at least 98%, or at least 99% identity with any one of SEQ ID NOs: 40-43, 45-54, 56, 59-61, 64, 65, 67, 68, 70, and 72-76.

In some embodiments, an integrase described herein further comprises a nuclear localization signal (NLS). Exemplary NLS sequences are known to those having ordinary skill in the art. In some embodiments, an amino acid sequence of a NLS comprises or consists essentially of the amino acid sequence of any one of CCAAAGAAAAAGCGGAAAGTG (SV40, SEQ ID NO: 77), PKKKRKV (SEQ ID NO: 78), SV40: PKKKRKV (SEQ ID NO: 168), Pho: PYLNKRKGKP (SEQ ID NO: 169), c-Myc: PAAKRVKLD (SEQ ID NO: 170), Nucleoplasmin: KRPAATKKAGQAKKKK (SEQ ID NO: 171), Nucleoplasmin derivative: PAAKKKKLD (SEQ ID NO: 172), ERK5: RKPVTAQERQREREEKRRRR (SEQ ID NO: 173), H2B: GKKRSKV (SEQ ID NO: 175), and v-Jun: KSRKRKL (SEQ ID NO: 174).

In some embodiments, an integrase described herein further comprise an amino acid linker (e.g., that separates the amino acid sequence of the integrase from the amino acid sequence of a NLS). In some embodiments, the amino acid linker is a GS linker. Exemplary GS linkers are known to those having ordinary skill in the art. For example, a GS linker may comprise the amino acid sequence GS (or one or more repetitions thereof, such as at least two, at least three, at least four, or at least five repetitions thereof). In some embodiments, a GS linker comprises the amino acid sequence GGGS (SEQ ID NO: 176) (or one or more repetitions thereof, such as at least two, at least three, at least four, or at least five repetitions thereof). In some embodiments, a GS linker comprises the amino acid sequence GGGGS (SEQ ID NO: 177) (or one or more repetitions thereof, such as at least two, at least three, at least four, or at least five repetitions thereof). In some embodiments, a GS linker comprises the amino acid sequence SGGGGS (SEQ ID NO: 178) (or one or more repetitions thereof, such as at least two, at least three, at least four, or at least five repetitions thereof). In some embodiments, a GS linker comprises the amino acid sequence GGSGGGGS (SEQ ID NO: 179) (or one or more repetitions thereof, such as at least two, at least three, at least four, or at least five repetitions thereof).

In some embodiments, a polypeptide having integrase activity comprises, from N- to C-terminus: (i) the amino acid sequence of the integrase; (ii) an amino acid linker; and (iii) a NLS. In some embodiments, a polypeptide having integrase activity comprises, from N- to C-terminus: (i) a NLS (ii) the amino acid sequence of the integrase; and (iii) an amino acid linker.

### B. Polynucleic Acids Encoding a Polypeptide Having Integrase Activity

In some aspects, the disclosure relates to a polynucleic acid encoding a polypeptide having integrase activity, as described in Part IA.

In some embodiments, a polynucleic acid comprises a nucleic acid sequence of any one of SEQ ID NOs: 1-38 or a nucleic acid sequence having at least 80% identity with any one of SEQ ID NOs: 1-38. In some embodiments, a polynucleic acid encodes a polypeptide having integrase activity, wherein the polynucleic acid comprises a nucleic acid sequence having at least 85%, at least 90%, at least 95%, at least 96%, at least 97%, at least 98%, or at least 99% identity with any one of SEQ ID NOs: 1-38.

In some embodiments, the polynucleic acid encodes a polypeptide having integrase activity in a mammalian cell. For example, in some embodiments, a polynucleic acid encodes a polypeptide having integrase activity, wherein polynucleic acid comprises a nucleic acid sequence of any one of comprises a nucleic acid sequence of any one of SEQ ID NOs: 2-5, 7-16, 18, 21-23, 26, 27, 29, 30, 32, and 34-38 or a nucleic acid sequence having at least 80% identity with any one of SEQ ID NOs: 2-5, 7-16, 18, 21-23, 26, 27, 29, 30, 32, and 34-38. In some embodiments, the polynucleic acid encodes a polypeptide having integrase activity, wherein the polynucleic acid comprises a nucleic acid sequence having at least 85%, at least 90%, at least 95%, at least 96%, at least 97%, at least 98%, or at least 99% identity with any one of SEQ ID NOs: 2-5, 7-16, 18, 21-23, 26, 27, 29, 30, 32, and 34-38.

In some embodiments, an integrase described herein further comprises a nuclear localization signal (NLS). In some embodiments, a nucleic acid sequence encoding a NLS comprises or consists essentially of the nucleic acid sequence of SEQ ID NO: 77.

In some embodiments, an integrase described herein further comprise an amino acid linker. In some embodiments, the amino acid linker is a GS linker. Such a GS linker may be encoded by a nucleic acid sequence that comprises or consists essentially of the nucleic acid sequence GGTTCA.

In some embodiments, a polynucleic acid encoding a polypeptide having integrase activity comprises, from 5′ to 3′: (i) a nucleic acid sequence encoding the integrase; (ii) a nucleic acid sequence encoding an amino acid linker; and (iii) a nucleic acid sequence encoding a NLS.

### II. Engineered Cells

In some aspects, the disclosure relates to engineered cells comprising one or more genomic landing pads. As used herein, the term “landing pad” refers to a heterologous polynucleic acid sequence (i.e., a polynucleic acid sequence that is not found in the cell naturally) that facilitates the targeted insertion of a “payload” sequence into a specific locus (or multiple loci) of the cell's genome. Accordingly, the landing pad is integrated into the genome of the cell. A fixed integration site is desirable to reduce the variability between experiments that may be caused by positional epigenetic effects or proximal regulatory elements. The ability to control payload copy number is also desirable to modulate expression levels of the payload without changing any genetic components.

In some embodiments, the landing pad is located at a safe harbor site in the genome of the engineered cell. As used herein, the term “safe harbor site” refers to a location in the genome where genes or genetic elements can be introduced without disrupting the expression or regulation of adjacent genes and/or adjacent genomic elements do not disrupt expression or regulation of the introduced genes or genetic elements. Examples of safe harbor sites are known to those having skill in the art and include, but are not limited to, AAVS1, ROSA26, COSMIC, H11, CCR5, and LiPS-A3S. See e.g., Gaidukov et al., Nucleic Acids Res. 2018 May 4; 46(8): 4072-4086; U.S. Pat. No. 8,980,579 B2; U.S. Pat. No. 10,017,786 B2; U.S. Pat. No. 9,932,607 B2; Pub. No.: US 2013/280222 A; Pub. No.: WO 2017/180669 A1—the entireties of which are incorporated herein. In some embodiments, the safe harbor site is a known site. In other embodiments, the safe harbor site is a previously undisclosed site. See “Methods of Identifying High-Expressing Genomic Loci and Uses Thereof” herein. In some embodiments, an engineered cell described herein comprises a landing pad that is integrated at a safe harbor locus selected from the group consisting of AAVS1, ROSA26, COSMIC, H11, CCR5, and LiPS-A3S.

In some embodiments, the engineered cell is derived from a HEK293 cell, HeLa S3 cell, T-cell, induced pluripotent stem cell (iPSC), natural killer (NK) cell or human embryonic stem cell. In some embodiments, the engineered HEK293 cell, HeLa S3 cell, T-cell, induced pluripotent stem cell (iPSC), natural killer (NK) cell or human embryonic stem cell comprises a landing pad that is integrated at a safe harbor locus selected from the group consisting of AAVS1, ROSA26, CCR5, and LiPS-A3S.

In some embodiments, the engineered cell is derived from a CHO cell. In some embodiments, the engineered CHO cell comprises a landing pad that is integrated at a safe harbor locus selected from the group consisting of ROSA26, COSMIC, and H11.

In some embodiments, the engineered cell described herein comprises at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, at least 70, at least 80, at least 90, at least 100, at least 150, at least 200, at least 300, at least 400, or at least 500 landing pads.

Each of the landing pads described herein comprises at least one recombination site. Recombination sites for various integrases have been identified previously. For example, a landing pad may comprise a recombination site corresponding to a Bxb1 integrase, lambda-integrase, Cre recombinase, Flp recombinase, gamma-delta resolvase, Tn3 resolvase, φC31 integrase, or R4 integrase. Exemplary recombination site sequences are known in the art (e.g., attP, attB, attR, attL, Lox, and Frt). In some embodiments, a landing pad comprises a recombination site having a nucleic acid sequence of any one of SEQ ID NOs: 79-159 or a nucleic acid sequence having at least 80% identity with any one of SEQ ID NOs: 79-159, 166, and 167. In some embodiments, a landing pad comprises a recombination site having a nucleic acid sequence having at least 85%, at least 90%, at least 95%, at least 96%, at least 97%, at least 98%, or at least 99% identity with any one of SEQ ID NOs: 79-159, 166, and 167.

When exposed to an appropriate integrase, a recombination site will recombine with a “cognate,” “complementary,” or “corresponding” recombination site (e.g., of a donor polynucleic acid). Exemplary cognate recombination sites for various integrases are provided in TABLE 2 (providing attB and attP sites for each integrase; for example, SEQ ID NO: 79 and SEQ ID NO: 80 are cognate recombination sites) and TABLE 3. A recombination site will not recombine with a non-cognate or an “orthogonal recombination site.”

Orthogonal recombination sites are critical for using multiple recombinases at the same time. A landing pad may employ orthogonal recombination sites to completely exchange a defined genomic sequence with a defined payload sequence flanked by recombination sites that are complementary to the recombination sites of the landing pad (but orthogonal with respect to each other), known as recombinase mediated cassette exchange (RMCE). These RMCE landing pads were first designed to implement orthogonal recombination sites of two different recombinases that needed to be expressed simultaneously. More recently, two pairs of orthogonal recombination sites for the same recombinase can be achieved by mutating the spacer sequence for one pair of sites. If a recombinase is promiscuous in terms of recognition of its cognate recombination site, it may also integrate into sites that have some sequence identity to the cognate sites leading to undesired off-target recombination. These off-target “pseudo” recognition sites may create unintended recombination products for recognition sites otherwise thought to be orthogonal. Furthermore, pseudo recognition sites can lead to instability of the host genome, resulting in toxicity by the recombinase after prolonged expression.

In some embodiments, a landing pad comprises two or more orthogonal recombination sites. In some embodiments, a landing pad comprises two orthogonal recombination sites have the same nucleic acid sequence. In some embodiments, a landing pad comprises two orthogonal recombination sites having different nucleic acid sequences. In some embodiments, the orthogonal recombination sites having different nucleic acid sequences are recognized by different integrases. In some embodiments, the orthogonal recombination sites having different nucleic acid sequences are recognized by the same integrase. For example, a landing pad may comprise a Bxb1-GA attP recombination site (SEQ ID NO: 147) and a Bxb1-GT attP recombination site (SEQ ID NO: 166).

Exemplary orthogonal recombination sites are provided below (Part IIA).

The landing pads described herein may comprise one or more expression cassettes. An expression cassette comprises a nucleic acid sequence of a promoter operably linked to a nucleic acid sequence encoding a product(s) (an RNA product(s) and/or a polypeptide product(s)). In some embodiments, multiple products are encoded within a single expression cassette. For example, in some embodiments, a single promoter drives expression of a polycistronic RNA encoding for multiple products (an RNA product(s) and/or a polypeptide product(s)). A polycistronic RNA may comprise a nucleic acid sequence of an internal ribosomal entry site (IRES) and/or a nucleic acid sequence of a viral 2A peptide (V2A or 2A).

An IRES may comp++++rises the nucleic acid sequence
of SEQ ID NO: 160:
CCCCTCTCCCTCCCCCCCCCCTAACGTTACTGGCCGAAGCCGCTTGGAA
TAAGGCCGGTGTGCGTTTGTCTATATGTTATTTTCCACCATATTGCCGT
CTTTTGGCAATGTGAGGGCCCGGAAACCTGGCCCTGTCTTCTTGACGAG
CATTCCTAGGGGTCTTTCCCCTCTCGCCAAAGGAATGCAAGGTCTGTTG
AATGTCGTGAAGGAAGCAGTTCCTCTGGAAGCTTCTTGAAGACAAACAA
CGTCTGTAGCGACCCTTTGCAGGCAGCGGAACCCCCCACCTGGCGACAG
GTGCCTCTGCGGCCAAAAGCCACGTGTATAAGATACACCTGCAAAGGCG
GCACAACCCCAGTGCCACGTTGTGAGTTGGATAGTTGTGGAAAGAGTCA
AATGGCTCTCCTCAAGCGTATTCAACAAGGGGCTGAAGGATGCCCAGAA
GGTACCCCATTGTATGGGATCTGATCTGGGGCCTCGGTGCACATGCTTT
ACATGTGTTTAGTCGAGGTTAAAAAAACGTCTAGGCCCCCCGAACCACG
GGGACGTGGTTTTCCTTTGAAAAACACGATGATAATATG
An IRES may comprise the nucleic acid sequence
of SEQ ID NO: 161:
CCCCCCCCCCTAACGTTACTGGCCGAAGCCGCTTGGAATAAGGCCGGTG
TGCGTTTGTCTATATGTTATTTTCCACCATATTGCCGTCTTTTGGCAAT
GTGAGGGCCCGGAAACCTGGCCCTGTCTTCTTGACGAGCATTCCTAGGG
GTCTTTCCCCTCTCGCCAAAGGAATGCAAGGTCTGTTGAATGTCGTGAA
GGAAGCAGTTCCTCTGGAAGCTTCTTGAAGACAAACAACGTCTGTAGCG
ACCCTTTGCAGGCAGCGGAACCCCCCACCTGGCGACAGGTGCCTCTGCG
GCCAAAAGCCACGTGTATAAGATACACCTGCAAAGGCGGCACAACCCCA
GTGCCACGTTGTGAGTTGGATAGTTGTGGAAAGAGTCAAATGGCTCTCC
TCAAGCGTATTCAACAAGGGGCTGAAGGATGCCCAGAAGGTACCCCATT
GTATGGGATCTGATCTGGGGCCTCGGTGCACATGCTTTACATGTGTTTA
GTCGAGGTTAAAAAAACGTCTAGGCCCCCCGAACCACGGGGACGTGGTT
TTCCTTTGAAAAACACGATGATAATAGTTATC
A viral 2A peptide may comprise the amino acid
sequence of ATNFSLLKQAGDVEENPGP (SEQ ID NO: 162)
or EGRGSLLTCGDVEENPGP (SEQ ID NO: 163).

In some embodiments, a landing pad comprises only one expression cassette. In some embodiments, a landing pad comprises at least two, at least 3, at least 4 or at least five expression cassettes. In some embodiments, a landing pad comprises 2, 3, 4, or five expression cassettes. When a landing pad comprises multiple expression cassettes, the cassettes can be positioned in various orientations. Exemplary landing pads having multiple expression cassettes are provided below (see Part IIE).

As described herein, a promoter is “operably linked” to a nucleic acid coding sequence when the position of the promoter relative to the nucleic acid coding sequence is such that binding of a transcriptional activator to the promoter can induce expression of the coding sequence. A promoter of an expression cassette may be a constitutive promoter or an inducible promoter.

A promoter may be a constitutive promoter (i.e., an unregulated promoter that allows for continual transcription). Examples of constitutive promoters are known in the art and include, but are not limited to, cytomegalovirus (CMV) promoters, elongation factor 1α (EF1α) promoters, simian vacuolating virus 40 (SV40) promoters, ubiquitin-C (UBC) promoters, U6 promoters, and phosphoglycerate kinase (PGK) promoters. See e.g., Ferreira et al., Tuning gene expression with synthetic upstream open reading frames. Proc. Natl. Acad. Sci. U.S.A. 2013 July; 110(28): 11284-89; Pub. No.: US 2014/377861 A1; Qin, Jane Yuxia, et al. Systematic comparison of constitutive promoters and the doxycycline-inducible promoter. PloS One 5.5 (2010): e10611.—the entireties of which are incorporated herein by reference.

Alternatively, a promoter may be an inducible promoter (i.e., only activates transcription under specific circumstances). An inducible promoter may be a chemically inducible promoter, a temperature inducible promoter, or a light inducible promoter. Examples of inducible promoters are known in the art and include, but are not limited to, tetracycline/doxycycline inducible promoters, cumate inducible promoters, ABA inducible promoters, CRY2-CIB1 inducible promoters, DAPG inducible promoters, and mifepristone inducible promoters. See e.g., Stanton et al., ACS Synth. Biol. 2014 Dec. 19; 3(12): 880-91; Liang et al., Sci. Signal. 2011 Mar. 15; 4(164): rs2; U.S. Pat. No. 7,745,592 B2; U.S. Pat. No. 7,935,788 B2—the entireties of which are incorporated herein by reference.

In some embodiments, the expression cassette comprises a nucleic acid sequence encoding a landing pad marker. As used herein, the term “landing pad marker” refers to a gene product that can be used to select for engineered cells comprising the landing pad. In some embodiments, the landing pad marker comprises an antibiotic resistance protein. Examples of antibiotic resistance proteins are known in the art (e.g., facilitating puromycin, hygromycin, neomycin, zeocin, blasticidin, or phleomycin selection). See e.g., Pub. No.: WO 1997/15668 A2; Pub. No.: WO 1997/43900 A1—the entireties of which are incorporated here by reference. In some embodiments, a landing pad marker comprises a fluorescent protein. Examples of fluorescent proteins are known in the art (e.g., TagBFP, EBFP2, EGFP, EYFP, mKO2, or Sirius). See e.g., U.S. Pat. No. 5,874,304; Patent No.: EP 0969284 A1; Pub. No.: US 2010/167394 A—the entireties of which are incorporated here by reference. In some embodiments, a landing pad marker comprises HSV-TK. In some embodiments, a landing pad marker further comprises a counter-selection marker (see Part IIC).

HSV-TK may comprise the nucleic acid sequence of
SEQ ID NO: 164:
ATGGCCTCTTATCCTGGACACCAGCACGCCAGCGCCTTTGATCAGGCTG
CCAGATCTAGAGGCCACAGCAACAGAAGAACAGCCCTGCGGCCTCGGAG
ACAGCAAGAGGCTACAGAAGTTCGGCCCGAGCAGAAGATGCCCACACTG
CTGAGAGTGTACATCGACGGCCCTCACGGCATGGGCAAGACCACAACAA
CACAGCTGCTGGTGGCCCTGGGCAGCAGAGATGATATCGTGTACGTGCC
CGAGCCTATGACCTATTGGAGAGTGCTGGGCGCCAGCGAGACAATCGCC
AACATCTACACCACACAGCACCGGCTGGATCAGGGCGAAATTTCTGCTG
GCGACGCCGCCGTGGTTATGACATCTGCCCAGATCACCATGGGCATGCC
TTACGCCGTGACAGATGCTGTGCTGGCCCCTCACATTGGCGGAGAAGCC
GGATCTTCTCATGCCCCTCCACCAGCTCTGACCCTGATCTTCGACAGAC
ACCCTATCGCTCATCTGCTGTGCTACCCTGCCGCCAGATACCTGATGGG
CAGCATGACACCTCAGGCCGTGCTGGCTTTCGTGGCCCTGATTCCTCCT
ACACTGCCCGGCACCAATATCGTGCTGGGAGCCCTGCCTGAGGACCGGC
ACATTGATAGACTGGCCAAGAGACAGCGGCCTGGCGAGAGACTGGATCT
GGCTATGCTGGCCGCCATCAGAAGAGTGTACGGCCTGCTGGCCAACACC
GTGCGGTATCTTCAATGTGGCGGCTCTTGGAGAGAGGACTGGGGACAGC
TTTCTGGCACAGCAGTTCCTCCACAAGGCGCCGAGCCTCAGTCTAATGC
TGGACCCAGACCTCACATCGGCGACACCCTGTTTACCCTGTTCAGAGCC
CCTGAGCTGCTGGCTCCTAACGGCGACCTGTACAACGTGTTCGCCTGGG
CTCTTGACGTGCTGGCAAAGCGGCTGAGATCCATGCACGTGTTCATCCT
GGACTACGATCAGTCCCCTGCCGGCTGTAGAGATGCTCTGCTGCAGCTG
ACAAGCGGCATGGTGCAGACCCACGTTACAACCCCTGGCAGCATCCCCA
CCATCTGTGACCTGGCCAGAACCTTCGCCAGAGAGATGGGCGAAGCCAA
CTGA
HSV-TK may comprise the amino acid sequence of
SEQ ID NO: 165:
MASYPGHQHASAFDQAARSRGHSNRRTALRPRRQQEATEVRPEQKMPTL
LRVYIDGPHGMGKTTTTQLLVALGSRDDIVYVPEPMTYWRVLGASETIA
NIYTTQHRLDQGEISAGDAAVVMTSAQITMGMPYAVTDAVLAPHIGGEA
GSSHAPPPALTLIFDRHPIAHLLCYPAARYLMGSMTPQAVLAFVALIPP
TLPGTNIVLGALPEDRHIDRLAKRQRPGERLDLAMLAAIRRVYGLLANT
VRYLQCGGSWREDWGQLSGTAVPPQGAEPQSNAGPRPHIGDTLFTLFRA
PELLAPNGDLYNVFAWALDVLAKRLRSMHVFILDYDQSPAGCRDALLQL
TSGMVQTHVTTPGSIPTICDLARTFAREMGEAN

In some embodiments, an engineered cell described herein comprises a landing pad comprising: a persistent promoter and/or a persistent WPRE (see Part IIB); a counter-selection marker (see Part IIC); an expression cassette encoding an integrase (see Part IID); or a combination thereof.

In some embodiments, an engineered cell described herein further comprises an integrase molecule comprising a nucleic acid sequence of a promoter (constitutive or inducible, as described herein) operably linked to a nucleic acid sequence encoding for an integrase that binds to a recombination site of a landing pad of the engineered cell. Such an integrase may be as described above in Part I. Such an integrase molecule may be transiently present in the engineered cell. Alternatively, such an integrase molecule may be stably integrated within the genome of the engineered cell.

In some embodiments, the engineered cell described herein comprises a first integrase molecule encoding a first integrase and a second integrase molecule encoding a second integrase. In some embodiments, the first integrase and the second integrase target orthogonal recombination sites.

### A. Exemplary Orthogonal Recombination Sites

In some embodiments, a landing pad comprises a pair of orthogonal recombination sites.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 79; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 79. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 79; and (ii) the second recombination site comprises the nucleic acid sequence of any one of SEQ ID NOs: 81-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 80; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 80. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 80; and (ii) the second recombination site comprises the nucleic acid sequence of any one of SEQ ID NOs: 81-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 81; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 81. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 81; and (ii) the second recombination site comprises the nucleic acid sequence of any one of SEQ ID NOs: 79-80, 83-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 82; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 82. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 82; and (ii) the second recombination site comprises the nucleic acid sequence of any one of SEQ ID NOs: 79-80, 83-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 83; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 83. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 83; and (ii) the second recombination site comprises the nucleic acid sequence of any one of SEQ ID NOs: 79-82, 85-166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 84; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 84. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 84; and (ii) the second recombination site comprises the nucleic acid sequence of any one of SEQ ID NOs: 79-82, 85-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 85; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 85. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 85; and (ii) the second recombination site comprises the nucleic acid sequence of any one of SEQ ID NOs: 79-84, 87-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 86; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 86. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 86; and (ii) the second recombination site comprises the nucleic acid sequence of any one of SEQ ID NOs: 79-84, 87-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 87; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 87. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 87; and (ii) the second recombination site comprises the nucleic acid sequence of any one of SEQ ID NOs: 79-86, 89-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 88; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 88. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 88; and (ii) the second recombination site comprises the nucleic acid sequence of any one of SEQ ID NOs: 79-86, 89-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 89; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 89. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 89; and (ii) the second recombination site comprises the nucleic acid sequence of any one of SEQ ID NOs: 79-88, 91-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 90; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 90. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 90; and (ii) the second recombination site comprises the nucleic acid sequence of any one of SEQ ID NOs: 79-88, 91-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 91; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 91. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 91; and (ii) the second recombination site comprises the nucleic acid sequence of any one of SEQ ID NOs: 79-90, 93-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 92; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 92. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 92; and (ii) the second recombination site comprises the nucleic acid sequence of any one of SEQ ID NOs: 79-90, 93-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 93; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 93. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 93; and (ii) the second recombination site comprises the nucleic acid sequence of any one of SEQ ID NOs: 79-92, 95-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 94; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 94. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 94; and (ii) the second recombination site comprises the nucleic acid sequence of any one of SEQ ID NOs: 79-92, 95-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 95; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 95. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 95; and (ii) the second recombination site comprises the nucleic acid sequence of any one of SEQ ID NOs: 79-94, 97-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 96; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 96. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 96; and (ii) the second recombination site comprises the nucleic acid sequence of any one of SEQ ID NOs: 79-94, 97-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 97; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 97. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 97; and (ii) the second recombination site comprises the nucleic acid sequence of any one of SEQ ID NOs: 79-96, 99-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 98; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 98. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 98; and (ii) the second recombination site comprises the nucleic acid sequence of any one of SEQ ID NOs: 79-96, 99-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 99; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 99. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 99; and (ii) the second recombination site comprises the nucleic acid sequence of any one of SEQ ID NOs: 79-98, 101-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 100; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 100. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 100; and (ii) the second recombination site comprises the nucleic acid sequence of any one of SEQ ID NOs: 79-98, 101-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 101; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 101. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 101; and (ii) the second recombination site comprises the nucleic acid sequence of any one of SEQ ID NOs: 79-100, 103-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 102; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 102. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 102; and (ii) the second recombination site comprises the nucleic acid sequence of any one of SEQ ID NOs: 79-100, 103-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 103; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 103. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 103; and (ii) the second recombination site comprises the nucleic acid sequence of any one of SEQ ID NOs: 79-102, 105-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 104; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 104. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 104; and (ii) the second recombination site comprises the nucleic acid sequence of any one of SEQ ID NOs: 79-102, 105-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 105; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 105. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 105; and (ii) the second recombination site comprises the nucleic acid sequence of any one of SEQ ID NOs: 79-104, 107-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 106; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 106. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 106; and (ii) the second recombination site comprises the nucleic acid sequence of any one of SEQ ID NOs: 79-104, 107-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 107; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 107. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 107; and (ii) the second recombination site comprises the nucleic acid sequence of any one of SEQ ID NOs: 79-106, 109-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 108; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 108. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 108; and (ii) the second recombination site comprises the nucleic acid sequence of any one of SEQ ID NOs: 79-106, 109-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 109; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 109. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 109; and (ii) the second recombination site comprises the nucleic acid sequence of any one of SEQ ID NOs: 79-108, 111-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 110; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 110. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 110; and (ii) the second recombination site comprises the nucleic acid sequence of any one of SEQ ID NOs: 79-108, 111-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 111; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 111. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 111; and (ii) the second recombination site comprises the nucleic acid sequence of any one of SEQ ID NOs: 79-110, 113-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 112; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 112. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 112; and (ii) the second recombination site comprises the nucleic acid sequence of any one of SEQ ID NOs: 79-110, 113-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 113; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 113. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 113; and (ii) the second recombination site comprises the nucleic acid sequence of any one of SEQ ID NOs: 79-112, 115-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 114; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 114. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 114; and (ii) the second recombination site comprises the nucleic acid sequence of any one of SEQ ID NOs: 79-112, 115-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 115; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 115. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 115; and (ii) the second recombination site comprises the nucleic acid sequence of any one of SEQ ID NOs: 79-114, 117-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 116; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 116. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 116; and (ii) the second recombination site comprises the nucleic acid sequence of any one of SEQ ID NOs: 79-114, 117-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 117; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 117. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 117; and (ii) the second recombination site comprises the nucleic acid sequence of any one of SEQ ID NOs: 79-116, 119-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 118; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 118. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 118; and (ii) the second recombination site comprises the nucleic acid sequence of any one of SEQ ID NOs: 79-116, 119-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 119; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 119. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 119; and (ii) the second recombination site comprises the nucleic acid sequence of any one of SEQ ID NOs: 79-118, 121-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 120; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 120. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 120; and (ii) the second recombination site comprises the nucleic acid sequence of any one of SEQ ID NOs: 79-118, 121-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 121; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 121. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 121; and (ii) the second recombination site comprises the nucleic acid sequence of any one of SEQ ID NOs: 79-120, 123-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 122; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 122. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 122; and (ii) the second recombination site comprises the nucleic acid sequence of any one of SEQ ID NOs: 79-120, 123-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 123; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 123. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 123; and (ii) the second recombination site comprises the nucleic acid sequence of any one of SEQ ID NOs: 79-122, 125-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 124; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 124. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 124; and (ii) the second recombination site comprises the nucleic acid sequence of any one of SEQ ID NOs: 79-122, 125-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 125; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 125. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 125; and (ii) the second recombination site comprises the nucleic acid sequence of any one of SEQ ID NOs: 79-124, 127-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 126; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 126. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 126; and (ii) the second recombination site comprises the nucleic acid sequence of any one of SEQ ID NOs: 79-124, 127-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 127; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 127. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 127; and (ii) the second recombination site comprises the nucleic acid sequence of any one of SEQ ID NOs: 79-126, 129-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 128; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 128. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 128; and (ii) the second recombination site comprises the nucleic acid sequence of any one of SEQ ID NOs: 79-126, 129-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 129; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 129. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 129; and (ii) the second recombination site comprises the nucleic acid sequence of any one of SEQ ID NOs: 79-128, 131-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 130; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 130. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 130; and (ii) the second recombination site comprises the nucleic acid sequence of any one of SEQ ID NOs: 79-128, 131-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 131; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 131. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 131; and (ii) the second recombination site comprises the nucleic acid sequence of any one of SEQ ID NOs: 79-130, 133-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 132; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 132. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 132; and (ii) the second recombination site comprises the nucleic acid sequence of any one of SEQ ID NOs: 79-130, 133-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 133; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 133. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 133; and (ii) the second recombination site comprises the nucleic acid sequence of any one of SEQ ID NOs: 79-132, 135-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 134; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 134. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 134; and (ii) the second recombination site comprises the nucleic acid sequence of any one of SEQ ID NOs: 79-132, 135-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 135; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 135. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 135; and (ii) the second recombination site comprises the nucleic acid sequence of any one of SEQ ID NOs: 79-134, 137-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 136; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 136. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 136; and (ii) the second recombination site comprises the nucleic acid sequence of any one of SEQ ID NOs: 79-134, 137-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 137; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 137. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 137; and (ii) the second recombination site comprises the nucleic acid sequence of any one of SEQ ID NOs: 79-136, 139-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 138; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 138. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 138; and (ii) the second recombination site comprises the nucleic acid sequence of any one of SEQ ID NOs: 79-136, 139-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 139; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 139. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 139; and (ii) the second recombination site comprises the nucleic acid sequence of any one of SEQ ID NOs: 79-138, 141-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 140; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 140. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 140; and (ii) the second recombination site comprises the nucleic acid sequence of any one of SEQ ID NOs: 79-138, 141-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 141; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 141. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 141; and (ii) the second recombination site comprises the nucleic acid sequence of any one of SEQ ID NOs: 79-140, 143-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 142; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 142. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 142; and (ii) the second recombination site comprises the nucleic acid sequence of any one of SEQ ID NOs: 79-140, 143-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 143; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 143. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 143; and (ii) the second recombination site comprises the nucleic acid sequence of any one of SEQ ID NOs: 79-142, 145-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 144; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 144. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 144; and (ii) the second recombination site comprises the nucleic acid sequence of any one of SEQ ID NOs: 79-142, 145-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 145; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 145. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 145; and (ii) the second recombination site comprises the nucleic acid sequence of any one of SEQ ID NOs: 79-144, 147-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 146; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 146. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 146; and (ii) the second recombination site comprises the nucleic acid sequence of any one of SEQ ID NOs: 79-144, 147-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 147; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 147. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 147; and (ii) the second recombination site comprises the nucleic acid sequence of any one of SEQ ID NOs: 79-146, 149-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 148; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 148. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 148; and (ii) the second recombination site comprises the nucleic acid sequence of any one of SEQ ID NOs: 79-146, 149-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 149; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 149. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 149; and (ii) the second recombination site comprises the nucleic acid sequence of any one of SEQ ID NOs: 79-148, 150-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 150; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 150. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 150; and (ii) the second recombination site comprises the nucleic acid sequence of any one of SEQ ID NOs: 79-149, 151-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 151; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 151. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 151; and (ii) the second recombination site comprises the nucleic acid sequence of any one of SEQ ID NOs: 79-150, 152-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 152; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 152. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 152; and (ii) the second recombination site comprises the nucleic acid sequence of any one of SEQ ID NOs: 79-151, 153-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 153; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 153. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 153; and (ii) the second recombination site comprises the nucleic acid sequence of any one of SEQ ID NOs: 79-152, 154-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 154; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 154. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 154; and (ii) the second recombination site comprises the nucleic acid sequence of any one of SEQ ID NOs: 79-153, 155-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 155; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 155. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 155; and (ii) the second recombination site comprises the nucleic acid sequence of any one of SEQ ID NOs: 79-154, 156-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 156; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 156. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 156; and (ii) the second recombination site comprises the nucleic acid sequence of any one of SEQ ID NOs: 79-155, 157-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 157; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 157. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 157; and (ii) the second recombination site comprises the nucleic acid sequence of any one of SEQ ID NOs: 79-156, 158-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 158; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 158. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 158; and (ii) the second recombination site comprises the nucleic acid sequence of any one of SEQ ID NOs: 79-157, 159-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 159; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 159. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 159; and (ii) the second recombination site comprises the nucleic acid sequence of any one of SEQ ID NOs: 79-158, 160-159, 166, and 167.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 166; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 166. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 166; and (ii) the second recombination site comprises the nucleic acid sequence of any one of SEQ ID NOs: 79-159.

In some embodiments, a landing pad comprises a first recombination site and a second recombination site, wherein the first recombination site and the second recombination site are orthogonal to each other, and wherein: (i) the first recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 167; and (ii) the second recombination site comprises a nucleic acid sequence having at least 80%, at least 85%, at least 90%, at least 95%, at least 96%, at least 97%, at least 98%, or at least 99% identity with the nucleic acid sequence of SEQ ID NO: 167. In some embodiments, a landing pad comprises a first recombination site and a second recombination site, wherein the first recombination site and the second recombination site are orthogonal to each other, and wherein: (i) the first recombination site comprises the nucleic acid sequence of SEQ ID NO: 167; and (ii) the second recombination site comprises the nucleic acid sequence of any one of SEQ ID NOs: 79-159.

### B. Landing Pads Having a Persistent Promoter and/or a Persistent WPRE

In some embodiments, an engineered cell described herein has a landing pad comprising a persistent promoter (constitutive or inducible, as described herein) and/or a persistent Woodchuck Hepatitis Virus Post-transcriptional Regulatory Element (WPRE). As used herein, the term “persistent promoter” refers to a landing pad promoter that is positioned 5′ to a recombination site of the landing pad and that is capable of driving expression of a promoter-less payload. In such embodiments, a payload that one seeks to integrate at the landing pad need not contain a promoter, because once integrated, the landing pad persistent promoter can drive expression of the payload. Similarly, the term “persistent WPRE,” as used herein, refers to a WPRE that is positioned 3′ to a recombination site of the landing pad and that is capable of being operably linked to a payload upon its integration at the landing pad.

In some embodiments, a landing pad comprises only one recombination site (e.g., a recombination site having a nucleic acid sequence of any one of SEQ ID NOs: 79-159 or a nucleic acid sequence having at least 80%, at least 85%, at least 90%, at least 95%, at least 96%, at least 97%, at least 98%, or at least 99% identity with any one of SEQ ID NOs: 79-159).

In some embodiments, a landing pad comprises a pair of orthogonal recombination sites (e.g., as described in Part IIA).

In some embodiments, a landing pad comprises a persistent promoter. For example, in some embodiments, a landing pad comprises an expression cassette comprising, from 5′ to 3′: (i) a nucleic acid sequence of a persistent promoter; (ii) a nucleic acid sequence of a first recombination site; and (iii) a nucleic acid encoding a product (e.g., a RNA product or a polypeptide product). In some embodiments, a landing pad further comprises (iv) a nucleic acid sequence of a second recombination site, wherein the nucleic acid sequence of the second recombination site is positioned 3′ to the nucleic acid sequence encoding the product. In some embodiments, the expression cassette comprises a nucleic acid sequence encoding a landing pad marker as described herein (e.g., an antibiotic marker or a fluorescent marker).

In some embodiments, a landing pad comprises a persistent WPRE. For example, in some embodiments, a landing pad comprises, from 5′ to 3′: (i) a nucleic acid sequence of a first recombination site; and (ii) a nucleic acid sequence encoding a persistent WPRE. In some embodiments, a landing pad comprises, from 5′ to 3′: (i) a nucleic acid sequence of a first recombination site; (ii) a nucleic acid sequence of a second recombination site; and (iii) a nucleic acid sequence encoding a persistent WPRE. In some embodiments, a persistent polyA sequence is used in the place of the WPRE.

In some embodiments, a landing pad comprises a persistent promoter and a persistent WPRE. For example, in some embodiments, a landing pad comprises, from 5′ to 3′: (i) a nucleic acid sequence of a persistent promoter; (ii) a nucleic acid sequence of a first recombination site; and (iii) a nucleic acid sequence of a persistent WPRE. In some embodiments, a landing pad comprises, from 5′ to 3′: (i) a nucleic acid sequence of a persistent promoter; (ii) a nucleic acid sequence of a first recombination site; (iii) a nucleic acid sequence of a second recombination site; and (iv) a nucleic acid sequence of a persistent WPRE. In some embodiments, a landing pad comprises, from 5′ to 3′: (i) a nucleic acid sequence of a persistent promoter; (ii) a nucleic acid sequence of a first recombination site; (iii) a nucleic acid sequence encoding a landing pad marker, operably linked to the promoter of (i); and (iv) a nucleic acid sequence of a second recombination site; and (v) a nucleic acid sequence of a persistent WPRE.

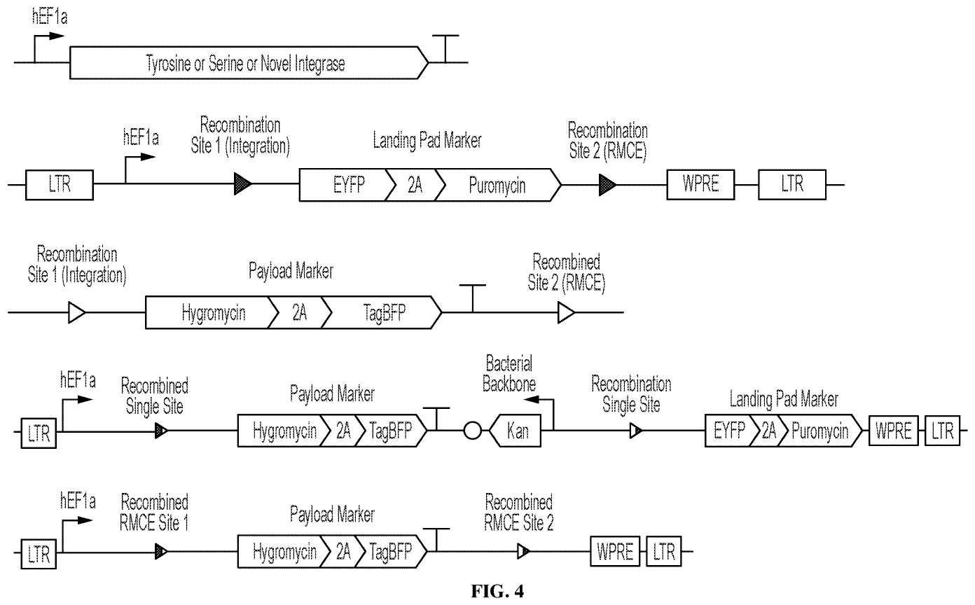

In some embodiments, a landing pad architecture is as depicted in FIG. 4 (third track).

### C. Landing Pads Having a Counter-Selection Marker

In some embodiments, an engineered cell described herein comprises a landing pad having a counter-selection marker and a pair of recombination sites (e.g., orthogonal recombination sites, as described in Part IIA). As used herein, the term “counter-selection marker” refers to a landing pad marker (as described herein) that is shared with a donor molecule. Such a counterselection marker can be used to isolate clones that have undergone successful RMCE. In some embodiments, a counter-selection marker comprises: an antibiotic resistance protein, a fluorescent protein, HSV-TK, or a combination thereof. In some embodiments, a counter-selection marker comprises HSV-TK wildtype or HSV-TK mutants as discussed in Black, Margaret E., et al. “Creation of drug-specific herpes simplex virus type 1 thymidine kinase mutants for gene therapy.” Proceedings of the National Academy of Sciences 93.8 (1996): 3525-3529, which is incorporated by reference in its entirety.

In some embodiments, an engineered cell comprises a landing pad comprising, from 5′ to 3′: (i) a nucleic acid sequence of a first recombination site; (ii) a landing pad marker comprising the nucleic acid sequence of a counter-selection marker; and (iii) a nucleic acid sequence of a second recombination site; wherein the landing pad further comprises (iv) a nucleic acid sequence of a promoter (constitutive or inducible, as described herein) positioned 5′ or 3′ to the first recombination site and which is operably linked to the nucleic acid sequence of the counter-selection marker. In some embodiments, the nucleic acid sequence of the promoter is positioned 5′ to the nucleic acid sequence of the first recombination site.

In some embodiments, a landing pad marker further comprises a selectable marker that is not a counter-selection marker (i.e., not shared with a corresponding donor molecule), such as a nucleic acid sequence encoding for an antibiotic resistance protein, a fluorescent protein, or both.

In some embodiments, a landing pad marker further comprises a nucleic acid sequence encoding for a viral 2A peptide or an IRES. For example, in some embodiments, a landing pad marker encodes for a polycistronic mRNA comprising, from 5′ to 3′: (i) a nucleic acid sequence encoding for a fluorescent protein; (ii) a nucleic acid sequence encoding for an antibiotic resistance protein; (iii) a nucleic acid sequence encoding for a viral 2A peptide; and (iv) a nucleic acid sequence encoding for the counter-selection marker.

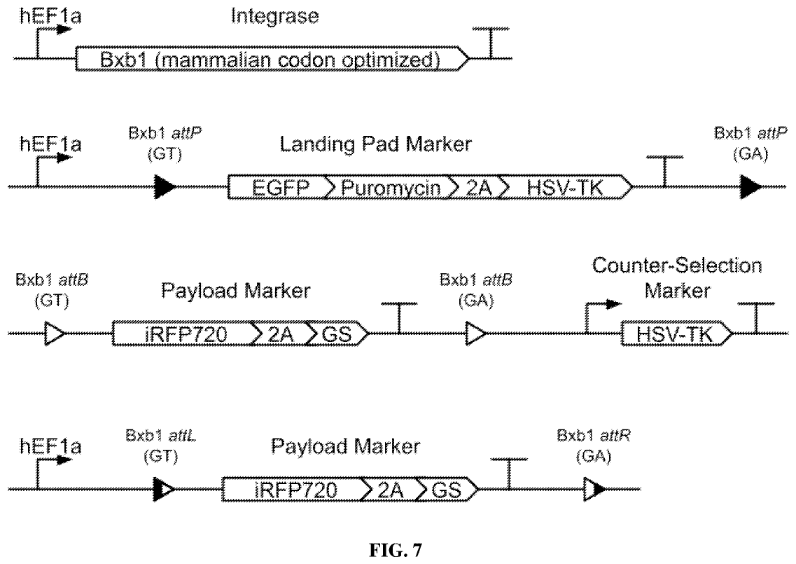

In some embodiments, a landing pad architecture is as depicted in FIG. 7 (second track).

### D. Landing Pads Having a Cassette Encoding an Integrase

In some embodiments, an engineered cell described herein comprises a landing pad having an expression cassette encoding an integrase, such as an integrase as described in Part 1. For example, in some embodiments, an engineered cell comprises a landing pad, wherein the landing pad comprises, from 5′ to 3′: (i) a nucleic acid sequence of a first recombination site; (ii) a nucleic sequence encoding for an integrase; and (iii) a nucleic acid sequence of a second recombination site; wherein the landing pad further comprises (iv) a nucleic acid sequence of a first promoter positioned 5′ or 3′ to the nucleic acid sequence of the first recombination site and which is operably linked to the nucleic acid sequence encoding for the integrase.

In some embodiments, a landing pad comprises, from 5′ to 3′: (i) a nucleic acid sequence of a first recombination site; (ii) a nucleic sequence encoding for a polycistronic mRNA comprising the nucleic acid sequence of the integrase and a nucleic acid sequence encoding for a landing pad marker (as described herein); and (iii) a nucleic acid sequence of a second recombination site; wherein the landing pad further comprises (iv) a nucleic acid sequence of a first promoter positioned 5′ or 3′ to the nucleic acid sequence of the first recombination site and which is operably linked to the nucleic acid sequence encoding for the polycistronic mRNA. In some embodiments, the nucleic acid sequence of the first promoter is positioned 5′ to the nucleic acid sequence of the first recombination site. In some embodiments, the landing pad marker is a counter-selection marker. In some embodiments, the landing pad marker comprises: a viral 2A peptide; an IRES; or a combination thereof. In some embodiments, the polycistronic mRNA further comprises: a nucleic acid sequence encoding for a viral 2A peptide; a nucleic acid sequence encoding for an IRES; or a combination thereof. In some embodiments, the polycistronic mRNA comprises, from 5′ to 3′: (i) a nucleic acid sequence encoding for the landing pad marker; (ii) a nucleic acid sequence encoding for an IRES; and (iii) the nucleic acid sequence encoding for the integrase.

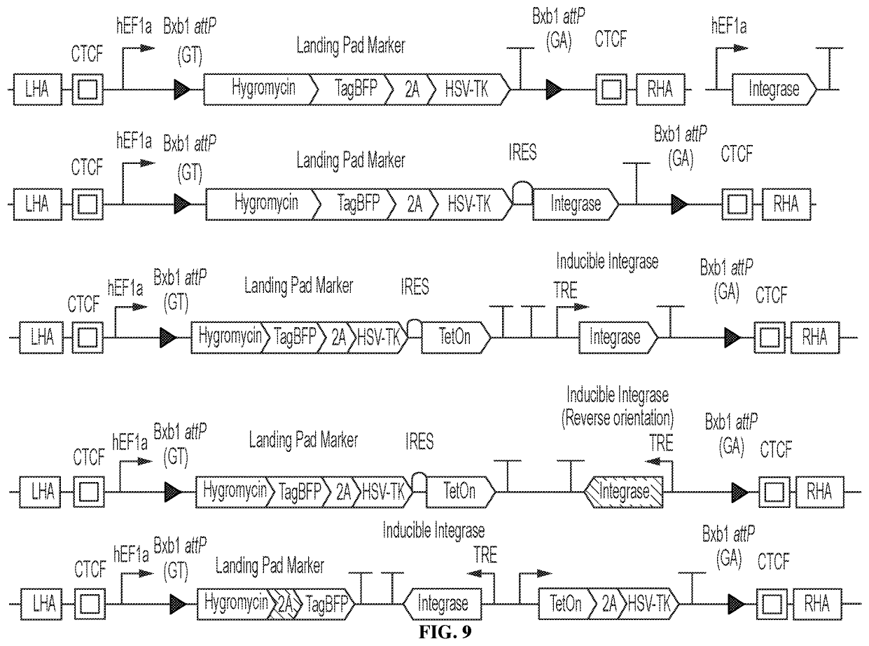

In some embodiments, a landing pad architecture is as depicted in FIG. 9 (second track).

### E. Landing Pads Having Multiple Expression Cassettes

In some embodiments, a landing pad comprises multiple expression cassettes.

### 1. Landing Pads Comprising Two Expression Cassettes

In some embodiments, a landing pad comprises two expression cassettes (a first expression cassette and a second expression cassette). In some embodiments, the first and the second expression cassettes are positioned in the same orientation (i.e., expression is from the same DNA strand). In some embodiments, the first and the second expression cassettes are positioned in a convergent orientation (i.e., expression is from opposite DNA strands and is convergent, →←). In some embodiments, the first and the second expression cassettes are positioned in a divergent orientation (i.e., expression is from opposite DNA strands and is divergent, →←).

In some embodiments, the landing pad comprises: (a) a first expression cassette comprising the nucleic acid sequence of the first promoter and the nucleic acid sequence encoding for an integrase (e.g., as described herein, for example in Part I); and (b) a second expression cassette comprising a nucleic acid sequence of a second promoter operably linked to a nucleic acid sequence encoding for a landing pad marker (e.g., as described herein). In some embodiments, the first expression cassette is 5′ to the second expression cassette. In other embodiments, the first expression cassette is 3′ to the second expression cassette.

In some embodiments, a landing pad comprises, from 5′ to 3′: (a) a first expression cassette comprising a nucleic acid sequence of a first promoter operably linked to a nucleic acid sequence encoding for a polycistronic mRNA, wherein the polycistronic mRNA comprises: (i) a nucleic acid sequence encoding for a landing pad marker (as described herein); and (ii) a nucleic acid sequence encoding for a transcriptional activator; (b) a second expression cassette comprising a nucleic acid sequence of a second promoter operably linked to a nucleic acid sequence encoding for an integrase (as described herein, for example Part I), wherein the second promoter is a chemically inducible promoter that is bound by the transcriptional activator of (a), when the transcriptional activator is expressed in the presence of a small molecule inducer; wherein the landing pad further comprises: (c) a first recombination site positioned 5′ to the nucleic acid sequence encoding for the polycistronic mRNA of (a); and (d) a second recombination site positioned 3′ to the second expression cassette of (b). In some embodiments, the second recombination site is positioned 3′ to the first promoter.

In some embodiments, the landing pad marker comprises a counter-selection marker. In some embodiments, the landing pad marker comprises: a viral 2A peptide; an IRES; or a combination thereof. In some embodiments, the nucleic acid sequence encoding for the landing pad marker and the nucleic acid sequence encoding for the transcriptional activator are separated by a nucleic acid sequence encoding for a viral 2A peptide or an IRES. In some embodiments, the landing pad marker is encoding on a polycistronic mRNA comprising, from 5′ to 3′: (i) a nucleic acid sequence encoding for an antibiotic resistance protein; (ii) a nucleic acid sequence encoding for a fluorescent protein; (iii) a nucleic acid sequence encoding for a viral 2A peptide; and (iv) a nucleic acid sequence encoding for the counter-selection marker.

In some embodiments, a landing pad architecture is as depicted in FIG. 9 (third or fourth track).

### 2. Landing Pads Comprising Three Expression Cassettes

In some embodiments, a landing pad comprises three expression cassettes (a first expression cassette, a second expression cassette, and a third expression cassette). In some embodiments, each of the cassettes are positioned in the same orientation (i.e., expression from each cassette is from the same DNA strand). In some embodiments, one of the three cassettes is positioned in an opposite orientation (i.e., expression of one of the three cassettes is from the opposite DNA strand). Exemplary orientations for the three cassettes are as follows: →→→; ←→→; →←→; and →→←, wherein each arrow in a triplicate may be the first expression cassette, the second expression cassette, or the third expression cassette.

In some embodiments, a landing pad comprises: (a) a first expression cassette comprising the nucleic acid sequence of the first promoter and the nucleic acid sequence encoding for an integrase (as described herein, for example in Part I); (b) a second expression cassette comprising a nucleic acid sequence of a second promoter operably linked to a nucleic acid sequence encoding for a landing pad marker (as described herein); and (c) a third expression cassette comprising a nucleic acid sequence of a third promoter operably linked to a nucleic acid sequence encoding for an auxiliary gene.

In some embodiments, the auxiliary gene comprises a counter-selection marker. In some embodiments, the landing pad marker is encoding on a polycistronic mRNA comprising, from 5′ to 3′: (i) a nucleic acid sequence encoding for a fluorescent protein; (ii) a nucleic acid sequence encoding for an antibiotic resistance protein; (iii) a nucleic acid sequence encoding for a viral 2A peptide; and (iv) a nucleic acid sequence encoding for the counter-selection marker.

In some embodiments, the first expression cassette is 5′ to one or both of the second expression cassette and the third expression cassette.

In some embodiments, the second expression cassette is 5′ to one or both of the first expression cassette and the third expression cassette.

In some embodiments, the third expression cassette is 5′ to one or both of the first expression cassette and the second expression cassette.

In some embodiments, the first expression cassette, the second expression cassette, and the third expression cassette are encoded in the same orientation. In some embodiments, the first expression cassette, the second expression cassette, and the third expression cassette are not all encoded in the same orientation. In some embodiments, the first expression cassette, the second expression cassette, and the third expression cassette are encoded in alternating orientations.

In some embodiments, the first promoter is a chemically inducible promoter. In some embodiments, the landing pad further comprises a nucleic acid sequence encoding for a transcriptional activator that binds to the chemically inducible promoter when expressed in the presence of a small molecule inducer.

In some embodiments, a landing pad comprises: (a) a first expression cassette comprising a nucleic acid sequence of a first promoter operably linked to a nucleic acid sequence encoding for a landing pad marker; (b) a second expression cassette comprising a nucleic acid sequence of a second promoter operably linked to a nucleic acid sequence encoding for a transcriptional activator; (c) a third expression cassette comprising a nucleic acid sequence of a third promoter operably linked to a nucleic acid sequence of an integrase, wherein the third promoter is a chemically inducible promoter that is bound by the transcriptional activator of (b), when the transcriptional activator is expressed in the presence of a small molecule inducer; wherein the third expression cassette is 3′ to the first expression set, the second expression cassette, or both; and wherein the landing pad further comprises: (d) a first recombination; and (e) a second recombination site; wherein cassette exchange at the first and second recombination sites results in excision of: the nucleic acid sequence encoding for a landing pad marker; the nucleic acid sequence encoding for a transcriptional activator; and the third expression cassette. In some embodiments, cassette exchange at the first and second recombination sites also results in excision of the first promoter, optionally wherein cassette exchange also results in excision of the second promoter. In some embodiments, cassette exchange at the first and second recombination sites also results in excision of the second promoter, optionally wherein cassette exchange also results in excision of the first promoter.

In some embodiments, the first expression cassette and the second expression cassette are 5′ to the expression cassette. In some embodiments, the third expression cassette is 5′ to the second expression cassette. In some embodiments, the third expression cassette is 5′ to the first expression cassette.

In some embodiments the landing pad marker comprises a counter-selection marker. In some embodiments, the landing pad marker further comprises: a viral 2A peptide; an IRES; or a combination thereof. In some embodiments, the landing pad marker is encoding on a polycistronic mRNA comprising, from 5′ to 3′: (i) a nucleic acid sequence encoding for an antibiotic resistance protein; (ii) a nucleic acid sequence encoding for a viral 2A peptide; and (iii) a nucleic acid sequence encoding for a fluorescent protein.

In some embodiments, the second expression cassette comprises a nucleic acid sequence encoding for an mRNA comprising the nucleic acid sequence of the integrase.

In some embodiments, the third expression cassette comprises a nucleic acid sequence encoding for a polycistronic mRNA comprising the nucleic acid sequence of the transcriptional activator and a nucleic acid sequence of a counter-selection marker. In some embodiments, the polycistronic mRNA further comprises a nucleic acid sequence encoding for a viral 2A peptide, a nucleic acid sequence encoding for an IRES, or a combination thereof.

In some embodiments, the first expression cassette, the second expression cassette, and the third expression cassette are in the same orientation. In some embodiments, the first expression cassette, the second expression cassette, and the third expression cassette are not in the same orientation. In some embodiments, the first expression cassette, the second expression cassette, and the third expression cassette are in alternating orientations.

In some embodiments, a landing pad architecture is as depicted in FIG. 9 (fifth track).

### III. Kits

In some aspects, the disclosure relates to kits comprising an engineered cell described herein (see Part I).

In some embodiments a kit further comprises a donor molecule. In some embodiments, a donor molecule comprises from 5′ to 3′: (i) a nucleic acid sequence of a first recombination site, which corresponds to the first recombination site of the landing pad of the engineered cell; and (ii) a multiple cloning site. In some embodiments, a donor molecule comprising from 5′ to 3′: (i) a nucleic acid sequence of a first recombination site, which corresponds to the first recombination site of the landing pad of the engineered cell; (ii) a multiple cloning site; and (iii) a nucleic acid sequence of a second recombination site, which corresponds to the second recombination site of the landing pad of the engineered cell. Exemplary multiple cloning sites are known to those having ordinary skill in the art.

In some embodiments, a donor molecule comprises an expression cassette comprising a promoter (constitutive or inducible, as described herein) that is operably linked to a counter-selection marker. In some embodiments, the counter selection marker is HSV-TK. In some embodiments, the kit further comprises ganciclovir.

In some embodiments, a kit further comprises an integrase molecule. In some embodiments, the integrase molecule comprises DNA molecule encoding an integrase comprising a nucleic acid sequence of a promoter (constitutive or inducible, as described herein) operably linked to a nucleic acid sequence encoding for an integrase (e.g., an integrase as described in Part I) that binds to the a recombination site of a landing pad of the engineered cell and a recombination site of the donor molecule. In some embodiments, a single polynucleic acid comprises the donor molecule and the integrase molecule.

In some embodiments, the integrase molecule comprises an mRNA encoding an integrase as described herein. In some embodiments, the integrase molecule comprises an integrase protein as described herein.

In embodiments—wherein the engineered cell, the inducible promoter, and/or the integrase molecule comprises a chemically inducible promoter—the kit may further comprise a corresponding small molecule inducer.

### IV. Methods of Integrating a Nucleic Acid Sequence of Interest into a Cell Genome

In some aspects, the disclosure relates to methods of integrating a nucleic acid sequence of interest into a cell genome.

In some embodiments, a method comprises: (a) introducing a donor molecule into the engineered cell described herein (see Part I), wherein the donor molecule comprises, from 5′ to 3′: (i) a nucleic acid sequence of a recombination site, which corresponds to a recombination site of a landing pad of the engineered cell; and (ii) a nucleic acid sequence of interest; and (b) expressing an integrase that recognizes the recombination site of the landing pad and the recombination site of the donor molecule, thereby inducing integration of the nucleic acid sequence of interest of the donor molecule into the landing pad of the engineered cell. In some embodiments, step (b) occurs prior to step (a). In some embodiments, step (b) occurs concurrently with step (a). In some embodiments, step (b) occurs after step (a).

In some embodiments, after integration, the nucleic acid sequence of interest is operably linked to the promoter of the landing pad of the engineered cell. In some embodiments, prior to integration, the nucleic acid sequence of interest is not operably linked to a promoter.

In some embodiments, a method comprises: (a) introducing a donor molecule into the engineered cell described herein (see Part I), wherein the donor molecule comprises, from 5′ to 3′: (i) a nucleic acid sequence of a recombination site, which corresponds to a recombination site of a landing pad of the engineered cell; and (ii) a nucleic acid sequence of interest; (b) introducing an integrase molecule into the engineered cell, wherein the integrase molecule comprises a nucleic acid sequence of a promoter (constitutive or inducible, as described herein) operably linked to a nucleic acid sequence encoding for an integrase (e.g., as described in Part I) that binds to the first recombination sites of the landing pad and the donor molecule; and (c) expressing the integrase of the integrase molecule, thereby inducing integration of the nucleic acid sequence of interest of the donor molecule into the landing pad of the engineered cell. In some embodiments, step (c) occurs prior to step (a). In some embodiments, step (c) occurs concurrently with step (a). In some embodiments, step (c) occurs after step (a).

In some embodiments, the landing pad of the engineered cell comprises a nucleic acid sequence of a second recombination site; the donor molecule further comprises a nucleic acid sequence of a second recombination site, which corresponds to the second recombination site of the landing pad of the engineered cell; and wherein the integrase binds to the first and second recombination sites of the landing pad and the donor molecule.

In some embodiments, after integration, the nucleic acid sequence of interest is operably linked to the promoter of the landing pad of the engineered cell. In some embodiments, prior to integration, the nucleic acid sequence of interest is not operably linked to a promoter.

In some embodiments, the donor molecule further comprises an expression cassette comprising a nucleic acid sequence of a promoter operably linked to a nucleic acid sequence of a counter-selection marker. In some embodiments, the counter-selection marker of the landing pad of the engineered cell is HSV-TK and the counter-selection marker of the donor molecule is HSV-TK. In such instances, the method may further comprise contacting the engineered cell with ganciclovir.

In some embodiments, the engineered cell comprises a landing pad having a chemically inducible promoter, the donor molecule comprises an inducible promoter, and/or the integrase molecule comprises an inducible promoter. In such instances, the method may further comprise contacting the engineered cell with a small molecule corresponding to the chemically inducible promoter.

### EXAMPLES

### Example 1. Functionality of Prophage Integrases in Mammalian Cells

Previously, bacterial prophages were mined for serine integrases, which resulted in the identification of 34 novel integrases with associated recognition sites (Yang et al. Nat Methods. 2014 December; 11(12): 1261-6). Eleven of these integrases were tested in E. coli and were found to be orthogonal to each other and to FimE and HbiF. Two integrases (Int1 and Int6) were not functional in E. coli. Those integrases found functional were then used as components in genetic circuits.

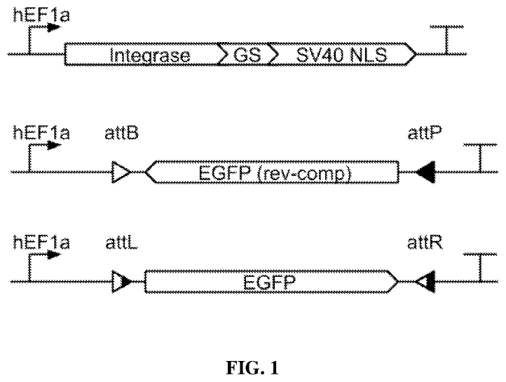

To test if these previously identified prophage integrases are functional in mammalian cells, each integrase was codon optimized for expression in Chinese hamster ovary (CHO) cells (TABLE 1). Next, the SV40 nuclear localization signal (NLS) was appended to the C-terminal end of each integrase (full nucleic acid sequence: CCAAAGAAAAAGCGGAAAGTG, SEQ ID NO: 77; full amino acid sequence: PKKKRKV, SEQ ID NO: 78), separated by a GS linker (full nucleic acid sequence: GGTTCA full amino acid sequence: GS). We expressed each mammalian integrase in pTwist-EF1-Alpha (Twist Biosciences), containing the hEF1a promoter and SV40 polyA (FIG. 1, top track). We did not synthesize or test Int1 or Int6 because these integrases were not found functional in E. coli (Yang et al. Nat Methods. 2014 December; 11(12): 1261-6).

We designed a reporter plasmid that expresses EGFP in the presence of a functional integrase (FIG. 1, middle track). The reporter contains a reverse-complemented EGFP coding sequence downstream of a hEF1a promoter in pTwist-EF1-Alpha. The inverted EGFP is flanked by an attB and attP site in opposite orientations, so that recombination by the corresponding integrase will act as a switch that ‘flips’ the EGFP gene into the correct frame for expression (FIG. 1, lower track). The activity of each integrase was determined by comparing the median fluorescence of the EGFP reporter to the TagBFP transfection marker, normalized to the activity of Bxb1 integrase (Table 5).

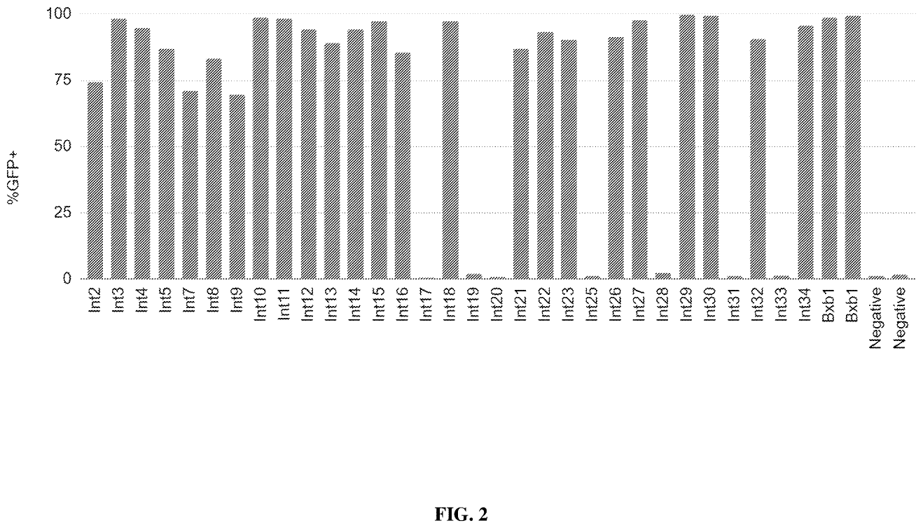

In transient tests, 24 out of the 31 tested integrases were able to perform recombination on the reporter plasmid in mammalian cells (FIG. 2). For these tests, adherent HEK293FT cells were co-transfected with a 600 ng DNA mixture of an integrase expression plasmid, an EGFP reporter plasmid, and a transfection marker plasmid expressing constitutive TagBFP at a 1:1:1 molar ratio. Control samples implementing the Bxb1 mammalian integrase and a corresponding EGFP reporter were also prepared as a positive control, as well as cells transfected with only the TagBFP marker plasmid as a negative control. 48 hours after transfection, all samples were trypsinized and the percentage of EGFP positive cells that passed a TagBFP positive gate was determined by flow cytometry (as the % GFP+). Samples Int2 to Int13 and Int14 to Int34 were tested in batches on two separate days. Calibration beads and duplicate positive and negative controls were run on each day, and deemed comparable to each other without normalization. Integrase Int24 was not tested in this experiment.

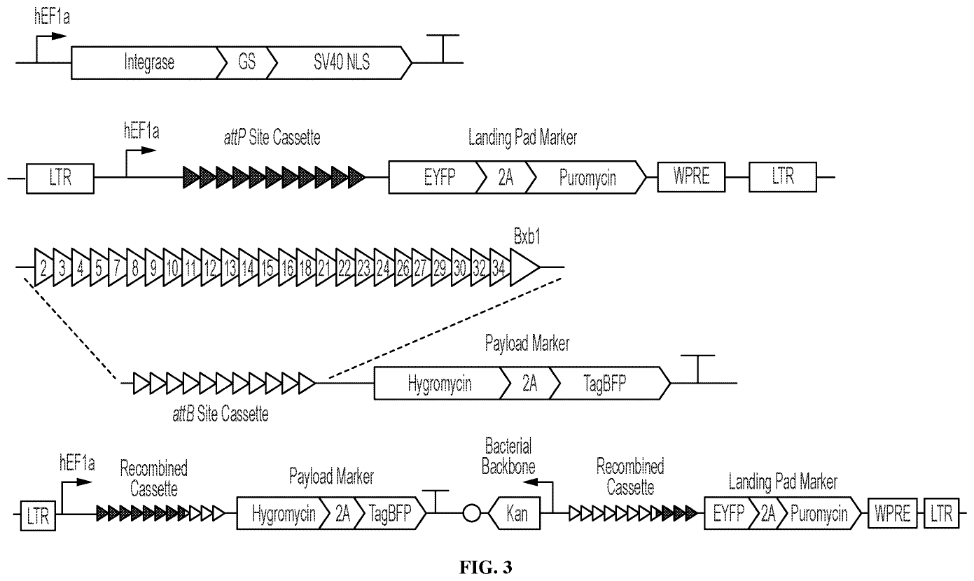

The 24 integrases that were found to be functional in mammalian cells can be used in a landing pad system to screen for high efficiency genomic recombination with low toxicity, high specificity, and high stability. A single cell line containing a stably integrated landing pad with a cassette of every candidate attP recombination site can be constructed by a low MOI lentiviral infection. A single integration cassette can be used to reduce variability that may be caused by creating 24 individual cell lines for each recombinase (FIG. 3).

This stable pool of single-copy landing pad cells can be transfected with each mammalian integrase and a reporter payload containing a cassette of every corresponding attB recombination site (TABLES 2 and 3). The payload (and bacterial backbone) can be inserted between the hEF1a promoter and the landing pad fluorescent protein upon successful recombination. Initial tests with tyrosine recombinase landing pads indicate that successful recombination can be indicated by a greatly diminished level of the landing pad fluorescent protein expression, in addition to expression of the payload fluorescent protein. The efficiency and stability of integration can be determined by monitoring the percentage of cells with integrated payload across many passages. The toxicity of each mammalian integrase can be predicted by measuring the viability of each pool after transfection. A mammalian integrase can be thought to have low specificity if the payload is integrated at pseudo-sites within the mammalian genome, indicated by a high copy number integration of the payload. Furthermore, stable concurrent expression of both the payload and landing pad fluorescent proteins would indicate that the payload is integrated at sites other than the desired recombined site.

TABLE 1
Codon optimized integrase nucleotide sequences. Nucleotide and amino acid sequences
for all integrases tested. Int1-Int34 also included a C-terminal GS linker and NLS.
Nucleotide sequences were codon optimized for mammalian systems.

| SEQ ID NO: | Name | Nucleotide Sequence |
|---|---|---|
| 1 | Int1 | ATGACAAACCCCGCCAGCAGGCCTAAGGCCTACTCCTACATCAGAATGTCCTCCG |
| | | CCATCCAGATCAAGGGCGACTCCTTCCGGCGGCAGGCCGAGGCTTCCGCCAAGT |
| | | ACGCTGCCGAGCACGACCTGGATCTGATCGACGATTACAAACTGGCCGATCTGG |
| | | GGGTGTCCGCCTTCAAGTCCGACAACCTGACCACCGGCGCTCTGGGGCGGTTCGT |
| | | GGCCGAGTGCGAGGCGGGAGAAATCGAGGCTGGATCCTTTCTGCTGATCGAATC |
| | | CCTGGACAGGCTGTCGAGAGACAAGATCCTGGACGCCTTCAGCCTGTTTGCCAGA |
| | | ATTCTGAAAACCGGTGTTAAGATCGTCACCCTGTCTGACGGCCAAGTGTACGACG |
| | | GCTCCAGCGACCAGGTGGGCTCTATCTACTACGCTATCAGCGTGATGATCCGGAG |
| | | CAACGACGAGTCTAAAATCAAGTCCACCAGAGGACTGGCCAACTGGTCCCAGAA |
| | | GAGAAAGCTGGCTGCAGAACACGGCGTGAAGATGTCCTCCCAGTGTCCCGCCTG |
| | | GCTGAAGCTGTCTGTGGATAGAAAGTCCTACCTGATCGACAAGGAAAGGGCTAA |
| | | GATCGTGCAGAGAATCTTCGAGGCCTCTGCCTCTGGCAAAGGCGCCAATCTGATC |
| | | ACCAAGGAACTGAACCGGGACAAGGTGCCTACCTTCGGCAGAGGCGCCCTGTGG |
| | | GCCGAAGCCTTTGTGTCCAAGACCCTGCGGAACCGGGCCGTGTTAGGAGAGTTCC |
| | | AGCCTGGCCAGTACGTGTCTGGTAAGAGACAGCCCGCTGGCGACCCAATCCCTG |
| | | GCTACTTCCCTCCTGTGATCGAAGAGGAGCTGTTCGATATCGTGCAAGCCTCCCT |
| | | GAGAGGCCGCCTCCTCGCTGGCGGCAGAAGAGGCGAGGGCCAGTCCAACATCTT |
| | | CACCCATGTAGCCTTCTGCGGCTACTGCGGCTCCAAGATGAGACACAGAAGCAA |
| | | GGGCAGCAGAGTGAAGGGCAACCCCCCTCACAGATACCTGACCTGTTTCAACAG |
| | | ATTCAACGGCCCAGGCTGCGACTGCAAGCCCCTGCCTTACGCCGCTTTCGAGCGC |
| | | TCTTTCCTGACTTTCGTGCGGGATGTGGACCTGAGAGGCCTGCTGGAAGGCGCCA |
| | | AGAGAAAGTCCGAGGCCAAGACCATCGCTGACAGAATCACCGTGAACGAGGAA |
| | | AAAGTCAGAAAAGCTGATGAGAGAATCCGCGACTACCTGATCAAGATCGAAGGA |
| | | GCTCCTGACCTGGCCGAGATCTTCATGGAACGGATCAGAGAGCTGAAGGCTGAG |
| | | AAGGACGACCTGGTCAGATCTATCGAAGAGTCCAACGACGCTCTGTCCAAGATC |
| | | AAATCTGACAACGTGACAGACGAGGAGCTGGCTAGCTTGATCTCTACCTTTCAGA |
| | | ACCCTTGCGGAGAGAATCGGATCAGACTGGCCGACCGGATAAAGTCCATCATCG |
| | | AGAGAATCGACGTGTATCCCAACGGCGAAATCCGGAAGGACGACCCTGCCATCG |
| | | ATCTGGTCCGGGCTTCTGGCGATCCTGACGCTGAGAAGATCATCGCCGCCATGAA |
| | | CGCCGGCTCTAGACTGAAGGACGACCCTTACTTCATCGTGACCTTCCGGAATGGC |
| | | GCTGTGCAGACCGTGGTGCCTAACCCTTCCAACCCTGATGATATTCGGGTTTCTGT |
| | | GTACGCAGGCGAAAAGACCCGACGGGTGGAAGGCTCTGCCTATGAGTACGAGTC |
| | | CGAT |
| 39 | | MTNPASRPKAYSYIRMSSAIQIKGDSFRRQAEASAKYAAEHDLDLIDDYKLADLGVS |
| | | AFKSDNLTTGALGRFVAECEAGEIEAGSFLLIESLDRLSRDKILDAFSLFARILKTGVKI |
| | | VTLSDGQVYDGSSDQVGSIYYAISVMIRSNDESKIKSTRGLANWSQKRKLAAEHGVK |
| | | MSSQCPAWLKLSVDRKSYLIDKERAKIVQRIFEASASGKGANLITKELNRDKVPTFGR |
| | | GALWAEAFVSKTLRNRAVLGEFQPGQYVSGKRQPAGDPIPGYFPPVIEEELFDIVQAS |
| | | LRGRLLAGGRRGEGQSNIFTHVAFCGYCGSKMRHRSKGSRVKGNPPHRYLTCFNRF |
| | | NGPGCDCKPLPYAAFERSFLTFVRDVDLRGLLEGAKRKSEAKTIADRITVNEEKVRK |
| | | ADERIRDYLIKIEGAPDLAEIFMERIRELKAEKDDLVRSIEESNDALSKIKSDNVTDEEL |
| | | ASLISTFQNPCGENRIRLADRIKSIIERIDVYPNGEIRKDDPAIDLVRASGDPDAEKIIAA |
| | | MNAGSRLKDDPYFIVTFRNGAVQTVVPNPSNPDDIRVSVYAGEKTRRVEGSAYEYES |
| | | D |
| 2 | Int2 | ATGCCTATCGCCCCTGAGTTCCTGTCTCTGGCCTACCCCGGACAAGAGTTCCCTGC |
| | | CTACCTGTACGGCAGAGCCTCTAGAGATCCTAAGCGGAAGGGCAGATCTGTGCA |
| | | GAGCCAGCTGGACGAAGGCAGAGCCACATGCCTGGATGCCGGCTGGCCTATTGC |
| | | CGGCGAATTTAAGGACGTGGATCGGTCCGCTTCTGCTTACGCCAGACGGACACGG |
| | | GACGAATTCGAGGAGATGATCGCTGGCATCCAGGCCGGAGAGTGCAGGATTCTG |
| | | GTCGCCTTCGAGGCAAGCAGATACTACCGGGACCTGGAGGCTTATGTTCGGCTGC |
| | | GGAGAGTGTGCAGAGAGGCCGGCGTCCTCCTGTGCTACAACGGCCAGGTGTACG |
| | | ACCTGTCCAAGTCCGCCGACAGAAAGGCCACCGCTCAGGACGCTGTGAACGCCG |
| | | AGGGAGAAGCTGACGACATCAGAGAACGGAACCTGAGAACCACCAGACTGAAT |
| | | GCTAAGAGAGGCGGCGCCCACGGCCCTGTGCCTGATGGCTACAAGAGAAGATAC |
| | | GACCCCGACTCTGGCGACCTGGTGGACCAGATCCCTCATCCTGATAGAGCGGGCC |
| | | TGATCACCGAGATCTTCCGGCGCGCTGCCGCTGCTGAGCCCCTGGCTGCTATCTG |
| | | TCGGGATCTGAACGAGAGAGGCGAGACAACCCACAGGGGAAAAGCTTGGCAGA |
| | | GACACCACCTGCACGCCATCCTGAGAAATCCCGCCTACATCGGCCACCGGAGGC |
| | | ATCTGGGCGTGGACACCGGCAAAGGTATGTGGGCTCCTATCTGCGACGACGAGG |
| | | ACTTCGCCGAAACCTTCCAGGCCGTGCAGGAGATCTTATCTTTGCCAGGCAGACA |
| | | GCTGTCTCCTGGCCCAGAAGCTCAGCACCTGCAGACCGGAATCGCCCTGTGTGGC |
| | | GAGCACCCTGACGAGCCTCCTCTGAGATCCGTGACCGTGCGCGGCCGGACCAACT |
| | | ACAACTGCTCCACCAGATATGATGTGGCCATGAGAGAAGATCGGATGGACGCCT |
| | | TCGTGGAAGAGTCCGTGATCACCTGGCTGGCCTCCGACGAAGCCGTGGCTGCCTT |
| | | TGAGGACAACACCGACGATGAGCGGACACGGAAGGCCCGGATCCGGCTGAAGGT |
| | | GCTGGAGGAACAGCTGGAAGCCGCCCAGAAGCAGGCTAGAACCCTGCGGCCTGA |
| | | CGGCATGGGCATGCTGCTGTCCATCGACTCCCTGGCTGGCCTGGAAGCCGAGCTG |
| | | ACCCCTCAAATCGACAAGGCCAGACAAGAATCCCGGAGCCTGCACGTGCCCGCT |
| | | CTGCTGAGAGATCTGCTGGGCAAGCCTAGAGCCGACGTCGACCGGGCCTGGAAC |
| | | GAGGCTCTAACCCTGCCCCAGCGGCGGATGATCCTAAGAATGGTGGTGACCATC |
| | | AGACTGTTCAAAGCTGGCTCTAGAGGCGTGCGGGCCATCGAGCCTGGCCGGATC |
| | | ACCCTGTCCTACGTGGGCGAGCCAGGCTTCAAGCCCGTGGGCGGCAACCGGGCC |
| | | AAGCAG |
| 40 | | MPIAPEFLSLAYPGQEFPAYLYGRASRDPKRKGRSVQSQLDEGRATCLDAGWPIAGE |
| | | FKDVDRSASAYARRTRDEFEEMIAGIQAGECRILVAFEASRYYRDLEAYVRLRRVCR |
| | | EAGVLLCYNGQVYDLSKSADRKATAQDAVNAEGEADDIRERNLRTTRLNAKRGGA |
| | | HGPVPDGYKRRYDPDSGDLVDQIPHPDRAGLITEIFRRAAAAEPLAAICRDLNERGET |
| | | THRGKAWQRHHLHAILRNPAYIGHRRHLGVDTGKGMWAPICDDEDFAETFQAVQEI |
| | | LSLPGRQLSPGPEAQHLQTGIALCGEHPDEPPLRSVTVRGRTNYNCSTRYDVAMRED |
| | | RMDAFVEESVITWLASDEAVAAFEDNTDDERTRKARIRLKVLEEQLEAAQKQARTL |
| | | RPDGMGMLLSIDSLAGLEAELTPQIDKARQESRSLHVPALLRDLLGKPRADVDRAW |
| | | NEALTLPQRRMILRMVVTIRLFKAGSRGVRAIEPGRITLSYVGEPGFKPVGGNRAKQ |
| 3 | Int3 | ATGAGAAAGGTGGCCATCTACAGCCGGGTGTCCACCATCAACCAGGCCGAAGAG |
| | | GGCTATTCTATCCAGGGCCAAATCGAGGCCCTGACCAAGTACTGCGAGGCTATGG |
| | | AATGGAAGATCTACAAAAACTACTCCGACGCCGGCTTCTCCGGAGGCAAGCTCG |
| | | AAAGACCCGCTATAACCGAGCTGATTGAGGACGGCAAGAACAACAAGTTTGACA |
| | | CCATCCTGGTGTACAAGCTGGATCGGCTGTCCCGGAACGTGAAGGACACACTCTA |
| | | CCTGGTTAAAGATGTGTTCACCGCTAACAACATCCACTTCGTGTCTCTTAAGGAG |
| | | AACATCGATACTTCCTCTGCCATGGGAAACCTGTTCCTGACCCTGCTGTCTGCTAT |
| | | CGCCGAGTTCGAGAGAGAACAGATCAAGGAGCGGATGCAGTTCGGTGTGATGAA |
| | | CCGGGCTAAGTCCGGCAAAACAACAGCTTGGAAAACCCCTCCTTACGGCTACAG |
| | | ATACAACAAGGACGAAAAGACCCTGTCTGTCAACGAGCTGGAAGCCGCCAACGT |
| | | CAGACAGATGTTCGACATGATCATCTCCGGCTGTAGCATCATGTCCATCACCAAC |
| | | TACGCCCGGGACAACTTTGTGGGCAACACCTGGACCCACGTGAAGGTGAAGCGG |
| | | ATCCTGGAAAACGAAACCTACAAGGGCCTGGTCAAGTACAGAGAGCAGACATTT |
| | | TCTGGCGACCACCAGGCAATCATCGATGAGAAAACCTACAATAAGGCCCAGATC |
| | | GCTCTGGCTCATAGAACCGACACCAAGACAAACACCAGACCATTCCAGGGCAAG |
| | | TACATGCTGTCTCATATCGCCAAGTGCGGCTACTGTGGCGCTCCTCTGAAAGTGT |
| | | GCACCGGCAGAGCCAAGAACGATGGCACCAGACGGCAAACCTACGTGTGCGTGA |
| | | ACAAGACCGAGTCCCTGGCCAGAAGGAGCGTGAATAATTATAACAACCAGAAGA |
| | | TCTGCAACACCGGCCGCTACGAGAAGAAGCACATCGAGAAGTATGTGATCGACG |
| | | TGCTGTACAAGCTGCAGCACGACAAAGAGTACCTGAAAAAGATCAAAAAGGACG |
| | | ATAATATCATCGACATCACCCCTCTGAAGAAAGAAATCGAGATCATCGACAAGA |
| | | AGATCAACAGACTGAACGACCTGTACATCAACGATCTGATCGATCTGCCCAAGCT |
| | | GAAAAAGGATATCGAGGAACTGAACCACCTGAAGGACGACTACAACAAGGCCAT |
| | | CAAGCTGAACTACCTGGACAAGAAGAATGAGGATTCTCTGGGCATGCTGATGGA |
| | | CAACCTGGACATCCGGAAAAGCTCCTACGACGTGCAGTCCAGAATCGTGAAGCA |
| | | GCTGATCGACAGAGTGGAAGTGACCATGGACAATATCGACATTATCTTCAAGTTC |
| 41 | | MRKVAIYSRVSTINQAEEGYSIQGQIEALTKYCEAMEWKIYKNYSDAGFSGGKLERP |
| | | AITELIEDGKNNKFDTILVYKLDRLSRNVKDTLYLVKDVFTANNIHFVSLKENIDTSS |
| | | AMGNLFLTLLSAIAEFEREQIKERMQFGVMNRAKSGKTTAWKTPPYGYRYNKDEKT |
| | | LSVNELEAANVRQMFDMIISGCSIMSITNYARDNFVGNTWTHVKVKRILENETYKGL |
| | | VKYREQTFSGDHQAIIDEKTYNKAQIALAHRTDTKTNTRPFQGKYMLSHIAKCGYCG |
| | | APLKVCTGRAKNDGTRRQTYVCVNKTESLARRSVNNYNNQKICNTGRYEKKHIEKY |
| | | VIDVLYKLQHDKEYLKKIKKDDNIIDITPLKKEIEIIDKKINRLNDLYINDLIDLPKLKK |
| | | DIEELNHLKDDYNKAIKLNYLDKKNEDSLGMLMDNLDIRKSSYDVQSRIVKQLIDRV |
| | | EVTMDNIDIIFKF |
| 4 | Int4 | ATGATCACAACCAGAAAGGTTGCCATCTATGTGAGAGTGTCCACCACCAACCAG |
| | | GCTGAAGAAGGCTACTCCATCCAGGGCCAGATCGACTCCCTGATTAAGTACTGCG |
| | | AGGCTATGGGCTGGATCATCTACGAGGAGTACACCGACGCTGGCTTCTCCGGCGG |
| | | AAAAATCGATCGGCCTGCCATGAGTAAGCTGATCACCGATGCCAAGCACAAGAG |
| | | ATTCGATACAATCCTGGTGTACAAGCTGGACAGACTGAGCAGATCCGTGCGGGA |
| | | CACACTGTACCTGGTCAAGGATGTGTTCAACCAGAACAACATCCACTTCGTGTCC |
| | | CTGCAGGAGAATATCGACACCTCCAGCGCCATGGGAAACCTGTTCCTGACCCTGC |
| | | TCTCTGCTATCGCCGAGTTCGAGAGAGAGCAGATCACCGAGCGGATGACCATGG |
| | | GCAAGATCGGCAGAGCCAAGTCTGGCAAGACCATGGCCTGGACCTACACCCCTT |
| | | TTGGCTACGACTATAACAAAGAGAAGGGCGAGCTGATCCTGGATCCTGCTAAGG |
| | | CCCCCATCGTGAAGATGATCTACACCGACTACCTGAAGGGTATGAGCATCCAAA |
| | | AGATCGTGGACAAACTAAACAAGATGGACTACAACGGCAAGGACTGCACCTGGT |
| | | TCCCACACGGCGTGAAACATCTGCTGGACAATCCTGTGTACTACGGCATGACTAG |
| | | ATATAACAACAAGCTGTTTCCTGGCAACCACCAGCCAATCATCACCAAGGAACTG |
| | | TTTGACAAGACCCAGCGCGAGAGACAGAGAAGAAGGCTGGGCATCGAAGAGAA |
| | | TCACTACACCATACCTTTCCAGGCCAAATACATGCTGTCTAAGTTCCTGAGATGC |
| | | AGACAGTGCGGCTCTAGAATGGGCCTGGAGCTGGGCAGACCTCGGAAGAAAGAG |
| | | GGAAAGCGGTCCAAGAAGTACTACTGTCTGAACTCCAGGCCCAAGAGAACCGCC |
| | | TCCTGCGACACCCCTCTGTACGATGCTGAAACCCTGGAAGATTACGTGCTGCACG |
| | | AGATCGCCAAAATCCAGAAGGACCCTTCTATCGCTTCTCGGCAAAAACACATCGA |
| | | AGATCATGAATTGAAATACAAGCGGGAACGGATCGAGGCCAACATCAACAAGAC |
| | | CGTGAACCAGCTGTCCAAGCTGAACAACCTGTACCTGAATGACCTGATCACCCTC |
| | | GAGGACCTGAAAACCCAGACCAACACCCTGATTGCTAAGAAGCGACTGCTGGAA |
| | | AACGAGCTGGACAAGACCTGTGACAACGACGACGAGCTCGACAGACAAGAGAC |
| | | AATCGCCGACTTCCTGGCTCTGCCTGACGTGTGGACAATGGATTACGAGGGCCAG |
| | | AAGTACGCCGTGGAACTGCTGGTGCAGAGAGTGAAGGTGGACCGGGACAACATC |
| | | GACATCCACTGGACCTTC |
| 42 | | MITTRKVAIYVRVSTTNQAEEGYSIQGQIDSLIKYCEAMGWIIYEEYTDAGFSGGKID |
| | | RPAMSKLITDAKHKRFDTILVYKLDRLSRSVRDTLYLVKDVFNQNNIHFVSLQENIDT |
| | | SSAMGNLFLTLLSAIAEFEREQITERMTMGKIGRAKSGKTMAWTYTPFGYDYNKEK |
| | | GELILDPAKAPIVKMIYTDYLKGMSIQKIVDKLNKMDYNGKDCTWFPHGVKHLLDN |
| | | PVYYGMTRYNNKLFPGNHQPIITKELFDKTQRERQRRRLGIEENHYTIPFQAKYMLSK |
| | | FLRCRQCGSRMGLELGRPRKKEGKRSKKYYCLNSRPKRTASCDTPLYDAETLEDYV |
| | | LHEIAKIQKDPSIASRQKHIEDHELKYKRERIEANINKTVNQLSKLNNLYLNDLITLED |
| | | LKTQTNTLIAKKRLLENELDKTCDNDDELDRQETIADFLALPDVWTMDYEGQKYAV |
| | | ELLVQRVKVDRDNIDIHWTF |
| 5 | Int5 | ATGCCTGGCATGACCACCGAAACCGGCCCCGATCCTGCCGGCCTGATCGACCTGT |
| | | TCTGCAGAAAAAGCAAAGCTGTCAAGTCCAGAGCCAATGGCGCTGGACAGCGGA |
| | | GAAAGCAAGAAATCTCCATCGCCGCCCAGGAAACCCTGGGCCGAAAGGTGGCTG |
| | | CCCTGCTCGGCATGCAGGTGCGGCATGTGTGGAAGGAAGTGGGATCTGCTTCTCG |
| | | GTTTAGAAAGGGCAAGGCTCGGGACGACCAGTCCAAGGCCCTGAAGGCCCTGGA |
| | | ATCTGGCGAGGTGGGCGCTCTGTGGTGCTACCGGCTGGATAGATGGGACAGAGG |
| | | CGGCGCTGGAGCCATCCTGAAGATCATCGAGCCTGAGGACGGCATGCCCCGGCG |
| | | GCTGCTGTTTGGCTGGGATGAGGACACCGGCAGACCTGTCCTGGACTCCACCAAC |
| | | AAGCGGGATCGGGGCGAGCTGATTAGACGGGCCGAGGAGGCCAGAGAAGAAGC |
| | | CGAAAAGCTGTCCGAGAGAGTCAGAGATACAAAAGCCCACCAGAGAGAGAACG |
| | | GCGAGTGGGTGAACGCCAGAGCCCCTTACGGCCTGAGAGTGGTGCTGGTGACCG |
| | | TGTCCGACGAGGAAGGCGACGAGTACGACGAGCGGAAGCTGGCTGCCGACGATG |
| | | AGGACGCTGGCGGCCCTGACGGTCTGACCAAGGCTGAAGCCGCTAGACTGGTGT |
| | | TCACCCTGCCTGTGACCGACAGACTCTCTTACGCCGGCACCGCTCACGCCATGAA |
| | | CACCAGAGAGATCCCATCTCCCACCGGCGGACCCTGGATCGCCGTTACCGTGCGG |
| | | GACATGATCCAGAACCCCGCCTACGCTGGCTGGCAGACCACAGGCAGACAGGAC |
| | | GGCAAGCAGCGGAGACTGACCTTCTATAACGGCGAAGGCAAACGCGTGTCCGTG |
| | | ATGCACGGCCCTCCTCTGGTCACAGACGAGGAGCAGGAAGCCGCCAAGGCAGCC |
| | | GTGAAGGGAGAGGATGGCGTGGGCGTGCCACTGGACGGCTCTGACCACGACACC |
| | | CGGCGGAAGCACCTGCTGTCTGGCCGGATGCGGTGTCCTGGCTGTGGCGGCAGCT |
| | | GCTCCTACTCCGGCAACGGCTACAGATGCTGGCGGTCCTCCGTGAAGGGCGGCTG |
| | | CCCTGCTCCAACCTACGTGGCTCGCAAGTCTGTGGAAGAGTATGTGGCCTTCCGG |
| | | TGGGCTGCCAAGCTGGCCGCCTCCGAGCCTGACGATCCTTTCGTGATCGCCGTGG |
| | | CCGATCGGTGGGCCGCTCTGACCCACCCTCAGGCTTCCGAAGATGAGAAGTACGC |
| | | CAAGGCCGCAGTGAGGGAGGCCGAGAAGAACCTGGGCAGACTGCTAAGAGACA |
| | | GACAGAATGGCGTGTACGATGGACCTGCCGAACAGTTCTTCGCCCCTGCTTACCA |
| | | GGAGGCTCTGTCTACACTGCAGGCCGCTAAGGACGCCGTGTCTGAGTCCTCCGCC |
| | | TCTGCCGCTGTGGACGTGAGCTGGATCGTGGACAGCAGCGACTACGAGGAACTG |
| | | TGGCTGAGAGCTACCCCTACCATGAGAAACGCTATCATCGACACATGCATCGACG |
| | | AGATCTGGGTCGCGAAAGGCCAGAGAGGCAGACCTTTCGACGGGGACGAGAGA |
| | | GTGAAGATCAAGTGGGCCGCTAGGACT |
| 43 | | MPGMTTETGPDPAGLIDLFCRKSKAVKSRANGAGQRRKQEISIAAQETLGRKVAALL |
| | | GMQVRHVWKEVGSASRFRKGKARDDQSKALKALESGEVGALWCYRLDRWDRGG |
| | | AGAILKIIEPEDGMPRRLLFGWDEDTGRPVLDSTNKRDRGELIRRAEEAREEAEKLSE |
| | | RVRDTKAHQRENGEWVNARAPYGLRVVLVTVSDEEGDEYDERKLAADDEDAGGP |
| | | DGLTKAEAARLVFTLPVTDRLSYAGTAHAMNTREIPSPTGGPWIAVTVRDMIQNPAY |
| | | AGWQTTGRQDGKQRRLTFYNGEGKRVSVMHGPPLVTDEEQEAAKAAVKGEDGVG |
| | | VPLDGSDHDTRRKHLLSGRMRCPGCGGSCSYSGNGYRCWRSSVKGGCPAPTYVAR |
| | | KSVEEYVAFRWAAKLAASEPDDPFVIAVADRWAALTHPQASEDEKYAKAAVREAE |
| | | KNLGRLLRDRQNGVYDGPAEQFFAPAYQEALSTLQAAKDAVSESSASAAVDVSWIV |
| | | DSSDYEELWLRATPTMRNAIIDTCIDEIWVAKGQRGRPFDGDERVKIKWAART |
| 6 | Int6 | ATGCAGCTGGACGCCACCCTGACACTGCGGGACGAGGGCCTGAGCGCTTTCCAC |
| | | CAGAGACACATCAAGCAGGGTGCTCTGGGAGTGTTCCTGAGAGCTATCGAGGAC |
| | | GGCCGGATCCAGCCTGGCTCCGTGCTGATCGTGGAAGGCCTGGACAGACTCTCTA |
| | | GAGCCGAGCCCATCCAAGCTCAGGCCCAGCTGGCCCAGATCATCAACGCCGGCA |
| | | TCACCGTGGTGACCGCCTCTGATGGCCGAGAGTACAACCGGGAAAGACTGAAAG |
| | | CCCAACCTATGGACCTTGTGTACTCCCTGCTGGTGATGATCAGAGCTCACGAGGA |
| | | ATCCGACACCAAGTCCAAGCGGGTGAAGGCCGCCATCAGGCGGCAGTGCGAGGG |
| | | CTGGGTCGCTGGCACATGGCGGGGCATCATCCGGAACGGCAAGGACCCTCACTG |
| | | GGTCAGACTGGGCGAGCACGGCAAGTTCGAGCATGTGCCTGAGCGGGTGCTGGC |
| | | TGTGCGGACAATGATCGACCTGTTCCTGGAAGGCCACGGCGCCATCGAGATCACC |
| | | AGGCGGCTGACCGAGCAGAACCTGTACGTGTCCAACGCCGGCAACTACTCTGTG |
| | | CACATGTACAGAATCGTGAGAAACCAGGCTCTGATCGGCGAGAAGAGAATCTCC |
| | | GTGGATGGAGAAGAGTTCCGGCTGGACGGCTACTACCCTCCAATCCTGACCAGA |
| | | GAAGAATTTGCCGAACTGCAGCAGACCATGTCCGAGAGAGGCAGACGGAAGGGC |
| | | AAAGGCGAGATCCCTAACATCATCACAGGACTGTCCATCACAGTGTGCGGCTATT |
| | | GTGGCAGAGCCATGACCACCCAGAACTCTAAGGCTCGCGCCCCTAAGGGAAAAA |
| | | GCGTGGTCAGACGGCTGTCCTGCCCCATGAATTCCTTCAACGAGGGATGTCCTAT |
| | | CGGCGGCTCTTGCGAGTCTGAGATCGTCGAGAGAGCCCTCATGAGATACTGCTCC |
| | | GACCAGTTCAATCTGTCTCGGTTGCTGGAGGGCGACGACGGCACCGCCCGGCGG |
| | | ACCGCTCAACTGGCTGTGGCTAGACAAAGAGCATCTGACATCGAAGCCCAGATC |
| | | CAGCGCGTGACCGACGCCCTCCTGAGCGACGACGGCAAGGCTCCTGCCGCCTTTA |
| | | CCCGCAGAGCTCGCGAGCTGGAAACCCAGCTGGAGGAACAGAGAAGAGAGATC |
| | | GAGGCTCTGGAACACCAGATCGCCGCTAGCTCTGCTCATGGCATCCCCGCCGCCG |
| | | CTGAGGCCTGGGCTCAGCTGGTTGACGGCGTGCTGGCCCTGGACTACGATGCTCG |
| | | GATGAAGGCCAGACAGCTGGTGGCCGATACCTTCAGAAAGATCGTGGTGTACCA |
| | | GAGGGGCTTCGCCCCAATCGACGATGCTGCTGCCGACAGATGGAAGAGATCCGG |
| | | CACCATCGGCCTGATGCTGGTCACCAAGAGAGGAGGCATGCGGCTGCTGAACGT |
| | | GGACCGGAGAACCGGCTGCTGGCAGGCCGAGGATGACCTGGATCCTTCTCTGATT |
| | | CCTTCCGATGGCCTGCCCATGCTGCCTCTGGATGCC |
| 44 | | MQLDATLTLRDEGLSAFHQRHIKQGALGVFLRAIEDGRIQPGSVLIVEGLDRLSRAEPI |
| | | QAQAQLAQIINAGITVVTASDGREYNRERLKAQPMDLVYSLLVMIRAHEESDTKSKR |
| | | VKAAIRRQCEGWVAGTWRGIIRNGKDPHWVRLGEHGKFEHVPERVLAVRTMIDLFL |
| | | EGHGAIEITRRLTEQNLYVSNAGNYSVHMYRIVRNQALIGEKRISVDGEEFRLDGYYP |
| | | PILTREEFAELQQTMSERGRRKGKGEIPNIITGLSITVCGYCGRAMTTQNSKARAPKG |
| | | KSVVRRLSCPMNSFNEGCPIGGSCESEIVERALMRYCSDQFNLSRLLEGDDGTARRTA |
| | | QLAVARQRASDIEAQIQRVTDALLSDDGKAPAAFTRRARELETQLEEQRREIEALEH |
| | | QIAASSAHGIPAAAEAWAQLVDGVLALDYDARMKARQLVADTFRKIVVYQRGFAPI |
| | | DDAAADRWKRSGTIGLMLVTKRGGMRLLNVDRRTGCWQAEDDLDPSLIPSDGLPM |
| | | LPLDA |
| 7 | Int7 | ATGAAAGTGGCCATCTACGTGCGGGTTTCCACCGACGAGCAGGCCAAAGAAGGT |
| | | TTCAGCATCCCTGCTCAAAGAGAGCGGCTGAGAGCCTTCTGCGCCTCTCAAGGCT |
| | | GGGAGATCGTGCAGGAGTACATCGAGGAGGGCTGGTCCGCTAAGGATCTGGACA |
| | | GACCTCAGATGCAGCGGCTGCTGAAGGACATCAAGAAGGGCAATATCGATATCG |
| | | TGCTGGTGTACAGACTGGATAGGCTGACCAGATCTGTGCTGGATCTGTACCTGCT |
| | | GCTCCAGACCTTCGAGAAGTACAACGTGGCCTTTCGGTCTGCCACCGAGGTGTAC |
| | | GATACAAGCACCGCCATGGGCAGACTGTTTATCACTCTGGTCGCTGCTCTGGCTC |
| | | AGTGGGAAAGAGAGAACCTGGCCGAGAGAGTGAAGTTCGGCATCGAACAGATG |
| | | ATCGACGAGGGCAAGAAGCCAGGCGGCCATTCTCCTTACGGCTACAAGTTTGAC |
| | | AAGGATTTCAACTGTACCATCATCGAGGAAGAAGCTGATGTGGTGCGGATGATTT |
| | | ACAGAATGTACTGCGACGGCTATGGCTATAGATCCATCGCCGACAGACTGAACG |
| | | AGCTGATGGTTAAGCCTAGAATCGCCAAGGAGTGGAACCACAACTCCGTCAGAG |
| | | ATATTCTGACCAACGACATCTACATCGGCACCTACAGATGGGGCGACAAGGTGG |
| | | TGCCTAACAACCACCCCCCCATCATCTCCGAGACACTGTTTAAGAAGGCCCAGAA |
| | | AGAGAAGGAGAAGCGGGGAGTGGACCGGAAGAGAGTGGGCAAGTTCCTGTTCA |
| | | CCGGCCTGCTGCAGTGTGGCAACTGCGGCGGACACAAGATGCAGGGCCACTTCG |
| | | ACAAGCGCGAGCAGAAAACCTACTACCGGTGCACCAAGTGCCACCGGATCACCA |
| | | ACGAGAAGAACATCTTGGAACCTCTGCTGGATGAGATCCAGCTGCTGATCACCTC |
| | | TAAGGAGTACTTCATGTCCAAGTTCAGCGACAGATACGACCAGCAAGAAGTGGT |
| | | CGACGTGTCCGCTCTCACAAAAGAGCTCGAGAAGATCAAGCGGCAGAAGGAAAA |
| | | GTGGTACGACCTGTACATGGACGACCGGAATCCTATCCCCAAAGAGGAGCTGTTC |
| | | GCCAAGATCAACGAGCTGAACAAGAAAGAAGAGGAAATCTACTCCAAGCTGTCT |
| | | GAAGTGGAAGAGGACAAAGAGCCTGTGGAAGAAAAGTACAACAGACTGTCCAA |
| | | GATGATCGACTTCAAGCAGCAGTTCGAGCAGGCTAATGACTTCACCAAAAAGGA |
| | | ACTGCTGTTCTCTATCTTCGAGAAGATCGTGATCTATCGGGAGAAGGGAAAGCTG |
| | | AAAAAGATTACACTGGACTACACCCTGAAG |
| 45 | | MKVAIYVRVSTDEQAKEGFSIPAQRERLRAFCASQGWEIVQEYIEEGWSAKDLDRPQ |
| | | MQRLLKDIKKGNIDIVLVYRLDRLTRSVLDLYLLLQTFEKYNVAFRSATEVYDTSTA |
| | | MGRLFITLVAALAQWERENLAERVKFGIEQMIDEGKKPGGHSPYGYKFDKDENCTII |
| | | EEEADVVRMIYRMYCDGYGYRSIADRLNELMVKPRIAKEWNHNSVRDILTNDIYIGT |
| | | YRWGDKVVPNNHPPIISETLFKKAQKEKEKRGVDRKRVGKFLFTGLLQCGNCGGHK |
| | | MQGHFDKREQKTYYRCTKCHRITNEKNILEPLLDEIQLLITSKEYFMSKFSDRYDQQE |
| | | VVDVSALTKELEKIKRQKEKWYDLYMDDRNPIPKEELFAKINELNKKEEEIYSKLSE |
| | | VEEDKEPVEEKYNRLSKMIDFKQQFEQANDFTKKELLFSIFEKIVIYREKGKLKKITLD |
| | | YTLK |
| 8 | Int8 | ATGAAAGTGGCCGTGTACTGCAGAGTGTCCACCCTCGAGCAGAAGGAGCACGGC |
| | | CATTCTATTGAGGAACAAGAGCGGAAGCTGAAGTCCTTCTGCGACATCAACGACT |
| | | GGACAGTGTACGACACCTACATCGACGCTGGATACTCTGGCGCCAAGCGGGACA |
| | | GACCTGAGCTGCAGCGGCTGATGAACGATATCAACAAGTTCGACCTGGTGCTGGT |
| | | CTACAAGCTGGACCGGCTGACCAGAAACGTGCGGGATCTGCTGGACCTGCTGGA |
| | | AATCTTCGAGAAGAACGACGTCAGCTTCAGATCCGCCACCGAGGTGTACGACAC |
| | | CACCACCGCTATGGGCCGGCTGTTCGTGACCCTGGTGGGCGCTATGGCCGAGTGG |
| | | GAGAGAGAGACAATCAGAGAACGGACCCAGATGGGCAAGCTGGCCGCTCTGAG |
| | | AAAGGGCATCATGCTGACCACACCACCTTTTTACTACGACAGAGTGGACAACAA |
| | | GTTCGTGCCTAACAAGTACAAGGACGTGATCCTGTGGGCCTACGACGAGGCCAT |
| | | GAAGGGCCAGTCCGCTAAGGCCATCGCCAGGAAGCTGAACAACTCCGACATCCC |
| | | TCCCCCTAACAATACCCAGTGGCAGGGCAGAACCATTACCCACGCCCTGCGCAAC |
| | | CCTTTCACCAGAGGCCACTTCGATTGGGGCGGCGTGCACATCGAAAATAACCATG |
| | | AGCCTATCATCACCGATGAGATGTACGAGAAAGTCAAGGATAGACTGAATGAGA |
| | | GAGTGAACACCAAGAAGGTCCGACACACCTCCATCTTCAGAGGAAAGCTCGTGT |
| | | GTCCTGTGTGCAACGCCAGACTGACACTGAATTCTCACAAGAAGAAGTCCAACTC |
| | | CGGCTACATCTTTGTGAAGCAGTACTACTGTAACAACTGCAAGGTGACCCCTAAC |
| | | CTGAAACCTGTGTACATCAAAGAGAAAGAAGTGATCAAAGTGTTCTACAACTAC |
| | | CTGAAAAGATTCGACCTGGAAAAGTACGAAGTGACACAGAAACAGAACGAACCT |
| | | GAGATCACCATCGATATCAATAAGGTGATGGAACAGCGGAAGAGATACCACAAG |
| | | CTGTACGCCTCTGGACTGATGCAAGAAGATGAACTGTTTGATCTGATCAAGGAAA |
| | | CCGACCAGACCATCGCTGAGTACGAGAAGCAGAACGAGAACCGGGAGGTGAAA |
| | | CAGTATGACATCGAAGATATCAAGCAGTATAAGGACCTGCTGCTGGAAATGTGG |
| | | GACATCTCCTCTGACGAGGACAAGGAGGACTTCATCAAGATGGCTATCAAGAAC |
| | | ATCTACTTCGAGTATATCATCGGCACCGGCAACACCTCTCGGAAGCGGAACAGCC |
| | | TAAAGATCACTAGCATCGAGTTCTAC |
| 46 | | MKVAVYCRVSTLEQKEHGHSIEEQERKLKSFCDINDWTVYDTYIDAGYSGAKRDRP |
| | | ELQRLMNDINKFDLVLVYKLDRLTRNVRDLLDLLEIFEKNDVSFRSATEVYDTTTAM |
| | | GRLFVTLVGAMAEWERETIRERTQMGKLAALRKGIMLTTPPFYYDRVDNKFVPNKY |
| | | KDVILWAYDEAMKGQSAKAIARKLNNSDIPPPNNTQWQGRTITHALRNPFTRGHFD |
| | | WGGVHIENNHEPIITDEMYEKVKDRLNERVNTKKVRHTSIFRGKLVCPVCNARLTLN |
| | | SHKKKSNSGYIFVKQYYCNNCKVTPNLKPVYIKEKEVIKVFYNYLKRFDLEKYEVTQ |
| | | KQNEPEITIDINKVMEQRKRYHKLYASGLMQEDELFDLIKETDQTIAEYEKQNENRE |
| | | VKQYDIEDIKQYKDLLLEMWDISSDEDKEDFIKMAIKNIYFEYIIGTGNTSRKRNSLKI |
| | | TSIEFY |
| 9 | Int9 | ATGAAAGTGGCTATCTACACCAGAGTGTCCACACTGGAACAGAAAGAGAAGGGC |
| | | CACTCCATCGAGGAGCAGGAAAGAAAGCTGAGAGCCTACTCCGACATCAACGAC |
| | | TGGAAGATCCACAAGGTGTACACAGATGCTGGCTACTCTGGCGCTAAGAAAGAT |
| | | AGACCTGCCCTGCAAGAGATGCTGAACGAGATCGACAACTTCGACCTGGTGCTG |
| | | GTTTATAAGCTGGACCGGCTGACAAGATCCGTGAAAGATCTGCTGGAAATCCTGG |
| | | AACTGTTCGAGAACAAGAACGTGTTGTTCAGATCCGCCACCGAGGTGTACGACA |
| | | CCACCAGCGCTATGGGCAGACTGTTTGTGACCCTGGTCGGCGCCATGGCTGAGTG |
| | | GGAACGGACCACCATCCAGGAGAGAACCGCCATGGGCAGACGGGCCTCTGCTAG |
| | | AAAAGGCCTGGCCAAGACCGTGCCTCCATTCTACTACGACCGGGTGAACGATAA |
| | | GTTCGTGCCCAACGAGTACAAGAAGGTGCTGCGGTTCGCCGTGGAAGAGGCCAA |
| | | GAAGGGCACCTCTCTGAGAGAGATCACCATCAAACTTAACAACTCTAAGTACAA |
| | | GGCCCCTCTGGGTAAGAACTGGCACCGGTCTGTGATCGGCAACGCTCTGACCTCC |
| | | CCTGTGGCCAGGGGCCATCTGGTGTTCGGCGACATCTTCGTGGAAAACACCCACG |
| | | AGGCTATCATCTCTGAGGAAGAATATGAAGAGATCAAACTGCGCATCTCCGAAA |
| | | AGACCAACAGCACCATCGTGAAGCACAACGCCATCTTCCGGTCCAAGCTCCTGTG |
| | | CCCCAATTGTAACCAGAAGCTCACACTGAACACCGTGAAGCACACCCCTAAAAA |
| | | CAAGGAAGTGTGGTACAGCAAGCTGTACTTTTGCTCCAACTGCAAGAATACCAA |
| | | GAACAAGAATGCCTGCAATATCGATGAGGGCGAGGTCCTGAAACAGTTCTACAA |
| | | CTACCTGAAGCAGTTTGATCTGACCTCCTACAAGATCGAGAACCAGCCTAAGGAG |
| | | ATCGAGGACGTGGGAATCGACATTGAAAAGCTGCGGAAAGAGCGGGCCAGATGT |
| | | CAGACTCTGTTCATCGAAGGAATGATGGACAAGGACGAGGCCTTCCCTATCATCA |
| | | GCCGGATCGACAAGGAAATCCATGAGTACGAGAAGCGGAAGGATAATGACAAG |
| | | GGAAAGACATTCAACTACGAGAAGATCAAGAACTTCAAATACTCTCTGCTGAAC |
| | | GGCTGGGAGCTGATGGAGGACGAGCTGAAAACCGAATTTATCAAGATGGCCATC |
| | | AAGAACATCCACTTCGAGTACGTCAAGGGCATCAAGGGCAAGAGACAGAACTCC |
| | | CTGAAGATCACCGGCATCGAGTTCTAT |
| 47 | | MKVAIYTRVSTLEQKEKGHSIEEQERKLRAYSDINDWKIHKVYTDAGYSGAKKDRP |
| | | ALQEMLNEIDNFDLVLVYKLDRLTRSVKDLLEILELFENKNVLFRSATEVYDTTSAM |
| | | GRLFVTLVGAMAEWERTTIQERTAMGRRASARKGLAKTVPPFYYDRVNDKFVPNE |
| | | YKKVLRFAVEEAKKGTSLREITIKLNNSKYKAPLGKNWHRSVIGNALTSPVARGHLV |
| | | FGDIFVENTHEAIISEEEYEEIKLRISEKTNSTIVKHNAIFRSKLLCPNCNQKLTLNTVK |
| | | HTPKNKEVWYSKLYFCSNCKNTKNKNACNIDEGEVLKQFYNYLKQFDLTSYKIENQ |
| | | PKEIEDVGIDIEKLRKERARCQTLFIEGMMDKDEAFPIISRIDKEIHEYEKRKDNDKGK |
| | | TFNYEKIKNFKYSLLNGWELMEDELKTEFIKMAIKNIHFEYVKGIKGKRQNSLKITGI |
| | | EFY |
| 10 | Int10 | ATGATCACAACCAACAAGGTGGCTATCTACGTCAGAGTGTCCACCACAAATCAA |
| | | GTGGAAGAAGGCTACTCCATCGACGAGCAGAAGGACAAGCTCTCCTCCTACTGT |
| | | GACATCAAGGATTGGAACGTGTACAAGGTGTACACCGACGGCGGCTTTTCCGGA |
| | | AGCAACACCGATAGACCTGCCCTGGAATCTCTGATCAAGGATGCAAAGAAGCGG |
| | | AAGTTCGACACCGTGCTGGTGTACAAGCTGGACAGACTGTCCAGATCCCAGAAG |
| | | GACACCCTGCACCTGATCGAGGACGTGTTCATCAAGAACGGCATCGAGTTTCTGT |
| | | CCCTGCAAGAGAACTTCGATACATCTACCCCATTCGGCAAGGCCATGATCGGTCT |
| | | GCTGTCTGTGTTCGCCCAGCTGGAGAGAGAACAGATCAAAGAGCGGATGCAGCT |
| | | CGGCAAGCTGGGCAGAGCTAAGTCTGGAAAGTCCATGATGTGGGCCAAAACCAG |
| | | CTACGGCTACGACTACCACAAGGAAACCGGCACCGTGACGATCAACCCCGCTCA |
| | | GGCTCTGACAATCAAGTTTATCTTCGAGTCTTACCTGAGAGGCAGATCCATCACC |
| | | AAGCTGAGAGATGACCTGAACGAGAAGTACCCTAAGCACGTGCCTTGGTCCTAC |
| | | AGAGCCGTGAGAACCATCCTGGACAATCCTGTGTACTGTGGCTTCAACCAGTACA |
| | | AGGGCGAGATCTACCCCGGCAACCACGAGCCTATCATCTCCAAAGAGGAGTACG |
| | | ACAAGACCCAGTCCGAGCTGAAGATCCGGCAGCGGACCGCTGCTGAGAACGTGA |
| | | ACCCTCGCCCCTTCCAGGCCAAGTACATCCTGTCTGGCATTGCCCAGTGCGGATA |
| | | TTGCGGCGCTCCTCTGAAAATCATGCTGGGCGTCAAGAGAAAGGACGGATCTCG |
| | | GCTGAAGAAATACGAGTGCCACCAGAGACATCCTAGAACCCTGAGAGGCGTGAC |
| | | CACCTACAACGACAATAAGAAGTGCGACTCGGGCTTCTACTACAAGGACAAGCT |
| | | CGAGGCCTATGTGCTGAAGGAAATCTCTAAGCTGCAGGACGACGCCGATTACCT |
| | | GGATAAGATCTTCAGCGGCGACAACGCCGAGACAATCGACCGCGAGAGCTATAA |
| | | GAAGCAGATCGAAGAACTGTCCAAAAAACTGAGCAGACTGAACGACCTGTACAT |
| | | CGACGACCGGATCACCCTGGAGGAACTGCAGTCTAAGTCTGCCGAATTCATCTCC |
| | | ATGCGGGGCACCCTGGAAACCGAGTTGGAAAACGATCCTGCTCTGCGGAAGAAC |
| | | AAGCGGAAAGCCGACATGAGAAAGCTGCTGAACGCTGAAAAGGTGTTCTCTATG |
| | | GACTACGAGTCCCAGAAAGTTCTGGTGCGGAGACTGATCAACAAAGTGAAGGTC |
| | | ACCGCCGAGGATATCGTGATCAACTGGAAGATC |
| 48 | | MITTNKVAIYVRVSTTNQVEEGYSIDEQKDKLSSYCDIKDWNVYKVYTDGGFSGSNT |
| | | DRPALESLIKDAKKRKFDTVLVYKLDRLSRSQKDTLHLIEDVFIKNGIEFLSLQENFDT |
| | | STPFGKAMIGLLSVFAQLEREQIKERMQLGKLGRAKSGKSMMWAKTSYGYDYHKE |
| | | TGTVTINPAQALTIKFIFESYLRGRSITKLRDDLNEKYPKHVPWSYRAVRTILDNPVYC |
| | | GFNQYKGEIYPGNHEPIISKEEYDKTQSELKIRQRTAAENVNPRPFQAKYILSGIAQCG |
| | | YCGAPLKIMLGVKRKDGSRLKKYECHQRHPRTLRGVTTYNDNKKCDSGFYYKDKL |
| | | EAYVLKEISKLQDDADYLDKIFSGDNAETIDRESYKKQIEELSKKLSRLNDLYIDDRIT |
| | | LEELQSKSAEFISMRGTLETELENDPALRKNKRKADMRKLLNAEKVFSMDYESQKV |
| | | LVRRLINKVKVTAEDIVINWKI |
| 11 | Int11 | ATGCTGAGATGCGCCATCTACATCAGAGTGTCCACCGAGGAGCAGGCCATGCAC |
| | | GGCCTGTCCATGGACGCTCAGAAAGCCGATCTGACCGACTACGCTAAGAAGCAC |
| | | AACTACGAGATCATCGACTACTACGTGGACTCCGGCAAGACCGCCAGAAAGAGA |
| | | CTGTCCAAGCGCAAGGACCTGCAGCGGATGATCGAGGACGTCAAGCTGAACAAG |
| | | ATCGACATCATCATCTTTACCAAGCTGGACAGGTGGTTCCGGAACGTGCGGGACT |
| | | ACTACAAGATCCAAGAGGTGCTGGAGGACCACAACGTCGACTGGAAAACCATCT |
| | | TCGAGAATTACGATACCTCTACCGCTAACGGCAGACTGCACATCAACATCATGCT |
| | | GTCCGTGGCTCAGGACGAGGCCGACAGAACCTCCGAAAGAATCAAACGGGTGTT |
| | | CGAGAACAAGCTGAAGAACAACGAGCCTACATCTGGCTCTCTGCCTATCGGCTAC |
| | | AAGATCAAAGAGAAGTCCATCATTATCGATGAGGAAAAGGCCCCTATCGCCAAG |
| | | GATGTGTTCGATTTCTACTACTACCACCAGTCCCAGACCAAGGTGTTCAAAGAAA |
| | | TCCTCAACAAATACAACCTGTCTCTGTGCGAAAAGACCATCCGGAGAATGCTGGA |
| | | GAATAAGCTGTACATCGGCATCTACAGAGAGCACGAGAACTTCTGTCCTCCTCTG |
| | | ATCGACAAGAACAAGTTCGACGAAGTGCAGCTGATTCTGAAGAGGCGGAACATC |
| | | AAGTATATCCCTACTAAGCGGATCTTTCTGTTCACCAGCCTGCTGATCTGCAAGG |
| | | AGTGTAGACATAAGATGATCGGCAACGCCCAGATCAGAAACACAAAGGCTGGAA |
| | | AGATCGAGTACATCTTGTACCGGTGCAACCAATCTTACGCTCGGCACACCTGCAA |
| | | CCACAGAAAGGTGATCTATGAAAACAAGATCGAAACCTATCTGCTGAACAACAT |
| | | CGAGTCCGAGCTGAAAAAGTTTATCTACGACTACGAGCTGGAAGATATCCCCAA |
| | | GGTGAAGAACAAAGTGAACAAAACAAATATCAAGCGGAAGCTGGAAAAGCTGA |
| | | AAGAACTGTACATCAACGACCTCATCGACATCGACATGTACAAAGAGGATTACA |
| | | AGAAGTACACCGAGATCCTGAATACCAAAGAAGAAAAGATCGAACAGAGAAAC |
| | | CTGCAGCCTCTGAAGGACTTCCTGAACTCCGACTTCAAGTCTCTGTACTCCTCCAT |
| | | CTCTAGAGAAGAGAAGCGGCTGCTGTGGAGAGGCATAATCAGCGAGATCCAGAT |
| | | CGACTGCAATAACGATATCACCATCATCCCCCATCCA |
| 49 | | MLRCAIYIRVSTEEQAMHGLSMDAQKADLTDYAKKHNYEIIDYYVDSGKTARKRLS |
| | | KRKDLQRMIEDVKLNKIDIIIFTKLDRWFRNVRDYYKIQEVLEDHNVDWKTIFENYD |
| | | TSTANGRLHINIMLSVAQDEADRTSERIKRVFENKLKNNEPTSGSLPIGYKIKEKSIIID |
| | | EEKAPIAKDVFDFYYYHQSQTKVFKEILNKYNLSLCEKTIRRMLENKLYIGIYREHEN |
| | | FCPPLIDKNKFDEVQLILKRRNIKYIPTKRIFLFTSLLICKECRHKMIGNAQIRNTKAGK |
| | | IEYILYRCNQSYARHTCNHRKVIYENKIETYLLNNIESELKKFIYDYELEDIPKVKNKV |
| | | NKTNIKRKLEKLKELYINDLIDIDMYKEDYKKYTEILNTKEEKIEQRNLQPLKDFLNS |
| | | DFKSLYSSISREEKRLLWRGIISEIQIDCNNDITIIPHP |
| 12 | Int12 | ATGAAGGTGGCCATCTACACTAGAGTGTCCTCGGCTGAGCAGGCCAACGAGGGA |
| | | TACTCCATCCACGAGCAAAAGAAGAAGCTCATCTCCTACTGCGAAATCCACGACT |
| | | GGAACGAGTACAAAGTGTTCACCGACGCCGGCATCTCTGGCGGCTCTATGAAGC |
| | | GGCCTGCTCTGCAGAAACTGATGAAACATCTGTCTAGCTTCGACCTGGTGCTGGT |
| | | GTACAAGCTGGACAGACTGACCAGAAACGTGCGCGACCTGCTGGATATGCTCGA |
| | | AGAATTCGAGCAGTACAACGTATCTTTCAAGTCCGCCACCGAAGTGTTCGACACC |
| | | ACCTCTGCTATCGGCAAGCTGTTCATCACCATGGTGGGCGCTATGGCCGAGTGGG |
| | | AAAGAGAAACCATCAGAGAGCGGAGCCTGTTTGGATCTCGGGCCGCTGTGCGGG |
| | | AAGGCAACTACATCAGAGAGGCTCCTTTCTGCTACGACAACATCGAGGGCAAGC |
| | | TGCATCCAAACGAATACGCCAAGGTGATCGATCTGATCGTGTCCATGTTCAAGAA |
| | | GGGCATCTCCGCCAATGAGATCGCCAGACGGCTGAACTCCTCCAAGGTGCACGT |
| | | GCCTAACAAAAAGTCCTGGAACCGGAACAGCCTGATCCGGCTCATGAGATCTCC |
| | | CGTTCTGCGGGGCCACACCAAGTACGGCGACATGCTGATCGAGAACACCCATGA |
| | | GCCTGTGCTGTCCGAACACGACTACAATGCTATCAATAATGCCATCTCCAGCAAG |
| | | ACCCACAAGTCCAAGGTCAAGCACCACGCCATCTTCAGAGGAGCCCTGGTGTGTC |
| | | CTCAGTGCAACAGAAGGCTGCACCTGTACGCTGGCACAGTGAAGGACCGGAAGG |
| | | GCTACAAGTACGATGTCAGAAGATACAAGTGCGAGACATGTTCTAAGAACAAGG |
| | | ACGTGAAGAACGTGTCCTTCAACGAGTCTGAGGTGGAAAACAAGTTCGTGAACC |
| | | TGCTGAAGTCTTACGAGCTGAACAAGTTCCACATCCGGAAAGTGGAACCCGTGA |
| | | AAAAGATCGAGTATGATATCGACAAGATCAACAAGCAGAAGATCAACTACACCA |
| | | GATCTTGGTCCCTGGGCTATATCGAGGACGACGAGTACTTCGAGCTGATGGAGGA |
| | | GATCAACGCCACAAAGAAGATGATCGAGGAACAGACAACCGAGAACAAGCAGT |
| | | CTGTCAGCAAAGAGCAGATCCAGTCCATCAACAACTTTATCCTGAAAGGCTGGG |
| | | AGGAACTGACCATCAAGGATAAAGAGGAGCTGATCCTGTCCACCGTGGACAAGA |
| | | TAGAGTTCAATTTCATTCCTAAGGATAAGAAGCACAAAACCAACACCCTGGACAT |
| | | CAACAACATCCACTTTAAGTTT |
| 50 | | MKVAIYTRVSSAEQANEGYSIHEQKKKLISYCEIHDWNEYKVFTDAGISGGSMKRPA |
| | | LQKLMKHLSSFDLVLVYKLDRLTRNVRDLLDMLEEFEQYNVSFKSATEVFDTTSAIG |
| | | KLFITMVGAMAEWERETIRERSLFGSRAAVREGNYIREAPFCYDNIEGKLHPNEYAK |
| | | VIDLIVSMFKKGISANEIARRLNSSKVHVPNKKSWNRNSLIRLMRSPVLRGHTKYGD |
| | | MLIENTHEPVLSEHDYNAINNAISSKTHKSKVKHHAIFRGALVCPQCNRRLHLYAGT |
| | | VKDRKGYKYDVRRYKCETCSKNKDVKNVSFNESEVENKFVNLLKSYELNKFHIRKV |
| | | EPVKKIEYDIDKINKQKINYTRSWSLGYIEDDEYFELMEEINATKKMIEEQTTENKQS |
| | | VSKEQIQSINNFILKGWEELTIKDKEELILSTVDKIEFNFIPKDKKHKTNTLDINNIHFKF |
| 13 | Int13 | ATGGCCGTGGGCATCTACATCAGAGTGTCCACCCAGGAGCAGGCCTCTGAAGGC |
| | | CATTCCATCGAGTCCCAGAAAAAGAAACTGGCTTCCTACTGCGAGATCCAGGGCT |
| | | GGGACGACTACCGGTTCTACATCGAGGAAGGCATCTCCGGCAAGAACACAAATC |
| | | GGCCTAAGCTGAAGCTGCTGATGGAACACATCGAGAAGGGAAAGATCAACATCC |
| | | TGCTGGTGTACAGACTGGATAGACTGACAAGATCTGTGATCGACCTGCACAAGCT |
| | | GCTGAACTTCCTGCAAGAGCACGGCTGTGCCTTCAAGTCCGCCACCGAGACATAC |
| | | GACACCACCACTGCCAACGGCAGAATGTCCATGGGCATCGTGTCCCTGCTGGCTC |
| | | AGTGGGAAACCGAGAACATGTCCGAGCGGATCAAGTTGAATCTGGAACATAAGG |
| | | TGCTGGTCGAGGGCGAAAGAGTGGGAGCCATCCCTTACGGCTTCGACCTGTCTGA |
| | | TGATGAAAAGCTGGTGAAGAACGAGAAGTCTGCTATCCTGCTGGACATGGTCGA |
| | | ACGGGTGGAAAATGGATGGTCCGTGAACAGAATCGTGAACTATCTGAACCTGAC |
| | | CAACAACGACCGCAACTGGAGCCCTAACGGCGTGCTGAGGCTGCTGCGGAATCC |
| | | TGCTCTGTACGGCGCTACCAGATGGAACGATAAGATCGCCGAGAACACCCACGA |
| | | GGGAATCATCAGCAAAGAGAGATTCAACCGGCTGCAGCAGATCCTCGCCGACAG |
| | | ATCCATCCACCACCGGCGGGACGTGAAGGGCACCTATATCTTCCAAGGCGTGCTG |
| | | AGATGTCCTGTGTGCGACCAGACCCTGTCCGTGAACCGGTTTATTAAGAAGAGAA |
| | | AGGACGGCACCGAGTATTGTGGTGTGCTGTACCGGTGCCAGCCTTGCATCAAGCA |
| | | GAACAAGTACAACCTGGCCATCGGCGAGGCCAGATTTCTGAAGGCCCTGAACGA |
| | | GTACATGTCTACCGTGGAATTCCAGACGGTTGAAGATGAGGTGATACCCAAGAA |
| | | GTCTGAGAGAGAGATGCTGGAGTCTCAGCTGCAGCAGATCGCTCGGAAGCGGGA |
| | | AAAGTACCAGAAGGCTTGGGCCAGTGATCTGATGAGCGATGACGAGTTCGAGAA |
| | | GCTGATGGTGGAAACCAGAGAAACCTACGACGAGTGCAAGCAGAAGCTCGAGTC |
| | | CTGCGAGGACCCAATCAAAATCGACGAAACCTACCTGAAAGAAATCGTGTACAT |
| | | GTTCCACCAGACATTCAACGACCTGGAATCCGAGAAGCAGAAAGAGTTCATCAG |
| | | CAAGTTCATCAGAACCATCAGATACACCGTGAAGGAGCAGCAGCCCATCAGACC |
| | | TGACAAGTCTAAGACCGGCAAGGGCAAACAAAAAGTGATCATCACCGAAGTGGA |
| | | ATTTTACCAG |
| 51 | | MAVGIYIRVSTQEQASEGHSIESQKKKLASYCEIQGWDDYRFYIEEGISGKNTNRPKL |
| | | KLLMEHIEKGKINILLVYRLDRLTRSVIDLHKLLNFLQEHGCAFKSATETYDTTTANG |
| | | RMSMGIVSLLAQWETENMSERIKLNLEHKVLVEGERVGAIPYGFDLSDDEKLVKNE |
| | | KSAILLDMVERVENGWSVNRIVNYLNLTNNDRNWSPNGVLRLLRNPALYGATRWN |
| | | DKIAENTHEGIISKERFNRLQQILADRSIHHRRDVKGTYIFQGVLRCPVCDQTLSVNRF |
| | | IKKRKDGTEYCGVLYRCQPCIKQNKYNLAIGEARFLKALNEYMSTVEFQTVEDEVIP |
| | | KKSEREMLESQLQQIARKREKYQKAWASDLMSDDEFEKLMVETRETYDECKQKLES |
| | | CEDPIKIDETYLKEIVYMFHQTFNDLESEKQKEFISKFIRTIRYTVKEQQPIRPDKSKTG |
| | | KGKQKVIITEVEFYQ |
| 14 | Int14 | ATGACAGTGGGCATCTATATCAGAGTGTCCACCGAGGAACAGGTCAAGGAGGGC |
| | | TTCTCCATTAGCGCTCAGAAAGAAAAGCTGAAGGCCTACTGCACCGCTCAAGGCT |
| | | GGGAGGACTTCAAGTTCTACGTGGACGAAGGCAAGTCTGCCAAGGACATGCACC |
| | | GGCCCCTGCTCCAAGAGATGATCTCTCATATCAAGAAGGGACTGATCGATACCGT |
| | | GCTGGTGTACAAGCTGGACAGACTGACAAGATCCGTGGTGGATCTGCACAACCT |
| | | GCTGTCCATCTTCGACGAATTCAACTGCGCCTTCAAGTCCGCCACAGAAGTGTAC |
| | | GACACCTCCAGCGCCATGGGCAGATTCTTCATCACAATCATCTCCTCCGTGGCCC |
| | | AGTTCGAGCGCGAAAACACCTCCGAAAGAGTGAGCTTTGGCATGGCCGAGAAGG |
| | | TCAGACAGGGCGAGTACATCCCTCTGGCTCCTTTCGGCTATACCAAGGGCACCGA |
| | | CGGAAAGCTGATCGTCAACAAGATCGAGAAAGAAATCTTCCTGCAGGTGGTTGA |
| | | GATGGTGTCTACCGGCTACTCTCTGCGGCAGACCTGCGAGTACCTGACCAACATC |
| | | GGCCTGAAAACCCGGAGATCTAATGATGTGTGGAAGGTGAGCACCCTGATCTGG |
| | | ATGCTGAAGAACCCCGCCGTGTACGGCGCCATCAAGTGGAATAACGAGATCTAC |
| | | GAGAACACCCACGAGCCTCTGATCGACAAGGCTACCTTCAACAAAGTGGCTAAG |
| | | ATCCTGTCTATCAGATCCAAGTCCACCACCTCTAGAAGAGGCCACGTGCACCATA |
| | | TCTTTAAGAACCGGCTTATCTGCCCAGCATGTGGAAAGCGGCTGTCTGGCCTGCG |
| | | GACCAAGTACATCAACAAGAATAAGGAAACTTTCTACAACAACAACTACAGATG |
| | | TGCTACCTGCAAGGAGCACAGACGGCCTGCTGTGCAGATCTCCGAGCAGAAGAT |
| | | CGAGAAGGCCTTTATCGACTACATCTCCAACTACACCCTGAACAAGGCCAACATC |
| | | AGCTCTAAGAAGCTGGACAACAACTTAAGGAAGCAGGAAATGATCCAGAAAGA |
| | | GATCATCAGCCTGCAGCGGAAGAGAGAGAAGTTCCAGAAAGCCTGGGCCGCCGA |
| | | CCTGATGAACGACGATGAGTTCTCCAAACTGATGATCGATACAAAGATGGAAAT |
| | | CGACGCTGCTGAGGACCGGAAGAAAGAATACGACGTGTCCCTCTTCGTGTCTCCT |
| | | GAAGATATCGCCAAGCGGAACAACATCCTGCGGGAGCTGAAGATCAACTGGACC |
| | | TCTCTGTCCCCTACCGAGAAAACCGATTTTATTTCCATGTTCATCGAAGGCATCGA |
| | | GTACGTGAAGGACGACGAGAATAAGGCTGTGATCACCAAGATCTCTTTCCTG |
| 52 | | MTVGIYIRVSTEEQVKEGFSISAQKEKLKAYCTAQGWEDFKFYVDEGKSAKDMHRP |
| | | LLQEMISHIKKGLIDTVLVYKLDRLTRSVVDLHNLLSIFDEFNCAFKSATEVYDTSSA |
| | | MGRFFITIISSVAQFERENTSERVSFGMAEKVRQGEYIPLAPFGYTKGTDGKLIVNKIE |
| | | KEIFLQVVEMVSTGYSLRQTCEYLTNIGLKTRRSNDVWKVSTLIWMLKNPAVYGAIK |
| | | WNNEIYENTHEPLIDKATFNKVAKILSIRSKSTTSRRGHVHHIFKNRLICPACGKRLSG |
| | | LRTKYINKNKETFYNNNYRCATCKEHRRPAVQISEQKIEKAFIDYISNYTLNKANISSK |
| | | KLDNNLRKQEMIQKEIISLQRKREKFQKAWAADLMNDDEFSKLMIDTKMEIDAAED |
| | | RKKEYDVSLFVSPEDIAKRNNILRELKINWTSLSPTEKTDFISMFIEGIEYVKDDENKA |
| | | VITKISFL |
| 15 | Int15 | ATGAAGGCCGCCATCTATATCAGAGTGTCCACCCAGGAACAGATCGAGAATTAC |
| | | AGTATCCAGGCTCAGACCGAGAAACTGACCGCTCTGTGCAGATCCAAGGACTGG |
| | | GACGTGTACGATATCTTCATCGACGGAGGCTACTCTGGCTCCAACATGAACAGAC |
| | | CCGCCCTGAATGAGATGCTGTCTAAGCTGCACGAAATCGACGCCGTGGTGGTGTA |
| | | CAGGCTGGACAGACTGTCCAGATCCCAGAGAGATACCATCACACTGATCGAAGA |
| | | GTACTTCCTGAAGAACAACGTGGAATTCGTGTCCCTCAGCGAAACCCTGGACACT |
| | | AGCTCTCCATTTGGCAGAGCCATGATCGGCATCCTGTCTGTGTTCGCCCAGCTGG |
| | | AAAGAGAGACAATCCGGGACAGAATGGTCATGGGCAAGATCAAGCGGATCGAG |
| | | GCTGGCCTGCCTCTGACAACCGCCAAGGGCAGAACATTCGGCTATGATGTGATCG |
| | | ACACCAAGCTGTACATCAACGAGGAAGAAGCTAAGCAGCTGCAGATGATCTACG |
| | | ACATTTTCGAGGAAGAGAAGTCCATCACCACCCTGCAGAAGAGACTCAAAAAAC |
| | | TGGGCTTCAAGGTGAAGTCCTACTCCTCCTACAACAACTGGCTGACCAACGACCT |
| | | GTACTGCGGCTACGTGTCCTACGCCGACAAAGTCCATACCAAGGGCGTGCACGA |
| | | GCCTATCATCTCTGAAGAACAGTTCTACAGAGTGCAGGAGATCTTCAGCCGGATG |
| | | GGCAAAAATCCTAACATGAACCGGGATTCTGCTAGCCTGCTCAACAATCTGGTCG |
| | | TTTGTGGCAAGTGTGGACTGGGATTTGTGCACAGAAGAAAGGACACCATCTCCA |
| | | GAGGTAAGAAGTACCACTACCGGTACTACAGCTGCAAGACCTACAAGCACACCC |
| | | ATGAGCTGGAGAAGTGCGGCAACAAGATCTGGCGGGCTGACAAGCTGGAAGAAT |
| | | TGATCATCGATCGCGTGAACAACTATTCCTTCGCTTCTCGGAACGTGGACAAAGA |
| | | GGACGAGCTGGACAACCTGAACGAGAAGCTGAAAACCGAGCACAAGAAAAAGA |
| | | AGCGGCTGTTCGACCTGTACATCTCCGGCTCTTACGAGGTGTCTGAGCTGGATGC |
| | | TATGATGGCCGACATCGATGCCCAAATCAACTACTACGAGGCCCAGATCGAAGC |
| | | CAACGAGGAACTGAAGAAGAACAAGAAAATTCAAGAGAATCTGGCTGATCTGGC |
| | | CACCGTGGACTTTGACTCCCTAGAGTTCCGGGAAAAGCAGCTGTACCTGAAGTCT |
| | | CTGATCAACAAGATCTACATCGACGGCGAGCAGGTGACCATCGAGTGGCTG |
| 53 | | MKAAIYIRVSTQEQIENYSIQAQTEKLTALCRSKDWDVYDIFIDGGYSGSNMNRPAL |
| | | NEMLSKLHEIDAVVVYRLDRLSRSQRDTITLIEEYFLKNNVEFVSLSETLDTSSPFGRA |
| | | MIGILSVFAQLERETIRDRMVMGKIKRIEAGLPLTTAKGRTFGYDVIDTKLYINEEEAK |
| | | QLQMIYDIFEEEKSITTLQKRLKKLGFKVKSYSSYNNWLTNDLYCGYVSYADKVHTK |
| | | GVHEPIISEEQFYRVQEIFSRMGKNPNMNRDSASLLNNLVVCGKCGLGFVHRRKDTIS |
| | | RGKKYHYRYYSCKTYKHTHELEKCGNKIWRADKLEELIIDRVNNYSFASRNVDKED |
| | | ELDNLNEKLKTEHKKKKRLFDLYISGSYEVSELDAMMADIDAQINYYEAQIEANEEL |
| | | KKNKKIQENLADLATVDFDSLEFREKQLYLKSLINKIYIDGEQVTIEWL |
| 16 | Int16 | ATGAAGGGCGAGTCTGAGCTGGACAAGAAGGCCGCCATCTACATCAGAGTTTCT |
| | | ACACAAGAGCAGGCTACAGAGGGCTATTCGATCCAGGCACAAACCGACAGACTG |
| | | ATCAAGTACGTGGAAGCCAAGGACTTTATCCTGTATAAGAAGTATATCGACGCCG |
| | | GCTACAGCGCTTCTAAGCTCGAAAGACCCGCTATGCAGGATCTCATCCAGGACGT |
| | | CCAAAGCAAGAAAGTGGACGTGGTCATCGTGTACAAGCTGGATAGACTGTCTAG |
| | | ATCTCAGAAGGATACCATGTACCTGATCGAGGACATCTTCCGGCCTAACGACGTG |
| | | GAACTGATCTCTATGCAGGAAAGCTTTGACACCTCCACCGCCTTCGGCTCTGCCA |
| | | CCGTGGGCATGCTGTCCGTGTTCGCCCAACTGGAGAGGAAGTCCATCTCCGAAAG |
| | | AATGATCACAGGCAGAGTGGAGCGGGCTAAGAAAGGCTTCTACCACACCGGCGG |
| | | CCAGGACAGACCTCCAGCTGGCTACCAGTTCAACTCCGACAACCAGCTGATCATC |
| | | AACGAGTACGAGGCCGCTGCTATCAAGGACCTGTTTCGGCTGTACAACGACGGC |
| | | CTGGGAAAGTCTAGCATCTCCGAGTACCTGAAGAAGAACTACCCCGGAAAAAAC |
| | | AAGTGGCTGCCTTCTTCTATCGATCGGATGCTGAAGAACTCCCTGTACATCGGCA |
| | | AGGTGAAGTTCTCCGGCGCCGAGTACGACGGCATCCATGAGCCTATCATAGACG |
| | | AAGTGACCTTCTACAAGACCCAGAAGGAGATCGCCAGACGGAAGCAGACCAACA |
| | | CCAAGAGATACAACTACGTGGCCCTGCTGGGCGGCCTGTGCGAGTGCGGCATCT |
| | | GTGGCGCTAAGATGGCCAACAGACGGGCCGTGGGACGCAAGGGTAAGGTGTACC |
| | | GGTACTACAGATGCTACTCCAAGAAAGGATCTCCTAAGCACATGATGAAAACCG |
| | | ATGGCTGCTCCTCCAAGGCCCAGCAGCAGTTCATCATCGACGAGGCTGTGATTAA |
| | | CAACCTGAAGAACATCGACGTCGAAGCCGAACTGAAACGCAGATCTGCTCCTCA |
| | | GACCAATACCTCTCTGATCTCCAGCCAGATCGAGAGCATCGATAAGCAGATTAAC |
| | | AAGCTGATCGACCTGTTCCAGGTGGACTCCATGCCTCTGGATGTGATCAGCGAGA |
| | | AGATCGATAAGCTGAACAAAGAGAAGCAGTCCATGGAAAAACTGCTGGAACGG |
| | | AAGAATAAGCTGGACAAAACCGAGCTGCAGCACAGATTCGATGTGCTGAAGTCC |
| | | TTCGACTGGGACAATTCCAGTATCGAGTCCAAGCGGGTGGTGATCGAGATGCTGG |
| | | TGCAGAAAGTGATCATTCACGACAACTCCATCGAAATCATCCTGGTGGAA |
| 54 | | MKGESELDKKAAIYIRVSTQEQATEGYSIQAQTDRLIKYVEAKDFILYKKYIDAGYSA |
| | | SKLERPAMQDLIQDVQSKKVDVVIVYKLDRLSRSQKDTMYLIEDIFRPNDVELISMQE |
| | | SFDTSTAFGSATVGMLSVFAQLERKSISERMITGRVERAKKGFYHTGGQDRPPAGYQ |
| | | FNSDNQLIINEYEAAAIKDLFRLYNDGLGKSSISEYLKKNYPGKNKWLPSSIDRMLKN |
| | | SLYIGKVKFSGAEYDGIHEPIIDEVTFYKTQKEIARRKQTNTKRYNYVALLGGLCECG |
| | | ICGAKMANRRAVGRKGKVYRYYRCYSKKGSPKHMMKTDGCSSKAQQQFIIDEAVI |
| | | NNLKNIDVEAELKRRSAPQTNTSLISSQIESIDKQINKLIDLFQVDSMPLDVISEKIDKL |
| | | NKEKQSMEKLLERKNKLDKTELQHRFDVLKSFDWDNSSIESKRVVIEMLVQKVIIHD |
| | | NSIEIILVE |
| 17 | Int17 | ATGCGGACCAACGAGCACAACTTCCACAACATCGAGGAGGAGATTAAGCACGTG |
| | | GCCGTGTACCTGAGACTGTCCCGGGGTGAGGATGAGAGCGAGCTGGATAACCAC |
| | | AAGACTCGGCTGCTGAACAGATGTGAACTCAACAACTGGTCCTACGAGCTGTATA |
| | | AGGAAATCGGATCTGGCTCTACCATCGATGATAGACCTGTGATGCAGAAACTGCT |
| | | GACCGATGTGGAAAAGAACCTGTACGACGCCGTGCTGGTGGTGGACCTGGATAG |
| | | GCTGTCGAGAGGCAACGGCACCGACAACGACAGAATCCTGTATTCCATGAAAGT |
| | | GTCCGAAACCCTGATCGTGGTGGAATCCCCCTACCAGGTGCTGGACGCTAACAAC |
| | | GAGTCCGACGAAGAGATCATCCTGTTTAAGGGCTTCTTCGCCCGGTTCGAGTTCA |
| | | AGCAGATCAATAAGCGGATGAGAGAGGGCAAGAAGCTGGCTCAGAGCAGAGGC |
| | | CAGTGGGTCAACTCCGTGACACCCTACGGCTACATCGTTAACAAGACCACCAAG |
| | | AAACTGACCCCTTCTGAAGAGGAAGCCAAAGTGGTGATCATGATCAAGGACTTC |
| | | TTCTTTGAAGGCAAGAGCACCTCCGACATCGCTTGGGAGCTGAACAAGAGAAAG |
| | | ATCAAGCCTAGACGGGCTACAGAATGGCGGTCCTCCTCTATCGCCAATATCCTGC |
| | | AGAATGAAGTGTACGTGGGCAACATCGTGTACAACAAGTCTGTCGGAAACAAGA |
| | | AGCCCTCTAAGTCCAAGACCAGAGTGACCACCCCATACAGACGGCTGCCTGAGG |
| | | AGGAGTGGCGGCGCGTGTACAACGCCCACCAGCCTCTGTACTCTAAGGAAGAGT |
| | | TCGACCGGATCAAGCAGTACTTCGAGTGCAACGTCAAGAGCCATAAGGGATCCG |
| | | AGGTGCGCACCTACGCCCTGACCGGCCTGTGCAAGACCCCTGACGGCAAGACCA |
| | | TGAGAGTGACCCAGGGCAAGAAGGGCACCGACGACGACCTGTATCTGTTCCCTA |
| | | AGAAGAACAAGCACGGCGACAGCAGTATCTACAAGGGCATTTCCTACAACGTCG |
| | | TGTACGAGACACTCAAAGAGGTGATCTTGCAAGTGAAAGACTACCTGGACTCTGT |
| | | GCTGGACCAGAACGAAAATAAGGACCTGGTGGAAGAACTGAAAGAGGAACTGA |
| | | TGAAGAAGGAGGATGAACTGGAAACAATCCAGAAGGCCAAGAATCGGATCGTG |
| | | CAAGGCTTTCTGATCGGCCTGTACGACGAGCAGGACTCCATCGAGTTGAAGGTGG |
| | | AGAAGGAGAAAGAGATCGACGAAAAGGAAAAGGAGATCGAGGCTATCAAGATG |
| | | AAGATCGACAATGCAAAAACCGTGAACAACTCCATCAAAAAAACCAAGATCGAG |
| | | AGACTGCTGTCTGACGTGCAGTCTGCCGAGTCTGAGAAAGAAATCAACCGGTTCT |
| | | ACAAGACCCTGATCAAGGAGATCATCGTGGATAGAACCGATGAAAACGAGGCTA |
| | | AGATCAAGGTCAACTTCCTG |
| 55 | | MRTNEHNFHNIEEEIKHVAVYLRLSRGEDESELDNHKTRLLNRCELNNWSYELYKEI |
| | | GSGSTIDDRPVMQKLLTDVEKNLYDAVLVVDLDRLSRGNGTDNDRILYSMKVSETLI |
| | | VVESPYQVLDANNESDEEIILFKGFFARFEFKQINKRMREGKKLAQSRGQWVNSVTP |
| | | YGYIVNKTTKKLTPSEEEAKVVIMIKDFFFEGKSTSDIAWELNKRKIKPRRATEWRSS |
| | | SIANILQNEVYVGNIVYNKSVGNKKPSKSKTRVTTPYRRLPEEEWRRVYNAHQPLYS |
| | | KEEFDRIKQYFECNVKSHKGSEVRTYALTGLCKTPDGKTMRVTQGKKGTDDDLYLF |
| | | PKKNKHGDSSIYKGISYNVVYETLKEVILQVKDYLDSVLDQNENKDLVEELKEELMK |
| | | KEDELETIQKAKNRIVQGFLIGLYDEQDSIELKVEKEKEIDEKEKEIEAIKMKIDNAKT |
| | | VNNSIKKTKIERLLSDVQSAESEKEINRFYKTLIKEIIVDRTDENEAKIKVNFL |
| 18 | Int18 | ATGATCACAACAAACAAGGTGGCCATCTACGTGCGGGTGTCTACCACCAACCAA |
| | | GTGGAGGAAGGCTACTCCATCGACGAGCAGAAGGACAAGCTGGAGGCTTACTGC |
| | | AAGATCAAAGACTGGAAGATCTACGATGTGTACGTGGATGGCGGCTTCAGCGGC |
| | | GCCAACACCCAGCGGCCTGAGCTGGAACGGCTGATCTCCGACGTGAAGCGGAAG |
| | | AAGGTGGACATCGTGCTGGTGTATAAGCTGGACAGACTGTCTAGATCCCAGAAG |
| | | GACACACTGTTTCTGATCGAGGATGTGTTCGCCAAGAACGACGTGGCTTTCATCA |
| | | GCCTGCAGGAGAACTTCGACACCTCCACCCCTTTCGGAAAGGCCTCTATAGGCAT |
| | | GCTGTCTGTGTTTGCTCAGCTGGAGCGGGAGCAGATCAAGGAAAGAATGATGCT |
| | | GGGCAAAGAAGGCAGAGCCAAGAATGGCAAGTCCATGTCTTGGACCACCATCGC |
| | | CTTCGGCTACGACTACTCTAAGGAAACCGGCGTGCTGTCCGTGAACCCTACCCAG |
| | | GCTCTGATCGTCAACCGGATCTTCACCGAGTACCTGAACGGCAAGCCTGTGGTGA |
| | | AAATCATCCGGGACCTGAACGCCGAGGGCCATGTGGGCAGAAAGCGGCCTTGGG |
| | | GCGAGACAATCACCAAGTACCTGCTGAAGAACGAGACATACCTGGGCAAGGTTA |
| | | AGTATAAAGACAAGGTGTACGAGGGCCAGCACGAGCCCATCATCACCCAAGAGC |
| | | TGTTCGATCTGGTGCAGCTGGAAGTGGAGCGGAGACAGATCTCCGCCTACGAAA |
| | | AGTACAACAACCCCAGACCATTCAGAGCTAAGTACATGCTGAGCGGCCTGATGA |
| | | AGTGCGGATACTGTGGCGCTTCTCTGGGCCTGAGATACACCAGAAAGGACAAGA |
| | | ACGGCATCTCTCACCACAAGTACCAGTGCCGGAATCGGCACTCCAAGGACCTGG |
| | | AAAAAAGATGCGAGTCTGGCTGGTACTCCAAAGAGGAACTCGAGCGCGGAGTGA |
| | | TCAAGGAACTGGAACGTATCAAGTTCGATCCTAAGTATAAGAATGAAACCCTGG |
| | | CCAAGAAAGAGGAAACCATCAAAGTGGAAGAGATCAAGAAGCAGCTGGAGCGG |
| | | ATCAACAACCAGGTGTCCAAACTGACCGAGCTGTACCTCGATGAGATCATCACCA |
| | | GGAAGGAGCTTGATGAAAAGAACGACAAGATCAAGACCGAAAGACAATTCCTG |
| | | GAGGAGCAGCTGGAGAACCAGAAGTCCAACGTGCTCTCCATCAGAAAGCGGAAA |
| | | CTGACCAGACTGCTGAAGGATTTTGACGTCGAGAAGCTGTCCTACGAGGACGCCT |
| | | CTAAGATTGTCAAGAACATCATCAAAGAAATCATCGTGACTAAGGACGGCATGT |
| | | CCATCACCCTGGACTTC |
| 56 | | MITTNKVAIYVRVSTTNQVEEGYSIDEQKDKLEAYCKIKDWKIYDVYVDGGFSGAN |
| | | TQRPELERLISDVKRKKVDIVLVYKLDRLSRSQKDTLFLIEDVFAKNDVAFISLQENF |
| | | DTSTPFGKASIGMLSVFAQLEREQIKERMMLGKEGRAKNGKSMSWTTIAFGYDYSK |
| | | ETGVLSVNPTQALIVNRIFTEYLNGKPVVKIIRDLNAEGHVGRKRPWGETITKYLLKN |
| | | ETYLGKVKYKDKVYEGQHEPIITQELFDLVQLEVERRQISAYEKYNNPRPFRAKYML |
| | | SGLMKCGYCGASLGLRYTRKDKNGISHHKYQCRNRHSKDLEKRCESGWYSKEELER |
| | | GVIKELERIKFDPKYKNETLAKKEETIKVEEIKKQLERINNQVSKLTELYLDEIITRKEL |
| | | DEKNDKIKTERQFLEEQLENQKSNVLSIRKRKLTRLLKDFDVEKLSYEDASKIVKNIIK |
| | | EIIVTKDGMSITLDF |
| 19 | Int19 | ATGGGCAAGTCTATCACCGTGATCCCAGCTAAAAAAGTGCAGACCTCTGTGCTGC |
| | | ATCAAGACCGGAAGAAGATCAAGGTGGCCGCCTACTGTCGGGTGTCCACCGACC |
| | | AGGAGGAGCAGCTGTCCTCCTATGAAAACCAGGTGAACTACTACAGAGAGTTCA |
| | | TCTCCAAGCACGAGGACTACGAGCTGGTGGACATCTACGCCGACGAGGGCATCT |
| | | CCGCAACCAACACCAAGAAGCGGGACGCCTTCAACCGGCTGATCCAAGACTGTA |
| | | GGGCCGGAAAGGTCGACAGAATACTGGTGAAGTCCATCTCGAGATTCGCCAGAA |
| | | ACACACTGGATTGCATCAAGTACGTGCGGGAGCTGAAGGAACTGGGCGTGGGCG |
| | | TGACCTTCGAGAAAGAGAACATCGACAGCCTGGATAGTAAGGGCGAGGTTCTGC |
| | | TGACCATTCTGAGCTCTCTGGCTCAGGACGAGTCTCGATCTATCTCTGAGAACGC |
| | | CACCTGGGGCATCAGAAAGAAGTTCGAGAGAGGCGAAGTGCGCGTCAATACAAC |
| | | AAAGTTCATGGGCTACGACAAGGACGAGAACGGCAGACTGATCATCAACCCTCA |
| | | ACAGGCTGAAACCGTCAAGTTTATCTACGAGAAATTTCTGGAGGGCTACTCCCCC |
| | | GAGTCCATCGCCAAGTACCTGAACGACAATGAGATCCCTGGCTGGACCGGCAAG |
| | | GCCAACTGGTACCCTTCTGCCATCCAGAAGATGCTGCAGAACGAGAAGTACAAG |
| | | GGCGACGCTCTGCTGCAGAAAACCTTTACCGTGGACTTCCTGACCAAGAAGAGA |
| | | GTGCAGAACGATGGACAGGTGAACCAGTACTACGTGGAAAATTCTCACGAGGCC |
| | | ATCATCGACGAAGAGACATGGGAAACAGTGCAGCTCGAGATGGCCAGAAGAAA |
| | | GACCTACAGAGATGAGCACCAGCTGAAATCCTACATCATGCAGTCCGAGGATAA |
| | | CCCCTTCACCACCAAGGTGTTCTGCGGCGCTTGTGGCTCCGCTTTCGGCCGGAAG |
| | | AACTGGGCTACCTCCAGAGGAAAGCGGAAAGTGTGGCAGTGCAACAACAGATAC |
| | | CGGATCAAGGGAGTCGAAGGCTGCTACAGCTCCCACCTGGACGAGGCTACCCTC |
| | | GAACAGATCTTCCTGAAAGCCCTGGAACTGCTGTCCGAAAACATCGACCTGCTGG |
| | | ATGGCAAGTGGGAGAAGATCCTGGCCGAGAACAGACTGCTTGATAAGCACTATA |
| | | GCATGGCTTTATCTGATCTGCTGCGGCAGGAACAGATCGACTTCAATCCTTCCGA |
| | | CATGTGCAGAGTGCTGGACCACATCCGGATCGGCCTGGATGGCGAAATCACCGT |
| | | GTGCCTGCTGGAAGGTACCGAGGTGGACCTG |
| 57 | | MGKSITVIPAKKVQTSVLHQDRKKIKVAAYCRVSTDQEEQLSSYENQVNYYREFISK |
| | | HEDYELVDIYADEGISATNTKKRDAFNRLIQDCRAGKVDRILVKSISRFARNTLDCIK |
| | | YVRELKELGVGVTFEKENIDSLDSKGEVLLTILSSLAQDESRSISENATWGIRKKFERG |
| | | EVRVNTTKFMGYDKDENGRLIINPQQAETVKFIYEKFLEGYSPESIAKYLNDNEIPGW |
| | | TGKANWYPSAIQKMLQNEKYKGDALLQKTFTVDFLTKKRVQNDGQVNQYYVENS |
| | | HEAIIDEETWETVQLEMARRKTYRDEHQLKSYIMQSEDNPFTTKVFCGACGSAFGRK |
| | | NWATSRGKRKVWQCNNRYRIKGVEGCYSSHLDEATLEQIFLKALELLSENIDLLDGK |
| | | WEKILAENRLLDKHYSMALSDLLRQEQIDFNPSDMCRVLDHIRIGLDGEITVCLLEGT |
| | | EVDL |
| 20 | Int20 | ATGAGAACAGTCAGACGCATCCAGCCTATCAAGTCTCCTTGCAAGCCTAGATTCA |
| | | AAGTGGCCGCCTATGCTAGAGTGTCCGACTCACGCCTGCACCACTCTCTGTCCAC |
| | | CCAGATCTCCTACTACAACAGACTGATCCAGGCCCATCCTGATTGGGAGTTGGTC |
| | | GGAATCTACTACGACGAGGGAATTTCCGGCAAAGAGCAGTCCAACAGACAGGGC |
| | | TTCCTGAATCTGATCAAGGACTGCGAGGACGGCAAGATCGATAGAATCATCACC |
| | | AAGTCCATCGCCAGATTTGGACGGAACACCGTGGAACTGCTGACCACCGTGCGG |
| | | CAGCTGAGACTGAAGAACATCGGCGTGACCTTCGAGAAGGAAAACATCGACAGC |
| | | CTGTCCTCTGAAGGCGAGCTGATGCTGACACTGCTGGCTTCTGTGGCCCAGGAAG |
| | | AGTCCCAGAACCTGTCTGAGAATATCAGATGGCGGATCCAGAAGAAGTTCGAAA |
| | | AGGGAATCCCTCACACCCCTCAGGACATGTACGGCTATCGGTGGGATGGCGAAC |
| | | AGTACCAGATCGAACCCAACGAGGCCAAGGTGATCCGGAAGGTGTTCAAGTGGT |
| | | ACCTGGACGGCGACTCCGTGCAGCAGATCGTGGACAAGCTGAACCAGGAGCAGG |
| | | TGCTGACCCGGCTCGGCAACCCCTTCACCGTGGCTAGCATCAGAGAGTTCTTCAA |
| | | GCAGGAAGCTTACTTTGGTAGACTCGTGCTGCAGAAAACCTACAGAGAAGCCTTC |
| | | TCCAGAAATCCAAAGAGGAACAAAGGCCAGAGAAACAAGTACATCATCGAGAA |
| | | CGCTCACGAGCCCATCGTTACAAAGGAATACTTCGACCTGGTGCTGCATGAGAAA |
| | | GAGCGAAGAAACCAACTGATGCACCAAGAGTCTCACCTGAACAAGGGCATCTTC |
| | | CGGGATAAGATCTCTTGCTCCGAGTGCGGCTGTCTGATGATCGTGAAAGTCGATT |
| | | CCAAGCAAGTGAACAAGACCGTGCGGTACTACTGCAGAACCAGAAACCGGTTCG |
| | | GCGCTTCTTCCTGCAGCTGTCGGACCCTGGGCGAGAAGCGGCTGCTGGCCAGCTT |
| | | TAAATCCAAGCTGGGCATCGTGCCTGACAAGGAGTGGGTGGAAAACAACATCAA |
| | | GCACATCGAGTACGACTTCGGCTACCGGATCCTGCGGGTGACACCTGTGAAGGG |
| | | CAGAAAGTACCTGATCGAGATCAGAGAGGGCAGATAC |
| 58 | | MRTVRRIQPIKSPCKPRFKVAAYARVSDSRLHHSLSTQISYYNRLIQAHPDWELVGIY |
| | | YDEGISGKEQSNRQGFLNLIKDCEDGKIDRIITKSIARFGRNTVELLTTVRQLRLKNIG |
| | | VTFEKENIDSLSSEGELMLTLLASVAQEESQNLSENIRWRIQKKFEKGIPHTPQDMYG |
| | | YRWDGEQYQIEPNEAKVIRKVFKWYLDGDSVQQIVDKLNQEQVLTRLGNPFTVASI |
| | | REFFKQEAYFGRLVLQKTYREAFSRNPKRNKGQRNKYIIENAHEPIVTKEYFDLVLHE |
| | | KERRNQLMHQESHLNKGIFRDKISCSECGCLMIVKVDSKQVNKTVRYYCRTRNRFG |
| | | ASSCSCRTLGEKRLLASFKSKLGIVPDKEWVENNIKHIEYDFGYRILRVTPVKGRKYLI |
| | | EIREGRY |
| 21 | Int21 | ATGCGGAACAAGGTTGCCATCTACGTCCGGGTGTCCACAGCTAGCCAGGCCGAC |
| | | GAGGGCTACTCCATCGACGAACAGAAAAGCAAGCTGGAGGCCTACTGCGAGATC |
| | | AAGGACTGGAAGATCTACGACACCTACATCGATGGCGGCTTCTCCGGGGCCAAC |
| | | ACCCAGAGGCCCGAACTGGAACGGCTGATTTCTGATGCCAAGCGGAAGAAGATT |
| | | GATATCGTGCTGGTGTACAAGCTGGACAGACTGTCCAGATCTCAAAAGGACACA |
| | | CTGTTCCTGATCGAGGATGTGTTCGCTAAGAACGACGTGGCTTTCATCAGCCTGC |
| | | AGGAGAACTTCGACACCTCTACCCCTTTCGGCAAGGCCTCCATCGGCATGCTGTC |
| | | CGTGTTCGCCCAGCTGGAGCGCGAACAGATCAAAGAGCGGATGATGCTGGGCAA |
| | | AGAGGGCAGAGCCAAGAATGGCAAGTCCATGTCTTGGACCACCATCCCTTTTGGC |
| | | TACGACTACTCCAAAGAGACAGGCATCCTGAGCGTGAACCCCACCCAAGCTCTG |
| | | ATCGTGAAGAGAATCTTCACCGAGTACCTGAACGGCAAATCTGTGGTGAAGATC |
| | | ATCCGGGACCTGAATGCCGAGGGCCATGTGGGCCGGAAGCGGCCTTGGGGCGAA |
| | | ACCATCACCAAGTATCTGCTGAAAAACGAAACCTACCTCGGAAAGTCTAAGTAT |
| | | AAGGGCAAGGTATTCGAAGGCCAGCACGACGCCATCATCTCTCAGGAACTGTTT |
| | | GATCTGGTGCAGCTGGAAGTGGAGAAGAGACAGATCTCCGCCTTCGAGAAGTAC |
| | | AACAACCCTAGACCTTTCCGGGCTAAGTACATGCTGTCTGGCCTAATGAAGTGCG |
| | | GCTACTGCGGCGCTTCTCTGGGACTCTACGTGGCCCCTAAGAACAAGAACGGCGT |
| | | GAGCAAGTACAAGTACCAGTGTAGACACCGGTACCACAAGGACAAAGCCATCAG |
| | | ATGCAACTCCGGATGGTACTCCAAGGACGAGCTGGAGAAAAGAGTGATCAAAGA |
| | | GCTCGAGCGGCTGAAGTTCGATCCTAAGTACAAGAAAGAAACCCTGGCCAAGAA |
| | | AGATGAGACAATTAAGGTGGAGGACATCAAGAAGCAGCTGGAAAGAATCAATA |
| | | AGCAGGTGTCCAAGCTGACCGAGCTGTACCTGGACGAGGTGATCACCAGAAAGG |
| | | ACCTGGACGAAAAGAACGCCAAGATCAAGACCGAAAGACAGTACCTGGAGGAG |
| | | CAGCTGGAGAACCAGAAGTCCAACGTGATGTCCATCCGAAAGCGGAAGCTGTCT |
| | | AGACTGCTGAAGGACTTCGACATCGAGAAGCTGTCCTACGAGGAAGCTTCTAAG |
| | | ATCGTGAAGTCCGTCATCAAGGAAATCGTCGTGACCAAGGACGACATGACCATC |
| | | ACTCTGGATTTT |
| 59 | | MRNKVAIYVRVSTASQADEGYSIDEQKSKLEAYCEIKDWKIYDTYIDGGFSGANTQR |
| | | PELERLISDAKRKKIDIVLVYKLDRLSRSQKDTLFLIEDVFAKNDVAFISLQENFDTSTP |
| | | FGKASIGMLSVFAQLEREQIKERMMLGKEGRAKNGKSMSWTTIPFGYDYSKETGILS |
| | | VNPTQALIVKRIFTEYLNGKSVVKIIRDLNAEGHVGRKRPWGETITKYLLKNETYLGK |
| | | SKYKGKVFEGQHDAIISQELFDLVQLEVEKRQISAFEKYNNPRPFRAKYMLSGLMKC |
| | | GYCGASLGLYVAPKNKNGVSKYKYQCRHRYHKDKAIRCNSGWYSKDELEKRVIKE |
| | | LERLKFDPKYKKETLAKKDETIKVEDIKKQLERINKQVSKLTELYLDEVITRKDLDEK |
| | | NAKIKTERQYLEEQLENQKSNVMSIRKRKLSRLLKDFDIEKLSYEEASKIVKSVIKEIV |
| | | VTKDDMTITLDF |
| 22 | Int22 | ATGAAGGTGGCCACTTACGTGCGCGTGTCCACCGACGAGCAGGCTAAGGAGGGC |
| | | TTCTCCATCCCCGCCCAAAGAGAGCGGCTGAGAGCCTTCTGCGAGTCTCAGGGAT |
| | | GGGAAATCGTGGAAGAGTACATCGAAGAGGGCTGGTCCGCCAAAGACCTGGACA |
| | | GACCTCAGATGCAGCGGCTGCTCAAGGATATCAAGAAGGGCAATATCGACATCG |
| | | TGCTGGTGTACAGGCTGGATAGACTGACCCGGTCTGTGCTGGATCTGTACCTGCT |
| | | GCTGCAGACCTTTGAGAAGTACAACGTGGCTTTCAGATCCGCTACCGAGGTGTAC |
| | | GACACCTCTACCGCCATGGGCAGACTGTTCATTACCCTTGTGGCCGCCCTGGCTC |
| | | AGTGGGAGCGGGAGAACCTGGCCGAGAGAGTGAAGTTCGGCATCGAGCAGATG |
| | | ATCGACGAGGGAAAGAAGCCTGGCGGCCACTCTCCATACGGATACAAGTTTGAC |
| | | AAGGACTTCAACTGCACCATCATCGAGGATGAGGCCAACACCGTGCGGATGATT |
| | | TACAGAATGTACTGCGACGGCTACGGCTACCACTCCATCGCTAAGCGCCTGAATG |
| | | AGCTGGGCATCAAGCCTAGAATCGCCAAAGAGTGGAACCACAACAGCGTCCGGG |
| | | ACATCCTGACCAACGACATCTACATCGGCACCTATAGATGGGGCAACAAGGTTGT |
| | | GCTGAACAACCATCCTCCTATCATCTCCGAGACACTGTTCAGAAAGGTGCAGAAA |
| | | GAAAAAGAAAAGCGGCGGGTGGACCGGACCAGAGTGGGCAAGTTTCTGCTGACA |
| | | GGCCTGCTGTACTGTGGCAATTGCAACGGCCACAAGATGCAGGGCACCTTTGACA |
| | | AAAGAGAACAGAAAACCTACTACCGGTGTCTGAAGTGCAACCGGATCACCAACG |
| | | AGAAGAACATCCTGGAACCTCTGCTGGATGAGATCCAGCTGCTGATCACATCTAA |
| | | AGAGTACTTCATGTCCAAGTTCTCCGACCAGTACGATCAAAAGGAGGAAGTGGA |
| | | CGTGTCTGCTCTGAAGAAGGAGCTCGAAAAGATCAAGAGACAGAAGGAAAAGTG |
| | | GTACGACCTGTACATGGACGACAGAAACCCCATCCCTAAGGAAGATCTGTTCGCC |
| | | AAGATCAACGAGCTGAACAAGAAGGAAGAAGAGATCTATAACAAGCTGAACGA |
| | | GGTCGAACCCGAGGACAAGGAGCCTGTCGAAGAAAAGTACAACAGACTGAGCA |
| | | AGATGATCGACTTCAAGCAGCAGTTCGAGCAAGCTAATGATTTCACCAAGAAAG |
| | | AACTGCTGTTCAGCATCTTCGAAAAAATCGTGATCTATCGGGAGAAGGGCAAGCT |
| | | GAAAAAGATCACCCTGGACTACACCCTGAAG |
| 60 | | MKVATYVRVSTDEQAKEGFSIPAQRERLRAFCESQGWEIVEEYIEEGWSAKDLDRPQ |
| | | MQRLLKDIKKGNIDIVLVYRLDRLTRSVLDLYLLLQTFEKYNVAFRSATEVYDTSTA |
| | | MGRLFITLVAALAQWERENLAERVKFGIEQMIDEGKKPGGHSPYGYKFDKDENCTII |
| | | EDEANTVRMIYRMYCDGYGYHSIAKRLNELGIKPRIAKEWNHNSVRDILTNDIYIGT |
| | | YRWGNKVVLNNHPPIISETLFRKVQKEKEKRRVDRTRVGKFLLTGLLYCGNCNGHK |
| | | MQGTFDKREQKTYYRCLKCNRITNEKNILEPLLDEIQLLITSKEYFMSKFSDQYDQKE |
| | | EVDVSALKKELEKIKRQKEKWYDLYMDDRNPIPKEDLFAKINELNKKEEEIYNKLNE |
| | | VEPEDKEPVEEKYNRLSKMIDFKQQFEQANDFTKKELLFSIFEKIVIYREKGKLKKITL |
| | | DYTLK |
| 23 | Int23 | ATGCTGCGCGTGGCTCTGTATATCAGAGTGTCTACCGAGGAGCAGGCCCTGAACG |
| | | GCGACAGCATCCGGACCCAGATCGAGGCCCTGGAACAGTACTCCAAGGAGAACG |
| | | ACTTCAACATCGTGGGCAAGTACATCGACGAGGGCTGTTCTGCCACCAACCTGAA |
| | | GCGGCCTAATCTGCAAAGACTGCTGCGGGACGTGGAAAAAGACAAAGTGGACCT |
| | | GGTGCTGATGACTAAGATCGATCGGCTGTCTAGAGGAGTCAAGAACTACTACAA |
| | | GATCATGGAAACACTGGAGAAGCACAAGTGCGACTGGAAAACCATCCTGGAAAA |
| | | CTACGACTCCTCCACCGCCGCTGGCAGACTCCACATCAACATCATGCTGTCCGTG |
| | | GCCGAGAACGAGGCTGCTCAGACCTCCGAGAGAATCAAGTTCGTGTTCCAGGAC |
| | | AAGTTGAGAAGAAAGGAAGTGATCTCTGGTACAATCCCCATCGGCTACAAAATC |
| | | GAGAATAAGCATCTGGTGATCGATAAAGAGAAGAAGTACATTGTGAAGGCCATC |
| | | TTCGACGAGTACGAGAAGTCTGGCTCCGTTAGGACCCTGATCGAAACCATCAACA |
| | | ACCTGCACGGCGAACTGTACTCCTATAACAAGATCAAGAACATCCTGAGAAACG |
| | | AGCTGTACATCGGCATTTACAATAAGAGAGGCTTCTACGTGGAGGACTACTGCGA |
| | | GCCTATCATCAGCAAGAAGCAGTTCAAGCAGATCCAGCGGATCCTGGAAAAGAA |
| | | TAAGAAAACCACACCAAACAAGAACATCCACTACCACATCTTCAGCGGCCTGCT |
| | | CAAGTGCAAGGAGTGTGGCTACACCCTGAAGGGCAACTCCTCCAACGTGGGAGA |
| | | GAAGCTGTACCTGTCTTACAGATGCTCCACCTTTTACCTGAACAAGAACTGCGTG |
| | | CACAACGTGACCCACAACGAGAAGCATATTGAGAACTATCTGCTGACCAACCTG |
| | | AAGCCTCAGCTGCACAAGCACATGGTGAAGCTGGAAGCCCAGAACGAAAAGATC |
| | | AGACGGAACAAAAAGTCCAACAAGAAGGATGAGAAAAAGAAAATCATGAAGAA |
| | | ACTGGATAAGATCAAGGACCTGTACCTGGAGGACCTGATCGATAAAGAAACCTA |
| | | CCGGAAGGACTACGAGAAGCTGCAGTCCCAGCTGGACAACATCACCGAGGAACA |
| | | AGAGTCTCAGATCATCGACACCTCTCACATCAAGAAGTTTCTGGACATCGACATC |
| | | AATGAGATGTACTCTGATCTGAGCAGAGTCGAGCGGCGGAGATTCTGGCTGTCCA |
| | | TCATAGACTACATCGAGATCGATAACAACAAAAACATCACCATCAACTTCATC |
| 61 | | MLRVALYIRVSTEEQALNGDSIRTQIEALEQYSKENDFNIVGKYIDEGCSATNLKRPN |
| | | LQRLLRDVEKDKVDLVLMTKIDRLSRGVKNYYKIMETLEKHKCDWKTILENYDSST |
| | | AAGRLHINIMLSVAENEAAQTSERIKFVFQDKLRRKEVISGTIPIGYKIENKHLVIDKE |
| | | KKYIVKAIFDEYEKSGSVRTLIETINNLHGELYSYNKIKNILRNELYIGIYNKRGFYVE |
| | | DYCEPIISKKQFKQIQRILEKNKKTTPNKNIHYHIFSGLLKCKECGYTLKGNSSNVGEK |
| | | LYLSYRCSTFYLNKNCVHNVTHNEKHIENYLLTNLKPQLHKHMVKLEAQNEKIRRN |
| | | KKSNKKDEKKKIMKKLDKIKDLYLEDLIDKETYRKDYEKLQSQLDNITEEQESQIIDT |
| | | SHIKKFLDIDINEMYSDLSRVERRRFWLSIIDYIEIDNNKNITINFI |
| 24 | Int24 | ATGAAGATCACCCTGCTGTACTACATCAAGAAGTTCAACATCTACTGCAACAGAT |
| | | ACCTGAGCCAGCAGATCAACATCTCCGTGGACATCATCGGCTTCTACCAGTTCAA |
| | | GAACGTCACCAACTCTGTGACCGACGTGCTGAAGAGAGGTGATAATCTGGACAG |
| | | AATCTGTATCTACCTGCGGAAGTCCAGAGCCGATGAAGAACTGGAAAAGACCAT |
| | | CGGAGTGGGCGAAACCCTGAGCAAGCACAGAAAGGCTCTGCTGAAGTTCGCCAA |
| | | GGAAAAGAAGCTGAATATCATGGAAATCAAAGAGGAAATAGTGTCCGCTGACTC |
| | | CATCTTCTTCAGACCTAAGATGATCGAACTGCTGAAGGAGGTGGAGAACAACCA |
| | | GTACACCGGCGTGCTGGTTATGGACATCCAGAGACTGGGCAGAGGCGACACCGA |
| | | GGACCAGGGCATCATTGCTAGAATCTTCAAGGAGTCTCACACCAAGATCATCACC |
| | | CCTATGAAAACCTACGACCTGGACGACGATTTGGACGAGGACTACTTTGAGTTCG |
| | | AGAGTTTCATGGGCCGCAAAGAGTACAAGATGATCAAGAAGCGGATGCAGGGCG |
| | | GCAGAGTGCGGTCCGTGGAAGATGGCAACTACATCGCCACCAATCCTCCATTTGG |
| | | CTACGACATCCACTGGATCAACAAGTCCAGGACACTGAAGTTCAACTCCAAGGA |
| | | ATCTGAGATCGTGAAACTGATCTTTAAACTGTATACCGAGGGAAATGGCGCTGGC |
| | | ACCATCTCCAACTACCTGAACTCCCTGGGCTATAAGACCAAGTTCGGCAACAACT |
| | | TCAGCAACTCTTCTATCATCTTCATCCTGAAGAACCCTGTGTACATCGGAAAGAT |
| | | CACCTGGAAGAAGAAGGACATCAGAAAGTCCAAGGATCCTCACAAGGTCAAAGA |
| | | TACCCGGACCAGAGACAAGTCCGAGTGGATCATCGCCGACGGCAAGCACGAGCC |
| | | TATCATCGACGAAAAGATCTGGAACAAGGCTCAAGAGATCCTGAACAACAAGTA |
| | | CCACATCCCTTACAAGATCGCCAACGGCCCCGCTAACCCTCTGGCCGGAGTGGTG |
| | | ATCTGCTCCAAGTGCAACTCCAAAATGGTGATGCGGAAGTACGGCAAGAAGCTG |
| | | CCTCATCTGATCTGCAATAACAAGGAGTGTAACAATAAGTCCGCCAGATTCGACT |
| | | ACATCGAGAAGGCCGTGCTGGAAGGCCTGGACGAGTATCTGAAGAACTACAAAG |
| | | TGAACGTGAAGGCCAACAACAAAACCAGCGATATCGAGCCCTACGAGCAGCAGT |
| | | CTAACGCCCTGAACAAAGAGCTGATCCTCCTGAACGAGCAGAAACTGAAGCTAT |
| | | TCGACTTTTTGGAAAGAGAGATCTACACAGAAGAGATCTTTCTTGAGAGATCTAA |
| | | GAACCTGGATGAGCGGATCAACACCACCACACTGGCTATAAACAAGATCAAGAA |
| | | AATTCTGGACAACGAGAAAAAGAAGAACAACAAGAACGACATCGTCAAGTTCGA |
| | | GAAAATCCTGGAAGGCTACAAGAAAACCAACGATATCCAGAAGAAAAATGAACT |
| | | GATGAAATCTCTGGTGTTCAAGATCGAGTATAAGAAAGAACAGCACCAGCGGAA |
| | | CGACGGCCTGCTGTACATCTACTTCCTGAGCTTCTGCGTGCGGTGCATCTCCTACC |
| | | TGACACAATTCATTTCCTTCTTCGTGTACCCCTACCGGATCCTGGAGATCTACCTG |
| | | ACCTTCTCTTTTTTCATCATCTCTTACGAGCAT |
| 62 | | MKITLLYYIKKFNIYCNRYLSQQINISVDIIGFYQFKNVTNSVTDVLKRGDNLDRICIY |
| | | LRKSRADEELEKTIGVGETLSKHRKALLKFAKEKKLNIMEIKEEIVSADSIFFRPKMIE |
| | | LLKEVENNQYTGVLVMDIQRLGRGDTEDQGIIARIFKESHTKIITPMKTYDLDDDLDE |
| | | DYFEFESFMGRKEYKMIKKRMQGGRVRSVEDGNYIATNPPFGYDIHWINKSRTLKFN |
| | | SKESEIVKLIFKLYTEGNGAGTISNYLNSLGYKTKFGNNFSNSSIIFILKNPVYIGKITW |
| | | KKKDIRKSKDPHKVKDTRTRDKSEWIIADGKHEPIIDEKIWNKAQEILNNKYHIPYKI |
| | | ANGPANPLAGVVICSKCNSKMVMRKYGKKLPHLICNNKECNNKSARFDYIEKAVLE |
| | | GLDEYLKNYKVNVKANNKTSDIEPYEQQSNALNKELILLNEQKLKLFDFLEREIYTEE |
| | | IFLERSKNLDERINTTTLAINKIKKILDNEKKKNNKNDIVKFEKILEGYKKTNDIQKKN |
| | | ELMKSLVFKIEYKKEQHQRNDGLLYIYFLSFCVRCISYLTQFISFFVYPYRILEIYLTFS |
| | | FFIISYEH |
| 25 | Int25 | ATGCGGATCTGCATGTACCTGCGGAAGTCCAGAGCTGATGAGGAACTGGAAAAG |
| | | ACCCTGGGCGAAGGCGAGACTCTGAGCAAGCACAGAAAGGCTCTGCTGAAGTTC |
| | | GCCAAGGAGAAAAATCTGAATATCGTGGAGATCAAAGAGGAAATCGTGTCTGGT |
| | | GAGTCCCTGTTCTTCAGACCTAAGATGCTGGAACTGCTGAAAGAAATCGAGAAC |
| | | AAACAGTACTCCGGCGTGCTCGTGATGGACATGCAGAGACTGGGAAGGGGAAAC |
| | | ATGCAGGACCAGGGCATCATCCTCGAGACATTTAAGAAATCTAACACCAAGATC |
| | | ATCACCCCTATGAAAACCTACGACCTGTCTAACGACTTCGACGAAGAGTACTCTG |
| | | AGTTCGAGGCCTTCATGTCCCGGAAGGAACTTAAGATGATCAATCGGCGGATGC |
| | | AAGGCGGCAGAGTGCGGAGCGTCGAGGACGGCAACTACATCGCTACCAACGCCC |
| | | CCTACGGCTACGACATCCACTGGATCAACAAGGCCAGAACCCTGAAGCCCAACC |
| | | AGAAGGAATCTGAAATCGTCAAGCTGATCTTCAAGCTCTACATCGAGGGCAACG |
| | | GCGCTGGCACCATCGCTAAGCATCTGAACAGCCTGGGCTATAAGACCAAGTTCG |
| | | GCAACTCCTTCAACAACTCCTCCATCATCTTCATTCTGAAAAACCCTGTGTATATC |
| | | GGCAAGATCACCTGGAAGAAAAAGGACATTCGGAAGTCCAAGGATCCTAACAAA |
| | | GTGAAGGACACCCGGACCAGAGACAAGTCTGAGTGGATCATCGTGGACGGCAAG |
| | | CACGACCCTATCATCGACCAGATCACCTGGAAGCAGGCTCAAGAGATCCTGAAT |
| | | AACCGGTACCACGTGCCTTACAAGCTGGTCAACGGCCCTGCCAACCCCCTGGCCG |
| | | GCCTGATCATCTGTACCACCTGCAAGTCCAAGATGGTGATGAGAAAGCTGAGAG |
| | | GCACCGACAGAATCCTGTGCAAGAACAACAAGTGCAACAACATCTCCAACAGAT |
| | | TCGATGCCGTGGAAAAGTCCGTGGTGGAATCTCTGGAAAACTACCTGAAGGCCT |
| | | ACAAGGTGAACCTGCCTGAGCTGAACAAGACCTCCAACCTGAAACTGTACGAGC |
| | | AGCAGATCAGCACACTGAAGAAAGAACTGAAAATTTTGAACGAACAGAAACTGA |
| | | AGCTGTTCGATTTTCTGGAGCGCGGAATCTACGACGAGGATACCTTCCTGAAGAG |
| | | ATCTAAGAACCTGGACGAGAGAATCGAGATCACCAACGAGTCTCTGTCTAATCTG |
| | | AATCAGATCATCGCCAAGGAGAACAAGGCCATCAAGAAAGAAGATATCATCAAG |
| | | TTTGAGAAGGTGCTGGATAGCTACAAGTCCACCGCTGACATCCGGCTGAAAAAC |
| | | GAGCTGATGAAAACCTTAATCTTCAAGATCGAGTACACCAAGAACAAGAAGGGC |
| | | AATGACTTCAAGATCAAGGTGTTCCCTAAGCTGAAGCCACTGAACATC |
| 63 | | MRICMYLRKSRADEELEKTLGEGETLSKHRKALLKFAKEKNLNIVEIKEEIVSGESLF |
| | | FRPKMLELLKEIENKQYSGVLVMDMQRLGRGNMQDQGIILETFKKSNTKIITPMKTY |
| | | DLSNDFDEEYSEFEAFMSRKELKMINRRMQGGRVRSVEDGNYIATNAPYGYDIHWI |
| | | NKARTLKPNQKESEIVKLIFKLYIEGNGAGTIAKHLNSLGYKTKFGNSFNNSSIIFILKN |
| | | PVYIGKITWKKKDIRKSKDPNKVKDTRTRDKSEWIIVDGKHDPIIDQITWKQAQEILN |
| | | NRYHVPYKLVNGPANPLAGLIICTTCKSKMVMRKLRGTDRILCKNNKCNNISNRFDA |
| | | VEKSVVESLENYLKAYKVNLPELNKTSNLKLYEQQISTLKKELKILNEQKLKLFDFLE |
| | | RGIYDEDTFLKRSKNLDERIEITNESLSNLNQIIAKENKAIKKEDIIKFEKVLDSYKSTA |
| | | DIRLKNELMKTLIFKIEYTKNKKGNDFKIKVFPKLKPLNI |
| 26 | Int26 | ATGATCGCCGCTATCTACTCTAGAAAGTCTAAATTCACCGGCAAGGGCGAGTCCG |
| | | TGGAAAACCAGATCGAAATGTGCAAGGAATACCTGAAGAGAAACTTCAATAACA |
| | | TCGATGACATCGAAATCTACGAGGACGAGGGCTTCTCTGGCAAGGACACCAACC |
| | | GGCCCAAGTTTAAGAAGATGATCAAGGCCGCTAAAAACAAGAAGTTCAACATCC |
| | | TCATCTGCTACCGGCTGGACAGAATCTCTCGCAACGTGGCTGATTTCAGCAATAC |
| | | CATCGAGGAGCTGCAGAAATACAACATCGACTTTATATCCATCAAGGAGCAGTTC |
| | | GATACCAGCACCCCAATGGGCAGAGCCATGATGAACATCGCTGCTGTGTTCGCCC |
| | | AGCTGGAGCGGGAAACCATCGCCGAGCGGATCAAGGACAACATGGTGGAACTGG |
| | | CCAAGACCGGACGGTGGCTGGGCGGCACCTCTCCTCTGGGCTACAAGTCCGAAC |
| | | CCATCGAGTACTCCAATGAGGACGGCAAGTCCAAGAAGATGTACAAGCTGACCG |
| | | AGGTTGAGAACGAGATGAACATCGTGAAGCTGATCTACAAGCTGTACCTGGAGA |
| | | AGAGAGGCTTTAGCTCTGTCGCCACCTACCTGTGCAAGAACAAGTACAAAGGCA |
| | | AGAACGGCGGCGAGTTCTCCAGAGAGACAGCTAGGCAAATCGTGATCAATCCTG |
| | | TGTACTGTATCTCCGACAAGACAATCTTCAAGTGGTTCAAATCCAAGGGCGCTAC |
| | | CACCTACGGCACACCTGACGGAATTCACGGCCTGATGGTGTACAACAAGCGGGA |
| | | AGGCGGAAAGAAGGACAAGCCTATCAACGAGTGGATCATCGCCGTGGGCAAGCA |
| | | TAGAGGAGTCATCTCCTCTGATATCTGGCTGAAGTGCCAAAATCTGATCCAGCAG |
| | | AACAACGCTAAGTCCTCCCCTAGATCCGGTACTGGAGAGAAGTTTCTGCTGTCCG |
| | | GCATGGTGGTGTGTAAGGAGTGCGGCTCCGGCATGAGCTCCTGGAGCCACTTCAA |
| | | CAAAAAAACCAACTTCATGGAAAGATACTACAGATGCAACCTGCGGAATAGAGC |
| | | CTCCAACCGGTGTTCCACCAAGATGCTGAATGCCTACAAGGCCGAGGAATACGT |
| | | GGCCAACTACCTCAAGGAACTAGATATCAACGCCATTAAAAAGATGTACCACTCT |
| | | AACAAGAAGAACATCATCGACTATGACGCCAAGTATGAGGTGAACAAGCTGAAC |
| | | AAGAGCATCGAGGAGAACAAGAAGATCATCCAGGGCATCATCAAGAAGATCGCT |
| | | CTGTTCGACGACCTGGATATCCTGGGCATGCTGAAGAACGAACTGGAGAGACTG |
| | | AAAAAAGAAAACGACGAGATGAAGATCAAACTGAAAGAACTGAAGTCCATCCT |
| | | GGAATTGGAGGATGAAGAGGAGATCTTCCTGTCTACCATGGAGGAGAACATCTC |
| | | TAACTTCAAAAAGTTCTACGACTTCGTGAACATCACCCAGAAGCGGATTCTGATC |
| | | AAGGGCCTGGTGGAAAGTATCGTGTGGGACACAGGCGGTGAGGAAAAGATCCTG |
| | | GAGATCAACCTGATCGGCTCTAACACCAAGCTGCCTTCCGGCAAGGTGAAGCGA |
| | | AGAGAG |
| 64 | | MIAAIYSRKSKFTGKGESVENQIEMCKEYLKRNFNNIDDIEIYEDEGFSGKDTNRPKF |
| | | KKMIKAAKNKKFNILICYRLDRISRNVADFSNTIEELQKYNIDFISIKEQFDTSTPMGR |
| | | AMMNIAAVFAQLERETIAERIKDNMVELAKTGRWLGGTSPLGYKSEPIEYSNEDGKS |
| | | KKMYKLTEVENEMNIVKLIYKLYLEKRGFSSVATYLCKNKYKGKNGGEFSRETARQ |
| | | IVINPVYCISDKTIFKWFKSKGATTYGTPDGIHGLMVYNKREGGKKDKPINEWIIAVG |
| | | KHRGVISSDIWLKCQNLIQQNNAKSSPRSGTGEKFLLSGMVVCKECGSGMSSWSHFN |
| | | KKTNFMERYYRCNLRNRASNRCSTKMLNAYKAEEYVANYLKELDINAIKKMYHSN |
| | | KKNIIDYDAKYEVNKLNKSIEENKKIIQGIIKKIALFDDLDILGMLKNELERLKKENDE |
| | | MKIKLKELKSILELEDEEEIFLSTMEENISNFKKFYDFVNITQKRILIKGLVESIVWDTG |
| | | GEEKILEINLIGSNTKLPSGKVKRRE |
| 27 | Int27 | ATGTCCAAAAAGGTGGCCATCTATACAAGAGTGTCCACCACCAACCAGGCCGAG |
| | | GAAGGCTACTCCATCGACGAGCAGATCGACAAGCTGAAAATGTACTGCGAGGCC |
| | | ATGGACTGGAAGGTGTCTGAGATCTACACCGACGCCGGCTTCACTGGCTCCAAGC |
| | | TGACCAGACCTGCCATGGAAAAGATGATCACCGACATCGGCCTGAAGAAGTTCG |
| | | ATACCGTGATCGTGTACAAGCTGGACAGACTGTCCAGGTCCGTGCGGGATACCCT |
| | | GTACCTGGTCAAGGATGTGTTCACCAAGAATGAGATCGACTTTATCAGCCTGTCT |
| | | GAGTCTATTGACACCTCCTCCGCTATGGGTTCTCTGTTCCTGACAATCCTGAGCGC |
| | | TATCAACGAGTTCGAGAGGGAGAACATAAAAGAACGGATGACCATGGGCAAGAT |
| | | CGGCAGAGCCAAGTCTGGAAAGTCCATGATGTGGGCTAAGACCGCCTTCGGCTA |
| | | CTCTCACAACCAAGAGACAGGCATCCTGGAAATCAACCCTCTGGAAGCTTCCATC |
| | | GTGGAACAGATCTTCAACGAGTACCTGAAGGGCACCTCTATCACAAAGCTGCGG |
| | | GACAAGCTGAACGAGGATGGCCACATCGCCAAGGAGCTGCCTTGGTCCTACAGA |
| | | ACCATCAGACAGACCCTGGACAACCCCGTGTACTGTGGATACATCAAGTACAAA |
| | | AACAACACCTTTGAGGGCCTGCACAAGCCCATCATCTCCCACGAAACCTACCTCA |
| | | GCGTGCAGAAAGAACTGGAAGCCAGACAACAGCAGACCTATGAGAAGAACAAT |
| | | AATCCTAGACCATTTCAAGCCAAGTATCTGCTGTCTGGCATCGCTAGATGCGGAT |
| | | ACTGTGGCGCTCCTCTCCGGATCGTGCTGGGCCATCGCCGGAAGGACGGCAGTAG |
| | | AACCATGAAGTACCAGTGCGTGAACAGATTCCCTCGCAAAACCAAGGGCGTGAC |
| | | CACATACAACGATAACAAGAAGTGCGACTCCGGCGCTTACGACATGCAGTGGAT |
| | | CGAGGACATCGTGCTGAAAACCCTGAACGGCTTCCAGAAGTCCGACAAAAAGCT |
| | | GCGGAAGATCCTGAATATCAAGGAAGAGTCCAAGGTGGACACCAGCGGATTTCA |
| | | GAAGCAGCTGAAGTCCATCAACAATAAGATCCAGAAGAACTCCGATCTGTACCT |
| | | CAACGACTTCATCACCATGGACGACCTGAAAAAGCGGACCGAGATGCTGCAGGG |
| | | CGAGAAGAAACTGATCCAGGCCAGAATCAACGAAGTGGATAAGCCTTCCACATC |
| | | TGAGATCTTCGACCTGGTCAAGTCTGAGCTGGGCGAAACCACCATCTCTAAGATC |
| | | TCCTACGAAGATAAGAAGAAGATCGTCAACAACCTGATCTCTAAAGTTGACGTG |
| | | ACCGCCGACAACATCGATATCATCTTCAAGTTCCAGCTGGCT |
| 65 | | MSKKVAIYTRVSTTNQAEEGYSIDEQIDKLKMYCEAMDWKVSEIYTDAGFTGSKLT |
| | | RPAMEKMITDIGLKKFDTVIVYKLDRLSRSVRDTLYLVKDVFTKNEIDFISLSESIDTS |
| | | SAMGSLFLTILSAINEFERENIKERMTMGKIGRAKSGKSMMWAKTAFGYSHNQETGI |
| | | LEINPLEASIVEQIFNEYLKGTSITKLRDKLNEDGHIAKELPWSYRTIRQTLDNPVYCG |
| | | YIKYKNNTFEGLHKPIISHETYLSVQKELEARQQQTYEKNNNPRPFQAKYLLSGIARC |
| | | GYCGAPLRIVLGHRRKDGSRTMKYQCVNRFPRKTKGVTTYNDNKKCDSGAYDMQ |
| | | WIEDIVLKTLNGFQKSDKKLRKILNIKEESKVDTSGFQKQLKSINNKIQKNSDLYLND |
| | | FITMDDLKKRTEMLQGEKKLIQARINEVDKPSTSEIFDLVKSELGETTISKISYEDKKKI |
| | | VNNLISKVDVTADNIDIIFKFQLA |
| 28 | Int28 | ATGAACGAGCAAAAGGACAAGCTGAAGAAATACTGCGAGATTAAGGACTGGAC |
| | | CATCGTCAAAGAGTACGTCGATCCTGGCCGGAGCGGCTCCAACATCAACAGACC |
| | | ATCCATGCAGCAGCTCATTAAGGACGCCGATACCGGCCTGTACGACGCTGTGCTG |
| | | GTGTACAAGCTGGACCGGCTGTCTAGATCTCAGAAGGACACCCTATATCTGATCG |
| | | AGGACGTGTTCCAGAAGAACAACATCCACTTCATCTCTCTGTCCGAGAACTTCGA |
| | | CACCTCCACCGCCTTTGGAAAGGCCATGATCGGCATCCTCTCCGTGTTCGCCCAG |
| | | CTGGAAAGAGAGCAGATCAAAGAGCGGATGTCTATGGGCAGAGTGGGCAGAGC |
| | | CAAATCCGGCAAAATCATGGAATTCAACAACCCCGCCTTTGGTTACGAGGTGGAT |
| | | GGCGACAACTACAAAGTGGACCCACTGCGGGCCGAGATCGTGAAGAGAATCTAC |
| | | AAGATGTACCTGAGCGGCACCTCTATCAACAAGATCAAGGAAACCCTGAACCTG |
| | | GAAGGCCACATCGGCAACAAGAAGAACTGGTCCGACACCAGAATCAGATATATC |
| | | CTGTCCAATCCCACCTACCTGGGAAAGATCCGGTACGACGGCAAAACCTACGAC |
| | | GGCAAGTTCTCCCCTATCATCGACGAGGAAACCTTCAACAAGACCCAGAACGAA |
| | | CTGAAAGAGAGACAGACCGCTACATACAAGAGATTCAACATGAAGCTACGCCCC |
| | | TTTCAGTCTAAGTACATGCTGTCTGGCCTGCTGAGGTGCGGCTACTGCGGCGCTA |
| | | CCCTGTTCGTGAACTCCTATGTGTACAACGGCAAGCGGAAGCTGCGATACAACTG |
| | | TCCTTCTACCTACAAGTCCAAGCAAAAAACACGGACATACAAGATCATGGACCC |
| | | CAACTGCCCTTTCAAGCTGGTGTACGCCAAGGATCTGGAACCTGCTGTGATCAAC |
| | | GAGATCAAGAATCTGGCTCTGAACCCTCAGTCCATCCAGAAGCCTGTGAAGAAG |
| | | AAACCTGATATCGATGTGGAAGCCATCCAGAAAGAGCTGGCCAAGGTGCGGAAG |
| | | CAGCAGCAGAGACTGATCGATCTGTACGTGATCAGCGACGACGTGAATATCGAC |
| | | AATATCAGCAAGAAGTCTGCCGACCTGAAGCTGCAAGAGGAGACACTGAAGAAG |
| | | CAGCTGGCTCCTCTGGAGGAGCCTAACGACGACGATAAGATCGTGGCCTTCAATG |
| | | AGATTCTGGCTCAGATCAAGGATATCGACTCCCTGGACTACGATAAGCAGAAGTT |
| | | CATCGTCAAGAAGCTGATCAAGAAAATCGACGTGTGGAACGACAACAAGATCAA |
| | | GATCCATTGGAACATC |
| 66 | | MNEQKDKLKKYCEIKDWTIVKEYVDPGRSGSNINRPSMQQLIKDADTGLYDAVLVY |
| | | KLDRLSRSQKDTLYLIEDVFQKNNIHFISLSENFDTSTAFGKAMIGILSVFAQLEREQIK |
| | | ERMSMGRVGRAKSGKIMEFNNPAFGYEVDGDNYKVDPLRAEIVKRIYKMYLSGTSI |
| | | NKIKETLNLEGHIGNKKNWSDTRIRYILSNPTYLGKIRYDGKTYDGKFSPIIDEETENK |
| | | TQNELKERQTATYKRFNMKLRPFQSKYMLSGLLRCGYCGATLFVNSYVYNGKRKL |
| | | RYNCPSTYKSKQKTRTYKIMDPNCPFKLVYAKDLEPAVINEIKNLALNPQSIQKPVKK |
| | | KPDIDVEAIQKELAKVRKQQQRLIDLYVISDDVNIDNISKKSADLKLQEETLKKQLAP |
| | | LEEPNDDDKIVAFNEILAQIKDIDSLDYDKQKFIVKKLIKKIDVWNDNKIKIHWNI |
| 29 | Int29 | ATGAAAACCGCCATCTACCTGAGAAAGTCTAGAGCCGATCTGGAGGCCGAGGCT |
| | | AGAGGCGAGGGCGAGACACTGGCCAAGCACCGGTCGACACTCCTGAAGATCGCC |
| | | AAGGAGATGAACCTGAACGTGCTGTCTGTGAGAGAAGAAATCGTGTCCGGCGAG |
| | | TCTCTGGTCAAGCGGCCCGAGATGCTGGCTCTGCTGGAAGAGATCGAGGACAAC |
| | | AAGTACGACGCCGTGCTGTGCATGGATATGGACAGACTGGGAAGGGGCGGCATG |
| | | AAGGAACAGGGAATCATCCTGGAAACCTTCAAGCGGTCCAACACCAAGATCATG |
| | | ACCCCTAGAAAGACCTACGACCTGAACGACGAGTGGGACGAGGAGTACTCTGAG |
| | | TTTGAGGCCTTCATGGCCAGAAAAGAACTTAAGATCATCACCAGAAGAATGCAG |
| | | AGAGGCCGGATCGCCAGCGTGGAAGCTGGCAACTATCTCGGCACCCACGCTCCA |
| | | TTCGGCTATGATATCCACCGGCTGAACAAAAGAGAGAGAACCCTGACAATCAAC |
| | | TCCGAGGAGGCCTCCGTGGTGCGGATGATCTTCGACTGGTACGCCAACGAGGAC |
| | | ATGGGCGCCAGTGCTATCCGGAACAAGCTGAACGACTTGGGCTACAAGTCCAAG |
| | | CTGGGCAATGACTGGAACCCCTACTCCATCCTGGATATCCTGAAGAACAACATCT |
| | | ACATCGGCAAAGTCACCTGGCAGAAACGTAAGGAAGTGAAGCGGCCTGATGCTG |
| | | TCAAGAGATCCTGTGCCAGACAGGACAAGTCCGATTGGATCATCGCTGACGGCA |
| | | AGCACGAGCCTATCATCCCTGAGTCCCTGTTCGAGCAGGCCCAAGAGAAGCTGA |
| | | ATTCTCGGTACCACGTGCCATACAATACCAACGGCATTAAGAACCCTCTGGCTGG |
| | | AATCATTAAGTGTAGCAAGTGCGGCTACTCCATGGTGCAGAGATACCCTAAGAAT |
| | | CGGAAGGAAACCATGGACTGCAAGCATAGAGGCTGCGAGAACAAGTCTAGCTAC |
| | | ACCGAGCTGATCGAGAAGCGCCTCCTGGAAGCTCTGAAGGAATGGTACATCAAC |
| | | TACAAGGCTGACTTTGAAGCTCACAAGCAGGGCGACAAGCTGAAGGAGACACAA |
| | | GTGATCCAGATGAACGAGGCTGCCCTGCGGAAGCTGGAAAAAGAACTGGTGGAC |
| | | GTGCAGAAGCAGAAGAACAACCTGCACGACCTGCTGGAGCGGGGCGTGTACACC |
| | | GTGGACATGTTCCTGGAAAGATCTCAGGTGATCTCCGACCGGATCAACGAGATCA |
| | | CCTCTACCATGGAAAACCTGAAAAAGGAGATCAAGACCGAAATCAAGAAGGAG |
| | | AAAGTGAAGAAGGACACCATCCCCCAGGTGGAGCATGTGCTGGACCTGTACTTC |
| | | AAGACTGACGATCCTAAGAAAAAGAATTCTCTGCTGAAGTCCGTGCTGGAAAAG |
| | | GCCGTGTACAAGAAAGAAAAATGGCAGAGACTGGACGACTTCGAGCTGGTTCTG |
| | | TACCCTAAGCTGCCTCAGGATGGAGACATC |
| 67 | | MKTAIYLRKSRADLEAEARGEGETLAKHRSTLLKIAKEMNLNVLSVREEIVSGESLV |
| | | KRPEMLALLEEIEDNKYDAVLCMDMDRLGRGGMKEQGIILETFKRSNTKIMTPRKTY |
| | | DLNDEWDEEYSEFEAFMARKELKIITRRMQRGRIASVEAGNYLGTHAPFGYDIHRLN |
| | | KRERTLTINSEEASVVRMIFDWYANEDMGASAIRNKLNDLGYKSKLGNDWNPYSIL |
| | | DILKNNIYIGKVTWQKRKEVKRPDAVKRSCARQDKSDWIIADGKHEPIIPESLFEQAQ |
| | | EKLNSRYHVPYNTNGIKNPLAGIIKCSKCGYSMVQRYPKNRKETMDCKHRGCENKS |
| | | SYTELIEKRLLEALKEWYINYKADFEAHKQGDKLKETQVIQMNEAALRKLEKELVD |
| | | VQKQKNNLHDLLERGVYTVDMFLERSQVISDRINEITSTMENLKKEIKTEIKKEKVKK |
| | | DTIPQVEHVLDLYFKTDDPKKKNSLLKSVLEKAVYKKEKWQRLDDFELVLYPKLPQ |
| | | DGDI |
| 30 | Int30 | ATGTACCGGCCAGAGAGCCTGGACGTGTGCATCTATCTGCGCAAGTCTCGGAAA |
| | | GATGTGGAAGAAGAACGGCGGGCTATTGAAGAGGGCTCCTCCTACAACGCCCTG |
| | | GAAAGACACAGAAAGAGACTGTTCGCCATCGCTAAGGCCGAGAACCACAACATC |
| | | ATCGACATCTTCGAGGAAGTGGCCTCTGGGGAGTCTATCCAAGAGCGGCCTCAG |
| | | ATGCAGCAGCTGCTGCGGAAGTTGGAAGGCAACGAGATTGACGGAGTGCTGGTC |
| | | ATCGATCTGGATAGGCTGGGCAGAGGCGATATGCTGGACGCTGGCATGATTGAC |
| | | AGAGCCTTCAGATACTCCTCTACCAAGATCATCACCCCTACCGACGTGTACGACC |
| | | CCGACGACGAGTCCTGGGAGCTCGTGTTCGGCATCAAGAGCCTGATCTCCAGACA |
| | | AGAACTGAAGTCCATCACCAAGAGGCTGCAGAACGGCCGGATCGATTCCGTGAA |
| | | AGAAGGCAAGCACATCGGTAAGAAACCACCTTACGGCTACCTGAAGGATGAGAA |
| | | CCTGAGACTGTACCCTGATCCTGAGAAAGCTTGGATCGTGAAGAAGATCTTCGAG |
| | | CTGATGTGCGACGGCAAAGGCAGACAGATGATCGCCGCTGAGCTGGACAGACTG |
| | | GGCATCGACCCTCCTGTGACCAAGCGGGGCGCCTGGGACTCTTCTACAATTACCT |
| | | CTATCATCAAGAACGAGGTGTACACCGGCGTGATCGTGTGGGGAAAGTTCAAGC |
| | | ACAAGAAGCGGAACGGCAAGTACACCAGACATAAGAATCCTCAAGAGAAGTGG |
| | | ATCATGTACGAGAACGCTCACGAGCCTATCATCTCTAAGGAACTGTTCGACGCCG |
| | | CCAACGAGGCCCATTCTTCGAGACACAAGCCCGCCGTGATCACTTCCAAGAAACT |
| | | GACCAACCCCCTGGCCGGCATCCTGAAGTGCAAGCTGTGTGGCTACACCATGCTG |
| | | ATCCAGACCCGGAAGGACCGGCCTCACAACTACCTGAGATGCAACAACCCCGCC |
| | | TGTAAAGGCAAGCAGAAGCAGTCTGTGTTCAACCTGGTTGAGGAAAAGCTCCTG |
| | | TATAGCCTGCAGCAGATCGTGGACGAGTACCAGGCTCAGAAGGTGGAAGAAGTG |
| | | GAGATCGACGATTCCAAGCTGATCTCCTTCAAGGAGAAGGCTATCATCTCCAAGG |
| | | AGAAGGAACTCAAAGAACTGCAGGCCCAGAAGGGCAACCTGCACGACCTGCTGG |
| | | AACAGGGCATCTACACAGTCGAGATCTTTCTGGAAAGACAGAAGAATCTGGTCG |
| | | AAAGAATCACCTCCATCGAGAACGACATCGAGGTGCTGCAGAAGGAGATCGAGA |
| | | CAGAGCAGATCAAGGAGCACAACAAGACCGAGTTTATCCCTGCTCTGAAAACAG |
| | | TGATCGAGAGCTACCATAAGACCACCAATATCGAGCTGAAGAATCAGCTGCTGA |
| | | AAACCATCCTGTCCACCGTGACCTACTACAGACACCCTGACTGGAAAACCAACG |
| | | AGTTCGAAATCCAGGTGTACTTTAAAATC |
| 68 | | MYRPESLDVCIYLRKSRKDVEEERRAIEEGSSYNALERHRKRLFAIAKAENHNIIDIFE |
| | | EVASGESIQERPQMQQLLRKLEGNEIDGVLVIDLDRLGRGDMLDAGMIDRAFRYSST |
| | | KIITPTDVYDPDDESWELVFGIKSLISRQELKSITKRLQNGRIDSVKEGKHIGKKPPYG |
| | | YLKDENLRLYPDPEKAWIVKKIFELMCDGKGRQMIAAELDRLGIDPPVTKRGAWDS |
| | | STITSIIKNEVYTGVIVWGKFKHKKRNGKYTRHKNPQEKWIMYENAHEPIISKELFDA |
| | | ANEAHSSRHKPAVITSKKLTNPLAGILKCKLCGYTMLIQTRKDRPHNYLRCNNPACK |
| | | GKQKQSVFNLVEEKLLYSLQQIVDEYQAQKVEEVEIDDSKLISFKEKAIISKEKELKEL |
| | | QAQKGNLHDLLEQGIYTVEIFLERQKNLVERITSIENDIEVLQKEIETEQIKEHNKTEFI |
| | | PALKTVIESYHKTTNIELKNQLLKTILSTVTYYRHPDWKTNEFEIQVYFKI |
| 31 | Int31 | ATGAAGTACCTGGCTCTGCATGAGAACTCCCGGATCGCCGTGTACAGCCGGAAGT |
| | | CCAGAGAGGACAGAGACTCCGAGGATACCCTGGCCAAGCACCGGAACGAGCTGG |
| | | AATACCTGATCAAGAGAGAAAACTTCAAAAACGTGCAGTGGTTCGAGAAGGTGG |
| | | TGTCCGGCGAAACCATCGACGAGCGGCCTATGTTCTCCCTGCTGCTGCCTAGAAT |
| | | TGAAAACGGCGAGTTCGACGCTGTGTGTGCCGTGGCCATGGACCGGCTGTCTAGA |
| | | GGCTCCCAGATCGATTCTGGAAGAATCCTGGAGGCCTTTAAGCAGTCCGGCACCC |
| | | TGTTCATCACCCCTAAGAAAACCTACGACCTGTCCATCGAGGGCGACGAGATGCT |
| | | GTCCGAGTTCGAATCCATCATCGCCAGATCTGAGTACAGAGCTATCAAGCGGAG |
| | | AACCATCAACGGCAAGAAGAATGCCACCCGCGAAGGCCGGCTGCACAGCGGATC |
| | | CGTGCCTTATGGTTACAAGTGGGACAAGAACCTGAAAGCTGCTGTCGTGGTGGA |
| | | AGAGAAGAAGAAGATCTATCGGATGATGATTAAGTGGTTTCTGGAAGAAGAGTA |
| | | CTCCTGCACCGTGATCGCTGAGATGCTGAATGAACTCAAGGTGCCCTCCCCCTCA |
| | | GGCAGATCTATCTGGTACGGCGAGGTGGTGTCTGAGATCCTGTCCAACGACTTCC |
| | | ACAGAGGATACGTGTGGTTCGGCAAGTATAAGAAGTCCAAGAGCAACAACAGCA |
| | | TCGTGCAGAACAAGAACCTGGATGAGGTTCTCATCGCCAAGGGCCACCATGAAA |
| | | CCATGAAAACCGATGAGGAGCACGCCCTGATCCTGAACCGGATCGAGAAGCTGC |
| | | GGACCTACAAGGTGGCTGGCAGACGGCTGAACATGAACACCCATAGACTGTCCG |
| | | GCATCGTGCGGTGTCCTTACTGCCACAAGGCTCAGGCCATCGAGCAACCAAAGG |
| | | GCAGACGGAAGCACGTGAGAAAGTGCCTGAGAAAGTCCGCTGAGAGGACCAAA |
| | | GAGTGCGAGGAAACAAAAGGCATCCACGAGGAAGTGCTGTTTCAGTCTATCATG |
| | | AAAGAGATCAAGAAATACAATGAGTCTCTGTTCTCTCCTACCGAGCAGGACGTG |
| | | AACGACGACTCCTACACTGCCCAGCTGATCGGCCTGAGGGAGAAGGCCGTGAAG |
| | | AAGGCTAAGGGCCGCATCGAGCGGATCAAAGAGATGTACCTGGACGGAGACATC |
| | | TCCAAAACCGAGTACAAGGAAAAGCTGAAGATCAGCCAAGAGACACTGCAGAA |
| | | GGCTGAGAACGAACTTGCCGAACTGATAGCCTCTACAGAGTTCCAGAACGCCCT |
| | | GTCTGCCGAGACAAAGAAAGAGAAGTGGTCCCACCACAAGGTGCAGGAAATGAT |
| | | CGAGAGCACCGACGGCATGTCCAACTCTGAAATCAACTTGATCCTGAAGATGCTG |
| | | ATCTCTCACGTGACCTACACCGTCGAAGATCTGGGCGATGGCACCAAGAATCTGA |
| | | ACATCAAGGTGTACTACAAC |
| 69 | | MKYLALHENSRIAVYSRKSREDRDSEDTLAKHRNELEYLIKRENFKNVQWFEKVVS |
| | | GETIDERPMFSLLLPRIENGEFDAVCAVAMDRLSRGSQIDSGRILEAFKQSGTLFITPK |
| | | KTYDLSIEGDEMLSEFESIIARSEYRAIKRRTINGKKNATREGRLHSGSVPYGYKWDK |
| | | NLKAAVVVEEKKKIYRMMIKWFLEEEYSCTVIAEMLNELKVPSPSGRSIWYGEVVSE |
| | | ILSNDFHRGYVWFGKYKKSKSNNSIVQNKNLDEVLIAKGHHETMKTDEEHALILNRI |
| | | EKLRTYKVAGRRLNMNTHRLSGIVRCPYCHKAQAIEQPKGRRKHVRKCLRKSAERT |
| | | KECEETKGIHEEVLFQSIMKEIKKYNESLFSPTEQDVNDDSYTAQLIGLREKAVKKAK |
| | | GRIERIKEMYLDGDISKTEYKEKLKISQETLQKAENELAELIASTEFQNALSAETKKEK |
| | | WSHHKVQEMIESTDGMSNSEINLILKMLISHVTYTVEDLGDGTKNLNIKVYYN |
| 32 | Int32 | ATGGACCCTCAGCACAAGCCTACCCGGGCTCTGATCGTGATCCGGCTGTCCCGGC |
| | | TGACAGACGAAACCACCTCTCCTGAGCGGCAGCTGGAGGCCTGCGAGAGATTCT |
| | | GCGCCGCAAGAGGCTGGGAGGTCGTGGGCGTGGCTGAAGATCTGGACGTGTCTG |
| | | CTGGAACCACCAGCCCCTTCGAGCGGCCTTCTCTGAGCCAGTGGATCGGCGATGG |
| | | TAAGGACAACCCAGGAAGAATCGGCGAGTTCGACACCGTGGTTTTCTACAGAGT |
| | | GGATCGGCTCGTGCGGAGAGTGCGGCACCTGCACGACGTGATCGCCTGGAGCGA |
| | | GCGCTTCGATGTGAACATGGTGTCCGCCACCGAGTCTCACTTCGACCTGTCCACA |
| | | ACCATTGGCGCTCTGATCGCTCAGCTGGTGGCCTCCTTCGCCGAGATGGAACTGG |
| | | AGGGCATCTCTCAGAGAGCTACCTCTGCTCACAGACACAACGTGCAGCTGGGCA |
| | | AGTTCGTGGGCGGCTCCCCTCCTTTCGGCTACATGCCTGAAGAAACCCCTGATGG |
| | | CTGGCGGCTGGTGCACGATCCCGACGTCGTGCCCATCATCCTCGAGGTGGTGGAC |
| | | AGAGTCCTGGAAGGCGAACCCCTGAGAAGAATCACCGACGATCTGAACGCCCGG |
| | | GGCGCTACAACCGCCCGGGACCTGGTGAAGCAGAGAAAGGGCAAAGAAACCGA |
| | | GGGCCACAAGTGGCACTCCAACGTGCTGAAGCGGCGGCTGATGTCCCCTGCCAT |
| | | GCTGGGCTACGCCCTGAGAAGAGAACCTCTGACCGACTCCAAGGGCAAGCCCAA |
| | | ACTGTCTGCCAAGGGCGCCAAGCTGTACGGCCCTGAGGAAATCGTGAGAGGACC |
| | | TGACGGCCTGCCTGTGCAACGCGCTGAGCCTATCCTGCCTAAGCCTCTGTTCGAC |
| | | CGGGTGGTGGCTGAGCTAGAAGCTAGAGAGCTACAGAAAGAGCCTACCAAGCGG |
| | | ATCAACTCCATGCTGTTGAGAGTGCTGTACTGCGGCGTGTGTGGCCAGCCTGTCT |
| | | ACCGGGCAAAAGGACAGGGCGGTAGATCCGACAGATACCGGTGCAGATCCATCC |
| | | AGGATGGCGCCAACTGTGGCAACCCCTCCGTGCTGACCTATGAGCTGGACGACCT |
| | | GGTGGAAGAGTCTATCCTGGTGCTGATGGGCGACTCTGAGAGACTGGCCCATGTG |
| | | TGGAACCCTGGCGAGGACAATGCTAGCGAGCTGGCTGAAGTGGAAGCCCGGCTG |
| | | GCCGACAGAACCGGCCTGATCGGAGTGGGAGCCTACAAGGCTGGCACCCCCCAG |
| | | AGAGCCACCCTGGATACCCTGATCGAGGCTGATGCCAAGCTGTACGAGAGGCTG |
| | | AAGGCCGCCACCCCTAGACCTGCTGGCTGGACCTGGGAACCAACAGGCGAAACC |
| | | TTCGCCGAGTGGTGGGCTGCTCTGGACACCGGCGCCAGAAATGTGTACCTGCGGA |
| | | ACATGGGGGTCAGAGTCACCTACGACAAGCGGCCTGTGCCAGAGCAGGTGTCCG |
| | | CCGGCGAGAAGCCTAGAGTGCATCTGGAACTGGGCGAAGTGCGGAAGATGGCCG |
| | | AACAAGTGGCTGTGACCGGCACCATCGGAACACTGACCAGAAACTACACAAGAC |
| | | TGGGAGAGATCGGCATCACCCACGTGGACATCGACGCCGGATCTGGCAAGGCCG |
| | | TGTTTGTGACAAAGTCCGGCGAGCGGTTCGAGCTCCCTCTGAACATCCCTGAGGA |
| | | A |
| 70 | | MDPQHKPTRALIVIRLSRLTDETTSPERQLEACERFCAARGWEVVGVAEDLDVSAGT |
| | | TSPFERPSLSQWIGDGKDNPGRIGEFDTVVFYRVDRLVRRVRHLHDVIAWSERFDVN |
| | | MVSATESHFDLSTTIGALIAQLVASFAEMELEGISQRATSAHRHNVQLGKFVGGSPPF |
| | | GYMPEETPDGWRLVHDPDVVPIILEVVDRVLEGEPLRRITDDLNARGATTARDLVKQ |
| | | RKGKETEGHKWHSNVLKRRLMSPAMLGYALRREPLTDSKGKPKLSAKGAKLYGPE |
| | | EIVRGPDGLPVQRAEPILPKPLFDRVVAELEARELQKEPTKRINSMLLRVLYCGVCGQ |
| | | PVYRAKGQGGRSDRYRCRSIQDGANCGNPSVLTYELDDLVEESILVLMGDSERLAH |
| | | VWNPGEDNASELAEVEARLADRTGLIGVGAYKAGTPQRATLDTLIEADAKLYERLK |
| | | AATPRPAGWTWEPTGETFAEWWAALDTGARNVYLRNMGVRVTYDKRPVPEQVSA |
| | | GEKPRVHLELGEVRKMAEQVAVIGTIGTLTRNYTRLGEIGITHVDIDAGSGKAVFVT |
| | | KSGERFELPLNIPEE |
| 33 | Int33 | ATGAAGGCTATCGCCATCTACGCCAGAAAGTCTCTGTTCACCGGCAAGGGCGACT |
| | | CCATTGGCGCCCAGGTGGACACCTGCAAGCGGTTCATCGACTACAAGTTCGCCAA |
| | | TGAGGACTATGAGATCCGGACATTTAAGGACGAAGGCTGGTCCGGCAAGACCAC |
| | | TGACAGACCAGATTTTACCAACATGGTGAACCTGATCAAGTCTAAGAAGATCGA |
| | | CTATGTCATCACCTACAAGCTGGACCGGATCGGCCGGACAGCTCGGGACCTGCAC |
| | | AACTTCCTGTACGAGCTGGATAATCTGGGAATCGTGTACCTGAGCGCCACCGAGC |
| | | CTTACGACACAACCACATCTGCCGGAAGATTCATGATCAGCATTCTGGCTGCTAT |
| | | GGCTCAGATGGAACGCGAAAGACTGGCCGAGAGAGTGAAGTCCGGCATGATCCA |
| | | GATCGCCAAGAAGGGAAGATGGCTGGGCGGCCAGTGTCCTCTGGGCTTCGACTC |
| | | TAAGAGAGAGATCTACATCGATGACATGGGGAAAGAGCGGCAGATGATGCGGCT |
| | | GACCCCTAACAAGGAGGAAATCAAGATCGTGAAGCTGATCTACGACAAGTACCT |
| | | GGAGATGGGATCCATGTCCCAAGTGCGGAAGTACTGCCTGGAAAACTCCATCAG |
| | | AGGCAAGAACGGCGGCGACTTCTCCACAAACACCCTGAAGCAGCTGCTGACCTC |
| | | TCCTATCTACGTCAAGTCCTCCGACAACATCTTCAAGTACCTGGAGTCTCAGAAT |
| | | ATCAATGTGTTCGGCACCCCCAACGGCAACGGCATGCTGACCTTCAACAAGACCA |
| | | AAGAGATCAGGATCGAGCGGGACAAGTCCGAGTGGATTGCTGCTGTGGGCAAGC |
| | | ACAAGGGCATCATCGACGATAACAAGTGGCTGCAGATCCAGCAGCAGCTGCAGC |
| | | AGCAGTCTGAAAAGCAGATCAAGAGCTCTGGCAGACAGGGCACGACCTCCACCG |
| | | GCCTGCTGTCCGGCATCATCAAGTGCTCCAAGTGCGGCAACAACCTGCTGATCAA |
| | | GACCGGACACAAGTCCAAGAAAAACCCTGGCACCACCTACTCCTACTACGTGTGT |
| | | GGCAAGAAGGATAACTCTTACGGCCATAAGTGCGACAACAAAAACGTGAGAACC |
| | | GACGAGGCCGACTCCGCCGTGATCACCCAGCTGAAACTGTACAACAAAGAACTG |
| | | CTCATCAAAAATCTCAAGGAAGCCCTGATCCAAAACGAAAAGACCGATACCGAC |
| | | AACATCGAGATCCTGGAGTCCAAATTAAAAGAAAAAGAGAAGGCCGTGTCCAAC |
| | | CTGGTGAAAAAGCTGTCTCTGATCGACGACGAGTCCATCAGCAATATCATCCTGA |
| | | ACGAGGTTACCAATATCAACAAGGAAATCAACGACATCAAGCTGCAATTGTCTA |
| | | ACGAGACACTGAAGATCAACGAAGTGACCAAGGCCACACTGGATACCGAGATCT |
| | | ACATCAAGATCCTGGAGAACTTTAACAAGAAGATCGACGATATCACCGACCCCA |
| | | TCGAAAAGATGAACTTGCTGAAGTCTGCTCTGGAATCCGTGGAATGGAACGGCG |
| | | ATTCTGGCGAGTTCAAGATCAACCTGATCGGCAGCAAAAAGAAA |
| 71 | | MKAIAIYARKSLFTGKGDSIGAQVDTCKRFIDYKFANEDYEIRTFKDEGWSGKTTDR |
| | | PDFTNMVNLIKSKKIDYVITYKLDRIGRTARDLHNFLYELDNLGIVYLSATEPYDTTT |
| | | SAGRFMISILAAMAQMERERLAERVKSGMIQIAKKGRWLGGQCPLGFDSKREIYIDD |
| | | MGKERQMMRLTPNKEEIKIVKLIYDKYLEMGSMSQVRKYCLENSIRGKNGGDFSTN |
| | | TLKQLLTSPIYVKSSDNIFKYLESQNINVFGTPNGNGMLTENKTKEIRIERDKSEWIAA |
| | | VGKHKGIIDDNKWLQIQQQLQQQSEKQIKSSGRQGTTSTGLLSGIIKCSKCGNNLLIK |
| | | TGHKSKKNPGTTYSYYVCGKKDNSYGHKCDNKNVRTDEADSAVITQLKLYNKELLI |
| | | KNLKEALIQNEKTDTDNIEILESKLKEKEKAVSNLVKKLSLIDDESISNIILNEVTNINK |
| | | EINDIKLQLSNETLKINEVTKATLDTEIYIKILENFNKKIDDITDPIEKMNLLKSALESVE |
| | | WNGDSGEFKINLIGSKKK |
| 34 | Int34 | ATGAAGGTTGCTATCTACACCAGAGTGTCCACCCTGGAGCAGCGGGAAAAGGGA |
| | | CACTCTATCGACGAGCAAGAGCGGAAACTGAGATCTTTCTGCGACATTAACGACT |
| | | GGACCGTGAAAGATGTGTACGTGGATGCTGGCTTCTCCGGAGCCAAGCGGGACA |
| | | GACCTGAGCTGACCAGACTCCTGGACGACATCTCCGAGTTCGACCTGGTGCTGGT |
| | | CTACAAGCTGGACCGGCTGACAAGAAGCGTCAGAGATCTGCTGGACCTGCTGGA |
| | | AGTGTTCGAGAACAATAACGTGGCCTTCAGATCTGCTACCGAGGTGTACGACACC |
| | | ACCACCGCCATCGGCAGACTGTTCGTGACACTCGTGGGCGCCATGGCCGAGTGG |
| | | GAGAGAGAGACAATCCGGGAAAGAAGCCTGATGGGCAAGAGAGCCGCTATTAA |
| | | GAAGGGCATGATCCTGACCGCTCCACCCTTCTACTACGACAGGGTGAACAACACC |
| | | TACATCCCTAACCAGTATAAGGATGTGGTCCTCGATGTGTACAACAAGGTCAAGA |
| | | AAGGCTACTCCATCGCTCATATCGCCAGACTGTACAACAACTCCGACGTGAAGCC |
| | | TCCTAACGGCAACGAGGAATGGACCACCCGGATGCTGATGCACGCCCTGAGAAA |
| | | CCCTGTGACCCGGGGCCACTACCAGTGGGGCGAGATCTACATCGAGGACTCTCAT |
| | | GAGCCTATCATCACAGACGAGATGTACAATACAATCATCGACCGCCTGGACAAG |
| | | CACACCAACACCAAGGTGGTGGCCCACACCTCCGTGTTTCGGGGCAAGCTGATCT |
| | | GCCCCAACTGTGGCTACGCTCTGACCCTGAACAGCCAGAAGAGAAAGCGGAAGA |
| | | ACGACACCATCGTGTACAAGACCTATTACTGCAATAACTGCAAGATCACCAAGG |
| | | GCATGAAGCCTCACCACATCACCGAGACAGAAACCCTGCGGGTGTTCAAGGACC |
| | | ACCTGTCCAAGATCGACCTGAAACAGTACGAAACCCAAGAGAAAGAGAAGCAGT |
| | | CTCACGTGACCATCGATCTGTCTAAAGTGATGGAACAGAGAAAGAGATACCACA |
| | | AGCTGTACGCCTCTGGCATGATGCAGGAAAACGAGCTGTTTGAACTGATCAAGG |
| | | AAACCGACGAAATGATCGAAGAGTACGAGAAGCAGCGCAAGCAGGTGGACGTG |
| | | AAAGAGTTCGACATCTGTAAGATCAAAGAAATCAAGGATGTGCTGCTGAAGTCC |
| | | TGGGACATCTTCACCCTGGAAGATAAGGCCGACTTCATCCAGATGTCCATCAAGG |
| | | CTATCAACATCGAGTATACCAAGCTGAAGCGGGGAAAGAGCTCTAATTCCATGA |
| | | AGATCAAGGATATCGAGTTTTAC |
| 72 | | MKVAIYTRVSTLEQREKGHSIDEQERKLRSFCDINDWTVKDVYVDAGFSGAKRDRP |
| | | ELTRLLDDISEFDLVLVYKLDRLTRSVRDLLDLLEVFENNNVAFRSATEVYDTTTAIG |
| | | RLFVTLVGAMAEWERETIRERSLMGKRAAIKKGMILTAPPFYYDRVNNTYIPNQYKD |
| | | VVLDVYNKVKKGYSIAHIARLYNNSDVKPPNGNEEWTTRMLMHALRNPVTRGHYQ |
| | | WGEIYIEDSHEPIITDEMYNTIIDRLDKHTNTKVVAHTSVFRGKLICPNCGYALTLNSQ |
| | | KRKRKNDTIVYKTYYCNNCKITKGMKPHHITETETLRVFKDHLSKIDLKQYETQEKE |
| | | KQSHVTIDLSKVMEQRKRYHKLYASGMMQENELFELIKETDEMIEEYEKQRKQVDV |
| | | KEFDICKIKEIKDVLLKSWDIFTLEDKADFIQMSIKAINIEYTKLKRGKSSNSMKIKDIE |
| | | FY |
| 35 | Cre | ATGTCCAATCTGCTGACCGTGCACCAGAACCTGCCTGCTCTGCCCGTGGACGCCA |
| | | CCAGCGACGAGGTGCGCAAGAACCTGATGGACATGTTCCGCGACCGCCAGGCCT |
| | | TCAGCGAGCACACCTGGAAGATGCTGCTGAGCGTGTGCCGCAGCTGGGCCGCCT |
| | | GGTGCAAGCTGAACAACCGCAAGTGGTTCCCCGCCGAGCCCGAGGACGTGCGCG |
| | | ACTACCTGCTGTACCTGCAGGCCCGCGGCCTGGCCGTGAAAACCATCCAGCAGCA |
| | | CCTGGGCCAGCTGAACATGCTGCACCGCCGCAGCGGCCTGcctAGGCCATCTGACT |
| | | CTAATGCCGTGTCTCTGGTCATGCGGCGGATCCGGAAAGAAAACGTGGACGCCG |
| | | GCGAGAGAGCTAAGCAGGCTCTGGCTTTCGAGAGAACCGACTTCGACCAAGTGC |
| | | GGTCCCTGATGGAAAACTCCGACCGGTGCCAGGATATCCGGAACCTGGCTTTTCT |
| | | GGGAATCGCCTACAACACCCTGCTGCGGATCGCTGAGATCGCCCGGATCAGAGT |
| | | GAAGGACATCTCTAGAACCGACGGCGGCAGAATGCTGATCCACATCGGCAGAAC |
| | | AAAGACCCTGGTGTCCACAGCTGGCGTGGAAAAGGCTCTGTCTCTGGGCGTGACC |
| | | AAGCTGGTGGAACGGTGGATTTCTGTGTCCGGCGTGGCCGACGATCCCAACAACT |
| | | ACCTGTTCTGCAGAGTCCGGAAGAACGGCGTGGCAGCCCCTTCTGCTACATCCCA |
| | | GCTGTCTACAAGAGCCCTGGAAGGCATCTTCGAGGCTACCCACAGACTGATCTAC |
| | | GGCGCCAAGGACGATAGCGGCCAGAGATATTTGGCTTGGAGCGGCCACTCCGCT |
| | | AGAGTGGGAGCTGCTAGAGATATGGCTAGAGCCGGCGTGTCCATTCCTGAGATC |
| | | ATGCAAGCTGGCGGCTGGACCAACGTGAACATCGTGATGAACTACATCCGCAAC |
| | | CTGGACTCCGAGACAGGCGCTATGGTTCGACTGCTGGAAGATGGCGAC |
| 73 | | MSNLLTVHQNLPALPVDATSDEVRKNLMDMFRDRQAFSEHTWKMLLSVCRSWAA |
| | | WCKLNNRKWFPAEPEDVRDYLLYLQARGLAVKTIQQHLGQLNMLHRRSGLPRPSDS |
| | | NAVSLVMRRIRKENVDAGERAKQALAFERTDFDQVRSLMENSDRCQDIRNLAFLGI |
| | | AYNTLLRIAEIARIRVKDISRTDGGRMLIHIGRTKTLVSTAGVEKALSLGVTKLVERWI |
| | | SVSGVADDPNNYLFCRVRKNGVAAPSATSQLSTRALEGIFEATHRLIYGAKDDSGQR |
| | | YLAWSGHSARVGAARDMARAGVSIPEIMQAGGWTNVNIVMNYIRNLDSETGAMVR |
| | | LLEDGD |
| 36 | nls- | ATGGCCCCAAAGAAGAAGCGGAAAGTGATGTCTCAGTTCGACATCCTGTGCAAG |
| | flpE | ACCCCTCCTAAGGTTCTGGTCAGACAGTTCGTGGAACGGTTCGAGCGGCCTTCTG |
| | | GCGAAAAGATCGCTTCCTGTGCCGCTGAGCTGACCTACCTGTGCTGGATGATCAC |
| | | CCACAACGGCACCGCCATCAAACGGGCCACCTTCATGTCCTACAACACCATCATC |
| | | TCCAACTCCCTGTCCTTCGACATCGTGAACAAGTCTCTGCAGTTCAAGTACAAGA |
| | | CACAGAAGGCTACCATCCTGGAAGCTTCCCTCAAGAAGCTGATCCCTGCTTGGGA |
| | | GTTCACAATCATCCCCTATAATGGCCAAAAGCACCAGTCTGATATTACAGATATC |
| | | GTGTCCAGCCTGCAGCTGCAGTTTGAGTCCTCCGAGGAGGCCGACAAGGGCAAT |
| | | AGTCACTCCAAGAAGATGCTGAAAGCCCTGCTGTCTGAAGGCGAGTCCATCTGG |
| | | GAAATCACCGAGAAGATCCTGAACTCCTTCGAGTACACCAGCAGATTCACCAAA |
| | | ACCAAGACCCTGTACCAGTTCCTCTTCCTGGCTACCTTCATCAACTGCGGCAGATT |
| | | CAGCGATATCAAGAATGTGGACCCCAAGAGCTTCAAGCTGGTGCAGAACAAGTA |
| | | CCTGGGCGTGATCATCCAGTGCCTGGTGACCGAAACCAAGACCTCTGTGAGCAG |
| | | GCACATCTACTTCTTTTCTGCTAGAGGCAGAATCGACCCACTGGTGTACCTGGAC |
| | | GAGTTCCTGAGAAACTCCGAGCCTGTGCTGAAGAGAGTGAACAGAACCGGCAAC |
| | | TCTTCTTCCAACAAGCAGGAGTATCAACTGCTGAAAGACAACCTGGTGCGGTCCT |
| | | ACAACAAGGCTCTGAAGAAAAATGCTCCTTACCCTATCTTCGCCATCAAGAACGG |
| | | ACCTAAGAGCCATATCGGCAGACACCTGATGACCTCCTTTCTGTCCATGAAAGGC |
| | | CTGACAGAACTGACCAACGTGGTGGGCAACTGGTCCGACAAGAGAGCCTCCGCC |
| | | GTGGCCCGGACCACCTACACTCATCAGATCACAGCCATCCCTGATCACTACTTTG |
| | | CCCTGGTGTCCAGATACTACGCCTACGACCCTATCTCCAAAGAGATGATCGCCTT |
| | | GAAGGACGAAACCAACCCCATCGAGGAATGGCAGCACATCGAGCAGCTGAAGG |
| | | GATCTGCGGAGGGCTCCATCCGGTACCCCGCTTGGAACGGCATCATCAGCCAAG |
| | | AGGTGCTGGACTACCTGTCCTCTTACATCAACCGCCGGATC |
| 74 | | MAPKKKRKVMSQFDILCKTPPKVLVRQFVERFERPSGEKIASCAAELTYLCWMITHN |
| | | GTAIKRATFMSYNTIISNSLSFDIVNKSLQFKYKTQKATILEASLKKLIPAWEFTIIPYN |
| | | GQKHQSDITDIVSSLQLQFESSEEADKGNSHSKKMLKALLSEGESIWEITEKILNSFEY |
| | | TSRFTKTKTLYQFLFLATFINCGRFSDIKNVDPKSFKLVQNKYLGVIIQCLVTETKTSV |
| | | SRHIYFFSARGRIDPLVYLDEFLRNSEPVLKRVNRTGNSSSNKQEYQLLKDNLVRSYN |
| | | KALKKNAPYPIFAIKNGPKSHIGRHLMTSFLSMKGLTELTNVVGNWSDKRASAVART |
| | | TYTHQITAIPDHYFALVSRYYAYDPISKEMIALKDETNPIEEWQHIEQLKGSAEGSIRY |
| | | PAWNGIISQEVLDYLSSYINRRI |
| 37 | Bxb1 | ATGAGAGCCCTGGTGGTCATCCGGCTGTCTAGAGTGACCGACGCTACCACCTCTC |
| | var.- | CTGAGCGGCAGCTGGAATCTTGCCAGCAGCTGTGTGCTCAGCGCGGATGGGATGT |
| | NLS | TGTGGGAGTCGCTGAGGACCTGGATGTGTCTGGTGCCGTGGATCCTTTCGACCGG |
| | | AAGCGGAGGCCTAACCTGGCTAGATGGCTGGCCTTTGAGGAACAGCCCTTCGAC |
| | | GTGATCGTGGCCTACAGAGTGGACCGGCTGACCCGGTCTATCAGACATCTGCAGC |
| | | AGCTGGTCCACTGGGCCGAAGATCACAAGAAACTGGTGGTGTCCGCCACCGAGG |
| | | CTCACTTCGATACCACCACACCTTTTGCCGCCGTCGTGATCGCTCTGATGGGAAC |
| | | CGTTGCTCAGATGGAACTGGAAGCCATCAAAGAGCGGAACAGATCCGCCGCTCA |
| | | CTTCAACATCAGAGCCGGCAAGTACCGGGGCTCTTTGCCTCCTTGGGGCTACCTG |
| | | CCAACAAGAGTGGATGGCGAATGGCGGCTGGTGCCTGATCCTGTGCAGCGGGAA |
| | | AGAATCCTGGAAGTGTACCACAGAGTGGTGGACAACCACGAGCCTCTGCACCTG |
| | | GTGGCCCACGACTTGAATAGAAGAGGCGTGCTGTCCCCTAAGGACTACTTCGCCC |
| | | AGCTGCAGGGCAGAGAGCCTCAGGGAAGAGAGTGGAGCGCTACCGCTCTGAAGC |
| | | GGTCCATGATCTCTGAGGCCATGCTGGGCTACGCTACCCTGAATGGAAAGACCGT |
| | | GCGGGACGATGATGGCGCCCCTCTTGTTAGAGCCGAGCCTATCCTGACCAGAGA |
| | | GCAGCTCGAAGCCCTGAGAGCTGAGCTGGTCAAGACCTCCAGAGCCAAGCCTGC |
| | | TGTGTCTACCCCTAGCCTGCTGCTGAGAGTGCTGTTCTGTGCTGTGTGTGGCGAGC |
| | | CCGCCTACAAGTTTGCTGGCGGCGGAAGAAAGCACCCCAGATACCGGTGTCGGT |
| | | CCATGGGCTTCCCTAAGCACTGTGGCAATGGCACCGTGGCCATGGCTGAGTGGGA |
| | | TGCCTTCTGCGAAGAACAGGTGCTGGATCTGCTGGGCGACGCCGAGAGACTGGA |
| | | AAAAGTGTGGGTGGCCGGCTCCGACTCTGCTGTGGAACTGGCTGAAGTGAACGC |
| | | CGAGCTGGTGGACCTGACCTCTCTGATCGGCTCTCCCGCTTATAGAGCTGGCTCC |
| | | CCTCAGAGAGAAGCCCTGGACGCTAGAATCGCTGCCCTGGCTGCTAGACAAGAG |
| | | GAACTCGAAGGCCTGGAAGCTCGGCCTTCAGGATGGGAGTGGCGAGAGACAGGC |
| | | CAGAGATTTGGCGACTGGTGGCGCGAGCAAGATACCGCCGCTAAGAACACCTGG |
| | | CTGCGGTCTATGAATGTGCGGCTGACCTTCGATGTGCGCGGAGGACTGACCAGAA |
| | | CCATCGACTTCGGCGACCTGCAAGAGTACGAGCAGCATCTGAGACTGGGCTCCGT |
| | | GGTGGAAAGACTGCACACCGGCATGTCCggttcaCCAAAGAAAAAGCGGAAAGTG |
| 75 | | MRALVVIRLSRVTDATTSPERQLESCQQLCAQRGWDVVGVAEDLDVSGAVDPFDRK |
| | | RRPNLARWLAFEEQPFDVIVAYRVDRLTRSIRHLQQLVHWAEDHKKLVVSATEAHF |
| | | DTTTPFAAVVIALMGTVAQMELEAIKERNRSAAHFNIRAGKYRGSLPPWGYLPTRVD |
| | | GEWRLVPDPVQRERILEVYHRVVDNHEPLHLVAHDLNRRGVLSPKDYFAQLQGREP |
| | | QGREWSATALKRSMISEAMLGYATLNGKTVRDDDGAPLVRAEPILTREQLEALRAE |
| | | LVKTSRAKPAVSTPSLLLRVLFCAVCGEPAYKFAGGGRKHPRYRCRSMGFPKHCGN |
| | | GTVAMAEWDAFCEEQVLDLLGDAERLEKVWVAGSDSAVELAEVNAELVDLTSLIG |
| | | SPAYRAGSPQREALDARIAALAARQEELEGLEARPSGWEWRETGQRFGDWWREQD |
| | | TAAKNTWLRSMNVRLTFDVRGGLTRTIDFGDLQEYEQHLRLGSVVERLHTGMSGSP |
| | | KKKRKV |
| 38 | Bxb1 | ATGAGAGCACTGGTGGTCATCCGACTGAGTAGGGTCACAGACGCAACAACAAGC |
| | var., | CCCGAGAGGCAGCTGGAATCATGTCAGCAGCTGTGCGCACAGCGAGGATGGGAC |
| | (no | GTGGTCGGAGTGGCAGAGGATCTGGACGTGAGCGGCGCTGTCGATCCATTCGAC |
| | NLS) | AGAAAGCGGAGGCCCAACCTGGCAAGGTGGCTGGCTTTCGAGGAACAGCCCTTT |
| | | GATGTGATCGTCGCCTACAGAGTGGACAGGCTGACACGCTCTATTCGACATCTGC |
| | | AGCAGCTGGTGCATTGGGCCGAGGACCACAAGAAACTGGTGGTCAGTGCAACTG |
| | | AAGCCCACTTCGATACCACAACTCCTTTTGCCGCTGTGGTCATCGCACTGATGGG |
| | | CACCGTGGCCCAGATGGAGCTGGAAGCTATCAAGGAGCGAAACCGGAGTGCAGC |
| | | CCATTTCAATATTCGGGCCGGGAAATACAGAGGATCACTGCCCCCTTGGGGCTAT |
| | | CTGCCTACCCGGGTGGATGGGGAGTGGAGACTGGTGCCAGACCCCGTCCAGAGA |
| | | GAGAGGATTCTGGAAGTGTACCACAGGGTGGTCGATAACCACGAACCACTGCAT |
| | | CTGGTCGCCCACGACCTGAATAGGCGCGGCGTGCTGAGCCCAAAAGATTATTTTG |
| | | CTCAGCTGCAGGGAAGGGAGCCACAGGGACGAGAATGGTCCGCTACCGCCCTGA |
| | | AGCGGAGCATGATCAGTGAGGCTATGCTGGGCTACGCAACTCTGAATGGGAAAA |
| | | CCGTCCGGGACGATGACGGAGCACCACTGGTGAGGGCTGAGCCTATTCTGACAC |
| | | GCGAGCAGCTGGAAGCTCTGCGGGCAGAACTGGTGAAAACCTCCAGAGCCAAAC |
| | | CTGCCGTGAGCACCCCAAGCCTGCTGCTGAGGGTGCTGTTCTGCGCCGTCTGTGG |
| | | GGAGCCAGCATACAAGTTTGCCGGCGGGGGAAGAAAACATCCCCGCTATCGATG |
| | | CCGGTCTATGGGATTCCCTAAGCACTGTGGAAACGGCACTGTGGCTATGGCCGAG |
| | | TGGGACGCCTTTTGTGAGGAACAGGTGCTGGATCTGCTGGGAGACGCCGAGAGG |
| | | CTGGAAAAAGTGTGGGTCGCTGGCAGCGACTCCGCTGTGGAGCTGGCAGAAGTC |
| | | AATGCCGAGCTGGTGGATCTGACCTCCCTGATCGGATCTCCTGCATATAGGGCAG |
| | | GCTCACCACAGCGAGAAGCTCTGGACGCACGAATTGCTGCACTGGCAGCTCGAC |
| | | AGGAGGAACTGGAGGGGCTGGAAGCACGACCTAGCGGATGGGAGTGGCGAGAA |
| | | ACAGGCCAGCGGTTTGGGGATTGGTGGAGAGAGCAGGACACAGCAGCCAAGAA |
| | | CACTTGGCTGAGAAGTATGAATGTCAGGCTGACTTTCGATGTGCGCGGCGGGCTG |
| | | ACCCGAACAATCGATTTTGGCGACCTGCAGGAGTATGAACAGCACCTGAGACTG |
| | | GGGAGCGTGGTCGAAAGACTGCACACTGGGATGTCA |
| 76 | | MRALVVIRLSRVTDATTSPERQLESCQQLCAQRGWDVVGVAEDLDVSGAVDPFDRK |
| | | RRPNLARWLAFEEQPFDVIVAYRVDRLTRSIRHLQQLVHWAEDHKKLVVSATEAHF |
| | | DTTTPFAAVVIALMGTVAQMELEAIKERNRSAAHFNIRAGKYRGSLPPWGYLPTRVD |
| | | GEWRLVPDPVQRERILEVYHRVVDNHEPLHLVAHDLNRRGVLSPKDYFAQLQGREP |
| | | QGREWSATALKRSMISEAMLGYATLNGKTVRDDDGAPLVRAEPILTREQLEALRAE |
| | | LVKTSRAKPAVSTPSLLLRVLFCAVCGEPAYKFAGGGRKHPRYRCRSMGFPKHCGN |
| | | GTVAMAEWDAFCEEQVLDLLGDAERLEKVWVAGSDSAVELAEVNAELVDLTSLIG |
| | | SPAYRAGSPQREALDARIAALAARQEELEGLEARPSGWEWRETGQRFGDWWREQD |
| | | TAAKNTWLRSMNVRLTFDVRGGLTRTIDFGDLQEYEQHLRLGSVVERLHTGMS |

TABLE 2
Table of accession numbers, source organism or known phage, and att
recombination sites for each integrase tested.

| | Old | New | | | | | |
|---|---|---|---|---|---|---|---|
| | NCBI | NCBI | NCBI | | | | |
| Name | AA | AA | Nucleotide | Organism | Phage | attB | attP |
| Int1 | YP_ | WP_ | NC_ | Rhodobacter | | ggaactccgccgggc | atggggtcacaatac |
| | 353073 | 023003660 | 007493.2: | Sphaeroides | | ccatctggtcgaaga | caatcatgttcaaga |
| | | | c1706259- | 2.4.1 | | agatgaaggggccca | atgtgaagggtattt |
| | | | 1704511 | | | ccatctgcctccggg | tacccttgtcgtttc |
| | | | | | | cc | ag |
| | | | | | | (SEQ ID NO: 79) | (SEQ ID NO: 80) |
| Int2 | CBG734 | CBG73463 | NC_ | Streptomyces | | ggacggcgcagaagg | gctcatgtatgtgtc |
| | 63 | | 013929.1: | scabiei 87.22 | | ggagtagctcttcgc | tacgcgagattctcg |
| | | | 7156189- | | | cggaccgtcgacata | cccgagaacttctgc |
| | | | 7157718 | | | ctgctcagctcgtc | aaggcactgctcttg |
| | | | | | | (SEQ ID NO: 81) | gct |
| | | | | | | | (SEQ ID NO: 82) |
| Int3 | NP_ | WP_ | NC_ | Streptococcus | Phi370.1 | gtttgtaaaggagac | atggataaaaaaata |
| | 2688 | 010922052 | 002737.2: | pyogenes M1 | | tgataatggcatgta | cagcgtttttcatgt |
| | 97 | | c531042- | GAS | | caactatactcgtcg | acaactatactagtt |
| | | | 529627 | | | gtaaaaaggcatctt | gtagtgcctaaataa |
| | | | | | | at | tgctt |
| | | | | | | (SEQ ID NO: 83) | (SEQ ID NO: 84) |
| Int4 | YP_ | WP_ | NC_ | Streptococcus | | ttccaaagagcgccc | caaaaattacaaagt |
| | 002747001 | 012679988 | 012471.1: | equi subsp. equi | | aacgcgacctgaaat | tttcaacccttgatt |
| | | | 1771390- | 4047 | | ttgaataagactgct | tgaattagcggtcaa |
| | | | 1772823 | | | gcttgtgtaaaggcg | ataatttgtaattcg |
| | | | | | | atgatt | ttt |
| | | | | | | (SEQ ID NO: 85) | (SEQ ID NO: 86) |
| Int5 | BAF035 | BAF03598 | AB251919.1: | Streptomyces | PhiK38 | gagcgccggatcagg | ccctaatacgcaagt |
| | 98 | | 505- | phage PhiK38-1 | | gagtggacggcctgg | cgataactctcctgg |
| | | | 2163 | | | gagcgctacacgctg | gagcgttgacaactt |
| | | | | | | tggctgcggtcggtg | gcgcaccctgatctg |
| | | | | | | c | |
| | | | | | | (SEQ ID NO: 87) | (SEQ ID NO: 88) |
| Int6 | BAG464 | BAG46462 | AP009386.1: | Burkholderia | | gatacggatgttcgt | agttgtctgataata |
| | 62 | | 1620691- | multivorans | | cgccggcacgctggt | tattttcggacacgc |
| | | | 1622250 | ATCC 17616 | | cacgctcggcaatcc | tcggcaacccgaacg |
| | | | | | | caagatcatgctgtt | agagtcaaaatacat |
| | | | | | | ct | tt |
| | | | | | | (SEQ ID NO: 89) | (SEQ ID NO: 90) |
| Int7 | YP_ | 24454WP_ | NC_ | Geobacillus sp. | | agacgagaaacgttc | gtgttataaacctgt |
| | 003251752 | 0135 | 013411.1: | Y412MC61 | | cgtccgtctgggtca | gtgagagttaagttt |
| | | | c601516- | | | gttgggcaaagttga | acatgcctaacctta |
| | | | 600128 | | | tgaccgggtcgtccg | acttttacgcaggtt |
| | | | | | | tt | cagctt |
| | | | | | | (SEQ ID NO: 91) | (SEQ ID NO: 92) |
| Int8 | BAE05705 | BAE05705 | AP006716.1: | Staphylococcus | | caatcatcagataac | ttaataaactatgga |
| | | | 2394908- | haemolyticus | | tatggcggcacgtgc | agtatgtacagtctt |
| | | | 2396293 | JCSC1435 | | attaaccacggttgt | gcaatgttgagtgaa |
| | | | | | | atcccgtctaaagta | caaacttccataata |
| | | | | | | ctcgt | aaat |
| | | | | | | (SEQ ID NO: 93) | (SEQ ID NO: 94) |
| Int9 | BAF67264 | BAF67264 | AP009351.1: | Staphylococcus | | tttatattgcgaaaa | gtggttgtttttgtt |
| | | | c1100283- | aureus str. | | ataattggcgaacga | ggaagtgtgtatcag |
| | | | 1098898 | Newman | | ggtaactggatacct | gtatctgcatagtta |
| | | | | | | catccgccaattaaa | ttccgaacttccaat |
| | | | | | | atttg | ta |
| | | | | | | (SEQ ID NO: 95) | (SEQ ID NO: 96) |
| Int10 | YP_ | WP_ | NC_ | Streptococcus | | agcacgctgataatc | ggaaaatataaataa |
| | 003880342 | 000633509 | 014498.1: | pneumoniae | | agcaagaccaccaac | ttttagtaacctaca |
| | | | 2029057- | 670-6B | | atttccaccaatgta | tctcaatcaaggata |
| | | | 2030502 | | | aaagctttaacctta | gtaaaactctcactc |
| | | | | | | gc | tt |
| | | | | | | (SEQ ID NO: 97) | (SEQ ID NO: 98) |
| Int11 | YP_ | WP_ | NC_ | Clostridium | | atggattttgcagat | gtttatatgtttact |
| | 001886479 | 012423712 | 010674.1: | botulinum B str. | | tcccagatgccccta | aataagacgctctca |
| | | | 2361091- | Eklund 17B | | cagaaagaggtacaa | acccataaagtctta |
| | | | 2362434 | | | aacatttattggaat | ttagtaaacatattt |
| | | | | | | taatt | caact |
| | | | | | | (SEQ ID NO: 99) | (SEQ ID NO: 100) |
| Int12 | YP_ | WP_ | NC_ | Staphylococcus | | gttcgtggtaactat | tttttgtatgttagt |
| | 005759947 | 014533238 | 017353.1: | lugdunensis | | gggtggtacaggtgc | tgtgtcactgggtag |
| | | | c888963- | N920143 | | cacattagttgtacc | acctaaatagtgaca |
| | | | 887581 | | | atttatgtttatgtg | caactgctattaaaa |
| | | | | | | gttaac | tttaa |
| | | | | | | (SEQ ID NO: 101) | (SEQ ID NO: 102) |
| Int13 | YP_ | WP_ | NC_ | Bacillus | | cgcatacattgttgt | caataacggttgtat |
| | 001376196 | 012095429 | 009674.1: | cytotoxicus | | tgtttttccagatcc | ttgtagaacttgacc |
| | | | 3019953- | NVH 391-98 | | agttggtcctgtaaa | agttgttttagtaac |
| | | | 3021377 | | | tataagcaatccatg | ataaatacaactccg |
| | | | | | | tgagt | aata |
| | | | | | | (SEQ ID NO: 103) | (SEQ ID NO: 104) |
| Int14 | NP_ | WP_ | NC_ | Listeria | | ttattgcaagaaaaa | ttatataaaatagtg |
| | 470568 | 010990844 | 003212.1: | innocua | | tgggttataagtaca | tttttgtaaagtaca |
| | | | c1247978- | Clip11262 | | catcaggttatagta | catcaccatatttga |
| | | | 1246563\| | | | atatcgaaaaaggaa | caaaaaacctataaa |
| | | | | | | gc | ta |
| | | | | | | (SEQ ID NO: 105) | (SEQ ID NO: 106) |
| Int15 | YP_ | WP_ | NC_ | Listeria | A118 | ctgtaactttttcgg | ttgtttagtccctcg |
| | 006685721 | 014930216 | 018588.1: | monocytogenes | | atcaagctatgaggg | ttttctctcgttgga |
| | | | 2418537- | SLCC2372 | | acgcaaagagggaac | cggagacgaatcgag |
| | | | 2419895 | | | taaacacttaattgg | aaactaaaattataa |
| | | | | | | tg | at |
| | | | | | | (SEQ ID NO: 107) | (SEQ ID NO: 108) |
| Int16 | YP_ | WP_ | NC_ | Enterococcus | | ttctggaccatgatg | gtatcttgatgtaca |
| | 006538656 | 010717149 | 018221.1: | faecalis D32 | | cgccacttccgaaat | acattactctttatt |
| | | | 2359751- | | | ttcaaaaagatcagt | ttcaaatacagaata |
| | | | 2361163 | | | ggtcaaacggctcat | atgttgcatataata |
| | | | | | | ta | tt |
| | | | | | | (SEQ ID NO: 109) | (SEQ ID NO: 110) |
| Int17 | YP_ | WP_ | NC_ | Staphylococcus | | acttccaattaaccc | ttatatttcgactta |
| | 189066 | 001260014 | 002976.3: | epidermidis | | ttcaccagccctata | attaagtacagttcc |
| | | | c1569306- | RP62A | | ccaagttcctgtcgc | acctagagatagact |
| | | | 1567768 | | | gcatcctccagctaa | aaataaagtattatt |
| | | | | | | t | a |
| | | | | | | (SEQ ID NO: 111) | (SEQ ID NO: 112) |
| Int18 | YP_ | WP_ | NC_ | Streptococcus | | tctggtgtagacgtt | tatttctgtatttta |
| | 002736920 | 000633503 | 012466.1: | pneumoniae JJA | | aaacgtccaatcaag | gtcaaagtaattaag |
| | | | 1783389- | | | ataactttattatac | ataagttagagttag |
| | | | 1784816 | | | atattttcttcctcc | taacagtattttaac |
| | | | | | | ta | tt |
| | | | | | | (SEQ ID NO: 113) | (SEQ ID NO: 114) |
| Int19 | FM864213 | CAR95427 | FM864213.1: | Streptococcus | PhiM461.1 | gtagatttgtttccc | tattagtatagaaga |
| | | | 49163- | phage phi-m46.1 | | cagacgcacacgtgg | aagctctcagcacac |
| | | | 50551 | | | agtgtgtaagtttac | gtggagtgtgttgct |
| | | | | | | ttgagaaacggagtt | ctctgctcgtaaagc |
| | | | | | | aa | ct |
| | | | | | | (SEQ ID NO: 115) | (SEQ ID NO: 116) |
| Int20 | YP_ | WP_ | NC_ | Streptococcus | | cttccagcacatcac | ggtattgtatcaatt |
| | 006082695 | 014638101 | 017621.1: | suis D12 | | ccacatggtctgtgt | tcagaactcacactt |
| | | | c1170236- | | | cggtgtgcgtcagca | cggtatgcgtactca |
| | | | 1169001 | | | ctagactatcaatcc | attttgatacaatta |
| | | | | | | ta | caa |
| | | | | | | (SEQ ID NO: 117) | (SEQ ID NO: 118) |
| Int21 | YP_ | WP_ | NC_ | Streptococcus | | taggaggaaaaaata | gttaataatatgtat |
| | 003445547 | 001244955 | 013853.1: | mitis B6 | | tgtataataaagtta | ttaagtctaacttat |
| | | | c399646- | | | tcatgattgggcgtt | catgacaaatttgac |
| | | | 398225 | | | tgacgtctacaccag | taaaatacaaaaagg |
| | | | | | | aat | c |
| | | | | | | (SEQ ID NO: 119) | (SEQ ID NO: 120) |
| Int22 | YP_ | WP_ | NC_ | Geobacillus | | caagaaacgttccgt | ttataaacctgtttt |
| | 004586821 | 013876366 | 015660.1: | thermo | | ctgtttgtgtcagct | aaagttaactttaca |
| | | | c741927- | glucosidasius | | gcgcgaaattaatga | tgcctaacattaact |
| | | | 740536 | C56-YS93 | | ccggatcgtttgttc | cttatacaggttaag |
| | | | | | | c | gt |
| | | | | | | (SEQ ID NO: 121) | (SEQ ID NO: 122) |
| Int23 | YP_ | WP_ | NC_ | Clostridium | | tattctaagtaatgt | tatataattatttgg |
| | 001089468 | 011861760 | 009089.1: | difficile 630 | | agttttaccacatcc | actaacatatagtat |
| | | | 3427501- | | | actaggtccgagtaa | ccacttggctattat |
| | | | 3428859 | | | acatagaaattcccc | tagttagtccaaata |
| | | | | | | t | aata |
| | | | | | | (SEQ ID NO: 123) | (SEQ ID NO: 124) |
| Int24 | YP_ | WP_ | NC_ | Clostridium | | actacttaatatatc | gttaggtgtatatca |
| | 005679179 | 014521361 | 017299.1: | botulinum | | cataagagaaatttc | tacctaacgcaattc |
| | | | 2735819- | H04402 065 | | atttccttctttgtc | attacatcacatatg |
| | | | 2737597 | | | tacccctataggatc | ttatacacctacttt |
| | | | | | | tt | aa |
| | | | | | | (SEQ ID NO: 125) | (SEQ ID NO: 126) |
| Int25 | YP_ | WP_ | NC_ | Clostridium | | tattcaattatgtgt | tatatacttatagat |
| | 001384783 | 012099404 | 009697.1: | botulinum A str. | | cgtaatttttatcta | actaaatatttttgt |
| | | | 2591621- | ATCC 19397 | | ttgcgacg | attgcgtaacttctt |
| | | | 2593135 | | | aaaaaacaccataaa | ctacacctgtaatat |
| | | | | | | attctaac | ct |
| | | | | | | (SEQ ID NO: 127) | (SEQ ID NO: 128) |
| Int26 | YP_ | WP_ | NC_ | Clostridium | | agaaatagacctttc | aaatataacctgtgt |
| | 001392519 | 012100936 | 009699.1: | botulinum F str. | | aactggacaaggtgc | attgaaacaaggtgc |
| | | | 3464125- | Langeland | | tgataaaactatgca | tgataaaaccctttc |
| | | | 3465762 | | | gcaagtcttaagtaa | ataaacacaagtaaa |
| | | | | | | a | ta |
| | | | | | | (SEQ ID NO: 129) | (SEQ ID NO: 130) |
| Int27 | YP_ | WP_ | NC_ | Lactococcus | High | aatactaataatagc | cttatctcaattaag |
| | 005869510 | 014570823 | 017486.1: | lactis subsp. | simi- | tagtacaattaacat | gtaactaaacgctta |
| | | | 2179142- | lactis CV56 | larity | ctctatcaaagtaaa | attgcgagtttttat |
| | | | 2180599 | | to | agcttttagctcttt | ttcgaaactcctttt |
| | | | | | TP901-1 | ct | |
| | | | | | | (SEQ ID NO: 131) | (SEQ ID NO: 132) |
| Int28 | YP_ | WP_ | NC_ | Lactobacillus | | aagtgtccaagctgg | tataatttcgtatat |
| | 001271396 | 003668055 | 009513.1: | reuteri DSM | | cccccgatcccagtt | tagatataaccggtt |
| | | | c870480- | 20016 | | tcaatagtttgggga | tcaattggaaatacc |
| | | | 869104 | | | atctttgtaagtggt | taatatacgaaaaaa |
| | | | | | | aa | gg |
| | | | | | | (SEQ ID NO: 133) | (SEQ ID NO: 134) |
| Int29 | YP_ | WP_ | NC_ | Bacillus | | tttgtagccattagg | cgtcaccttgttggc |
| | 001646422 | 012261582 | 010184.1: | weihen | | cgcattaggttgacg | gtaattagatttact |
| | | | 3672347- | stephanensis | | ccattaagccctaaa | ccaacagggtgatga |
| | | | 3673894 | KBAB4 | | gcatcattcgtogaa | caaagctaatgaatt |
| | | | | | | ac | tt |
| | | | | | | (SEQ ID NO: 135) | (SEQ ID NO: 136) |
| Int30 | YP_ | WP_ | NC_ | Bacillus cereus | | gtaatatgtttggat | ataatagtgtatatg |
| | 002336631 | 000286206 | 011658.1: | AH187 | | atggggaagtgaatc | gtagagaattaaacc |
| | | | c587458- | | | agtacaaccgccaca | agtttaatactccac |
| | | | 585908 | | | gtaccctcatgtcag | catgtacacgcagtg |
| | | | | | | cc | ag |
| | | | | | | (SEQ ID NO: 137) | (SEQ ID NO: 138) |
| Int31 | YP_ | WP_ | NC_ | Bacillus | | ttttttccgcctgtc | cttttttgttgtact |
| | 005549228 | 014472506 | 017191.1: | amyloliquefaciens | | gtaaccggatctgtt | taaacaataatgctt |
| | | | c1181305- | XH7 | | gtaacgattatcgga | gtaagaattattgat |
| | | | 1179764 | | | atgaccttgatgccg | tgagtacgacataaa |
| | | | | | | g | cc |
| | | | | | | (SEQ ID NO: 139) | (SEQ ID NO: 140) |
| Int32 | YP_ | WP_ | NC_ | Rhodococcus | | atcgcgcagaacggt | Ictatgtggtggtaat |
| | 706485 | 011598406 | 008268.1: | jostii RHA1 | | gcggtgatcagtgag | agcgagtaggggact |
| | | | 7055865- | | | tacgcaccgggcacg | actcgctccaggtac |
| | | | 7057607 | | | acaccggcgaagcat | attaacaccatgga |
| | | | | | | cg | (SEQ ID NO: 142) |
| | | | | | | (SEQ ID NO: 141) | |
| Int33 | 04732YP_ | WP_ | NC_ | Clostridium | | acgaaataaaagatt | aaaagaatccaaatt |
| | 0028 | 012705666 | 012563.1: | botulinum A2 | | gtatagatgctggta | atcgtactttaacat |
| | | | 2611695- | str. Kyoto | | ggaaacatgcccttg | agtgaatactgtcca |
| | | | 2613317 | | | tcatttagctgaaac | tcatgtataaaagta |
| | | | | | | ag | cg |
| | | | | | | (SEQ ID NO: 143) | (SEQ ID NO: 144) |
| Int34 | YP_ | WP_ | NC_ | Staphylococcus | | aatctgcaaacatgt | atttttgtacggaag |
| | 003472505 | 012991015 | 013893.1: | lugdunensis | | atggcggtacatgta | tagatactatctttc |
| | | | 2348540- | HKU09-01 | | tcaacattggttgta | aatatccatgttact |
| | | | 2349922 | | | ttcctacaaagacac | tagtgccatacaaaa |
| | | | | | | tcat | a |
| | | | | | | (SEQ ID NO: 145) | (SEQ ID NO: 146) |
| Bxb1- | | AAG59740.1 | NC_ | | Bxb1 | tcggccggcttgtcg | gtcgtggtttgtctg |
| GT | | | 002656.1: | | | acgacggcggtctcc | gtcaaccaccgcggt |
| | | | 29491- | | | gtcgtcaggatcatc | ctcagtggtgtacgg |
| | | | 30993 | | | cgggc | tacaaaccccgac |
| | | | | | | (SEQ ID NO: 147) | (SEQ ID NO: 148) |
| Bxb1- | | AAG59740.1 | NC_ | | Bxb1 | tcggccggcttgtcg | gtcgtggtttgtctg |
| GA | | | 002656.1: | | | acgacggcggactcc | gtcaaccaccgcgga |
| | | | 29491- | | | gtcgtcaggatcatc | ctcagtggtgtacgg |
| | | | 30993 | | | cgggc | tacaaaccccgac |
| | | | | | | (SEQ ID NO: 166) | (SEQ ID NO: 167) |
| Cre | | WP_ | | | P1 | NA | NA |
| | | 000067530.1 | | | bacterio- | | |
| | | | | | phage | | |
| Flp | | ADC44104.1 | | Saccharomyces | | NA | NA |
| | | | | cerevisiae | | | |

TABLE 3
Tyrosine recombinase site sequences and literature
sources for recombination sites used in the tyrosine
recombinase landing pads.

| SEQ | | | |
|---|---|---|---|
| ID | | Nucleotide | |
| NO: | Site | Sequence | Source |
| 149 | FRTwt | gaagttccta | Andrews et al. Cell. |
| | | ttcCgaagtt | 1985 April; 40(4): 795-803. |
| | | cctattcTCT | doi: 10.1016/0092-8674(85)90339-3. |
| | | AGAAAgtata | |
| | | ggaacttc | |
| 150 | FRT3 | gaagttccta | Bode. Biochemistry. |
| | | ttcCgaagtt | 1994 Nov. 1; 33(43): 12746-51. |
| | | cctattcTTC | doi: 10.1021/bi00209a003. |
| | | AAATAgtata | |
| | | ggaacttc | |
| 151 | FRT5 | gaagttccta | Schlake and Bode. Biochemistry. |
| | | ttcCgaagtt | 1994 Nov. 1; 33(43): 12746-51. |
| | | cctattcTTC | doi: 10.1021/bi00209a003. |
| | | AAAAGgtata | |
| | | ggaacttc | |
| 152 | FRT14 | gaagttccta | Turan et al. J Mol Biol. |
| | | ttcCgaagtt | 2010 Sep. 10; 402(1): 52-69. |
| | | cctattcTAT | doi: 10.1016/j.jmb.2010.07.015. |
| | | CAGAAgtata | |
| | | ggaacttc | |
| 153 | FRT15 | gaagttccta | Turan et al. J Mol Biol. |
| | | ttcCgaagtt | 2010 Sep. 10; 402(1): 52-69. |
| | | cctattcTTA | doi: 10.1016/j.jmb.2010.07.015. |
| | | TAGGAgtata | |
| | | ggaacttc | |
| 154 | loxP | ATAACTTCGT | Hoess et al. Proc Natl Acad Sci USA. |
| | | ATAatgtatg | 1982 June; 79(11): 3398-402. |
| | | cTATACGAAG | doi: 10.1073/pnas.79.11.3398. |
| | | TTAT | |
| 155 | loxN | ATAACTTCGT | Livet et al. Nature. |
| | | ATAgtatacc | 2007 Nov. 1; 450(7166): 56-62. |
| | | tTATACGAAG | doi: 10.1038/nature06293. |
| | | TTAT | |
| 156 | lox2272 | ATAACTTCGT | Lee and Saito. Gene. |
| | | ATAaagtatc | 1998 Aug. 17; 216(1): 55-65. |
| | | cTATACGAAG | doi: 10.1016/s0378-1119(98)00325-4. |
| | | TTAT | |
| 157 | lox66 | taccGTTCGT | Albert et al. Plant J. |
| | | ATAatgtatg | 1995 April; 7(4): 649-59. |
| | | cTATACGAAG | doi: 10.1046/j.1365-313x.1995.7040649.x. |
| | | TTAT | |
| 158 | lox71 | ATAACTTCGT | Albert et al. Plant J. |
| | | ATAatgtatg | 1995 April; 7(4): 649-59. |
| | | cTATACGAAc | doi: 10.1046/j.1365-313x.1995.7040649.x. |
| | | ggta | |
| 159 | loxKR3 | ATAACTTCGT | Araki et al. BMC Biotechnol. |
| | | ATAatgtatg | 2010 Mar. 31; 10:29. |
| | | cTATACcttG | doi: 10.1186/1472-6750-10-29. |
| | | TTAT | |

TABLE 5
Relative Activity of Int1-Int34

| Integrase | Normalized Reporter Expression |
|---|---|
| Int1 | |
| Int2 | 0.170 |
| Int3 | 1.113 |
| Int4 | 1.852 |
| Int5 | 0.152 |
| Int6 | |
| Int7 | 0.096 |
| Int8 | 0.068 |
| Int9 | 0.080 |
| Int10 | 5.489 |
| Int11 | 1.806 |
| Int12 | 0.821 |
| Int13 | 0.295 |
| Int14 | 0.248 |
| Int15 | 1.859 |
| Int16 | 0.210 |
| Int17 | 0.000 |
| Int18 | 1.758 |
| Int19 | 0.000 |
| Int20 | 0.000 |
| Int21 | 0.184 |
| Int22 | 0.945 |
| Int23 | 0.201 |
| Int24 | 0.000 |
| Int25 | 0.000 |
| Int26 | 0.204 |
| Int27 | 2.201 |
| Int28 | 0.000 |
| Int29 | 2.924 |
| Int30 | 1.292 |
| Int31 | 0.000 |
| Int32 | 0.137 |
| Int33 | 0.001 |
| Int34 | 0.408 |
| Bxb1(GA) | 1.000 |

### Example 2. Landing Pad Architectures

Landing pads can be constructed for the new mammalian integrases determined to function similarly or better than Bxb1. These novel integrases can be used in landing pads designed for site-specific integration of antibodies, stable viral vector payloads, massively parallel reporter assays (MPRAs), characterization of genetic parts, and other applications where specific control of the genetic copy number and locus is desired. Current designs include Bxb1, Cre, and Flp integrase landing pads inserted randomly by lentivirus and random integration, as well as CRISPR mediated insertion at the HEK293 safe harbors AAVS1, ROSA26, CCR5, and LiPS-A3S, as well as the CHO safe harbors ROSA26, COSMIC, and H11.

### Single and Double Site Landing Pads

The first set of landing pads tested were mediated by the Bxb1 serine integrase, then later designed for Cre, and Flp tyrosine integrases using the same architecture (FIG. 4). The landing pads were either inserted randomly into the genome or integrated by lentiviral transduction. These landing pads were tested using the Cre tyrosine recombinase then integrated by low MOI lentiviral transduction for stable integration. As expounded upon below, co-transfection of the Cre recombinase and a payload plasmid mediated either genomic insertion or full RMCE, depending on whether a single lox site or dual lox sites were present in the landing pad and corresponding payload. After 21 days of passaging the co-transfected pools, the final population of cells with stable payload integration was about 2% of the population.

Wells containing 1e6 suspension CHO cells were transduced with a 5-fold dilution series of raw lentivirus containing the Cre single-lox or double-lox landing pads (approximately 500 μL, 125 uL, 31 μL, 8 uL, 2 uL, or 0.5 uL lentivirus transduction in a 6-well plate, for a total volume of 2 mL per well). After 72 hours post-transduction, cells were run on a flow cytometer to calculate undiluted raw virus titer and MOI of each dilution. A transduction of approximately 8 uL was determined to achieve a MOI that did not exceed 0.01 for both the single-lox and double-lox site landing pads viruses. Cells of this dilution were puromycin selected for 20 days until viability fully recovered, by replacing media every 2 to 3 days with fresh media containing 10 μg/mL puromycin.

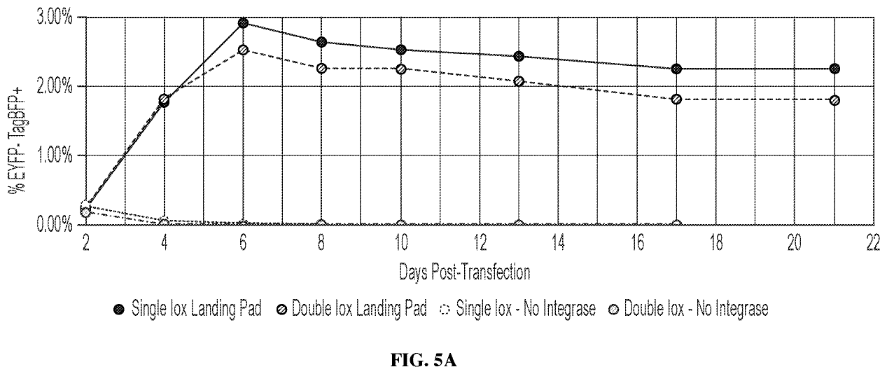

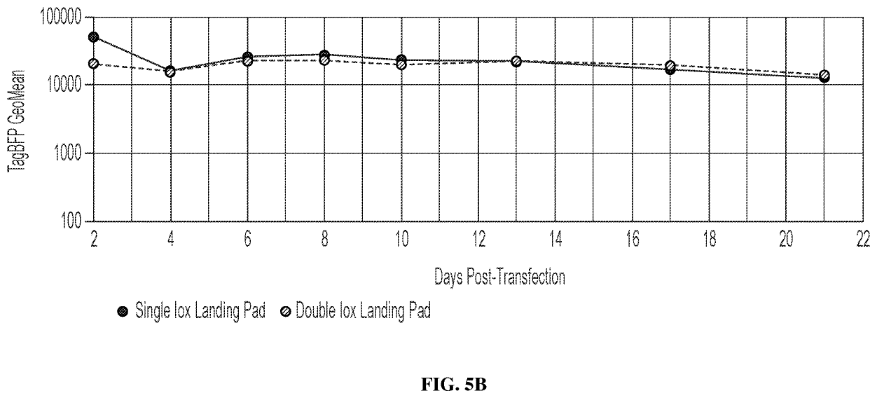

Wells containing 1e6 cells of each Cre landing pad cell line were co-transfected with a 1 ug DNA mixture of the Cre recombinase expression plasmid and a payload plasmid at 1:1 molar ratio (in a 24-well plate, for a total volume of 0.5 mL per well). As a negative control, cells were co-transfected with the payload plasmid and an inert plasmid in place of the Cre recombinase. Starting 48 hours post-transfection, cells were routinely passaged and measured on a flow cytometer for expression of the landing pad fluorescent protein EYFP and the payload fluorescent protein TagBFP (FIGS. 5A-5B). Cell density was maintained between 2e5 to 5e6 viable cells/mL. After 21 days of passaging cells, the population of stably integrated payload was determined to be approximately 2% of the total population, indicated by a loss of landing pad EYFP expression and a gain of payload TagBFP expression (TABLE 4). A subpopulation of cells expressing the payload TagBFP marker also expressed the landing pad EYFP marker, indicating that these cells had multiple copies of the landing pad initially, or that the payload was integrated by random integration. This subpopulation of EYFP and TagBFP positive cells ranged from 3% to 6% of the payload integrated cells (TABLE 4). This subpopulation may primarily be due to multiple copies of the landing pad, since the payload plasmid itself does not have a functional promoter, and any fluorescence observed in random integration would have to be driven by a promoter upstream of the integration site.

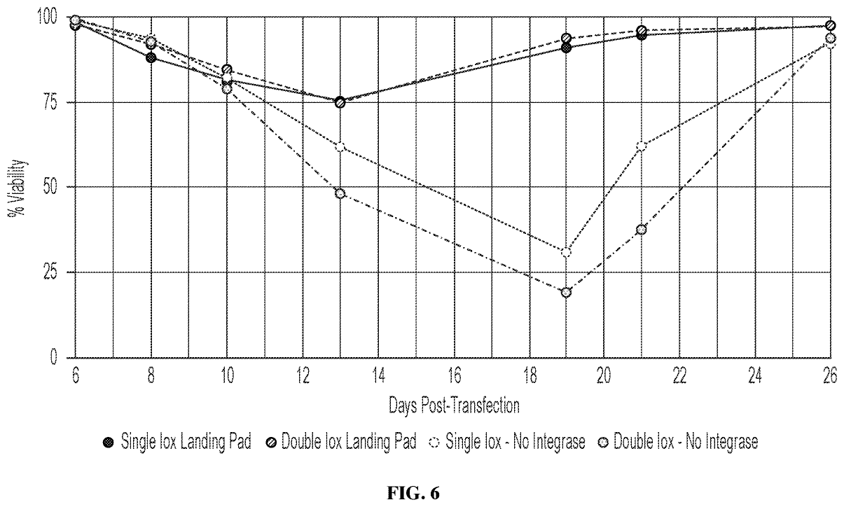

Simultaneously, at day 6 of the co-transfected cells being passaged, a split of the cells was placed under hygromycin selection until cells fully recovered. Antibiotic selection was performed by replacing media every 2 to 3 days with fresh media containing 400 μg/mL hygromycin until day 19 post-transfection, then 500 μg/mL hygromycin until day 26. Cells that were co-transfected with both payload and Cre recombinase plasmids recovered to above 90% viability after 19 days (FIG. 6). Cells co-transfected with the appropriate payload and no recombinase recovered after 26 days, presumably due to random integration of the payload. It was assumed that random integration mediated recovery because the TagBFP payload marker was not observed to be visible above background levels in the negative control samples, but an integration event of the promoter-less payload plasmid could still have been inserted downstream of a weak promoter.

Payload integrated by Cre recombinase was observed in approximately 2% of the total population without antibiotic selection, and 99% of the surviving cells after selection, with 0.8% or 2.6% of surviving cells still expressing the landing pad EYFP marker in single-lox or double-lox landing pads, respectively (TABLE 4). The payload marker TagBFP was almost undetectable in cells that survived hygromycin selection in the absence of Cre recombinase, at 0.23% expression in single-lox cells and 0.87% expression in double-lox landing pad cells, of which nearly all still expressed the landing pad EYFP marker.

TABLE 4
Final percentage of payload expressing cells and
off-target integration after 21 days of serial passage or
20 days of hygromycin antibiotic selection.

| | Serial Passage | | Hygromycin Selection | |
|---|---|---|---|---|
| | Total | Multicopy | Total | Multicopy |
| | Payload | LP or | Payload | LP or |
| | Expressing | off-target | Expressing | off-target |
| Single lox Landing | 2.3% | 3.8% | 99.2% | 0.8% |
| Pad | | | | |
| Double lox Landing | 1.9% | 6.3% | 99.3% | 2.6% |
| Pad | | | | |
| Single lox - No | 0% | NA | 0.23% | 89.7% |
| Integrase | | | | |
| Double lox - No | 0% | NA | 0.87% | 100% |
| integrase | | | | |

Double Site Landing Pads with Counter-Selection

To test the ability to use dual att-sites in RMCE a landing pad system was developed in which the landing pad contained a fluorescent marker, antibiotic selection, and counterselection flanked by Bxb1 att sites (FIG. 7). This architecture allows for the retention of the promoter, in this case hEF1a while exchanging the genetic material between the att-sites. This design limits RMCE to the genetic payload between att-sites which minimizes the introduction of potentially detrimental bacterial derived plasmid sequences.

In preliminary tests using a stable cell line with the landing pad randomly integrated (which are expounded upon below), it was observed that 100% of clones were positive for successful RMCE. Characterization by PCR targeted to the final product of successful RMCE and sequencing verification of PCR products of clones that survived ganciclovir counter-selection indicated that all clones screened had successfully undergone RMCE.

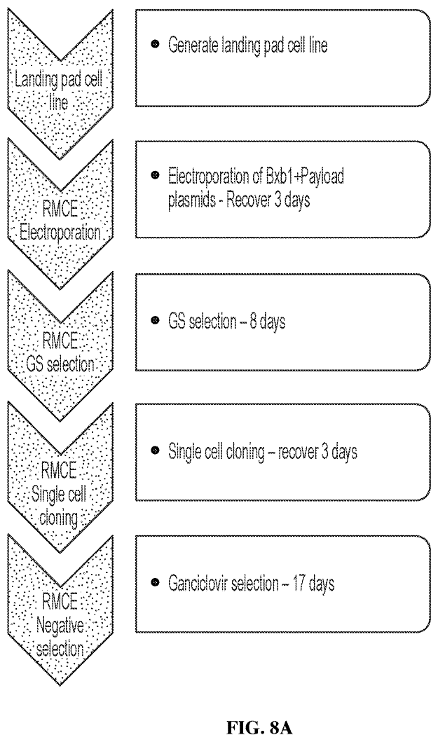

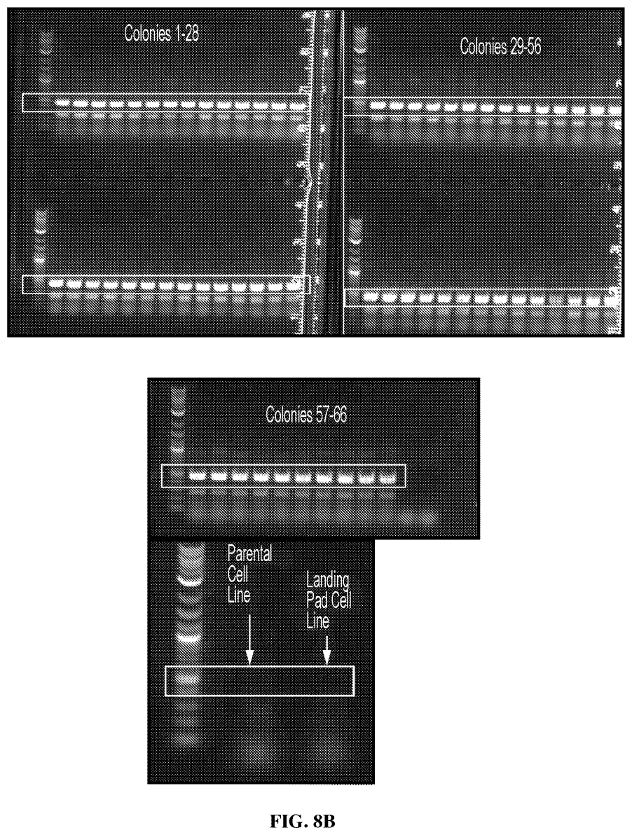

Stable cell lines were generated using random integration into a CHO glutamine synthetase (GS)-knockout cell line. The Bxb1 double att-site landing pad was electroporated into the cells and stable clones were selected using puromycin to generate the landing pad containing cell pool. To test the Bxb1 double att-site landing pads, Bxb1 and payload plasmids were electroporated into the stable cell pools and after 3 days of recovery cells were transferred into L-Glutamine free media (GS-Selection) for selection of recombination positive cells. After GS-selection the cells were single cell cloned using limiting dilution and negative selection through the use of Ganciclovir was used to remove non-targeted integrants (FIG. 8A). Surviving clones were screened using PCR spanning the landing pab hEF1a promoter and the payload iRFP. Sixty-six surviving clones were screened using PCR and all were positive for successful RMCE (FIG. 8B). The PCR band for a selected twenty-eight clones was sequenced and verified to be successful RMCE. The sequence of all twenty-eight clones aligns to the predicted RMCE sequence indicating successful recombination at the Bxb1 double att-site landing pad (data not shown).

### Double Site, Counter-Selectable, Integrase Expressing Landing Pads

To build on the previous designs, a system in which the integrase is expressed from the landing pad inducibly or constitutively, may increase efficiency of RMCE (FIG. 9). These designs minimize the number of plasmids transfected, and the inducible design allows for temporal adjustments to the expression of the integrase. In both cases, expression of the integrase before transfection of the payload is expected to increase efficiency.

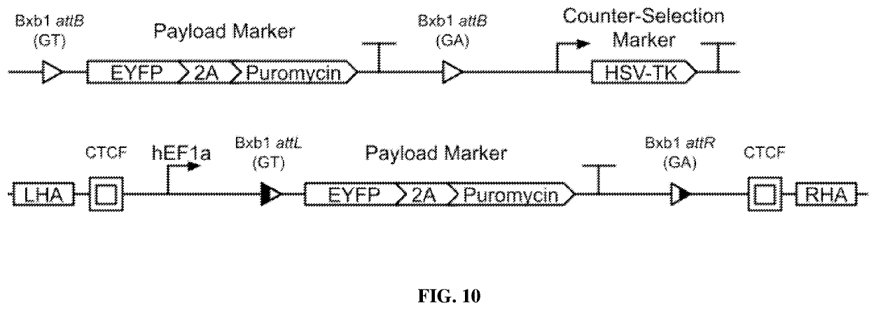

The integrase is constitutively expressed in the landing pad by an internal ribosome entry site (IRES) linker from EMCV virus (Genbank: MN542793.1, SEQ ID NO: 160). A left homology arm (LHA) or right homology arm (RHA) and CTCF insulator flank the landing pad to control the position integration site on the genome, and also to prevent silencing of the landing pad. Homology arms can be selected for loci known to be safe harbor sites, and also for loci known to inherently insulate for silencing. Notable sites in CHO are the orthologous ROSA26 locus from mice, H11, and COSMIC. In HEK293 cell, HeLa S3 cell, T-cell, induced pluripotent stem cell (iPSC), natural killer (NK) cell or human embryonic stem cell (hESC), notable sites are AAVS1, ROSA26, CCR5, and LiPS-A3S. A payload can be transfected to stable cell lines expressing the landing pad with a constitutive or inducible integrase (FIG. 10).

Integration of Orthogonal Recombination Sites into Landing Pads Using Payload Vectors

In some embodiments, further expansion of the system can include using the payload to introduce new recombinase sites (ex. attB) for use in multiple rounds of integration into targeted loci. In some embodiments, this system can be used with single or dual serine or tyrosine recombinases utilizing orthogonal recombinase sites. In some embodiments, the payload plasmid contains the cognate recombination site to the landing pad and an additional orthogonal recombination site is introduced into the cell. In some embodiments, the payload plasmid is integrated into the landing pad via the cognate recombination site present on the landing pad and brings with it the secondary recombination site for use in another round of targeted integration. In the case of serine integrases, after integration the original attP and attB sites are recombined and cannot participate in recombination without additional factors. In this way the number of orthogonal recombinase sites can be recombined to integrate multiple genes into the same targeted locus.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. A polypeptide having integrase activity and comprising, from N- to C-terminus: (i) an amino acid sequence of any one of SEQ ID NOs: 48, 40-43, 45-47, 49-54, 56, 59-61, 64, 65, 67, 68, 70, and 72 or an amino acid sequence having at least 95% identity with the amino acid sequence of any one of SEQ ID NOs: 48, 40-43, 45-47, 49-54, 56, 59-61, 64, 65, 67, 68, 70, and 72; (ii) an amino acid sequence of a GS linker; and (iii) an amino acid sequence of a nuclear localization signal (NLS).

2. A polypeptide having integrase activity and comprising, from N- to C-terminus: (i) an amino acid sequence of a nuclear localization signal (NLS) (ii) an amino acid sequence of a GS linker; and (iii) an amino acid sequence of any one of SEQ ID NOs: 48, 40-43, 45-47, 49-54, 56, 59-61, 64, 65, 67, 68, 70, and 72 or an amino acid sequence having at least 95% identity with the amino acid sequence of any one of SEQ ID NOs: 48, 40-43, 45-47, 49-54, 56, 59-61, 64, 65, 67, 68, 70, and 72.

3. The polypeptide of claim 1 or claim 2, wherein the GS linker is gly ser.

4. The polypeptide of any one of claims 1-3, wherein the amino acid sequence of the NLS comprises or consists essentially of the amino acid sequence of any one of SEQ ID NOs: 77-78 and 168-174.

5. A polynucleic acid encoding the polypeptide of any one of claims 1-4.

6. A polynucleic acid encoding an polypeptide having integrase activity, wherein the polynucleic acid comprises an expression cassette comprising, from 5′ to 3′: (i) a nucleic acid sequence of any one of SEQ ID NOs: 10, 2-5, 7-9, 11-16, 18, 21-23, 26, 27, 29, 30, 32, and 34 or a nucleic acid sequence having at least 95% identity with any one of SEQ ID NOs: 10 2-5, 7-9, 11-16, 18, 21-23, 26, 27, 29, 30, 32, and 34; (ii) a nucleic acid sequence encoding a GS linker; and (iii) a nucleic acid sequence encoding a nuclear localization signal (NLS).

7. A polynucleic acid encoding an polypeptide having integrase activity, wherein the polynucleic acid comprises an expression cassette comprising, from 5′ to 3′: (i) a nucleic acid sequence encoding a nuclear localization signal (NLS) (ii) a nucleic acid sequence encoding a GS linker; and (iii) a nucleic acid sequence of any one of SEQ ID NOs: 10, 2-5, 7-9, 11-16, 18, 21-23, 26, 27, 29, 30, 32, and 34 or a nucleic acid sequence having at least 95% identity with any one of SEQ ID NOs: 10, 2-5, 7-9, 11-16, 18, 21-23, 26, 27, 29, 30, 32, and 34.

8. The polynucleic acid of claim 6 or claim 7, wherein the nucleic acid sequence encoding the GS linker comprises or consists essentially of the nucleic acid sequence GGTTCA.

9. The polynucleic acid of any one of claims 6-8, wherein the nucleic acid sequence encoding the NLS comprises or consists essentially of the nucleic acid sequence of any one of SEQ ID NOs: 77-78 and 168-174.

10. An engineered cell comprising a chromosomal integration of a landing pad, wherein the landing pad comprises an expression cassette comprising, from 5′ to 3′: (i) a nucleic acid sequence of a promoter; (ii) a nucleic acid sequence of a first recombination site; and (iii) a nucleic acid sequence encoding for a landing pad marker, which is operably linked to the promoter of (i).

11. The engineered cell of claim 10, wherein the landing pad further comprises (iv) a nucleic acid sequence of a second recombination site, wherein the nucleic acid sequence of the second recombination site is positioned 3′ to the nucleic acid sequence encoding for the landing pad marker.

12. The engineered cell of claim 10 or claim 11, wherein the landing pad marker comprises an antibiotic resistance protein.

13. The engineered cell of any one of claims 10-12, wherein the landing pad marker comprises a fluorescent protein.

14. The engineered cell of anyone of claims 10-13, wherein the landing pad further comprises (v) a nucleic acid sequence encoding for a Woodchuck Hepatitis Virus Post-transcriptional Regulatory Element (WPRE) or a nucleic acid sequence encoding a polyA, which is operably linked to the nucleic acid sequence encoding for the landing pad marker.

15. The engineered cell of claim 14, wherein the landing pad comprises a nucleic acid sequence of a second recombination site, wherein the nucleic acid sequence of the second recombination site is positioned 5′ to the nucleic acid sequence encoding for the WPRE.

16. The engineered cell of claim 15, wherein the expression cassette comprises, from 5′ to 3′: (i) the nucleic acid of the promoter; (ii) the nucleic acid sequence of the first recombination site; (iii) the nucleic acid sequence encoding for the landing pad marker; (iv) a nucleic acid sequence of a second recombination site; and (v) the nucleic acid sequence encoding for the WPRE.

17. The engineered cell of any one of claims 10-16, wherein the engineered cell is derived from a HEK293 cell.

18. The engineered cell of claim 17, wherein the landing pad is integrated at a safe harbor locus selected from the group consisting of AAVS1, ROSA26, CCR5, and LiPS-A3S.

19. The engineered cell of any one of claims 10-16, wherein the engineered cell is derived from a CHO cell.

20. The engineered cell of claim 19, wherein the landing pad is integrated at a safe harbor locus selected from the group consisting of ROSA26, COSMIC, and H11.

21. The engineered cell of any one of claims 10-20, further comprising an integrase molecule comprising a nucleic acid sequence of a promoter operably linked to a nucleic acid sequence encoding for an integrase that binds to a recombination site of the landing pad.

22. The engineered cell of claim 21, wherein the promoter of the integrase molecule is a constitutive promoter.

23. The engineered cell of claim 21 or claim 22, wherein the integrase is a serine integrase.

24. The engineered cell of claim 21 or claim 22, wherein the integrase is a tyrosine integrase.

25. The engineered cell of claim 23 or claim 24, wherein the integrase comprises an amino acid sequence of any one of SEQ ID NOs: 48, 40-43, 45-47, 49-54, 56, 59-61, 64, 65, 67, 68, 70, and 72 or an amino acid sequence having at least 95% identity with the amino acid sequence of any one of SEQ ID NOs: 48, 40-43, 45-47, 49-54, 56, 59-61, 64, 65, 67, 68, 70, and 72.

26. The engineered cell of claim 25, wherein the integrase further comprises the amino acid sequence of a nuclear localization signal (NLS).

27. The engineered cell of claim 26, wherein the NLS comprises or consists essentially of the amino acid sequence of any one of SEQ ID NOs: 77-78 and 168-174.

28. The engineered cell of claim 26 or claim 27, wherein the integrase further comprises a GS linker.

29. A kit comprising:
(a) an engineered cell of any one of claims 21-28; and
(b) a donor molecule comprising from 5′ to 3′: (i) a nucleic acid sequence of a first recombination site, which corresponds to the first recombination site of the landing pad of the engineered cell; and (ii) a multiple cloning site.

30. A kit comprising:
(a) an engineered cell of any one of claims 10-20;
(b) a donor molecule comprising from 5′ to 3′: (i) a nucleic acid sequence of a first recombination site, which corresponds to the first recombination site of the landing pad of the engineered cell; and (ii) a multiple cloning site; and
(c) an integrase molecule comprising: (i) a nucleic acid sequence encoding for an integrase that binds to the first recombination sites of the landing pad and the donor molecule; or (ii) an amino acid sequence of an integrase that binds to the first recombination sites of the landing pad and the donor molecule;
optionally wherein a single polynucleic acid comprises the donor molecule and the integrase molecule.

31. The kit of claim 30, wherein the integrase molecule comprises a nucleic acid sequence of a promoter operably linked to a nucleic acid sequence encoding for an integrase, and wherein the promoter of the integrase molecule is a constitutive promoter.

32. The kit of claim 30 or claim 31, wherein the integrase is a serine integrase.

33. The kit of claim 30 or claim 31, wherein the integrase is a tyrosine integrase.

34. The kit of claim 30 or claim 31, wherein the integrase comprises an amino acid sequence of any one of SEQ ID NOs: 48, 40-43, 45-47, 49-54, 56, 59-61, 64, 65, 67, 68, 70, and 72 or an amino acid sequence having at least 95% identity with the amino acid sequence of any one of SEQ ID NOs: 48, 40-43, 45-47, 49-54, 56, 59-61, 64, 65, 67, 68, 70, and 72.

35. The kit of claim 34, wherein the integrase further comprises the amino acid sequence of a nuclear localization signal (NLS).

36. The kit of claim 35, wherein the NLS comprises or consists essentially of the amino acid sequence of any one of SEQ ID NOs: 77-78 and 168-174.

37. The kit of claim 35 or claim 36, wherein the integrase further comprises a GS linker.

38. The kit of any one of claims 29-37, wherein: the landing pad of the engineered cell comprises a nucleic acid sequence of a second recombination site, wherein the nucleic acid sequence of the second recombination site is positioned 3′ to the nucleic acid sequence encoding for the landing pad marker; and the donor molecule further comprises a nucleic acid sequence of a second recombination site, which corresponds to the second recombination site of the landing pad of the engineered cell.

39. The kit of claim 38, wherein the integrase binds to the first and second recombination sites of the landing pad and the donor molecule.

40. The kit of claim 38, wherein the kit comprises:
a first integrase molecule comprising: (i) a nucleic acid sequence encoding for a first integrase that binds to the first recombination sites of the landing pad and the donor molecule; (ii) or an amino acid sequence of a first integrase that binds to the first recombination sites of the landing pad and the donor molecule; and
a second integrase molecule comprising: (i) a nucleic acid sequence encoding for a second integrase that binds to the second recombination sites of the landing pad and the donor molecule; (ii) or an amino acid sequence of a second integrase that binds to the second recombination sites of the landing pad and the donor molecule;
optionally wherein a single polynucleic acid comprises the first integrase molecule and the second integrase molecule.

41. A method of integrating a nucleic acid sequence of interest into a cell genome, the method comprising:
(a) introducing a donor molecule into the engineered cell of any one of claims 21-28, wherein the donor molecule comprises from 5′ to 3′: (i) a nucleic acid sequence of a first recombination site, which corresponds to the first recombination site of the landing pad of the engineered cell; and (ii) a nucleic acid sequence of interest;
(b) expressing the integrase of the integrase molecule, thereby inducing integration of the nucleic acid sequence of interest of the donor molecule into the landing pad of the engineered cell;
wherein (a) occurs prior to, concurrently with, or after (b);
wherein, after integration, the nucleic acid sequence of interest is operably linked to the promoter of the landing pad of the engineered cell;
optionally, wherein, prior to integration, the nucleic acid sequence of interest is not operably linked to a promoter.

42. A method of integrating a nucleic acid sequence of interest into the genome of a cell comprising:
(a) introducing a donor molecule into the engineered cell of any one of claims 10-20, wherein the donor molecule comprises from 5′ to 3′: (i) a nucleic acid sequence of a first recombination site, which corresponds to the first recombination site of the landing pad of the engineered cell; and (ii) a nucleic acid sequence of interest;
(b) introducing an integrase molecule into the engineered cell, wherein the integrase molecule comprises: (i) a nucleic acid sequence encoding for an integrase that binds to the first recombination sites of the landing pad and the donor molecule; or (ii) an amino acid sequence of an integrase that binds to the first recombination sites of the landing pad and the donor molecule;
thereby inducing integration of the nucleic acid sequence of interest of the donor molecule into the landing pad of the engineered cell;
wherein, after integration, the nucleic acid sequence of interest is operably linked to the promoter of the landing pad of the engineered cell;
optionally wherein, prior to integration, the nucleic acid sequence of interest is not operably linked to a promoter; and
wherein (a) occurs prior to, concurrently with, or after (b).

43. The method of claim 42, wherein the integrase molecule comprises a nucleic acid sequence of a promoter operably linked to a nucleic acid sequence encoding for an integrase, and wherein the promoter of the integrase molecule is a constitutive promoter.

44. The method of claim 42 or claim 43, wherein the integrase is a serine integrase.

45. The method of claim 42 or claim 43, wherein the integrase is a tyrosine integrase.

46. The method of claim 42 or claim 43, wherein the integrase comprises an amino acid sequence of any one of SEQ ID NOs: 48, 40-43, 45-47, 49-54, 56, 59-61, 64, 65, 67, 68, 70, and 72 or an amino acid sequence having at least 95% identity with the amino acid sequence of any one of SEQ ID NOs: 48, 40-43, 45-47, 49-54, 56, 59-61, 64, 65, 67, 68, 70, and 72.

47. The method of claim 46, wherein the integrase further comprises the amino acid sequence of a nuclear localization signal (NLS).

48. The method of claim 47, wherein the NLS comprises or consists essentially of the amino acid sequence of any one of SEQ ID NOs: 77-78 and 168-174.

49. The method of claim 47 or claim 48, wherein the integrase further comprises a GS linker.

50. The method of any one of claims 41-49, wherein: the landing pad of the engineered cell comprises a nucleic acid sequence of a second recombination site, wherein the nucleic acid sequence of the second recombination site is positioned 3′ to the nucleic acid sequence encoding for the landing pad marker; and the donor molecule further comprises a nucleic acid sequence of a second recombination site, which corresponds to the second recombination site of the landing pad of the engineered cell.

51. The method of claim 50, wherein the integrase binds to the first and second recombination sites of the landing pad and the donor molecule.

52. A kit for performing the method of claim 50, wherein the kit comprises:
a first integrase molecule comprising: (i) a nucleic acid sequence encoding for a first integrase that binds to the first recombination sites of the landing pad and the donor molecule; (ii) or an amino acid sequence of a first integrase that binds to the first recombination sites of the landing pad and the donor molecule; and
a second integrase molecule comprising: (i) a nucleic acid sequence encoding for a second integrase that binds to the second recombination sites of the landing pad and the donor molecule; (ii) or an amino acid sequence of a second integrase that binds to the second recombination sites of the landing pad and the donor molecule;
optionally wherein a single polynucleic acid comprises the first integrase molecule and the second integrase molecule.

53. An engineered cell comprising a chromosomal integration of a landing pad, wherein the landing pad comprises from 5′ to 3′: (i) a nucleic acid sequence of a first recombination site; (ii) a landing pad marker comprising the nucleic acid sequence of a counter-selection marker; and (iii) a nucleic acid sequence of a second recombination site; wherein the landing pad further comprises (iv) a nucleic acid sequence of a promoter positioned 5′ or 3′ to the first recombination site and which is operably linked to the nucleic acid sequence of the counter-selection marker.

54. The engineered cell of claim 53, wherein the nucleic acid sequence of the promoter is positioned 5′ to the nucleic acid sequence of the first recombination site.

55. The engineered cell of claim 54, wherein the promoter is a constitutive promoter.

56. The engineered cell of any one of claims 53-55, wherein the landing pad marker further comprises a nucleic acid sequence encoding for an antibiotic resistance protein, a fluorescent protein, or both.

57. The engineered cell of claim 56, wherein the landing pad marker further comprises a nucleic acid sequence encoding for a viral 2A peptide.

58. The engineered cell of claim 57, wherein the landing pad marker is encoding on a polycistronic mRNA comprising, from 5′ to 3′: (i) a nucleic acid sequence encoding for a fluorescent protein; (ii) a nucleic acid sequence encoding for an antibiotic resistance protein; (iii) a nucleic acid sequence encoding for a viral 2A peptide; and (iv) a nucleic acid sequence encoding for the counter-selection marker.

59. The engineered cell of any one of claims 53-58, wherein the counter-selection marker comprises HSV-TK.

60. The engineered cell of any one of claims 53-59, wherein the engineered cell is derived from a HEK293 cell, HeLa S3 cell, T-cell, induced pluripotent stem cell (iPSC), natural killer (NK) cell or human embryonic stem cell.

61. The engineered cell of claim 61, wherein the landing pad is integrated at a safe harbor locus selected from the group consisting of AAVS1, ROSA26, CCR5, and LiPS-A3S.

62. The engineered cell of any one of claims 53-59, wherein the engineered cell is derived from a CHO cell.

63. The engineered cell of claim 62, wherein the landing pad is integrated at a safe harbor locus selected from the group consisting of ROSA26, COSMIC, and H11.

64. The engineered cell of any one of claims 53-63, further comprising a first integrase molecule comprising a nucleic acid sequence of a promoter operably linked to a nucleic acid sequence encoding for a first integrase that binds to a recombination site of the landing pad.

65. The engineered cell of claim 64, wherein the promoter of the first integrase molecule is a constitutive promoter.

66. The engineered cell of claim 64 or claim 65, wherein the first integrase is a serine integrase.

67. The engineered cell of claim 64 or claim 65, wherein the first integrase is a tyrosine integrase.

68. The engineered cell of claim 64 or claim 65, wherein the first integrase comprises an amino acid sequence of any one of SEQ ID NOs: 48, 40-43, 45-47, 49-54, 56, 59-61, 64, 65, 67, 68, 70, and 72 or an amino acid sequence having at least 95% identity with the amino acid sequence of any one of SEQ ID NOs: 48, 40-43, 45-47, 49-54, 56, 59-61, 64, 65, 67, 68, 70, and 72.

69. The engineered cell of claim 68, wherein the first integrase further comprises the amino acid sequence of a nuclear localization signal (NLS).

70. The engineered cell of claim 69, wherein the NLS comprises or consists essentially of the amino acid sequence of any one of SEQ ID NOs: 77-78 and 168-174.

71. The engineered cell of claim 69 or claim 70, wherein the first integrase further comprises a GS linker.

72. An engineered cell of any one of claims 64-71, further comprising a second integrase molecule, wherein the second integrase molecule comprises a nucleic acid sequence of a promoter operably linked to a nucleic acid sequence encoding for a second integrase that binds to a recombination site of the landing pad.

73. The cell of claim 72, wherein the first integrase and the second integrase bind to orthogonal recombination sites.

74. A kit comprising:
(a) an engineered cell of any one of claims 64-73; and
(b) a donor molecule comprising from 5′ to 3′: (i) a nucleic acid sequence of a first recombination site, which corresponds to the first recombination site of the landing pad of the engineered cell; (ii) a multiple cloning site; and (iii) a nucleic acid sequence of a second recombination site, which corresponds to the second recombination site of the landing pad of the engineered cell.

75. A kit comprising:
(a) an engineered cell of any one of claims 53-63; and
(b) a donor molecule comprising from 5′ to 3′: (i) a nucleic acid sequence of a first recombination site, which corresponds to the first recombination site of the landing pad of the engineered cell; (ii) a multiple cloning site; and (iii) a nucleic acid sequence of a second recombination site, which corresponds to the second recombination site of the landing pad of the engineered cell; and
(c) an integrase molecule comprising: (i) a nucleic acid sequence encoding for an integrase that binds to recombination sites of the landing pad and the donor molecule; or (ii) an amino acid sequence of an integrase that binds to the first recombination sites of the landing pad and the donor molecule;
optionally wherein a single polynucleic acid comprises the donor molecule and the integrase molecule.

76. The kit of claim 74 or claim 75, wherein the donor molecule further comprises an expression cassette comprising a nucleic acid sequence of a promoter operably linked to a nucleic acid sequence of a counter-selection marker.

77. The kit of claim 76, wherein the counter-selection marker is HSV-TK, and wherein the kit further comprises ganciclovir.

78. The kit of any one of claims 74-77, wherein the promoter of the integrase molecule is a constitutive promoter.

79. The kit of any one of claims 74-78, wherein the integrase is a serine integrase.

80. The kit of any one of claims 74-78, wherein the integrase is a tyrosine integrase.

81. The kit of any one of claims 74-80, wherein the integrase comprises an amino acid sequence of any one of SEQ ID NOs: 48, 40-43, 45-47, 49-54, 56, 59-61, 64, 65, 67, 68, 70, and 72 or an amino acid sequence having at least 95% identity with the amino acid sequence of any one of SEQ ID NOs: 48, 40-43, 45-47, 49-54, 56, 59-61, 64, 65, 67, 68, 70, and 72.

82. The kit of claim 81, wherein the integrase further comprises the amino acid sequence of a nuclear localization signal (NLS).

83. The kit of claim 82, wherein the NLS comprises or consists essentially of the amino acid sequence of any one of SEQ ID NOs: 77-78 and 168-174.

84. The kit of claim 81 or claim 82, wherein the integrase further comprises a GS linker.

85. A method of integrating a nucleic acid sequence of interest into a cell genome, the method comprising:
(a) introducing a donor molecule into the engineered cell of any one of claims 64-71, wherein the donor molecule comprises from 5′ to 3′: (i) a nucleic acid sequence of a first recombination site, which corresponds to the first recombination site of the landing pad of the engineered cell; (ii) a nucleic acid sequence of interest; and (iii) a nucleic acid sequence of a second recombination site, which corresponds to the second recombination site of the landing pad of the engineered cell; and
(b) expressing the integrase of the integrase molecule, thereby inducing integration of the nucleic acid sequence of interest of the donor molecule into the landing pad of the engineered cell;
wherein (b) occurs prior to, concurrently with, or after (a).

86. A method of integrating a nucleic acid sequence of interest into a cell genome, the method comprising:
(a) introducing a donor molecule into the engineered cell of any one of claims 53-63, wherein the donor molecule comprises from 5′ to 3′: (i) a nucleic acid sequence of a first recombination site, which corresponds to the first recombination site of the landing pad of the engineered cell; (ii) a nucleic acid sequence of interest; and (iii) a nucleic acid sequence of a second recombination site, which corresponds to the second recombination site of the landing pad of the engineered cell;
(b) introducing an integrase molecule into the engineered cell, wherein the integrase molecule comprises: (i) a nucleic acid sequence encoding for an integrase that binds to recombination sites of the landing pad and the donor molecule; or (ii) an amino acid sequence of an integrase that binds to the first recombination sites of the landing pad and the donor molecule;
thereby inducing integration of the nucleic acid sequence of interest of the donor molecule into the landing pad of the engineered cell;
wherein (a) occurs prior to, concurrently with, or after (b).

87. The method of claim 86, wherein the integrase molecule comprises a nucleic acid sequence of a promoter operably linked to a nucleic acid sequence encoding for an integrase, and wherein promoter of the integrase molecule is a constitutive promoter.

88. The method of claim 86 or claim 87, wherein the integrase is a serine integrase.

89. The method of claim 86 or claim 87, wherein the integrase is a tyrosine integrase.

90. The method of claim 86 or claim 87, wherein the integrase comprises an amino acid sequence of any one of SEQ ID NOs: 48, 40-43, 45-47, 49-54, 56, 59-61, 64, 65, 67, 68, 70, and 72 or an amino acid sequence having at least 95% identity with the amino acid sequence of any one of SEQ ID NOs: 48, 40-43, 45-47, 49-54, 56, 59-61, 64, 65, 67, 68, 70, and 72.

91. The method of claim 90, wherein the integrase further comprises the amino acid sequence of a nuclear localization signal (NLS).

92. The method of claim 91, wherein the NLS comprises or consists essentially of the amino acid sequence of any one of SEQ ID NOs: 77-78 and 168-174.

93. The method of claim 91 or claim 92, wherein the integrase further comprises a GS linker.

94. The method of any one of claims 85-93, wherein the donor molecule further comprises an expression cassette comprising a nucleic acid sequence of a promoter operably linked to a nucleic acid sequence of a counter-selection marker.

95. The method of claim 94, wherein:
(i) the counter-selection marker of the landing pad of the engineered cell is HSV-TK;
(ii) the counter-selection marker of the donor molecule is HSV-TK; or
(iii) a combination of (i) and (ii).

96. The method of claim 94, further comprising contacting the engineered cell with ganciclovir.

97. An engineered cell comprising a chromosomal integration of a landing pad, wherein the landing pad comprises, from 5′ to 3′: (i) a nucleic acid sequence of a first recombination site; (ii) a nucleic sequence encoding for an integrase; and (iii) a nucleic acid sequence of a second recombination site; wherein the landing pad further comprises (iv) a nucleic acid sequence of a first promoter positioned 5′ or 3′ to the nucleic acid sequence of the first recombination site and which is operably linked to the nucleic acid sequence encoding for the integrase.

98. The engineered cell of claim 97, wherein the landing pad comprises, from 5′ to 3′: (i) a nucleic acid sequence of a first recombination site; (ii) a nucleic sequence encoding for a polycistronic mRNA comprising the nucleic acid sequence of the integrase and a nucleic acid sequence encoding for a landing pad marker; and (iii) a nucleic acid sequence of a second recombination site; wherein the landing pad further comprises (iv) a nucleic acid sequence of a first promoter positioned 5′ or 3′ to the nucleic acid sequence of the first recombination site and which is operably linked to the nucleic acid sequence encoding for the polycistronic mRNA.

99. The engineered cell of claim 98, wherein the nucleic acid sequence of a first promoter is positioned 5′ to the nucleic acid sequence of the first recombination site.

100. The engineered cell of claim 98 or claim 99, wherein the landing pad marker comprises: an antibiotic resistance protein; a fluorescent protein; a counter-selection marker; or a combination thereof.

101. The engineered cell of any one of claims 98-100, wherein the landing pad marker comprises: a viral 2A peptide; an IRES; or a combination thereof.

102. The engineered cell of any one of claims 98-101, wherein the polycistronic mRNA further comprises: a nucleic acid sequence encoding for a viral 2A peptide; a nucleic acid sequence encoding for an IRES; or a combination thereof.

103. The engineered cell of claim 102, wherein the polycistronic mRNA comprises, from 5′ to 3′: (i) a nucleic acid sequence encoding for the landing pad marker; (ii) a nucleic acid sequence encoding for an IRES; and (iii) the nucleic acid sequence encoding for the integrase.

104. The engineered cell of claim 97, wherein the landing pad comprises: (a) a first expression cassette comprising the nucleic acid sequence of the first promoter and the nucleic acid sequence encoding for the integrases; and (b) a second expression cassette comprising a nucleic acid sequence of a second promoter operably linked to a nucleic acid sequence encoding for a landing pad marker.

105. The engineered cell of claim 104, wherein the landing pad marker comprises: an antibiotic resistance protein; a fluorescent protein; a counter-selection marker; or a combination thereof.

106. The engineered cell of claim 105, wherein the landing pad marker further comprises: a viral 2A peptide; an IRES; or a combination thereof.

107. The engineered cell of any one of claims 104-106, wherein the first expression cassette is 5′ to the second expression cassette.

108. The engineered cell of any one of claims 104-106, wherein the first expression cassette is 3′ to the second expression cassette.

109. The engineered cell of any one of claims 104-108, wherein the first expression cassette and the second expression cassette are encoded in the same orientation.

110. The engineered cell of any one of claims 104-108, wherein the first expression cassette and the second expression cassette are encoded in opposite orientations.

111. The engineered cell of claim 97, wherein the landing pad comprises: (a) a first expression cassette comprising the nucleic acid sequence of the first promoter and the nucleic acid sequence encoding for the integrases; (b) a second expression cassette comprising a nucleic acid sequence of a second promoter operably linked to a nucleic acid sequence encoding for a landing pad marker; and (c) a third expression cassette comprising a nucleic acid sequence of a third promoter operably linked to a nucleic acid sequence encoding for an auxiliary gene.

112. The engineered cell of claim 111, wherein the landing pad marker comprises: an antibiotic resistance protein; a fluorescent protein; a counter-selection marker; or a combination thereof.

113. The engineered cell of claim 112, wherein the landing pad marker further comprises: a viral 2A peptide; an IRES; or a combination thereof.

114. The engineered cell of any one of claims 111-113, wherein the auxiliary gene comprises a counter-selection marker.

115. The engineered cell of any one of claims 111-114, wherein the first expression cassette is 5′ to one or both of the second expression cassette and the third expression cassette.

116. The engineered cell of any one of claims 111-114, wherein the second expression cassette is 5′ to one or both of the first expression cassette and the third expression cassette.

117. The engineered cell of any one of claims 111-114, wherein the third expression cassette is 5′ to one or both of the first expression cassette and the second expression cassette.

118. The engineered cell of any one of claims 111-117, wherein the first expression cassette, the second expression cassette, and the third expression cassette are encoded in the same orientation.

119. The engineered cell of any one of claims 111-117, wherein the first expression cassette, the second expression cassette, and the third expression cassette are not all encoded in the same orientation.

120. The engineered cell of claim 119, wherein the first expression cassette, the second expression cassette, and the third expression cassette are encoded in alternating orientations.

121. The engineered cell of any one of claims 97-120, wherein the first promoter is a chemically inducible promoter.

122. The engineered cell of claim 121, wherein the landing pad further comprises a nucleic acid sequence encoding for a transcriptional activator that binds to the chemically inducible promoter when expressed in the presence of a small molecule inducer.

123. An engineered cell comprising a chromosomal integration of a landing pad, wherein the landing pad comprises, from 5′ to 3′:
(a) a first expression cassette comprising a nucleic acid sequence of a first promoter operably linked to a nucleic acid sequence encoding for a polycistronic mRNA, wherein the polycistronic mRNA comprises: (i) a nucleic acid sequence encoding for a landing pad marker; and (ii) a nucleic acid sequence encoding for a transcriptional activator;
(b) a second expression cassette comprising a nucleic acid sequence of a second promoter operably linked to a nucleic acid sequence encoding for an integrase, wherein the second promoter is a chemically inducible promoter that is bound by the transcriptional activator of (a), when the transcriptional activator is expressed in the presence of a small molecule inducer;
wherein the landing pad further comprises:
(c) a first recombination site positioned 5′ to the nucleic acid sequence encoding for the polycistronic mRNA of (a); and
(d) a second recombination site positioned 3′ to the second expression cassette of (b).

124. The engineered cell of claim 123, wherein the second recombination site is positioned 3′ to the first promoter.

125. The engineered cell of claim 123 or claim 124, wherein the landing pad marker comprises: an antibiotic resistance protein; a fluorescent protein; a counter-selection marker; or a combination thereof.

126. The engineered cell of any one of claims 123-125, wherein the landing pad marker further comprises: a viral 2A peptide; an IRES; or a combination thereof.

127. The engineered cell of claim 126, wherein the nucleic acid sequence encoding for the landing pad marker and the nucleic acid sequence encoding for the transcriptional activator are separated by a nucleic acid sequence encoding for a viral 2A peptide or an IRES.

128. The engineered cell of any one of claims 123-127, wherein the first expression cassette and the second expression cassette are in the same orientation.

129. The engineered cell of any one of claims 123-127, wherein the first expression cassette and the second expression cassette are in opposite orientations.

130. An engineered cell comprising a chromosomal integration of a landing pad, wherein the landing pad comprises:
(a) a first expression cassette comprising a nucleic acid sequence of a first promoter operably linked to a nucleic acid sequence encoding for a landing pad marker;
(b) a second expression cassette comprising a nucleic acid sequence of a second promoter operably linked to a nucleic acid sequence encoding for a transcriptional activator;
(c) a third expression cassette comprising a nucleic acid sequence of a third promoter operably linked to a nucleic acid sequence of an integrase, wherein the third promoter is a chemically inducible promoter that is bound by the transcriptional activator of (b), when the transcriptional activator is expressed in the presence of a small molecule inducer;
wherein the third expression cassette is 3′ to the first expression set, the second expression cassette, or both; and
wherein the landing pad further comprises:
(d) a first recombination; and
(e) a second recombination site;
wherein cassette exchange at the first and second recombination sites results in excision of: the nucleic acid sequence encoding for a landing pad marker; the nucleic acid sequence encoding for a transcriptional activator; and the third expression cassette.

131. The engineered cell of claim 130, wherein cassette exchange at the first and second recombination sites also results in excision of the first promoter, optionally wherein cassette exchange also results in excision of the second promoter.

132. The engineered cell of claim 130, wherein cassette exchange at the first and second recombination sites also results in excision of the second promoter, optionally wherein cassette exchange also results in excision of the first promoter.

133. The engineered cell of any one of claims 130-132, wherein the first expression cassette and the second expression cassette are 5′ to the expression cassette.

134. The engineered cell of any one of claims 130-133, wherein the third expression cassette is 5′ to the second expression cassette.

135. The engineered cell of any one of claims 130-134, wherein the third expression cassette is 5′ to the first expression cassette.

136. The engineered cell of any one of claims 130-135, wherein the landing pad marker comprises: an antibiotic resistance protein; a fluorescent protein; a counter-selection marker or a combination thereof.

137. The engineered cell of claim 136, wherein the landing pad marker further comprises: a viral 2A peptide; an IRES; or a combination thereof.

138. The engineered cell of any one of claims 130-137, wherein the second expression cassette comprises a nucleic acid sequence encoding for a polycistronic mRNA comprising the nucleic acid sequence of the transcriptional activator and a nucleic acid sequence of a counter-selection marker.

139. The engineered cell of claim 138, wherein the polycistronic mRNA further comprises a nucleic acid sequence encoding for a viral 2A peptide, a nucleic acid sequence encoding for an IRES, or a combination thereof.

140. The engineered cell of any one of claims 130-139, wherein the first expression cassette, the second expression cassette, and the third expression cassette are in the same orientation.

141. The engineered cell of any one of claims 130-140, wherein the first expression cassette, the second expression cassette, and the third expression cassette are not in the same orientation.

142. The engineered cell of claim 141, wherein the first expression cassette, the second expression cassette, and the third expression cassette are in alternating orientations.

143. The engineered cell of any one of claims 97-142, wherein the integrase is a serine integrase.

144. The engineered cell of any one of claims 97-142, wherein the integrase is a tyrosine integrase.

145. The engineered cell of any one of claims 97-142, wherein the landing pad marker is encoding on a polycistronic mRNA comprising, from 5′ to 3′: (i) a nucleic acid sequence encoding for a fluorescent protein; (ii) a nucleic acid sequence encoding for an antibiotic resistance protein; (iii) a nucleic acid sequence encoding for a viral 2A peptide; and (iv) a nucleic acid sequence encoding for the counter-selection marker.

146. The engineered cell of any one of claims 97-145, wherein the engineered cell is derived from a HEK293 cell, HeLa S3 cell, T-cell, induced pluripotent stem cell (iPSC), natural killer (NK) cell or human embryonic stem cell.

147. The engineered cell of claim 146, wherein the landing pad is integrated at a safe harbor locus selected from the group consisting of AAVS1, ROSA26, CCR5, and LiPS-A3S.

148. The engineered cell of any one of claims 97-145, wherein the engineered cell is derived from a CHO cell.

149. The engineered cell of claim 148, wherein the landing pad is integrated at a safe harbor locus selected from the group consisting of ROSA26, COSMIC, and H11.

150. A kit comprising:
(a) an engineered cell of any one of claims 97-149; and
(b) a donor molecule comprising from 5′ to 3′: (i) a nucleic acid sequence of a first recombination site, which corresponds to the first recombination site of the landing pad of the engineered cell; (ii) a multiple cloning site; and (iii) a nucleic acid sequence of a second recombination site, which corresponds to the second recombination site of the landing pad of the engineered cell.

151. The kit of claim 150, wherein the integrase is a serine integrase.

152. The kit of claim 151, wherein the serine integrase comprises any one of SEQ ID NOs: 48, 40-43, 45-47, 49-54, 56, 59-61, 64, 65, 67, 68, 70, 72, 75 and 76.

153. The kit of claim 150, wherein the integrase is a tyrosine integrase.

154. The kit of claim 150, wherein the landing pad marker is encoding on a polycistronic mRNA comprising, from 5′ to 3′: (i) a nucleic acid sequence encoding for a fluorescent protein; (ii) a nucleic acid sequence encoding for an antibiotic resistance protein; (iii) a nucleic acid sequence encoding for a viral 2A peptide; and (iv) a nucleic acid sequence encoding for the counter-selection marker.

155. A method of integrating a nucleic acid sequence of interest into a cell genome, the method comprising:
(a) introducing a donor molecule into the engineered cell of any one of claims I1-I51; wherein the donor molecule comprises from 5′ to 3′: (i) a nucleic acid sequence of a first recombination site, which corresponds to the first recombination site of the landing pad of the engineered cell; (ii) a nucleic acid sequence of interest; and (iii) a nucleic acid sequence of a second recombination site, which corresponds to the second recombination site of the landing pad of the engineered cell; and
(b) expressing the integrase, thereby inducing integration of the nucleic acid sequence of interest of the donor molecule into the landing pad of the engineered cell;
wherein (b) occurs prior to, concurrently with, or after (a).

156. The method of claim 155, wherein the integrase is a serine integrase.

157. The method of claim 156, wherein the serine integrase comprises any one of SEQ ID NOs: 48, 40-43, 45-47, 49-54, 56, 59-61, 64, 65, 67, 68, 70, 72, 75 and 76.

158. The method of claim 155, wherein the integrase is a tyrosine integrase.

159. The method of claim 155, wherein the landing pad marker is encoding on a polycistronic mRNA comprising, from 5′ to 3′: (i) a nucleic acid sequence encoding for a fluorescent protein; (ii) a nucleic acid sequence encoding for an antibiotic resistance protein; (iii) a nucleic acid sequence encoding for a viral 2A peptide; and (iv) a nucleic acid sequence encoding for the counter-selection marker.

160. An engineered cell comprising a chromosomal integration of a first landing pad, wherein the first landing pad comprises a nucleic acid sequence of a first recombination site having the nucleic acid sequence having at least 80%, at least 85%, at least 90%, at least 95%, at least 96%, at least 97%, at least 98%, or at least 99% identity with of any one of SEQ ID NOs: 79-148; and (ii) a nucleic acid sequence of a second recombination site, wherein the second recombination site is orthogonal to the first recombination site.

161. The engineered cell of claim 160, wherein the second recombination site comprises a nucleic acid having at least 80%, at least 85%, at least 90%, at least 95%, at least 96%, at least 97%, at least 98%, or at least 99% identity with of any one of SEQ ID NOs: 79-159, 166, and 167.

162. The engineered cell of claim 160 or claim 161, wherein the first nucleic acid sequence and the second nucleic acid sequence share at least 80%, at least 85%, at least 90%, at least 95%, at least 96%, at least 97%, at least 98%, or at least 99% identity.

163. The engineered cell of any one of claims 160-162, wherein the nucleic acid sequence of the first recombination site and the nucleic acid sequence of the second recombination site differ.

164. The engineered cell of any one of claims 160-163, wherein the first recombination site and the second recombination site are recognized by the same integrase.

165. The engineered cell of any one of claims 160-163, wherein the first recombination site and the second recombination site are recognized by different integrases.

166. The engineered cell of any one of claims 160-165, comprising a chromosomal integration of a second landing pad, wherein the second landing pad comprises: (i) a nucleic acid sequence of a third recombination site; and (ii) a nucleic acid sequence of a fourth recombination site.

167. The engineered cell of claim 166, wherein the first recombination site, the second recombination site, the third recombination site, and the fourth recombination site are all orthogonal with respect to each other.

168. The engineered cell of claim 166 or claim 167, wherein the third recombination site comprises a nucleic acid of any one of SEQ ID NOs: 79-159, 166, and 167.

169. The engineered cell of any one of claims 166-168, wherein the fourth recombination site comprises a nucleic acid of any one of SEQ ID NOs: 79-159, 166, and 167.

170. The engineered cell of any one of claims 160-169, wherein the first landing pad comprises a first expression cassette, the second landing pad comprises a second expression cassette, or a combination thereof.

171. The engineered cell of any one of claims 160-170, wherein the engineered cell is derived from a HEK293 cell.

172. The engineered cell of claim 171, wherein the engineered cell comprises a first landing pad and a second landing pad, and wherein the first landing pad and/or second landing pad is integrated at a safe harbor locus selected from the group consisting of AAVS1, ROSA26, CCR5, and LiPS-A3S, wherein the first landing pad and second landing are not integrated at the same locus.

173. The engineered cell of any one of claims 160-166, wherein the engineered cell is derived from a CHO cell.

174. The engineered cell of claim 173, wherein engineered cell comprises a first landing pad and a second landing pad, and wherein the first landing pad and/or second landing pad is integrated at a safe harbor locus selected from the group consisting of ROSA26, COSMIC, and H11, wherein the first landing pad and second landing are not integrated at the same locus.

175. The engineered cell of any one of claims 160-174, further comprising a polynucleotide comprising a nucleic acid sequence of a promoter operably linked to a nucleic acid sequence encoding for a first integrase that binds to the first recombination site of the first landing pad, the second recombination site of the first landing pad, or a combination thereof.

176. The engineered cell of claim 175, wherein the first integrase binds to the first recombination site and the second recombination site of the first landing pad.

177. The engineered cell of claim 175 or claim 176, wherein the first integrase comprises an amino acid sequence of any one of SEQ ID NOs: 48, 39-47 and 49-72 or an amino acid sequence having at least 95% identity with the amino acid sequence of any one of SEQ ID NOs: 48, 39-47 and 49-72.

178. The engineered cell of any one of claims 175-177, wherein the first integrase comprises an amino acid sequence of any one of SEQ ID NOs: 48, 39-43, 45-47, 49-54, 56, 59-61, 64, 65, 67, 68, 70, and 72 or an amino acid sequence having at least 95% identity with the amino acid sequence of any one of SEQ ID NOs: 48, 40-43, 45-47, 49-54, 56, 59-61, 64, 65, 67, 68, 70, and 72.

179. The engineered cell of any one of claims 175-178, wherein the first integrase comprises the amino acid sequence of a nuclear localization signal (NLS).

180. The engineered cell of claim 179, wherein the NLS comprises or consists essentially of the amino acid sequence of any one of SEQ ID NOs: 77-78 and 168-174.

181. The engineered cell of claim 179 or claim 180, wherein the first integrase further comprises a GS linker.

182. The engineered cell of any one of claims 160-174, further comprising: a polynucleotide comprising a nucleic acid sequence of a promoter operably linked to a nucleic acid sequence encoding for a first integrase that binds to the first recombination site of the first landing pad; and a polynucleotide comprising a nucleic acid sequence of a promoter operably linked to a nucleic acid sequence encoding for a second integrase that binds to the second recombination site of the first landing pad.

183. A kit comprising:
(a) an engineered cell of any one of claims 160-182; and
(b) a donor molecule comprising from 5′ to 3′: (i) a nucleic acid sequence of a first recombination site, which corresponds to the first recombination site of the first landing pad of the engineered cell; (ii) a multiple cloning site; and (iii) a nucleic acid sequence of a second recombination site, which corresponds to the second recombination site of the first landing pad of the engineered cell.

184. A method of integrating a nucleic acid sequence of interest into a cell genome, the method comprising:
(a) introducing a donor molecule into the engineered cell of any one of claims 175-181; wherein the donor molecule comprises from 5′ to 3′: (i) a nucleic acid sequence of a first recombination site, which corresponds to the first recombination site of a first landing pad of the engineered cell; (ii) the first nucleic acid sequence of interest; and (ii) a nucleic acid sequence of a second recombination site, which corresponds to the second recombination site of the first landing pad of the engineered cell;
(b) expressing the first integrase, thereby inducing integration of the first nucleic acid sequence of interest of the first donor molecule into the first landing pad of the engineered cell;
wherein (b) occurs prior to, concurrently with, or after (a).

185. A method of integrating a nucleic acid sequence of interest into a cell genome, the method comprising:
(a) introducing a donor molecule into the engineered cell of claim 182; wherein the donor molecule comprises from 5′ to 3′: (i) a nucleic acid sequence of a first recombination site, which corresponds to the first recombination site of a first landing pad of the engineered cell; (ii) the first nucleic acid sequence of interest; and (ii) a nucleic acid sequence of a second recombination site, which corresponds to the second recombination site of the first landing pad of the engineered cell;
(b) expressing the first integrase and the second integrase, thereby inducing integration of the first nucleic acid sequence of interest of the first donor molecule into the first landing pad of the engineered cell;
wherein (b) occurs prior to, concurrently with, or after (a).

186. A method of integrating a nucleic acid sequence of interest into a cell genome, the method comprising:
(a) introducing a donor molecule into the engineered cell of any one of claims 160-174, wherein the donor molecule comprises from 5′ to 3′: (i) a nucleic acid sequence of a first recombination site, which corresponds to the first recombination site of the first landing pad of the engineered cell; (ii) a nucleic acid sequence of interest; and (iii) a nucleic acid sequence of a second recombination site, which corresponds to the second recombination site of the first landing pad of the engineered cell;
(b) introducing an integrase molecule into the engineered cell, wherein the integrase molecule comprises: (i) a nucleic acid sequence encoding for an integrase that binds to the first recombination site and the second recombination site of the first landing pad and the first recombination site and the second recombination site of the donor molecule; or (ii) an amino acid sequence of an integrase that binds to the first recombination site and the second recombination site of the first landing pad and the first recombination site and the second recombination site of the donor molecule;
thereby inducing integration of the nucleic acid sequence of interest of the donor molecule into the landing pad of the engineered cell;
wherein (a) occurs prior to, concurrently with, or after (b).

187. A method of integrating a nucleic acid sequence of interest into a cell genome, the method comprising:
(a) introducing a donor molecule into the engineered cell of any one of claims 160-174, wherein the donor molecule comprises from 5′ to 3′: (i) a nucleic acid sequence of a first recombination site, which corresponds to the first recombination site of the first landing pad of the engineered cell; (ii) a nucleic acid sequence of interest; and (iii) a nucleic acid sequence of a second recombination site, which corresponds to the second recombination site of the first landing pad of the engineered cell;
(b) introducing one or more polynucleotides into the engineered cell, collectively comprising: (i) a nucleic acid sequence encoding for a first integrase that binds to the first recombination site of the first landing pad and the first recombination site of the donor molecule; and (ii) a nucleic acid sequence encoding for a second integrase that binds to the second recombination site of the first landing pad and the second recombination site of the donor molecule;
thereby inducing integration of the nucleic acid sequence of interest of the donor molecule into the landing pad of the engineered cell;
wherein (a) occurs prior to, concurrently with, or after (b).

188. A method of integrating a nucleic acid sequence of interest into a cell genome, the method comprising:
(a) introducing a donor molecule into the engineered cell of any one of claims 160-174, wherein the donor molecule comprises from 5′ to 3′: (i) a nucleic acid sequence of a first recombination site, which corresponds to the first recombination site of the first landing pad of the engineered cell; (ii) a nucleic acid sequence of interest; and (iii) a nucleic acid sequence of a second recombination site, which corresponds to the second recombination site of the first landing pad of the engineered cell;
(b) introducing: (i) a polypeptide comprising an amino acid sequence of a first integrase that binds to the first recombination site of the first landing pad and the first recombination site of the donor molecule; or (ii) a polypeptide comprising an amino acid sequence of a second integrase that binds to the second recombination site of the first landing pad and the second recombination site of the donor molecule;
thereby inducing integration of the nucleic acid sequence of interest of the donor molecule into the landing pad of the engineered cell;
wherein (a) occurs prior to, concurrently with, or after (b).
